SEQUENCE LISTING (460 sequences)

SEQ ID NO: 1 - protein, 107 aa, synthetic construct
  REGION 1..107: note = C-A11
AIEMTQSPSSLAASVGDTVTITCKASEDIRYGLNWYQQKLGIAPKLLIYGANTLEAGVPSRFSGSGSETDYTLTISSVQAEDAGIYYCQQGYSTPLTFGAGTKVEIK

SEQ ID NO: 2 - protein, 107 aa, synthetic construct
  REGION 1..107: note = hN2F
DIQMTQSPSSLSASVGDRVTITCKASEDIRYGLNWYQQKPGKAPKLLIYGANTLEAGVPSRFSGSGSGTDFTLTISSLQPEDFATYYCQQGYSTPLTFGGGTKVEIK

SEQ ID NO: 3 - protein, 107 aa, synthetic construct
  REGION 1..107: note = hN2Y
DIQMTQSPSSLSASVGDRVTITCKASEDIRYGLNWYQQKPGKAPKLLIYGANTLEAGVPSRFSGSGSGTDYTLTISSLQPEDFATYYCQQGYSTPLTFGGGTKVEIK

SEQ ID NO: 4 - protein, 120 aa, synthetic construct
  REGION 1..120: note = C-A11
QSLEESGGRLVTPGTPLTLTCTVSGFSLSTYAMSWVRQAPGKGLEWIGVIASNADTYYASWAKGRITISKTSTTVDLKITSPTTEDTATYFCARHPGSAGYGYAVYYAMDPWGPGTLVTV

SEQ ID NO: 5 - protein, 120 aa, synthetic construct
  REGION 1..120: note = hN2F
EVQLVESGGGLVQPGGSLRLSCTVSGFSLSTYAMSWVRQAPGKGLEWIGVIASNADTYYASWAKGRFTISRDNSKNTLYLQMNSLRAEDTAVYYCARHPGSAGYGYAVYYAMDPWGQGTL

SEQ ID NO: 6 - protein, 120 aa, synthetic construct; identical to SEQ ID NO: 5
  REGION 1..120: note = hN2Y

SEQ ID NO: 7 - protein, 214 aa, synthetic construct
  REGION 1..214: note = 3R4-H4
DIQMTQSPSSLSASVGDRVTITCQASQGIHYAINWYQQKPGKAPKLLIYGHNYLEEGVPSRFSGSGSGTDYTLTISSLQPEDFATYYCQQGYIVPIVFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 214 aa, synthetic construct
  REGION 1..214: note = 5R4-H1
DIQMTQSPSSLSASVGDRVTITCQASEPIHYAINWYQQKPGKAPKLLIYGTSYRLDGVPSRFSGSGSGTDFTLTISSLQPEDFATYYCQQGYTVPLHFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 214 aa, synthetic construct
  REGION 1..214: note = 5R4-F3
DIQMTQSPSSLSASVGDRVTITCQASEDIRYGLNWYQQKPGKAPKLLIYGTSYRRDGVPSRFSGSGSGTDYTLTISSLQPEDFATYYCQQGYNLPTRFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 214 aa, synthetic construct
  REGION 1..214: note = 5R4-E11
DIQMTQSPSSLSASVGDRVTITCKASEPIYYAINWYQQKPGKAPKLLIYGASGLADGVPSRFSGSGSGTDYTLTISSLQPEDFATYYCQQGYIVPIMFGGGTKVEIKRTVAAPSVSIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 214 aa, synthetic construct
  REGION 1..214: note = 6R4-H1
DIQMTQSPSSLSASVGDRVTITCQASQPIFYGIDWYQQKPGKAPKLLIYGTSYRAEGVPSRFSGSGSGTDYTLTISSLQPEDFATYYCQQGYIVPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 214 aa, synthetic construct
  REGION 1..214: note = 6R4-A7
DIQMTQSPSSLSASVGDRVTITCQASQQIYYAINWYQQKPGKAPKLLIYGANYREDGVPSRFSGSGSGTDYTLTISSLQPEDFATYYCQQGYLVPVGFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 214 aa, synthetic construct
  REGION 1..214: note = 6R4-G10
DIQMTQSPSSLSASVGDRVTITCQASERIYYAINWYQQKPGKAPKLLIYGANYRVDGVPSRFSGSGSGTDYTLTISSLQPEDFATYYCQQGYIVPTNFGGGTKVEIKRTVAAPSVFIFPPSDEQLKSGTASVVCLLNNFYPREAKVQWKVDNALQSGNSQESVAEQDSKDSTYSLSSTLTLSKADYEKHKVYACEVTHQGLSSPVTKSFNRGEC

SEQ ID NO: 14 - protein, 214 aa, synthetic construct
  REGION 1..214: note = 6R4-G11
DIQMTQSPSSLSASVGDRVTITCKASEDIRYGLNWYQQKPGKAPKLLIYGANHLREGVPSRFSGSGSGTDYTLTISSLQPEDFATYYCGWGYSVPLTFGGGTKVEIKRTVAAPSVFIFPPSDEQLKSGTASVVCLLNNFYPREAKVQWKVDNALQSGNSQESVTEQDSKDSTYSLSSTLTLGRADYEKHKVYACEVTHQGLSSPVTKSFNRGEC

SEQ ID NO: 15 - protein, 214 aa, synthetic construct
  REGION 1..214: note = 6R4-E12
DIQMTQSPSSLSASVGDRVTITCQASEKIHYAINWYQQKPGKAPKLLIYGHSHLEDGVPSRFSGSGSGTDYTLTISSLQPEDFATYYCQQGYIVPNSFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 214 aa, synthetic construct
  REGION 1..214: note = 6R4-E3
DIQMTQSPSSLSASVGDRVTITCQASQLIYYGINWYQQKPGKAPKLLIYGANYRVDGVPSRFSGSGSGTDYTLTISSLQPEDFATYYCQQGYSVPLDFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 214 aa, synthetic construct
  REGION 1..214: note = 6R4-H5
DIQMTQSPSSLSASVGDRVTITCKASEDIRYGLNWYQQKPGKAPKLLIYGHTYRLDGVPSRFSGSGSGTDYTLTISSLQPEDFATYYCQQGYVVPVGFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 214 aa, synthetic construct; identical to SEQ ID NO: 7
  REGION 1..214: note = 6R4-H11

SEQ ID NO: 19 - protein, 214 aa, synthetic construct
  REGION 1..214: note = 6R6-A7
DIQMTQSPSSLSASVGDRVTITCQASEKIYYAINWYQQKPGKAPKLLIYGHSYRLEGVPSRFSGSGSGTDYTLTISSLQPEDFATYYCQQGYIVPNRFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 214 aa, synthetic construct; identical to SEQ ID NO: 19
  REGION 1..214: note = 6R6-E1

SEQ ID NO: 21 - protein, 214 aa, synthetic construct
  REGION 1..214: note = 6R6-E2
DIQMTQSPSSLSASVGDRVTITCLASQPIYYGLNWYQQKPGKAPKLLIYGTSALVDGVPSRFSGSGSGTDYTLTISSLQPEDFATYYCQQGYIVPVSFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 214 aa, synthetic construct
  REGION 1..214: note = 6R6-E3
DIQMTQSPSSLSASVGDRVTITCQASENIRYAINWYQQKPGKAPKLLIYGANYRVDGVPSRFSGSGSGTDYTLTISSLQPEDFATYYCQQGYNVPTDFGGGTKVEIKRTVAAPSVFIFPPSDEQLKSGTASVVCLLNNFYPREAKVQWKVDNALQSGNSQESVTEQDSKDGTYSLSSTLTLSKADYEKHKVYACEVTHQGLSSPVTKSFNRGEC

SEQ ID NO: 23 - protein, 214 aa, synthetic construct
  REGION 1..214: note = 6R6-E4
DIQMTQSPSSLSASVGDRVTITCQASENIRYAVNWYQQKPGKAPKLLIYGANYRVDGVPSRFSGSGSGTDYTLTISSLQPEDFATYYCQQGYRVAIGFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 24 - protein, 214 aa, synthetic construct
  REGION 1..214: note = 6R6-F3
DIQMTQSPSSLSASVGDRVTITCQASEEVHYGINWYQQKPGKAPKLLIYGANYRVDGVPSRFSGSGSGTDYTLTISSLQPEDFATYYCQQGYNVPTDFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 214 aa, synthetic construct
  REGION 1..214: note = 6R6-F7
DIQMTQSPSSLSASVGDRVTITCQASERIYYGLNWYQQKPGKAPKLLIYGATGRADGVPSRFSGSGSGTDYTLTISSLQPEDFATYYCQQGYIVPSGFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 214 aa, synthetic construct
  REGION 1..214: note = 6R6-F9
DIQMTQSPSSLSASVGDRVTITCKASEDIRYGLNWYQQKPGKAPKLLIYGHTYRLDGVPSRFSGSGSGTDYTLTISSLQPEDFATYYCQQGYIVPNMFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 214 aa, synthetic construct
  REGION 1..214: note = 6R6-F11
DIQMTQSPSSLSASVGDRVTITCQASEQIYYGINWYQQKPGKAPKLLIYGATGRRDGVPSRFSGSGSGTDYTLTISSLQPEDFATYYCQQGYIVPTTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 214 aa, synthetic construct
  REGION 1..214: note = 6R6-F12
DIQMTQSPSSLSASVGDRVTITCQASQAIKYAINWYQQKPGKAPKLLIYGANYRGPGVPSRFSGSGSGTDYTLTISSLQPEDFATYYCQQGYIVPITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 214 aa, synthetic construct
  REGION 1..214: note = 3R6D11Q
DIQMTQSPSSLSASVGDRVTITCQASEPIYYAINWYQQKPGKAPKLLIYGQSHLQDGVPSRFSGSGSGTDYTLTISSLQPEDFATYYCQQGYRVPTFFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 214 aa, synthetic construct
  REGION 1..214: note = 6R6-E6Q
DIQMTQSPSSLSASVGDRVTITCQASEQVYYGLNWYQQKPGKAPKLLIYGANYRVDGVPSRFSGSGSGTDYTLTISSLQPEDFATYYCQQGYVVPNVFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 214 aa, synthetic construct; identical to SEQ ID NO: 22
  REGION 1..214: note = 6R8A-E11

SEQ ID NO: 32 - protein, 214 aa, synthetic construct
  REGION 1..214: note = 6R8A-F2
DIQMTQSPSSLSASVGDRVTITCQASEKVYYGVNWYQQKPGKAPKLLIYGTNYRLDGVPSRFSGSGSGTDSTLTISSLQPEDFATYYCQQGYNVPNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 214 aa, synthetic construct; identical to SEQ ID NO: 22
  REGION 1..214: note = 6R8A-E1

SEQ ID NO: 34 - protein, 214 aa, synthetic construct; identical to SEQ ID NO: 24
  REGION 1..214: note = 6R8A-A2

SEQ ID NO: 35 - protein, 214 aa, synthetic construct; identical to SEQ ID NO: 24
  REGION 1..214: note = 6R8C-G3

SEQ ID NO: 36 - protein, 214 aa, synthetic construct; identical to SEQ ID NO: 22
  REGION 1..214: note = 6R8C-G4

SEQ ID NO: 37 - protein, 214 aa, synthetic construct; identical to SEQ ID NO: 24
  REGION 1..214: note = 6R8C-G8

SEQ ID NO: 38 - protein, 214 aa, synthetic construct; identical to SEQ ID NO: 24
  REGION 1..214: note = 6R8C-D9

SEQ ID NO: 39 - protein, 214 aa, synthetic construct; identical to SEQ ID NO: 24
  REGION 1..214: note = 6R8C-D10

SEQ ID NO: 40 - protein, 214 aa, synthetic construct
  REGION 1..214: note = 6R8C-F10
DIQMTQSPSSLSASVGDRVTITCKASEDIRYGLNWYQQKPGKAPKLLIYGANYRVDGVPSRFSGSGSGTDYTLTISSLQPEDFATYYCQQGYNVPNFFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 455 aa, synthetic construct
  REGION 1..455: note = 3R4H4
EVQLVESGGGLVQPGGSLRLSCEASGFTFPLQAMSWVRQAPGKGLEWIGVIASNADTYYASWAKGRFTISRDNSKNTLYLQMNSLRAEDTAVYYCARHPGFAGYGYGHYYAMDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 455 aa, synthetic construct
  REGION 1..455: note = 5R4H1
EVQLVESGGGLVQPGGSLRLSCAASGYSVTHQAVTWVRQAPGKGLEWIGVIASNADTYYASWAKGRFTISRDNSKNTLYLQMNSLRAEDTAVYYCARHPGSAGYGYQYYYSMDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 455 aa, synthetic construct
  REGION 1..455: note = 5R4F3
EVQLVESGGGLVQPGGSLRLSCKASGFTFPKQAMTWVRQAPGKGLEWIGVIASNADTYYASWAKGRFTISRDNSKNTLYLQMNSLRAEDTAVYYCARHPGFAGYGYGHYYAMDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 455 aa, synthetic construct
  REGION 1..455: note = 5R4E11
EVQLVESGGGLVQPGGSLRLSCKASGFTFKPQAMTWVRQAPGKGLEWIGVIASNADTYYASWAKGRFTISRDNSKNTLYLQMNSLRAEDTAVYYCARHPGFAGYGYGHYYAMDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 455 aa, synthetic construct
  REGION 1..455: note = 6R4H1
EVQLVESGGGLVQPGGSLRLSCEASGYTFWSQAMTWVRQAPGKGLEWIGVIASNADTYYASWVKGRFTISRDNSKNTLYLQMNSLRAEDTAVYYCARHPGFAGYGYQYYYSMDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 455 aa, synthetic construct
  REGION 1..455: note = 6R4A7
EVQLVESGGGLVQPGGSLRLSCKASGISITEKAVTWVRQAPGKGLEWIGVIASNADTYYASWAKGRFTISRDNSKNTLYLQMNSLRAEDTAVYYCARHPGFAGYGYGHYYAMDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 455 aa, synthetic construct
  REGION 1..455: note = 6R4G10
EVQLVESGGGLVQPGRSLRLSCAASGYTIGPQAMTWVRQAPGKGLEWIGVIASNADTYYASWAKGRFTISRDNSKNTLYLQMNSLRAEDTAVYYCARHPGFAGYGYGHYYAMDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 455 aa, synthetic construct
  REGION 1..455: note = 6R4G11
EVQLVESGGGLVQPGGSLRLSCKASGYSLAPQAMTWVRQAPGKGLEWIGVIASNADTYYASWAKGRFTISRDNSKNTLYLQMNSLRAEDTAVYYCARHPGFAGYGYGHYYAMDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 455 aa, synthetic construct
  REGION 1..455: note = 6R4E12
EVQLVESGGGLVQLGGSLRLSCKASGYSLAPQAMTWVRQAPGKGLEWIGVIASNADTYYASWAKGRFTISRDNSKNTLYLQMNSLRAEDTAVYYCARHPGFAGYGYGHYYAMDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 455 aa, synthetic construct
  REGION 1..455: note = 6R4E3
EVQLVESGGGLVQPGGSLRLSCKASGYTFETQAMTWVRQAPGKGLEWIGVIASNADTYYASWAKGRFTISRDNSKNTLYLQMNSLRAEDTAVYYCARHPGSAGYGYQYYYAMDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 455 aa, synthetic construct
  REGION 1..455: note = 6R4H5
EVQLVESGGGLVQPGGSLRLSCAASGYTVKRQAVTWVRQAPGKGLEWIGVIASNADTYYASWAKGRFTISRDNSKNTLYLQMNSLRAEDTAVYYCARHPGSAGYGYQYYYAMDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 455 aa, synthetic construct
  REGION 1..455: note = 6R4H11
EVQLVESGGGLVQPGGSLRLSCKASGFTFKPQAMTWVRQAPGKGLEWIGVIASNADTYYASWAKGRFTISRDNSKNTLYLQMNSLRAEDTAVYYCARHPGSAGYGYQYYYAMDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 455 aa, synthetic construct
  REGION 1..455: note = 6R6A7
EVQLVESGGGLVQLGGSLRLSCKASGYSLAPQAMTWVRQAPGKGLEWIGVIASNADTYYASWAKGRFTISRDNSKNTLYLQMNSLRAEDTAVYYCARHPGSAGYGYQYYYSMDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 455 aa, synthetic construct
  REGION 1..455: note = 6R6E1
EVQLVESGGGLVQPGGSLRLSCEASGFTFPNQAMTWVRQAPGKGLEWIGVIASNADTYYASWAKGRFTISRDNSKNTLYLQMNSLRAEDTAVYYCARHPGFAGYGYGMYYSMDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 455 aa, synthetic construct
  REGION 1..455: note = 6R6E2
EVQLVESGGGLVQPGGSLRLSCKASGISVNTKALTWVRQAPGKGLEWIGVIASNADTYYASWAKGRFTISRDNSKNTLYLQMNSLRAEDTAVYYCARHPGFAGYGYGHYYAMDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 455 aa, synthetic construct
  REGION 1..455: note = 6R6E3
EVQLVESGGGLVQPGGSLRLSCKASGFTFSRQALTWVRQAPGKGLEWIGVIASNADTYYASWAKGRFTISRDNSKNTLYLQMNSLRAEDTAVYYCARHPGSAGYGYQYYYAMDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 455 aa, synthetic construct; identical to SEQ ID NO: 51
  REGION 1..455: note = 6R6E4

SEQ ID NO: 58 - protein, 455 aa, synthetic construct
  REGION 1..455: note = 6R6F3
EVQLVESGGGLVQPGGSLRLSCEASGISISPNAVTWVRQAPGKGLEWIGVIASNADTYYASWAKGRFTISRDNSKNTLYLQMNSLRAEDTAVYYCARHPGSAGYGYQYYYAMDPWGQGTLVTVSSASTKGPSVFPLAPPSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 455 aa, synthetic construct; identical to SEQ ID NO: 41
  REGION 1..455: note = 6R6F7

SEQ ID NO: 60 - protein, 455 aa, synthetic construct
  REGION 1..455: note = 6R6F9
EVQLVESGGGLVQPGGSLRLSCEASGIDLSPQAMTWVRQAPGKGLEWIGVIASNADTYYASWAKGRFTISRDNSKNTLYLQMNSLRAEDTAVYYCARHPGSAGYGYQYYYAMDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 455 aa, synthetic construct
  REGION 1..455: note = 6R6F11
EVQLVESGGGLVQPGGSLRLSCEASGYTFASQAMTWVRQAPGKGLEWIGVIASNADTYYASWAKGRFTISRDNSKNTLYLQMNSLRAEDTAVYYCARHPGFAGYGYGHYYAMDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 455 aa, synthetic construct; identical to SEQ ID NO: 56
  REGION 1..455: note = 6R6F12

SEQ ID NO: 63 - protein, 455 aa, synthetic construct; identical to SEQ ID NO: 49
  REGION 1..455: note = 3R6D11Q

SEQ ID NO: 64 - protein, 455 aa, synthetic construct
  REGION 1..455: note = 6R6E6Q
EVQLVESGGGLVQPGGSLRLSCKASGYSLAPQAMTWVRQAPGKGLEWIGVIASNADTYYASWAKGRFTISRDNSKNTLYLQMNSLRAEDTAVYYCARHPGSAGYGYQYYYAMDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 455 aa, synthetic construct; identical to SEQ ID NO: 56
  REGION 1..455: note = 6R8AE11

SEQ ID NO: 66 - protein, 455 aa, synthetic construct
  REGION 1..455: note = 6R8AF2
EVQLVESGGGLVQPGGSLRLSCKASGYSLSSQALTWVRQAPGKGLEWIGVIASNADTYYASWAKGRFTISRDNSKNTLYLQMNSLRAEDTAVYYCARHPGSAGYGYQYYYAMDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 455 aa, synthetic construct; identical to SEQ ID NO: 64
  REGION 1..455: note = 6R8AE1

SEQ ID NO: 68 - protein, 455 aa, synthetic construct
  REGION 1..455: note = 6R8AA2
EVQLVESGGGLVQPGGSLRLSCKASGYSLNKQALTWVRQAPGKGLEWIGVIASNADTYYASWAKGRFTISRDNSKNTLYLQMNSLRAEDTAVYYCARHPGSAGYGYQYYYAMDPWGQGTLVTVSSASTKGPSVFPLAPSSKSTSGDTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 455 aa, synthetic construct
  REGION 1..455: note = 6R8CG3
EVQLVESGGGLVQPGGSLRLSCKASGISLAPYAITWVRQAPGKGLEWIGVIASNADTYYASWAKGRFTISRDNSKNTLYLQMNSLRAEDTAVYYCARHPGSAGYGYQYYYSMDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 455 aa, synthetic construct; identical to SEQ ID NO: 56
  REGION 1..455: note = 6R8CG4

SEQ ID NO: 71 - protein, 455 aa, synthetic construct
  REGION 1..455: note = 6R8CG8
EVQLVESGGGLVQLGGSLRLSCKASGYSLAPQAMTWVRQAPGKGLEWIGVIASNADTYYASWAKGRFTISRDNSKNTLYLQMNSLRAEDTAVYYCARHPGSAGYGYQYYYAMDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 455 aa, synthetic construct; identical to SEQ ID NO: 56
  REGION 1..455: note = 6R8CD9

SEQ ID NO: 73 - protein, 455 aa, synthetic construct
  REGION 1..455: note = 6R8CD10
EVQLVESGGGLVQPGGSLRLSCTASGYSISHQAVTWVRQAPGKGLEWIGVIASNADTYYASWAKGRFTISRDNSKNTLYLQMNSLRAEDTAVYYCARHPGSAGYGYQYYYAMDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 455 aa, synthetic construct; identical to SEQ ID NO: 56
  REGION 1..455: note = 6R8CF10

SEQ ID NO: 75 - protein, 11 aa, synthetic construct
  REGION 1..11: note = 3R4-H4 LCDR1
QASQGIHYAIN

SEQ ID NO: 76 - protein, 11 aa, synthetic construct
  REGION 1..11: note = 5RF-H1 LCDR1
QASEPIHYAIN

SEQ ID NO: 77 - protein, 11 aa, synthetic construct
  REGION 1..11: note = 5R4-F3 LCDR1
QASEDIRYGLN

SEQ ID NO: 78 - protein, 11 aa, synthetic construct
  REGION 1..11: note = 5R4-E11 LCDR1
KASEPIYYAIN

SEQ ID NO: 79 - protein, 11 aa, synthetic construct
  REGION 1..11: note = 6R4H1 LCDR1
QASQPIFYGID

SEQ ID NO: 80 - protein, 11 aa, synthetic construct
  REGION 1..11: note = 6R4-A7 LCDR1
QASQQIYYAIN

SEQ ID NO: 81 - protein, 11 aa, synthetic construct
  REGION 1..11: note = 6R4-G10 LCDR1
QASERIYYAIN

SEQ ID NO: 82 - protein, 11 aa, synthetic construct
  REGION 1..11: note = 6R4-G11 LCDR1
KASEDIRYGLN

SEQ ID NO: 83 - protein, 11 aa, synthetic construct
  REGION 1..11: note = 6R4-E12 LCDR1
QASEKIHYAIN

SEQ ID NO: 84 - protein, 11 aa, synthetic construct
  REGION 1..11: note = 6R4-E3 LCDR1
QASQLIYYGIN

SEQ ID NO: 85 - protein, 11 aa, synthetic construct
  REGION 1..11: note = 6R4-H5 LCDR1
KASEDIRYGLN

SEQ ID NO: 86 - protein, 11 aa, synthetic construct
  REGION 1..11: note = 6R4-H11 LCDR1
QASQGIHYAIN

SEQ ID NO: 87 - protein, 11 aa, synthetic construct
  REGION 1..11: note = 6R6-A7 LCDR1
QASEKIYYAIN

SEQ ID NO: 88 - protein, 11 aa, synthetic construct
  REGION 1..11: note = 6R6-E1 LCDR1
QASQQIYYAIN

SEQ ID NO: 89 - protein, 11 aa, synthetic construct
  REGION 1..11: note = 6R6-E2 LCDR1
LASQPIYYGLN

SEQ ID NO: 90 - protein, 11 aa, synthetic construct
  REGION 1..11: note = 6R6-E3 LCDR1
QASENIRYAIN

SEQ ID NO: 91 - protein, 11 aa, synthetic construct
  REGION 1..11: note = 6R6-E4 LCDR1
QASENIRYAVN

SEQ ID NO: 92 - protein, 11 aa, synthetic construct
  REGION 1..11: note = 6R6-F3 LCDR1
QASEEVHYGIN

SEQ ID NO: 93 - protein, 11 aa, synthetic construct
  REGION 1..11: note = 6R6-F7 LCDR1
QASERIYYGLN

SEQ ID NO: 94 - protein, 11 aa, synthetic construct
  REGION 1..11: note = 6R6-F9 LCDR1
QASQAIKYAIN

SEQ ID NO: 95 - protein, 11 aa, synthetic construct
  REGION 1..11: note = 6R6-F11 LCDR1
QASEQIYYGIN

SEQ ID NO: 96 - protein, 11 aa, synthetic construct
  REGION 1..11: note = 6R6-F12 LCDR1
QASQAIKYAIN

SEQ ID NO: 97 - protein, 11 aa, synthetic construct
  REGION 1..11: note = 3R6D11Q LCDR1
QASEPIYYAIN

SEQ ID NO: 98 - protein, 11 aa, synthetic construct
  REGION 1..11: note = 6R6-E6Q LCDR1
QASEQVYYGLN

SEQ ID NO: 99 - protein, 11 aa, synthetic construct
  REGION 1..11: note = 6R8A-E11 LCDR1
QASENIRYAIN

SEQ ID NO: 100 - protein, 10 aa, synthetic construct
  REGION 1..10: note = 6R8A-F2 LCDR1
QASEKVYGVN

SEQ ID NO: 101 - protein, 11 aa, synthetic construct
  REGION 1..11: note = 6R8A-E11 LCDR1
QASEEVHYGIN

SEQ ID NO: 102 - protein, 11 aa, synthetic construct
  REGION 1..11: note = 6R8A-A2 LCDR1
QASEEVHYGIN

SEQ ID NO: 103 - protein, 11 aa, synthetic construct
  REGION 1..11: note = 6R8C-G3 LCDR1
QASEEVHYGIN

SEQ ID NO: 104 - protein, 11 aa, synthetic construct
  REGION 1..11: note = 6R8C-G4 LCDR1
QASENIRYAIN

SEQ ID NO: 105 - protein, 11 aa, synthetic construct
  REGION 1..11: note = 6R8C-G8 LCDR1
QASEEVHYGIN

SEQ ID NO: 106 - protein, 11 aa, synthetic construct
  REGION 1..11: note = 6R8C-D9 LCDR1
QASEEVHYGIN

SEQ ID NO: 107 - protein, 11 aa, synthetic construct
  REGION 1..11: note = 6R8C-D10 LCDR1
QASEEVHYGIN

SEQ ID NO: 108 - protein, 11 aa, synthetic construct
  REGION 1..11: note = 6R8C-F10 LCDR1
KASEDIRYGLN

SEQ ID NO: 109 - protein, 8 aa, synthetic construct
  REGION 1..8: note = 3R4-H4 LCDR2
YGHNYLEE

SEQ ID NO: 110 - protein, 8 aa, synthetic construct
  REGION 1..8: note = 5RF-H1 LCDR2
YGTSYRLD

SEQ ID NO: 111 - protein, 8 aa, synthetic construct
  REGION 1..8: note = 5R4-F3 LCDR2
YGTSYRRD

SEQ ID NO: 112 - protein, 8 aa, synthetic construct
  REGION 1..8: note = 5R4-E11 LCDR2
YGASGLAD

SEQ ID NO: 113 - protein, 8 aa, synthetic construct
  REGION 1..8: note = 6R4H1 LCDR2
YGTSYRAE

SEQ ID NO: 114 - protein, 8 aa, synthetic construct
  REGION 1..8: note = 6R4-A7 LCDR2
YGANYRED

SEQ ID NO: 115 - protein, 8 aa, synthetic construct
  REGION 1..8: note = 6R4-G10 LCDR2
YGANYRVD

SEQ ID NO: 116 - protein, 8 aa, synthetic construct
  REGION 1..8: note = 6R4-G11 LCDR2
YGANHLRE

SEQ ID NO: 117 - protein, 8 aa, synthetic construct
  REGION 1..8: note = 6R4-E12 LCDR2
YGHSHLED

SEQ ID NO: 118 - protein, 8 aa, synthetic construct
  REGION 1..8: note = 6R4-E3 LCDR2
YGANYRVD

SEQ ID NO: 119 - protein, 8 aa, synthetic construct
  REGION 1..8: note = 6R4-H5 LCDR2
YGHTYRLD

SEQ ID NO: 120 - protein, 8 aa, synthetic construct
  REGION 1..8: note = 6R4-H11 LCDR2
YGHNYLEE

SEQ ID NO: 121 - protein, 8 aa, synthetic construct
  REGION 1..8: note = 6R6-A7 LCDR2
YGHSYRLE

SEQ ID NO: 122 - protein, 8 aa, synthetic construct
  REGION 1..8: note = 6R6-E1 LCDR2
YGTSYRAE

SEQ ID NO: 123 - protein, 8 aa, synthetic construct
  REGION 1..8: note = 6R6-E2 LCDR2
YGTSALVD

SEQ ID NO: 124 - protein, 8 aa, synthetic construct
  REGION 1..8: note = 6R6-E3 LCDR2
YGANYRVD

SEQ ID NO: 125 - protein, 8 aa, synthetic construct
  REGION 1..8: note = 6R6-E4 LCDR2
YGANYRVD

SEQ ID NO: 126 - protein, 8 aa, synthetic construct
  REGION 1..8: note = 6R6-F3 LCDR2
YGANYRVD

SEQ ID NO: 127 - protein, 8 aa, synthetic construct
  REGION 1..8: note = 6R6-F7 LCDR2
YGATGRAD

SEQ ID NO: 128 - protein, 8 aa, synthetic construct
  REGION 1..8: note = 6R6-F9 LCDR2
YGANYRGP

SEQ ID NO: 129 - protein, 8 aa, synthetic construct
  REGION 1..8: note = 6R6-F11 LCDR2
YGATGRRD

SEQ ID NO: 130 - protein, 8 aa, synthetic construct
  REGION 1..8: note = 6R6-F12 LCDR2
YGANYRGP

SEQ ID NO: 131 - protein, 8 aa, synthetic construct
  REGION 1..8: note = 3R6D11Q LCDR2
YGQSHLQD

SEQ ID NO: 132 - protein, 8 aa, synthetic construct
  REGION 1..8: note = 6R6-E6Q LCDR2
YGANYRVD

SEQ ID NO: 133 - protein, 8 aa, synthetic construct
  REGION 1..8: note = 6R8A-E11 LCDR2
YGANYRVD

SEQ ID NO: 134 - protein, 8 aa, synthetic construct
  REGION 1..8: note = 6R8A-F2 LCDR2
YGTNYRLD

SEQ ID NO: 135 - protein, 8 aa, synthetic construct
  REGION 1..8: note = 6R8A-E11 LCDR2
YGANYRVD

SEQ ID NO: 136 - protein, 8 aa, synthetic construct
  REGION 1..8: note = 6R8A-A2 LCDR2
YGANYRVD

SEQ ID NO: 137 - protein, 8 aa, synthetic construct
  REGION 1..8: note = 6R8C-G3 LCDR2
YGANYRVD

SEQ ID NO: 138 - protein, 8 aa, synthetic construct
  REGION 1..8: note = 6R8C-G4 LCDR2
YGANYRVD

SEQ ID NO: 139 - protein, 8 aa, synthetic construct
  REGION 1..8: note = 6R8C-G8 LCDR2
YGANYRVD

SEQ ID NO: 140 - protein, 8 aa, synthetic construct
  REGION 1..8: note = 6R8C-D9 LCDR2
YGANYRVD

SEQ ID NO: 141 - protein, 8 aa, synthetic construct
  REGION 1..8: note = 6R8C-D10 LCDR2
YGANYRVD

SEQ ID NO: 142 - protein, 8 aa, synthetic construct
  REGION 1..8: note = 6R8C-F10 LCDR2
YGANYRVD

SEQ ID NO: 143 - protein, 9 aa, synthetic construct
  REGION 1..9: note = 3R4-H4 LCDR3
QQGYIVPIV

SEQ ID NO: 144 - protein, 9 aa, synthetic construct
  REGION 1..9: note = 5RF-H1 LCDR3
QQGYTVPLH

SEQ ID NO: 145 - protein, 9 aa, synthetic construct
  REGION 1..9: note = 5R4-F3 LCDR3
QQGYNLPTR

SEQ ID NO: 146 - protein, 9 aa, synthetic construct
  REGION 1..9: note = 5R4-E11 LCDR3
QQGYIVPIM

SEQ ID NO: 147 - protein, 9 aa, synthetic construct
  REGION 1..9: note = 6R4H1 LCDR3
QQGYIVPIT

SEQ ID NO: 148 - protein, 9 aa, synthetic construct
  REGION 1..9: note = 6R4-A7 LCDR3
QQGYLVPVG

SEQ ID NO: 149 - protein, 9 aa, synthetic construct
  REGION 1..9: note = 6R4-G10 LCDR3
QQGYIVPTN

SEQ ID NO: 150 - protein, 9 aa, synthetic construct
  REGION 1..9: note = 6R4-G11 LCDR3
GWGYSVPLT

SEQ ID NO: 151 - protein, 9 aa, synthetic construct
  REGION 1..9: note = 6R4-E12 LCDR3
QQGYIVPNS

SEQ ID NO: 152 - protein, 9 aa, synthetic construct
  REGION 1..9: note = 6R4-E3 LCDR3
QQGYSVPLD

SEQ ID NO: 153 - protein, 9 aa, synthetic construct
  REGION 1..9: note = 6R4-H5 LCDR3
QQGYVVPVG

SEQ ID NO: 154 - protein, 9 aa, synthetic construct
  REGION 1..9: note = 6R4-H11 LCDR3
QQGYIVPIV

SEQ ID NO: 155 - protein, 9 aa, synthetic construct
  REGION 1..9: note = 6R6-A7 LCDR3
QQGYIVPNR

SEQ ID NO: 156 - protein, 9 aa, synthetic construct
  REGION 1..9: note = 6R6-E1 LCDR3
QQGYIVPIT

SEQ ID NO: 157 - protein, 9 aa, synthetic construct
  REGION 1..9: note = 6R6-E2 LCDR3
QQGYIVPVS

SEQ ID NO: 158 - protein, 9 aa, synthetic construct
  REGION 1..9: note = 6R6-E3 LCDR3
QQGYNVPTD

SEQ ID NO: 159 - protein, 9 aa, synthetic construct
  REGION 1..9: note = 6R6-E4 LCDR3
QQGYRVAIG

SEQ ID NO: 160 - protein, 9 aa, synthetic construct
  REGION 1..9: note = 6R6-F3 LCDR3
QQGYNVPTD

SEQ ID NO: 161 - protein, 9 aa, synthetic construct
  REGION 1..9: note = 6R6-F7 LCDR3
QQGYIVPSG

SEQ ID NO: 162 - protein, 9 aa, synthetic construct
  REGION 1..9: note = 6R6-F9 LCDR3
QQGYIVPIT

SEQ ID NO: 163 - protein, 9 aa, synthetic construct
  REGION 1..9: note = 6R6-F11 LCDR3
QQGYIVPIT

SEQ ID NO: 164 - protein, 9 aa, synthetic construct
  REGION 1..9: note = 6R6-F12 LCDR3
QQGYIVPIT

SEQ ID NO: 165 - protein, 9 aa, synthetic construct
  REGION 1..9: note = 3R6D11Q LCDR3
QQGYRVPTF

SEQ ID NO: 166 - protein, 9 aa, synthetic construct
  REGION 1..9: note = 6R6-E6Q LCDR3
QQGYVVPNV

SEQ ID NO: 167 - protein, 9 aa, synthetic construct
  REGION 1..9: note = 6R8A-E11 LCDR3
QQGYNVPTD

SEQ ID NO: 168 - protein, 9 aa, synthetic construct
  REGION 1..9: note = 6R8A-F2 LCDR3
QQGYNVPNA

SEQ ID NO: 169 - protein, 9 aa, synthetic construct
  REGION 1..9: note = 6R8A-E11 LCDR3
QQGYNVPTD

SEQ ID NO: 170 - protein, 9 aa, synthetic construct
  REGION 1..9: note = 6R8A-A2 LCDR3
QQGYNVPTD

SEQ ID NO: 171 - protein, 9 aa, synthetic construct
  REGION 1..9: note = 6R8C-G3 LCDR3
QQGYNVPTD

SEQ ID NO: 172 - protein, 9 aa, synthetic construct
  REGION 1..9: note = 6R8C-G4 LCDR3
QQGYNVPTD

SEQ ID NO: 173 - protein, 9 aa, synthetic construct
  REGION 1..9: note = 6R8C-G8 LCDR3
QQGYNVPTD

SEQ ID NO: 174 - protein, 9 aa, synthetic construct
  REGION 1..9: note = 6R8C-D9 LCDR3
QQGYNVPTD

SEQ ID NO: 175 - protein, 9 aa, synthetic construct
  REGION 1..9: note = 6R8C-D10 LCDR3
QQGYNVPTD

SEQ ID NO: 176 - protein, 9 aa, synthetic construct
  REGION 1..9: note = 6R8C-F10 LCDR3
QQGYNVPNF

SEQ ID NO: 177 - protein, 13 aa, synthetic construct
  REGION 1..13: note = 3R4-H4 HCDR1
EASGFTFPLQAMS

SEQ ID NO: 178 - protein, 13 aa, synthetic construct
  REGION 1..13: note = 5RF-H1 HCDR1
AASGYSVTHQAVT

SEQ ID NO: 179 - protein, 13 aa, synthetic construct
  REGION 1..13: note = 5R4-F3 HCDR1
KASGFTFPKQAMT

SEQ ID NO: 180 - protein, 13 aa, synthetic construct
  REGION 1..13: note = 5R4-E11 HCDR1
KASGFTFKPQAMT

SEQ ID NO: 181 - protein, 13 aa, synthetic construct
  REGION 1..13: note = 6R4H1 HCDR1
EASGYTEWSQAMT

SEQ ID NO: 182 - protein, 13 aa, synthetic construct
  REGION 1..13: note = 6R4-A7 HCDR1
KASGISITEKAVT

SEQ ID NO: 183 - protein, 13 aa, synthetic construct
  REGION 1..13: note = 6R4-G10 HCDR1
AASGYTIGPQAMT

SEQ ID NO: 184 - protein, 13 aa, synthetic construct
  REGION 1..13: note = 6R4-G11 HCDR1
KASGYSLAPQAMT

SEQ ID NO: 185 - protein, 13 aa, synthetic construct
  REGION 1..13: note = 6R4-E12 HCDR1
KASGYSLAPQAMT

SEQ ID NO: 186 - protein, 13 aa, synthetic construct
  REGION 1..13: note = 6R4-E3 HCDR1
KASGYTFETQAMT

SEQ ID NO: 187 - protein, 13 aa, synthetic construct
  REGION 1..13: note = 6R4-H5 HCDR1
AASGYTVKRQAVT

SEQ ID NO: 188 - protein, 13 aa, synthetic construct
  REGION 1..13: note = 6R4-H11 HCDR1
KASGFTFKPQAMT

SEQ ID NO: 189 - protein, 13 aa, synthetic construct
  REGION 1..13: note = 6R6-A7 HCDR1
KASGYSLAPQAMT

SEQ ID NO: 190 - protein, 13 aa, synthetic construct
  REGION 1..13: note = 6R6-E1 HCDR1
EASGFTFPNQAMT

SEQ ID NO: 191 - protein, 13 aa, synthetic construct
  REGION 1..13: note = 6R6-E2 HCDR1
KASGISVNTKALT

SEQ ID NO: 192 - protein, 13 aa, synthetic construct
  REGION 1..13: note = 6R6-E3 HCDR1
KASGFTFSRQALT

SEQ ID NO: 193 - protein, 13 aa, synthetic construct
  REGION 1..13: note = 6R6-E4 HCDR1
AASGYTVKRQAVT

SEQ ID NO: 194 - protein, 13 aa, synthetic construct
  REGION 1..13: note = 6R6-F3 HCDR1
EASGISISPNAVT

SEQ ID NO: 195 - protein, 13 aa, synthetic construct
  REGION 1..13: note = 6R6-F7 HCDR1
EASGFTFPLQAMS

SEQ ID NO: 196 - protein, 13 aa, synthetic construct
  REGION 1..13: note = 6R6-F9 HCDR1
EASGIDLSPQAMT

SEQ ID NO: 197 - protein, 13 aa, synthetic construct
  REGION 1..13: note = 6R6-F11 HCDR1
EASGYTFASQAMT

SEQ ID NO: 198 - protein, 13 aa, synthetic construct
  REGION 1..13: note = 6R6-F12 HCDR1
KASGFTLAPQALT

SEQ ID NO: 199 - protein, 13 aa, synthetic construct
  REGION 1..13: note = 3R6D11Q HCDR1
KASGYSLAPQAMT

SEQ ID NO: 200 - protein, 13 aa, synthetic construct
  REGION 1..13: note = 6R6-E6Q HCDR1
KASGYSLAPQAMT

SEQ ID NO: 201 - protein, 13 aa, synthetic construct
  REGION 1..13: note = 6R8A-E11 HCDR1
KASGFTFSRQALT

SEQ ID NO: 202 - protein, 13 aa, synthetic construct
  REGION 1..13: note = 6R8A-F2 HCDR1
KASGYSLSSQALT

SEQ ID NO: 203 - protein, 13 aa, synthetic construct
  REGION 1..13: note = 6R8A-E11 HCDR1
KASGYSLAPQAMT

SEQ ID NO: 204 - protein, 13 aa, synthetic construct
  REGION 1..13: note = 6R8A-A2 HCDR1
KASGYSLNKQALT

SEQ ID NO: 205 - protein, 13 aa, synthetic construct
  REGION 1..13: note = 6R8C-G3 HCDR1
KASGISLAPYAIT

SEQ ID NO: 206 - protein, 13 aa, synthetic construct
  REGION 1..13: note = 6R8C-G4 HCDR1
KASGFTFSRQALT

SEQ ID NO: 207 - protein, 13 aa, synthetic construct
  REGION 1..13: note = 6R8C-G8 HCDR1
KASGYSLAPQAMT

SEQ ID NO: 208 - protein, 13 aa, synthetic construct
  REGION 1..13: note = 6R8C-D9 HCDR1
KASGFTFSRQALT

SEQ ID NO: 209 - protein, 13 aa, synthetic construct
  REGION 1..13: note = 6R8C-D10 HCDR1
TASGYSISHQAVT

SEQ ID NO: 210 - protein, 13 aa, synthetic construct
  REGION 1..13: note = 6R8C-F10 HCDR1
KASGFTFSRQALT

SEQ ID NO: 211 - protein, 18 aa, synthetic construct
  REGION 1..18: note = 3R4-H4 HCDR2
IGVIASNADTYYASWAKG

SEQ ID NO: 212 - protein, 18 aa, synthetic construct
  REGION 1..18: note = 5RF-H1 HCDR2
IGVIASNADTYYASWAKG

SEQ ID NO: 213 - protein, 18 aa, synthetic construct
  REGION 1..18: note = 5R4-F3 HCDR2
IGVIASNADTYYASWAKG

SEQ ID NO: 214 - protein, 18 aa, synthetic construct
  REGION 1..18: note = 5R4-E11 HCDR2
IGVIASNADTYYASWAKG

SEQ ID NO: 215 - protein, 18 aa, synthetic construct
  REGION 1..18: note = 6R4H1 HCDR2
IGVIASNADTYYASWVKG

SEQ ID NO: 216 - protein, 18 aa, synthetic construct
  REGION 1..18: note = 6R4-A7 HCDR2
IGVIASNADTYYASWAKG

SEQ ID NO: 217 - protein, 18 aa, synthetic construct
  REGION 1..18: note = 6R4-G10 HCDR2
IGVIASNADTYYASWAKG

SEQ ID NO: 218 - protein, 18 aa, synthetic construct
  REGION 1..18: note = 6R4-G11 HCDR2
IGVIASNADTYYASWAKG

SEQ ID NO: 219 - protein, 18 aa, synthetic construct
  REGION 1..18: note = 6R4-E12 HCDR2
IGVIASNADTYYASWAKG

SEQ ID NO: 220 - protein, 18 aa, synthetic construct
  REGION 1..18: note = 6R4-E3 HCDR2
IGVIASNADTYYASWAKG

SEQ ID NO: 221 - protein, 18 aa, synthetic construct
  REGION 1..18: note = 6R4-H5 HCDR2
IGVIASNADTYYASWAKG

SEQ ID NO: 222 - protein, 18 aa, synthetic construct
  REGION 1..18: note = 6R4-H11 HCDR2
IGVIASNADTYYASWAKG

SEQ ID NO: 223 - protein, 18 aa, synthetic construct
  REGION 1..18: note = 6R6-A7 HCDR2
IGVIASNADTYYASWAKG

SEQ ID NO: 224 - protein, 18 aa, synthetic construct
  REGION 1..18: note = 6R6-E1 HCDR2
IGVIASNADTYYASWAKG

SEQ ID NO: 225 - protein, 18 aa, synthetic construct
  REGION 1..18: note = 6R6-E2 HCDR2
IGVIASNADTYYASWAKG

SEQ ID NO: 226 - protein, 18 aa, synthetic construct
  REGION 1..18: note = 6R6-E3 HCDR2
IGVIASNADTYYASWAKG

SEQ ID NO: 227 - protein, 18 aa, synthetic construct
  REGION 1..18: note = 6R6-E4 HCDR2
IGVIASNADTYYASWAKG

SEQ ID NO: 228 - protein, 18 aa, synthetic construct
  REGION 1..18: note = 6R6-F3 HCDR2
IGVIASNADTYYASWAKG

SEQ ID NO: 229 - protein, 18 aa, synthetic construct
  REGION 1..18: note = 6R6-F7 HCDR2
IGVIASNADTYYASWAKG

SEQ ID NO: 230 - protein, 18 aa, synthetic construct
  REGION 1..18: note = 6R6-F9 HCDR2
IGVIASNADTYYASWAKG

SEQ ID NO: 231 - protein, 18 aa, synthetic construct
  REGION 1..18: note = 6R6-F11 HCDR2
IGVIASNADTYYASWAKG

SEQ ID NO: 232 - protein, 18 aa, synthetic construct
  REGION 1..18: note = 6R6-F12 HCDR2
IGVIASNADTYYASWAKG

SEQ ID NO: 233 - protein, 18 aa, synthetic construct
  REGION 1..18: note = 3R6D11Q HCDR2
IGVIASNADTYYASWAKG

SEQ ID NO: 234 - protein, 18 aa, synthetic construct
  REGION 1..18: note = 6R6-E6Q HCDR2
IGVIASNADTYYASWAKG

SEQ ID NO: 235 - protein, 18 aa, synthetic construct
  REGION 1..18: note = 6R8A-E11 HCDR2
IGVIASNADTYYASWAKG

SEQ ID NO: 236 - protein, 18 aa, synthetic construct
  REGION 1..18: note = 6R8A-F2 HCDR2
IGVIASNADTYYASWAKG

SEQ ID NO: 237 - protein, 18 aa, synthetic construct
  REGION 1..18: note = 6R8A-E11 HCDR2
IGVIASNADTYYASWAKG

SEQ ID NO: 238 - protein, 18 aa, synthetic construct
  REGION 1..18: note = 6R8A-A2 HCDR2
IGVIASNADTYYASWAKG

SEQ ID NO: 239 - protein, 18 aa, synthetic construct
  REGION 1..18: note = 6R8C-G3 HCDR2
IGVIASNADTYYASWAKG

SEQ ID NO: 240 - protein, 18 aa, synthetic construct
  REGION 1..18: note = 6R8C-G4 HCDR2
IGVIASNADTYYASWAKG

SEQ ID NO: 241 - protein, 18 aa, synthetic construct
  REGION 1..18: note = 6R8C-G8 HCDR2
IGVIASNADTYYASWAKG

SEQ ID NO: 242 - protein, 18 aa, synthetic construct
  REGION 1..18: note = 6R8C-D9 HCDR2
IGVIASNADTYYASWAKG

SEQ ID NO: 243 - protein, 18 aa, synthetic construct
  REGION 1..18: note = 6R8C-D10 HCDR2
IGVIASNADTYYASWAKG

SEQ ID NO: 244 - protein, 18 aa, synthetic construct
  REGION 1..18: note = 6R8C-F10 HCDR2
IGVIASNADTYYASWAKG

SEQ ID NO: 245 - protein, 19 aa, synthetic construct
  REGION 1..19: note = 3R4-H4 HCDR3
ARHPGFAGYGYGHYYAMDP

SEQ ID NO: 246 - protein, 19 aa, synthetic construct
  REGION 1..19: note = 5RF-H1 HCDR3
ARHPGSAGYGYQYYYSMDP

SEQ ID NO: 247 - protein, 19 aa, synthetic construct
  REGION 1..19: note = 5R4-F3 HCDR3
ARHPGFAGYGYGHYYAMDP

SEQ ID NO: 248 - protein, 19 aa, synthetic construct
  REGION 1..19: note = 5R4-E11 HCDR3
ARHPGFAGYGYGHYYAMDP

SEQ ID NO: 249 - protein, 19 aa, synthetic construct
  REGION 1..19: note = 6R4H1 HCDR3
ARHPGFAGYGYQYYYSMDP

SEQ ID NO: 250 - protein, 19 aa, synthetic construct
  REGION 1..19: note = 6R4-A7 HCDR3
ARHPGFAGYGYGHYYAMDP

SEQ ID NO: 251 - protein, 19 aa, synthetic construct
  REGION 1..19: note = 6R4-G10 HCDR3
ARHPGFAGYGYGHYYAMDP

SEQ ID NO: 252 - protein, 19 aa, synthetic construct
  REGION 1..19: note = 6R4-G11 HCDR3
ARHPGFAGYGYGHYYAMDP

SEQ ID NO: 253 - protein, 19 aa, synthetic construct
  REGION 1..19: note = 6R4-E12 HCDR3
ARHPGFAGYGYGHYYAMDP

SEQ ID NO: 254 - protein, 19 aa, synthetic construct
  REGION 1..19: note = 6R4-E3 HCDR3
ARHPGSAGYGYQYYYAMDP

SEQ ID NO: 255 - protein, 19 aa, synthetic construct
  REGION 1..19: note = 6R4-H5 HCDR3
ARHPGSAGYGYQYYYAMDP

SEQ ID NO: 256 - protein, 19 aa, synthetic construct
  REGION 1..19: note = 6R4-H11 HCDR3
ARHPGSAGYGYQYYYAMDP

SEQ ID NO: 257 - protein, 19 aa, synthetic construct
  REGION 1..19: note = 6R6-A7 HCDR3
ARHPGSAGYGYQYYYSMDP

SEQ ID NO: 258 - protein, 19 aa, synthetic construct
  REGION 1..19: note = 6R6-E1 HCDR3
ARHPGFAGYGYGMYYSMDP

SEQ ID NO: 259 - protein, 19 aa, synthetic construct
  REGION 1..19: note = 6R6-E2 HCDR3
ARHPGFAGYGYGHYYAMDP

SEQ ID NO: 260 - protein, 19 aa, synthetic construct
  REGION 1..19: note = 6R6-E3 HCDR3
ARHPGSAGYGYQYYYAMDP

SEQ ID NO: 261 - protein, 19 aa, synthetic construct
  REGION 1..19: note = 6R6-E4 HCDR3
ARHPGSAGYGYQYYYAMDP

SEQ ID NO: 262 - protein, 19 aa, synthetic construct
  REGION 1..19: note = 6R6-F3 HCDR3
ARHPGSAGYGYQYYYAMDP

SEQ ID NO: 263 - protein, 19 aa, synthetic construct
  REGION 1..19: note = 6R6-F7 HCDR3
ARHPGFAGYGYGHYYAMDP

SEQ ID NO: 264 - protein, 19 aa, synthetic construct
  REGION 1..19: note = 6R6-F9 HCDR3
ARHPGSAGYGYQYYYAMDP

SEQ ID NO: 265 - protein, 19 aa, synthetic construct
  REGION 1..19: note = 6R6-F11 HCDR3
ARHPGFAGYGYGHYYAMDP

SEQ ID NO: 266 - protein, 19 aa, synthetic construct
  REGION 1..19: note = 6R6-F12 HCDR3
ARHPGFAGYGYRHYYAMDP

SEQ ID NO: 267 - protein, 19 aa, synthetic construct
  REGION 1..19: note = 3R6D11Q HCDR3
ARHPGFAGYGYGHYYAMDP

SEQ ID NO: 268 - protein, 19 aa, synthetic construct
  REGION 1..19: note = 6R6-E6Q HCDR3
ARHPGSAGYGYQYYYAMDP

SEQ ID NO: 269 - protein, 19 aa, synthetic construct
  REGION 1..19: note = 6R8A-E11 HCDR3
ARHPGSAGYGYQYYYAMDP

SEQ ID NO: 270 - protein, 19 aa, synthetic construct
  REGION 1..19: note = 6R8A-F2 HCDR3
ARHPGSAGYGYQYYYAMDP

SEQ ID NO: 271 - protein, 19 aa, synthetic construct
  REGION 1..19: note = 6R8A-E11 HCDR3
ARHPGSAGYGYQYYYAMDP

SEQ ID NO: 272 - protein, 19 aa, synthetic construct
  REGION 1..19: note = 6R8A-A2 HCDR3
ARHPGSAGYGYQYYYAMDP

SEQ ID NO: 273 - protein, 19 aa, synthetic construct
  REGION 1..19: note = 6R8C-G3 HCDR3
ARHPGSAGYGYQYYYSMDP

SEQ ID NO: 274 - protein, 19 aa, synthetic construct
  REGION 1..19: note = 6R8C-G4 HCDR3
ARHPGSAGYGYQYYYAMDP

SEQ ID NO: 275 - protein, 19 aa, synthetic construct
  REGION 1..19: note = 6R8C-G8 HCDR3
ARHPGSAGYGYQYYYAMDP

SEQ ID NO: 276 - protein, 19 aa, synthetic construct
  REGION 1..19: note = 6R8C-D9 HCDR3
ARHPGSAGYGYQYYYAMDP

SEQ ID NO: 277 - protein, 19 aa, synthetic construct
  REGION 1..19: note = 6R8C-D10 HCDR3
ARHPGSAGYGYQYYYAMDP

SEQ ID NO: 278 - protein, 19 aa, synthetic construct
  REGION 1..19: note = 6R8C-F10 HCDR3
ARHPGSAGYGYQYYYAMDP

SEQ ID NO: 279 - protein, 214 aa, synthetic construct; identical to SEQ ID NO: 24
  REGION 1..214: note = 6R9A-A2

SEQ ID NO: 280 - protein, 214 aa, synthetic construct; identical to SEQ ID NO: 28
  REGION 1..214: note = 6R9B-B11

SEQ ID NO: 281 - protein, 214 aa, synthetic construct
  REGION 1..214: note = 6R9B-E9
DIQMTQSPSSLSASVGDRVTITCQASENIRYAVNWYQQKPGKAPKLLIYGANYRVDGVPSRFSGSGSGTDYTLTISSLQPEDFATYYCQQGYRVQILFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 282 - protein, 214 aa, synthetic construct; identical to SEQ ID NO: 24
  REGION 1..214: note = 6R9E-A8

SEQ ID NO: 283 - protein, 455 aa, synthetic construct
  REGION 1..455: note = 6R9A-A2
EVQLVESGGGLVQPGGSLRLSCTASGFSFPHQAMTWVRQAPGKGLEWIGVIASNADTYYASWAKGRFTISRDNSKNTLYLQMNSLRAEDTAVYYCARHPGSAGYGYQYYYAMDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 284 - protein, 455 aa, synthetic construct; identical to SEQ ID NO: 56
  REGION 1..455: note = 6R9B-B11

SEQ ID NO: 285 - protein, 455 aa, synthetic construct; identical to SEQ ID NO: 56
  REGION 1..455: note = 6R9B-E9

SEQ ID NO: 286 - protein, 455 aa, synthetic construct
  REGION 1..455: note = 6R9E-A8
EVQLVESGGGLVQPGGSLRLSCKASGSTISHQAVTWVRQAPGKGLEWIGVIASNADTYYASWAKGRFTISRDNSKNTLYLQMNSLRAEDTAVYYCARHPGSAGYGYQYYYAMDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 287 - protein, 11 aa, synthetic construct
  REGION 1..11: note = 6R9A-A2 LCDR1
QASEEVHYGIN

SEQ ID NO: 288 - protein, 11 aa, synthetic construct
  REGION 1..11: note = 6R9B-B11 LCDR1
QASQAIKYAIN

SEQ ID NO: 289 - protein, 11 aa, synthetic construct
  REGION 1..11: note = 6R9B-E9 LCDR1
QASENIRYAVN

SEQ ID NO: 290 - protein, 11 aa, synthetic construct
  REGION 1..11: note = 6RE-A8 LCDR1
QASEEVHYGIN

SEQ ID NO: 291 - protein, 8 aa, synthetic construct
  REGION 1..8: note = 6R9A-A2 LCDR2
YGANYRVD

SEQ ID NO: 292 - protein, 8 aa, synthetic construct
  REGION 1..8: note = 6R9B-B11 LCDR2
YGANYRGP

SEQ ID NO: 293 - protein, 8 aa, synthetic construct
  REGION 1..8: note = 6R9B-E9 LCDR2
YGANYRVD

SEQ ID NO: 294 - protein, 8 aa, synthetic construct
  REGION 1..8: note = 6RE-A8 LCDR2
YGANYRVD

SEQ ID NO: 295 - protein, 9 aa, synthetic construct
  REGION 1..9: note = 6R9A-A2 LCDR3
QQGYNVPTD

SEQ ID NO: 296 - protein, 9 aa, synthetic construct
  REGION 1..9: note = 6R9B-B11 LCDR3
QQGYIVPIT

SEQ ID NO: 297 - protein, 9 aa, synthetic construct
  REGION 1..9: note = 6R9B-E9 LCDR3
QQGYRVQIL

SEQ ID NO: 298 - protein, 9 aa, synthetic construct
  REGION 1..9: note = 6RE-A8 LCDR3
QQGYNVPTD

SEQ ID NO: 299 - protein, 13 aa, synthetic construct
  REGION 1..13: note = 6R9A-A2 HCDR1
TASGFSFPHQAMT

SEQ ID NO: 300 - protein, 13 aa, synthetic construct
  REGION 1..13: note = 6R9B-B11 HCDR1
KASGFTFSRQALT

SEQ ID NO: 301 - protein, 13 aa, synthetic construct
  REGION 1..13: note = 6R9B-E9 HCDR1
KASGFTFSRQALT

SEQ ID NO: 302 - protein, 13 aa, synthetic construct
  REGION 1..13: note = 6RE-A8 HCDR1
KASGSTISHQAVT

SEQ ID NO: 303 - protein, 18 aa, synthetic construct
  REGION 1..18: note = 6R9A-A2 HCDR2
IGVIASNADTYYASWAKG

SEQ ID NO: 304 - protein, 18 aa, synthetic construct
  REGION 1..18: note = 6R9B-B11 HCDR2
IGVIASNADTYYASWAKG

SEQ ID NO: 305 - protein, 18 aa, synthetic construct
  REGION 1..18: note = 6R9B-E9 HCDR2
IGVIASNADTYYASWAKG

SEQ ID NO: 306 - protein, 18 aa, synthetic construct
  REGION 1..18: note = 6RE-A8 HCDR2
IGVIASNADTYYASWAKG

SEQ ID NO: 307 - protein, 19 aa, synthetic construct
  REGION 1..19: note = 6R9A-A2 HCDR3
ARHPGSAGYGYQYYYAMDP

SEQ ID NO: 308 - protein, 19 aa, synthetic construct
  REGION 1..19: note = 6R9B-B11 HCDR3
ARHPGSAGYGYQYYYAMDP

SEQ ID NO: 309 - protein, 19 aa, synthetic construct
  REGION 1..19: note = 6R9B-E9 HCDR3
ARHPGSAGYGYQYYYAMDP

SEQ ID NO: 310 - protein, 19 aa, synthetic construct
  REGION 1..19: note = 6RE-A8 HCDR3
ARHPGSAGYGYQYYYAMDP

SEQ ID NO: 311 - protein, 214 aa, synthetic construct
  REGION 1..214: note = Light Chain Sequence of Clone S3R4F12
DIQMTQSPSSLSASVGDRVTITCLASEEIHYAINWYQQKPGKAPKLLIYGQSHLQDGVPSRFSGSGSGTDYTLTISSLQPEDFATYYCQQGYVVPILFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSSVTKSFNRGEC

SEQ ID NO: 312 - protein, 455 aa, synthetic construct
  REGION 1..455: note = Heavy Chain Sequence of Clone S3R4F12
EVQLVESGGGLVQPGGSLRLSCEASGYSIAPQAVSWVRQAPGKGLEWIGVIASNADTYYASWAKGRFTISRDNSKNTLYLQMNSLRAEDTAVYYCARHPGFAGYGYGMYYSMDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 313 - protein, 11 aa, synthetic construct
  REGION 1..11: note = S3R4F12 LCDR1
LASEEIHYAIN

SEQ ID NO: 314 - protein, 8 aa, synthetic construct
  REGION 1..8: note = S3R4F12 LCDR2
YGQSHLQD

SEQ ID NO: 315 - protein, 9 aa, synthetic construct
  REGION 1..9: note = S3R4F12 LCDR3
QQGYVVPIL

SEQ ID NO: 316 - protein, 13 aa, synthetic construct
  REGION 1..13: note = S3R4F12 HCDR1
EASGYSIAPQAVS

SEQ ID NO: 317 - protein, 18 aa, synthetic construct
  REGION 1..18: note = S3R4F12 HCDR2
IGVIASNADTYYASWAKG

SEQ ID NO: 318 - protein, 19 aa, synthetic construct
  REGION 1..19: note = S3R4F12 HCDR3
ARHPGFAGYGYGMYYSMDP

SEQ ID NO: 319 - protein, 11 aa, synthetic construct
  REGION 1..11: note = HxB10 LCDR1
QASEEVHYGIN

SEQ ID NO: 320 - protein, 8 aa, synthetic construct
  REGION 1..8: note = HxB10 LCDR2
YGANYRVD

SEQ ID NO: 321 - protein, 9 aa, synthetic construct
  REGION 1..9: note = HxB10 LCDR3
QQGYNVPTD

SEQ ID NO: 322 - protein, 13 aa, synthetic construct
  REGION 1..13: note = HxB10 HCDR1
TASGYSISHQAVT

SEQ ID NO: 323 - protein, 12 aa, synthetic construct
  REGION 1..12: note = HxB10 HCDR2
VIYADGASDYAS

SEQ ID NO: 324 - protein, 19 aa, synthetic construct
  REGION 1..19: note = HxB10 HCDR3
ARHPGDSGYGYHYYYSMDP

SEQ ID NO: 325 - protein, 11 aa, synthetic construct
  REGION 1..11: note = AR6-B9 LCDR1
QASENIRYAVN

SEQ ID NO: 326 - protein, 8 aa, synthetic construct
  REGION 1..8: note = AR6-B9 LCDR2
FGANYRWH

SEQ ID NO: 327 - protein, 9 aa, synthetic construct
  REGION 1..9: note = AR6-B9 LCDR3
QQGYNVPTD

SEQ ID NO: 328 - protein, 13 aa, synthetic construct
  REGION 1..13: note = AR6-B9 HCDR1
EASGSDLSHQALT

SEQ ID NO: 329 - protein, 12 aa, synthetic construct
  REGION 1..12: note = AR6-B9 HCDR2
VIYADGQGDYAS

SEQ ID NO: 330 - protein, 19 aa, synthetic construct
  REGION 1..19: note = AR6-B9 HCDR3
ARHPGDSGYGYHYYYSMDP

SEQ ID NO: 331 - protein, 11 aa, synthetic construct
  REGION 1..11: note = BR6-C10 LCDR1
QASEEVHYGIN

SEQ ID NO: 332 - protein, 8 aa, synthetic construct
  REGION 1..8: note = BR6-C10 LCDR2
YGANYRVD

SEQ ID NO: 333 - protein, 9 aa, synthetic construct
  REGION 1..9: note = BR6-C10 LCDR3
QQGYLVPGG

SEQ ID NO: 334 - protein, 13 aa, synthetic construct
  REGION 1..13: note = BR6-C10 HCDR1
TASGYSISHQAVT

SEQ ID NO: 335 - protein, 12 aa, synthetic construct
  REGION 1..12: note = BR6-C10 HCDR2
VIYADGASDYAS

SEQ ID NO: 336 - protein, 19 aa, synthetic construct
  REGION 1..19: note = BR6-C10 HCDR3
ARHPGDSGYGYHYYYSMDP

SEQ ID NO: 337 - protein, 11 aa, synthetic construct
  REGION 1..11: note = CR6-C8 LCDR1
QASEDIHYALN

SEQ ID NO: 338 - protein, 8 aa, synthetic construct
  REGION 1..8: note = CR6-C8 LCDR2
YGMNHLMD

SEQ ID NO: 339 - protein, 9 aa, synthetic construct
  REGION 1..9: note = CR6-C8 LCDR3
QQGYVVPII

SEQ ID NO: 340 - protein, 13 aa, synthetic construct
  REGION 1..13: note = CR6-C8 HCDR1
AASGYTISHQAVT

SEQ ID NO: 341 - protein, 12 aa, synthetic construct
  REGION 1..12: note = CR6-C8 HCDR2
VIASNADTYYAS

SEQ ID NO: 342 - protein, 19 aa, synthetic construct
  REGION 1..19: note = CR6-C8 HCDR3
ARHPGFAGYGYGMYYSMDP

SEQ ID NO: 343 - protein, 11 aa, synthetic construct
  REGION 1..11: note = GR4-B12 LCDR1
QASEEVHYGIN

SEQ ID NO: 344 - protein, 8 aa, synthetic construct
  REGION 1..8: note = GR4-B12 LCDR2
YGANYRWH

SEQ ID NO: 345 - protein, 9 aa, synthetic construct
  REGION 1..9: note = GR4-B12 LCDR3
QQGYNVPGH

SEQ ID NO: 346 - protein, 13 aa, synthetic construct
  REGION 1..13: note = GR4-B12 HCDR1
TASGYSISHQAVT

SEQ ID NO: 347 - protein, 12 aa, synthetic construct
  REGION 1..12: note = GR4-B12 HCDR2
VIYSDGRSDYAS

SEQ ID NO: 348 - protein, 19 aa, synthetic construct
  REGION 1..19: note = GR4-B12 HCDR3
ARHPGDSGYGYHYYYSMDP

SEQ ID NO: 349 - protein, 11 aa, synthetic construct
  REGION 1..11: note = GR4-E11 LCDR1
QASEEVHYGIN

SEQ ID NO: 350 - protein, 8 aa, synthetic construct
  REGION 1..8: note = GR4-E11 LCDR2
YGANYRWH

SEQ ID NO: 351 - protein, 9 aa, synthetic construct
  REGION 1..9: note = GR4-E11 LCDR3
QQGYNVPGH

SEQ ID NO: 352 - protein, 13 aa, synthetic construct
  REGION 1..13: note = GR4-E11 HCDR1
KASGYSISHQAVT

SEQ ID NO: 353 - protein, 12 aa, synthetic construct
  REGION 1..12: note = GR4-E11 HCDR2
IIYHDGRSDYAS

SEQ ID NO: 354 - protein, 19 aa, synthetic construct
  REGION 1..19: note = GR4-E11 HCDR3
ARHPGDSGYGYHYYYSMDP

SEQ ID NO: 355 - DNA, 321 bp, synthetic construct
  misc_feature 1..321: note = C-A11
  source 1..321: mol_type = other DNA
gccatcgagatgacccagtctccatcctccctggctgcatctgtgggagacacagtcaccatcacttgtaaggccagtgaggacattcgttatgggttaaactggtatcagcagaaactagggatagctcctaagctcctgatctatggggcaaacactttagaggctggggtcccatcgaggttcagtggcagcggatcagagaccgattacaccctcaccatcagcagcgtgcaggctgaagatgcaggaatttattactgtcagcaaggatatagtacccctttgacttttggagctggcaccaaggtggaaatcaaa

SEQ ID NO: 356 - DNA, 321 bp, synthetic construct
  misc_feature 1..321: note = hN2F
  source 1..321: mol_type = other DNA
gacatccagatgacccagtctccatcctccctgtctgcatctgtaggagacagagtcaccatcacttgcaaggccagtgaggacattcgttatgggttaaactggtatcagcagaaaccagggaaagcccctaagctcctgatctatggggcaaacactttagaggctggggtcccatcaaggttcagtggcagtggatctgggacagatttcactctcaccatcagcagtctgcaacctgaagattttgcaacttactactgtcagcaaggatatagtacccctttgactttcggcggagggaccaaggtggagatcaaa

SEQ ID NO: 357 - DNA, 321 bp, synthetic construct
  misc_feature 1..321: note = hN2Y
  source 1..321: mol_type = other DNA
gacatccagatgacccagtctccatcctccctgtctgcatctgtaggagacagagtcaccatcacttgcaaggccagtgaggacattcgttatgggttaaactggtatcagcagaaaccagggaaagcccctaagctcctgatctatggggcaaacactttagaggctggggtcccatcaaggttcagtggcagtggatctgggacagattacactctcaccatcagcagtctgcaacctgaagattttgcaacttactactgtcagcaaggatatagtacccctttgactttcggcggagggaccaaggtggagatcaaa

SEQ ID NO: 358 - DNA, 360 bp, synthetic construct
  misc_feature 1..360: note = C-A11
  source 1..360: mol_type = other DNA
cagtcgctggaggagtccgggggtcgcctggtcacgcctgggacacccctgacactcacctgcacagtctctggattctccctcagtacctatgcaatgagctgggtccgccaggctccagggaagggactggaatggatcggagtcattgctagtaatgctgacacatactacgcgagctgggcgaaaggccgaatcaccatctccaaaacctcgaccacggtggatctgaagatcaccagtccgacgaccgaggacacggccacctatttctgtgccagacatcctggtagtgctggttatggttatgctgtttattacgccatggacccctggggcccagggaccctcgtcaccgtc

SEQ ID NO: 359 - DNA, 360 bp, synthetic construct
  misc_feature 1..360: note = hN2F
  source 1..360: mol_type = other DNA
gaggtgcagctggtggagtctgggggaggcttggtacagcctggggggtccctgagactctcctgtacagtctctggattctccctcagtacctatgcaatgagctgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatcctggtagtgctggttatggttatgctgtttattacgccatggacccctggggccaaggaaccctg

SEQ ID NO: 360 - DNA, 360 bp, synthetic construct; identical to SEQ ID NO: 359
  misc_feature 1..360: note = hN2Y
  source 1..360: mol_type = other DNA

SEQ ID NO: 361 - DNA, 642 bp, synthetic construct
  misc_feature 1..642: note = 3R4-H4
  source 1..642: mol_type = other DNA
gacatccagatgacccagtctccatcctccctgtctgcatctgtaggagacagagtcaccatcacttgccaggccagtcaggggattcattatgcgattaattggtatcagcagaaaccagggaaagcccctaagctcctgatctatgggcataattatcttgaggagggggtcccatcaaggttcagtggcagtggatctgggacagactacactctcaccatcagcagtctgcaacctgaagattttgcaacttactactgtcaacaaggatatattgtgccgattgttttcggcggagggaccaaggtggagatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 362 - DNA, 642 bp, synthetic construct
  misc_feature 1..642: note = 5R4-H1
  source 1..642: mol_type = other DNA
gacatccagatgacccagtctccatcctccctgtctgcatctgtaggagacagagtcaccatcacttgccaggccagtgagccgattcattatgctattaattggtatcagcagaaaccagggaaagcccctaagctcctgatctatgggactagttatcgtttggatggggtcccatcaaggttcagtggcagtggatctgggacagatttcactctcaccatcagcagtctgcaacctgaagattttgcaacttactactgtcaacaaggatatactgtgcctttgcatttcggcggagggaccaaggtggagatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 363 - DNA, 642 bp, synthetic construct
  misc_feature 1..642: note = 5R4-F3
  source 1..642: mol_type = other DNA
gacatccagatgacccagtctccatcctccctgtctgcatctgtaggagacagagtcaccatcacttgccaggccagtgaggacattcgttatgggttaaactggtatcagcagaaaccagggaaagcccctaagctcctgatctatgggactagttatcgtcgtgatggggtcccatcaaggttcagtggcagtggatctgggacagattacactctcaccatcagcagtctgcaacctgaagattttgcaacttactactgtcaacaaggatataatcttccgactaggttcggcggagggaccaaggtggagatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 364 - DNA, 642 bp, synthetic construct
  misc_feature 1..642: note = 5R4-E11
  source 1..642: mol_type = other DNA
gacatccagatgacccagtctccatcctccctgtctgcatctgtaggagacagagtcaccatcacttgcaaagccagtgagcctatttattatgcgattaattggtatcagcagaaaccagggaaagcccctaagctcctgatctatggtgcgagtggtcttgcggatggggtcccatcaaggttcagtggcagtggatctgggacagattacactctcaccatcagcagtctgcaacctgaagattttgcaacttactactgtcaacaaggatatattgtgcctattatgttcggcggagggaccaaggtggagatcaaacggactgtggctgcaccatctgtctccatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 365 - DNA, 642 bp, synthetic construct
  misc_feature 1..642: note = 6R4-H1
  source 1..642: mol_type = other DNA
gacatccagatgacccagtctccatcctccctgtctgcatctgtaggagacagagtcaccatcacttgccaggccagtcagcctattttttatggtattgattggtatcagcagaaaccagggaaagcccctaagctcctgatctatgggactagttatcgtgcggagggggtcccatcaaggttcagtggcagtggatctgggacagattacactctcaccatcagcagtctgcaacctgaagattttgcaacttactactgtcaacaaggatatattgtgcctattacgttcggcggagggaccaaggtggagatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacggagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 366 - DNA, 642 bp, synthetic construct
  misc_feature 1..642: note = 6R4-A7
  source 1..642: mol_type = other DNA
gacatccagatgacccagtctccatcctccctgtctgcatctgtaggagacagagtcaccatcacttgccaggccagtcagcagatttattatgctattaattggtatcagcagaaaccagggaaagcccctaagctcctgatctatggtgcgaattatcgtgaggatggggtcccatcaaggttcagtggcagtggatctgggacagattacactctcaccatcagcagtctgcaacctgaagattttgcaacttactactgtcaacaaggatatctggttcctgttggtttcggcggagggaccaaggtggagatcaaacggactgtggccgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcggactacgagaaacacaaagtctacgcctgcgaagtcacccatcagggcctgagctcgcccgtcacaaagagcttcaacaggggagagtgt

SEQ ID NO: 367 - DNA, 642 bp, synthetic construct
  misc_feature 1..642: note = 6R4-G10
  source 1..642: mol_type = other DNA
gacatccagatgacccagtctccatcctccctgtctgcatctgtaggagacagagtcaccatcacttgccaggccagtgagcgtatttattatgcgattaattggtatcagcagaaaccagggaaagcccctaagctcctgatctatggtgcgaattatcgtgtggatggggtcccatcaaggttcagtggcagtggatctgggacagattacactctcaccatcagcagtctgcaacctgaagattttgcaacttactactgtcaacaaggatatattgttccgacgaatttcggcggagggaccaaggtggagatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcgcagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 368 - DNA, 642 bp, synthetic construct
  misc_feature 1..642: note = 6R4-G11
  source 1..642: mol_type = other DNA
gacatccagatgacccagtctccatcctccctgtctgcatctgtaggagacagagtcaccatcacttgcaaggccagtgaggacattcgttatgggttaaactggtatcagcagaaaccagggaaagcccctaagctcctgatctatggggctaatcatcttagggagggggtcccatcaaggttcagtggcagtggatctgggacagattacactctcaccatcagcagtctgcaacctgaagattttgcaacttactactgtggttgggggtatagtgttcctttgactttcggcggagggaccaaggtggagatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgggcagagcagactacgagaaacacaaagtctacgcctgcgaagtcacccatcagggcctgagctcgcccgtcacaaagagcttcaacaggggagagtgt

SEQ ID NO: 369 - DNA, 642 bp, synthetic construct
  misc_feature 1..642: note = 6R4-E12
  source 1..642: mol_type = other DNA
gacatccagatgacccagtctccatcctccctgtctgcatctgtaggagacagagtcaccatcacttgccaggccagtgagaagattcattatgcgattaattggtatcagcagaaaccagggaaagcccctaagctcctgatctatgggcatagtcatcttgaggatggggtcccatcaaggttcagtggcagtggatctgggacagattacactctcaccatcagcagtctgcaacctgaagattttgcaacttactactgtcaacaaggatatattgttccgaattctttcggcggagggaccaaggtggagatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 370 - DNA, 642 bp, synthetic construct
  misc_feature 1..642: note = 6R4-E3
  source 1..642: mol_type = other DNA
gacatccagatgacccagtctccatcctccctgtctgcatctgtaggagacagagtcaccatcacttgccaggccagtcagctgatttattatggtattaattggtatcagcagaaaccagggaaagcccctaagctcctgatctatggtgcgaattatcgtgtggatggggtcccatcaaggttcagtggcagtggatctgggacagattacactctcaccatcagcagtctgcaacctgaagattttgcaacttactactgtcaacaaggatatagtgttccgcttgatttcggcggagggaccaaggtggagatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 371 - DNA, 642 bp, synthetic construct
  misc_feature 1..642: note = 6R4-H5
  source 1..642: mol_type = other DNA
gacatccagatgacccagtctccatcctccctgtctgcatctgtaggagacagagtcaccatcacttgcaaggccagtgaggacattcgttatgggttaaactggtatcagcagaaaccagggaaagcccctaagctcctgatctatgggcatacttatcgtttggatggggtcccatcaaggttcagtggcagtggatctgggacagattacactctcaccatcagcagtctgcaacctgaagattttgcaacttactactgtcaacaaggatatgtggtgccggtggggttcggcggagggaccaaggtggagatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 372 - DNA, 642 bp, synthetic construct; identical to SEQ ID NO: 361
  misc_feature 1..642: note = 6R4-H11
  source 1..642: mol_type = other DNA

SEQ ID NO: 373 - DNA, 642 bp, synthetic construct
  misc_feature 1..642: note = 6R6-A7
  source 1..642: mol_type = other DNA
gacatccagatgacccagtctccatcctccctgtctgcatctgtaggagacagagtcaccatcacttgccaggccagtgagaagatttattatgcgattaattggtatcagcagaaaccagggaaagcccctaagctcctgatctatgggcatagttatcgtttggagggggtcccatcaaggttcagtggcagtggatctgggacagattacactctcaccatcagcagtctgcaacctgaagattttgcaacttactactgtcaacaaggatatattgtgcctaatcgtttcggcggagggaccaaggtggagatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 374 - DNA, 642 bp, synthetic construct; identical to SEQ ID NO: 373
  misc_feature 1..642: note = 6R6-E1
  source 1..642: mol_type = other DNA

SEQ ID NO: 375 - DNA, 642 bp, synthetic construct
  misc_feature 1..642: note = 6R6-E2
  source 1..642: mol_type = other DNA
gacatccagatgacccagtctccatcctccctgtctgcatctgtaggagacagagtcaccatcacttgcctggccagtcagcctatttattatggtcttaattggtatcagcagaaaccagggaaagcccctaagctcctgatctatgggacgagtgcgcttgtggatggggtcccatcaaggttcagtggcagtggatctgggacagattacactctcaccatcagcagtctgcaacctgaagattttgcaacttactactgtcaacagggatatattgtgcctgtgtcgttcggcggagggaccaaggtggagatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagttcgcccgtcacaaagagcttcaacaggggagagtgt

SEQ ID NO: 376 - DNA, 642 bp, synthetic construct
  misc_feature 1..642: note = 6R6-E3
  source 1..642: mol_type = other DNA
gacatccagatgacccagtctccatcctccctgtctgcatctgtaggagacagagtcaccatcacttgccaggccagtgagaatattaggtatgctattaattggtatcagcagaaaccagggaaagcccctaagctcctgatctatggtgcgaattatcgtgtggatggggtcccatcaaggttcagtggcagtggatctgggacagattacactctcaccatcagcagtctgcaacctgaagattttgcaacttactactgtcaacaaggatataatgtgcctactgatttcggcggagggaccaaggtggagatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacggcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 377 - DNA, 642 bp, synthetic construct
  misc_feature 1..642: note = 6R6-E4
  source 1..642: mol_type = other DNA
gacatccagatgacccagtctccatcctccctgtctgcatctgtaggagacagagtcaccatcacttgccaggccagtgagaatattaggtatgctgttaattggtatcagcagaaaccagggaaagcccctaagctcctgatctatggtgcgaattatcgtgtggatggggtcccatcaaggttcagtggcagtggatctgggacagattacactctcaccatcagcagtctgcaacctgaagattttgcaacttactactgtcaacaaggatatcgtgtggctattgggttcggcggagggaccaaggtggagatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagtacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 378 - DNA, 642 bp, synthetic construct
  misc_feature 1..642: note = 6R6-F3
  source 1..642: mol_type = other DNA
gacatccagatgacccagtctccatcctccctgtctgcatctgtaggagacagagtcaccatcacttgccaggccagtgaggaggttcattatgggattaattggtatcagcagaaaccagggaaagcccctaagctcctgatctatggtgcgaattatcgtgtggatggggtcccatcaaggttcagtggcagtggatctgggacagattacactctcaccatcagcagtctgcaacctgaagattttgcaacttactactgtcaacaaggatataatgtgcctactgatttcggcggagggaccaaggtggagatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 379 - DNA, 642 bp, synthetic construct
  misc_feature 1..642: note = 6R6-F7
  source 1..642: mol_type = other DNA
gacatccagatgacccagtctccatcctccctgtccgcatctgtaggagacagagtcaccatcacttgccaggccagtgagaggatttattatgggcttaattggtatcagcagaaaccagggaaagcccctaagctcctgatctatggggctactggtcgtgcggatggggtcccatcaaggttcagtggcagtggatctgggacagattacactctcaccatcagcagtctgcaacctgaagattttgcaacttactactgtcaacaaggatatattgtgccgtcggggttcggcggagggaccaaggtggagatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 380 - DNA, 642 bp, synthetic construct
  misc_feature 1..642: note = 6R6-F9
  source 1..642: mol_type = other DNA
gacatccagatgacccagtctccatcctccctgtctgcatctgtaggagacagagtcaccatcacttgcaaggccagtgaggacattcgttatgggttaaactggtatcagcagaaaccagggaaagcccctaagctcctgatctatgggcatacttatcgtttggatggggtcccatcaaggttcagtggcagtggatctgggacagattacactctcaccatcagcagtctgcaacctgaagattttgcaacttactactgtcaacaaggatatattgtgcctaatatgttcggcggagggaccaaggtggagatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 381 - DNA, 642 bp, synthetic construct
  misc_feature 1..642: note = 6R6-F11
  source 1..642: mol_type = other DNA
gacatccagatgacccagtctccatcctccctgtctgcatctgtaggagacagagtcaccatcacttgccaggccagtgagcagatttattatggtattaattggtatcagcagaaaccagggaaagcccctaagctcctgatctatggtgcgactggtcgtagggatggggtcccatcaaggttcagtggcagtggatctgggacagattacactctcaccatcagcagtctgcaacctgaagattttgcaacttactactgtcaacaaggatatattgtgcctacgacgttcggcggagggaccaaggtggagatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 382 - DNA, 642 bp, synthetic construct
  misc_feature 1..642: note = 6R6-F12
  source 1..642: mol_type = other DNA
gacatccagatgacccagtctccatcctccctgtctgcatctgtaggagacagagtcaccatcacttgccaggccagtcaggcgattaagtatgcgattaattggtatcagcagaaaccagggaaagcccctaagctcctgatctatggtgctaattatcgtgggccgggggtcccatcaaggttcagtggcagtggatctgggacagattacactctcaccatcagcagtctgcaacctgaagattttgcaacttactactgtcaacaaggatatattgtgcctattacgttcggcggagggaccaaggtggagatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 383 - DNA, 642 bp, synthetic construct
  misc_feature 1..642: note = 3R6D11Q
  source 1..642: mol_type = other DNA
gacatccagatgacccagtctccatcctccctgtctgcatctgtaggagacagagtcaccatcacttgccaggccagtgagccgatttattatgctattaattggtatcagcagaaaccagggaaagcccctaagctcctgatctatgggcagagtcatcttcaggatggggtcccatcaaggttcagtggcagtggatctgggacagattacactctcaccatcagcagtctgcaacctgaagattttgcaacttactactgtcaacaaggatatcgggtgcctacttttttcggcggagggaccaaggtggagatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 384 - DNA, 642 bp, synthetic construct
  misc_feature 1..642: note = 6R6-E6Q
  source 1..642: mol_type = other DNA
gacatccagatgacccagtctccatcctccctgtctgcatctgtaggagacagagtcaccatcacttgccaggccagtgagcaggtttattatggtcttaattggtatcagcagaaaccagggaaagcccctaagctcctgatctatggtgcgaattatcgtgtggatggggtcccatcaaggttcagtggcagtggatctgggacagattacactctcaccatcagcagtctgcaacctgaagattttgcaacttactactgtcaacaaggatatgttgttccgaatgtgttcggcggagggaccaaggtggagatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 385 - DNA, 642 bp, synthetic construct; identical to SEQ ID NO: 376
  misc_feature 1..642: note = 6R8A-E11
  source 1..642: mol_type = other DNA

SEQ ID NO: 386 - DNA, 642 bp, synthetic construct
  misc_feature 1..642: note = 6R8A-F2
  source 1..642: mol_type = other DNA
gacatccagatgacccagtctccatcctccctgtctgcatctgtaggagacagagtcaccatcacttgccaggccagtgagaaggtttattatggggttaattggtatcagcagaaaccagggaaagcccctaagctcctgatctatggtacgaattatcgtctggatggggtcccatcaaggttcagtggcagtggatctgggacagattccactctcaccatcagcagtctgcaacctgaagattttgcaacttactactgtcaacaaggatataatgttccgaatgcgttcggcggagggaccaaggtggagatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 387 - DNA, 642 bp, synthetic construct; identical to SEQ ID NO: 376
  misc_feature 1..642: note = 6R8A-E1
  source 1..642: mol_type = other DNA

SEQ ID NO: 388 - DNA, 642 bp, synthetic construct; identical to SEQ ID NO: 378
  misc_feature 1..642: note = 6R8A-A2
  source 1..642: mol_type = other DNA

SEQ ID NO: 389 - DNA, 642 bp, synthetic construct; identical to SEQ ID NO: 378
  misc_feature 1..642: note = 6R8C-G3
  source 1..642: mol_type = other DNA

SEQ ID NO: 390 - DNA, 642 bp, synthetic construct; identical to SEQ ID NO: 376
  misc_feature 1..642: note = 6R8C-G4
  source 1..642: mol_type = other DNA

SEQ ID NO: 391 - DNA, 642 bp, synthetic construct; identical to SEQ ID NO: 378
  misc_feature 1..642: note = 6R8C-G8
  source 1..642: mol_type = other DNA

SEQ ID NO: 392 - DNA, 642 bp, synthetic construct; identical to SEQ ID NO: 378
  misc_feature 1..642: note = 6R8C-D9
  source 1..642: mol_type = other DNA

SEQ ID NO: 393 - DNA, 642 bp, synthetic construct; identical to SEQ ID NO: 378
  misc_feature 1..642: note = 6R8C-D10
  source 1..642: mol_type = other DNA

SEQ ID NO: 394 - DNA, 642 bp, synthetic construct
  misc_feature 1..642: note = 6R8C-F10
  source 1..642: mol_type = other DNA
gacatccagatgacccagtctccatcctccctgtccgcatctgtaggagacagagtcaccatcacttgcaaggccagtgaggacattcgttatgggttaaactggtatcagcagaaaccagggaaagcccctaagctcctgatctatggtgcgaattatcgtgtggatggggtcccatcaaggttcagtggcagtggatctgggacagattacactctcaccatcagcagtctgcaacctgaagattttgcaacttactactgtcaacaaggatataatgtgcctaattttttcggcggagggaccaaggtggagatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 395 - DNA, 1365 bp, synthetic construct
  misc_feature 1..1365: note = 3R4H4
  source 1..1365: mol_type = other DNA
gaggtgcagctggtggagtctgggggaggcttggtacagcctggggggtccctgagactctcctgtgaggcttctggatttacttttcctcttcaggctatgagttgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatccgggttttgctggttatggttatgggcattattacgccatggacccctggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 396 - DNA, 1365 bp, synthetic construct
  misc_feature 1..1365: note = 5R4H1
  source 1..1365: mol_type = other DNA
gaggtgcagctggtggagtctgggggaggcttggtacagcctggggggtccctgagactctcctgtgcggcttctggatatagtgttacgcatcaggctgtgacttgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatcctggtagtgctggttatggttatcagtattattatagtatggacccctggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 397 - DNA, 1365 bp, synthetic construct
  misc_feature 1..1365: note = 5R4F3
  source 1..1365: mol_type = other DNA
gaggtgcagctggtggagtctgggggaggcttggtacagcctggggggtccctgagactctcctgtaaggcttctggatttacttttcctaagcaggctatgacttgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatccgggttttgctggttatggttatgggcattattacgccatggacccctggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 398 - DNA, 1365 bp, synthetic construct
  misc_feature 1..1365: note = 5R4E11
  source 1..1365: mol_type = other DNA
gaggtgcagctggtggagtctgggggaggcttggtacagcctggggggtccctgagactctcctgtaaggcttctggatttacttttaagccgcaggctatgacatgggtccgccaggctccagggaaggggctggagtggatcggagtcattgctagtaatgctgacacatactacgcgagctgggcgaaaggccgattcaccatctccagagacaattccaagaacacgctgtatctgcaaatgaacagcctgagagccgaggacacggccgtatattactgtgccagacatccgggttttgctggttatggttatgggcattattacgccatggacccctggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 399 - DNA, 1365 bp, synthetic construct
  misc_feature 1..1365: note = 6R4H1
  source 1..1365: mol_type = other DNA
gaggtgcagctggtggagtctgggggaggcttggtacagcctggggggtccctgagactctcctgtgaggcttctggatatactttttggtcgcaggctatgacttgggtccgccaggctccagggaaggggctggagtggatcggagtcattgctagtaatgctgacacatactacgcgagctgggtgaaaggccggttcaccatctccagagacaattccaagaacacgctgtatctgcaaatgaacagcctgagagccgaggacacggccgtatattactgtgccagacatccgggttttgctggttatggttatcagtattattatagtatggacccctggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 400 - DNA, 1365 bp, synthetic construct
  misc_feature 1..1365: note = 6R4A7
  source 1..1365: mol_type = other DNA
gaggtgcagctggtggagtctgggggaggcttggtacagcctggggggtccctgagactctcctgtaaggcttctggaattagtattacggagaaggctgtgacttgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatccgggttttgctggttatggttatgggcattattacgccatggacccctggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 401 - DNA, 1365 bp, synthetic construct
  misc_feature 1..1365: note = 6R4G10
  source 1..1365: mol_type = other DNA
gaggtgcagctggtggagtctgggggaggcttggtacagcctgggcggtccctgagactctcctgtgcggcttctggatatactattgggcctcaggcgatgacttgggtccgccaggctccagggaaggggctggagtggatcggagtcattgctagtaatgctgacacatactacgcgagctgggcgaaaggccgattcaccatctccagagacaattccaagaacacgctgtatctgcaaatgaacagcctgagagccgaggacacggccgtatattactgtgccagacatccgggttttgctggttatggttatgggcattattacgccatggacccctggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 402 - DNA, 1365 bp, synthetic construct
  misc_feature 1..1365: note = 6R4G11
  source 1..1365: mol_type = other DNA
gaggtgcagctggtggagtctgggggaggcttggtacagcctggggggtccctgagactctcctgtaaggcttctggatatagtcttgcgccgcaggcgatgacttgggtccgccaggctccagggaaggggctggagtggatcggagtcattgctagtaatgctgacacatactacgcgagctgggcgaaaggccgattcaccatctccagagacaattccaagaacacgctgtatctgcaaatgaacagcctgagagccgaggacacggccgtatattactgtgccagacatccgggttttgctggttatggttatgggcattattacgccatggacccctggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 403 - DNA, 1365 bp, synthetic construct
  misc_feature 1..1365: note = 6R4E12
  source 1..1365: mol_type = other DNA
gaggtgcagctggtggagtctgggggaggcttggtacagcttggggggtccctgagactctcctgtaaggcttctggatatagtcttgcgccgcaggcgatgacttgggtccgccaggctccagggaaggggctggagtggatcggagtcattgctagtaatgctgacacatactacgcgagctgggcgaaaggccgattcaccatctccagagacaattccaagaacacgctgtatctgcaaatgaacagcctgagagccgaggacacggccgtatattactgtgccagacatccgggttttgctggttatggttatgggcattattacgccatggacccctggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 404 - DNA, 1365 bp, synthetic construct
  misc_feature 1..1365: note = 6R4E3
  source 1..1365: mol_type = other DNA
gaggtgcagctggtggagtctgggggaggcttggtacagcctggggggtccctgagactctcctgtaaggcttctggatatacttttgagactcaggcgatgacttgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatcctggtagtgctggttatggttatcagtattattacgccatggacccctggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 405 - DNA, 1365 bp, synthetic construct
  misc_feature 1..1365: note = 6R4H5
  source 1..1365: mol_type = other DNA
gaggtgcagctggtggagtctgggggaggcttggtacagcctggggggtccctgagactctcctgtgcggcttctggatatactgttaagaggcaggctgtcacttgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatcctggtagtgctggttatggttatcagtattattacgccatggacccctggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 406 - DNA, 1365 bp, synthetic construct
  misc_feature 1..1365: note = 6R4H11
  source 1..1365: mol_type = other DNA
gaggtgcagctggtggagtctgggggaggcttggtacagcctggggggtccctgagactctcctgtaaggcttctggatttacttttaagccgcaggctatgacatgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatcctggtagtgctggttatggttatcagtattattacgccatggacccctggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 407 - DNA, 1365 bp, synthetic construct
  misc_feature 1..1365: note = 6R6A7
  source 1..1365: mol_type = other DNA
gaggtgcagctggtggagtctgggggaggcttggtacagcttggggggtccctgagactctcctgtaaggcttctggatatagtcttgcgccgcaggcgatgacttgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatcctggtagtgctggttatggttatcagtattattatagtatggacccctggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 408 - DNA, 1365 bp, synthetic construct
  misc_feature 1..1365: note = 6R6E1
  source 1..1365: mol_type = other DNA
gaggtgcagctggtggagtctgggggaggcttggtacagcctggggggtccctgagactctcctgtgaggcttctggatttacttttccgaatcaggctatgacttgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatccgggttttgctggttatggttatggtatgtattattcgatggacccctggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 409 - DNA, 1365 bp, synthetic construct
  misc_feature 1..1365: note = 6R6E2
  source 1..1365: mol_type = other DNA
gaggtgcagctggtggagtctgggggaggcttggtacagcctggggggtccctgagactctcctgtaaggcttctggaattagtgttaatactaaggcgctcacttgggtccgccaggctccagggaaggggctggagtggatcggagtcattgctagtaatgctgacacatactacgcgagctgggcgaaaggccgattcaccatctccagagacaattccaagaacacgctgtatctgcaaatgaacagcctgagagccgaggacacggccgtatattactgtgccagacatccgggttttgctggttatggttatgggcattattacgccatggacccctggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 410 - DNA, 1365 bp, synthetic construct
  misc_feature 1..1365: note = 6R6E3
  source 1..1365: mol_type = other DNA
gaggtgcagctggtggagtctgggggaggcttggtacagcctggggggtccctgagactctcctgtaaggcttctggatttacttttagtcgtcaggctctcacttgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatcctggtagtgctggttatggttatcagtattattacgccatggacccctggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 411 - DNA, 1365 bp, synthetic construct; identical to SEQ ID NO: 405
  misc_feature 1..1365: note = 6R6E4
  source 1..1365: mol_type = other DNA

SEQ ID NO: 412 - DNA, 1365 bp, synthetic construct
  misc_feature 1..1365: note = 6R6F3
  source 1..1365: mol_type = other DNA
gaggtgcagctggtggagtctgggggaggcttggtacagcctggggggtccctgagactctcctgtgaggcttctggaattagtatttcgcctaatgctgtgacttgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatcctggtagtgctggttatggttatcagtattattacgccatggacccctggggccaaggaaccctggtcaccgtctcctcagcctccaccaagggcccatcggtcttccccctggcacccccctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 413 - DNA, 1365 bp, synthetic construct; identical to SEQ ID NO: 395
  misc_feature 1..1365: note = 6R6F7
  source 1..1365: mol_type = other DNA

SEQ ID NO: 414 - DNA, 1365 bp, synthetic construct
  misc_feature 1..1365: note = 6R6F9
  source 1..1365: mol_type = other DNA
gaggtgcagctggtggagtctgggggaggcttggtacagcctggggggtccctgagactctcctgtgaggcttctggaattgatcttagtcctcaggctatgacatgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatcctggtagtgctggttatggttatcagtattattacgccatggacccctggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 415 - DNA, 1365 bp, synthetic construct
  misc_feature 1..1365: note = 6R6F11
  source 1..1365: mol_type = other DNA
gaggtgcagctggtggagtctgggggaggcttggtacagcctggggggtccctgagactctcctgtgaggcttctggatatacttttgcttcgcaggcgatgacttgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatccgggttttgctggttatggttatgggcattattacgccatggacccctggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 416 - DNA, 1365 bp, synthetic construct; identical to SEQ ID NO: 410
  misc_feature 1..1365: note = 6R6F12
  source 1..1365: mol_type = other DNA

SEQ ID NO: 417 - DNA, 1365 bp, synthetic construct
  misc_feature 1..1365: note = 3R6D11Q
  source 1..1365: mol_type = other DNA
gaggtgcagctggtggagtctgggggaggcttggtacagcttggggggtccctgagactctcctgtaaggcttctggatatagtcttgcgccgcaggcgatgacttgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatccgggttttgctggttatggttatgggcattattacgccatggacccctggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 418 - DNA, 1365 bp, synthetic construct
  misc_feature 1..1365: note = 6R6E6Q
  source 1..1365: mol_type = other DNA
gaggtgcagctggtggagtctgggggaggcttggtacagcctggggggtccctgagactctcctgtaaggcttctggatatagtcttgcgccgcaggcgatgacttgggtccgccaggctccagggaaggggctggagtggatcggagtcattgctagtaatgctgacacatactacgcgagctgggcgaaaggccgattcaccatctccagagacaattccaagaacacgctgtatctgcaaatgaacagcctgagagccgaggacacggccgtatattactgtgccagacatcctggtagtgctggttatggttatcagtattattacgccatggacccctggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 419 - DNA, 1365 bp, synthetic construct; identical to SEQ ID NO: 410
  misc_feature 1..1365: note = 6R8AE11
  source 1..1365: mol_type = other DNA

SEQ ID NO: 420 - DNA, 1365 bp, synthetic construct
  misc_feature 1..1365: note = 6R8AF2
  source 1..1365: mol_type = other DNA
gaggtgcagctggtggagtctgggggaggcttggtacagcctggggggtccctgagactctcctgtaaggcttctggatatagtcttagtagtcaggctctgacttgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatcctggtagtgctggttatggttatcagtattattacgccatggacccctggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 421 - DNA, 1365 bp, synthetic construct
  misc_feature 1..1365: note = 6R8AE1
  source 1..1365: mol_type = other DNA
gaggtgcagctggtggagtctgggggaggcttggtacagcctggggggtccctgagactctcctgtaaggcttctggatatagtcttgcgccgcaggcgatgacttgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatcctggtagtgctggttatggttatcagtattattacgccatggacccctggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 422 - DNA, 1365 bp, synthetic construct
  misc_feature 1..1365: note = 6R8AA2
  source 1..1365: mol_type = other DNA
gaggtgcagctggtggagtctgggggaggcttggtacagcctggggggtccctgagactctcctgtaaggcttctggatatagtcttaataagcaggctctcacttgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatcctggtagtgctggttatggttatcagtattattacgccatggacccctggggccaaggaaccctggtcaccgtctcctcagcctccaccaagggcccatcggtcttccccctggcaccctcctccaagagcacctctggggacacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 423 - DNA, 1365 bp, synthetic construct
  misc_feature 1..1365: note = 6R8CG3
  source 1..1365: mol_type = other DNA
gaggtgcagctggttgagtctgggggaggcttggtacagcctggggggtccctgagactctcctgtaaggcttctggaattagtcttgcgccgtatgctatcacttgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatcctggtagtgctggttatggttatcagtattattatagtatggacccctggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 424 - DNA, 1365 bp, synthetic construct; identical to SEQ ID NO: 410
  misc_feature 1..1365: note = 6R8CG4
  source 1..1365: mol_type = other DNA

SEQ ID NO: 425 - DNA, 1365 bp, synthetic construct
  misc_feature 1..1365: note = 6R8CG8
  source 1..1365: mol_type = other DNA
gaggtgcagctggtggagtctgggggaggcttggtacagcttggggggtccctgagactctcctgtaaggcttctggatatagtcttgcgccgcaggcgatgacttgggtccgccaggctccagggaaggggctggagtggatcggagtcattgctagtaatgctgacacatactacgcgagctgggcgaaaggccgattcaccatctccagagacaattccaagaacacgctgtatctgcaaatgaacagcctgagagccgaggacacggccgtatattactgtgccagacatcctggtagtgctggttatggttatcagtattattacgccatggacccctggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 426 - DNA, 1365 bp, synthetic construct; identical to SEQ ID NO: 410
  misc_feature 1..1365: note = 6R8CD9
  source 1..1365: mol_type = other DNA

SEQ ID NO: 427 - DNA, 1365 bp, synthetic construct
  misc_feature 1..1365: note = 6R8CD10
  source 1..1365: mol_type = other DNA
gaggtgcagctggtggagtctgggggaggcttggtacagcctggggggtccctgagactctcctgtacggcttctggatatagtatttcgcatcaggctgtgacttgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagtctgagagccgaggacacggccgtatattactgtgccagacatcctggtagtgctggttatggttatcagtattattacgccatggacccctggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 428 - DNA, 1365 bp, synthetic construct; identical to SEQ ID NO: 410
  misc_feature 1..1365: note = 6R8CF10
  source 1..1365: mol_type = other DNA

SEQ ID NO: 429 - DNA, 642 bp, synthetic construct; identical to SEQ ID NO: 378
  misc_feature 1..642: note = 6R9A-A2
  source 1..642: mol_type = other DNA

SEQ ID NO: 430 - DNA, 642 bp, synthetic construct
  misc_feature 1..642: note = 6R9A-A2
  source 1..642: mol_type = other DNA
acactctcccctgttgaagctctttgtgacgggcgagctcaggccctgatgggtgacttcgcaggcgtagactttgtgtttctcgtagtctgctttgctcagcgtcagggtgctgctgaggctgtaggtgctgtccttgctgtcctgctctgtgacactctcctgggagttacccgattggagggcgttatccaccttccactgtactttggcctctctgggatagaagttattcagcaggcacacaacagaggcagttccagatttcaactgctcatcagatggcgggaagatgaagacagatggtgcagccacagtccgtttgatctccaccttggtccctccgccgaaatcagtaggcacattatatccttgttgacagtagtaagttgcaaaatcttcaggttgcagactgctgatggtgagagtgtaatctgtcccagatccactgccactgaaccttgatgggaccccatccacacgataattcgcaccatagatcaggagcttaggggctttccctggtttctgctgataccaattaatcccataatgaacctcctcactggcctggcaagtgatggtgactctgtctcctacagatgcagacagggaggatggagactgggtcatctggatgtc

SEQ ID NO: 431 - DNA, 642 bp, synthetic construct; identical to SEQ ID NO: 382
  misc_feature 1..642: note = 6R9B-B11
  source 1..642: mol_type = other DNA

SEQ ID NO: 432 - DNA, 642 bp, synthetic construct
  misc_feature 1..642: note = 6R9B-B11
  source 1..642: mol_type = other DNA
acactctcccctgttgaagctctttgtgacgggcgagctcaggccctgatgggtgacttcgcaggcgtagactttgtgtttctcgtagtctgctttgctcagcgtcagggtgctgctgaggctgtaggtgctgtccttgctgtcctgctctgtgacactctcctgggagttacccgattggagggcgttatccaccttccactgtactttggcctctctgggatagaagttattcagcaggcacacaacagaggcagttccagatttcaactgctcatcagatggcgggaagatgaagacagatggtgcagccacagtccgtttgatctccaccttggtccctccgccgaacgtaataggcacaatatatccttgttgacagtagtaagttgcaaaatcttcaggttgcagactgctgatggtgagagtgtaatctgtcccagatccactgccactgaaccttgatgggacccccggcccacgataattagcaccatagatcaggagcttaggggctttccctggtttctgctgataccaattaatcgcatacttaatcgcctgactggcctggcaagtgatggtgactctgtctcctacagatgcagacagggaggatggagactgggtcatctggatgtc

SEQ ID NO: 433 - DNA, 642 bp, synthetic construct
  misc_feature 1..642: note = 6R9B-E9
  source 1..642: mol_type = other DNA
gacatccagatgacccagtctccatcctccctgtctgcatctgtaggagacagagtcaccatcacttgccaggccagtgagaatattaggtatgctgttaattggtatcagcagaaaccagggaaagcccctaagctcctgatctatggtgcgaattatcgtgtggatggggtcccatcaaggttcagtggcagtggatctgggacagattacactctcaccatcagcagtctgcaacctgaagattttgcaacttactactgtcaacaaggatatcgtgttcagattttgttcggcggagggaccaaggtggagatcaaacggactgtggctgcaccatctgtcttcatcttcccgccatctgacgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 434 - DNA, 642 bp, synthetic construct
  misc_feature 1..642: note = 6R9B-E9
  source 1..642: mol_type = other DNA
acactctcccctgttgaagctctttgtgacgggcgagctcaggccctgatgggtgacttcgcaggcgtagactttgtgtttctcgtagtctgctttgctcagcgtcagggtgctgctgaggctgtaggtgctgtccttgctgtcctgctctgtgacactctcctgggagttacccgattggagggcgttatccaccttccactgtactttggcctctctgggatagaagttattcagcaggcacacaacagaggcagttccagatttcaactgctcgtcagatggcgggaagatgaagacagatggtgcagccacagtccgtttgatctccaccttggtccctccgccgaacaaaatctgaacacgatatccttgttgacagtagtaagttgcaaaatcttcaggttgcagactgctgatggtgagagtgtaatctgtcccagatccactgccactgaaccttgatgggaccccatccacacgataattcgcaccatagatcaggagcttaggggctttccctggtttctgctgataccaattaacagcatacctaatattctcactggcctggcaagtgatggtgactctgtctcctacagatgcagacagggaggatggagactgggtcatctggatgtc

SEQ ID NO: 435 - DNA, 120 bp, synthetic construct
  misc_feature 1..120: note = 6R9E-A8
  source 1..120: mol_type = other DNA
gacatccagatgacccagtctccatcctccctgtctgcatctgtaggagacagagtcaccatcacttgccaggccagtgaggaggttcattatgggattaattggtatcagcagaaacca

SEQ ID NO: 436 - DNA, 120 bp, synthetic construct
  misc_feature 1..120: note = 6R9E-A8
  source 1..120: mol_type = other DNA
tggtttctgctgataccaattaatcccataatgaacctcctcactggcctggcaagtgatggtgactctgtctcctacagatgcagacagggaggatggagactgggtcatctggatgtc

SEQ ID NO: 437 - DNA, 1365 bp, synthetic construct
  misc_feature 1..1365: note = 6R9A-A2
  source 1..1365: mol_type = other DNA
gaggtgcagctggtggagtctgggggaggcttggtacagcctggggggtccctgagactctcctgtacggcttctggatttagttttccgcatcaggcgatgacttgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatcctggtagtgctggttatggttatcagtattattacgccatggacccctggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 438 - DNA, 1365 bp, synthetic construct
  misc_feature 1..1365: note = 6R9A-A2
  source 1..1365: mol_type = other DNA
tttacccggagacagggagaggctcttctgcgtgtagtggttgtgcagagcctcatgcatcacggagcatgagaagacgttcccctgctgccacctgctcttgtccacggtgagcttgctgtagaggaagaaggagccgtcggagtccagcacgggaggcgtggtcttgtagttgttctccggctgcccattgctctcccactccacggcgatgtcgctgggatagaagcctttgaccaggcaggtcaggctgacctggttcttggtcagctcatcccgggatgggggcagggtgtacacctgtggttctcggggctgccctttggctttggagatggttttctcgatgggggctgggagggctttgttggagaccttgcacttgtactccttgccattcagccagtcctggtgcaggacggtgaggacgctgaccacacggtacgtgctgttgtactgctcctcccgcggctttgtcttggcattatgcacctccacgccgtccacgtaccagttgaacttgacctcagggtcttcgtggctcacgtccaccaccacgcatgtgacctcaggggtccgggagatcatgagggtgtccttgggttttggggggaagaggaagactgacggtccccccaggagttcaggtgctgggcacggtgggcatgtgtgagttttgtcacaagatttgggctcaactttcttgtccaccttggtgttgctgggcttgtgattcacgttgcagatgtaggtctgggtgcccaagctgctggagggcacggtcaccacgctgctgagggagtagagtcctgaggactgtaggacagccgggaaggtgtgcacgccgctggtcagggcgcctgagttccacgacaccgtcaccggttcggggaagtagtccttgaccaggcagcccagggccgctgtgcccccagaggtgctcttggaggagggtgccagggggaagaccgatgggcccttggtggaggctgaggagacggtgaccagggttccttggccccaggggtccatggcgtaataatactgataaccataaccagcactaccaggatgtctggcacagtaatatacggccgtgtcctcggctctcaggctgttcatttgcagatacagcgtgttcttggaattgtctctggagatggtgaaccggcctttcgcccagctcgcgtagtatgtgtcagcattactagcaatgactccgatccactccagccccttccctggagcctggcggacccaagtcatcgcctgatgcggaaaactaaatccagaagccgtacaggagagtctcagggaccccccaggctgtaccaagcctcccccagactccaccagctgcacctc

SEQ ID NO: 439 - DNA, 1365 bp, synthetic construct; identical to SEQ ID NO: 410
  misc_feature 1..1365: note = 6R9B-B11
  source 1..1365: mol_type = other DNA

SEQ ID NO: 440 - DNA, 1365 bp, synthetic construct
  misc_feature 1..1365: note = 6R9B-B11
  source 1..1365: mol_type = other DNA
tttacccggagacagggagaggctcttctgcgtgtagtggttgtgcagagcctcatgcatcacggagcatgagaagacgttcccctgctgccacctgctcttgtccacggtgagcttgctgtagaggaagaaggagccgtcggagtccagcacgggaggcgtggtcttgtagttgttctccggctgcccattgctctcccactccacggcgatgtcgctgggatagaagcctttgaccaggcaggtcaggctgacctggttcttggtcagctcatcccgggatgggggcagggtgtacacctgtggttctcggggctgccctttggctttggagatggttttctcgatgggggctgggagggctttgttggagaccttgcacttgtactccttgccattcagccagtcctggtgcaggacggtgaggacgctgaccacacggtacgtgctgttgtactgctcctcccgcggctttgtcttggcattatgcacctccacgccgtccacgtaccagttgaacttgacctcagggtcttcgtggctcacgtccaccaccacgcatgtgacctcaggggtccgggagatcatgagggtgtccttgggttttggggggaagaggaagactgacggtccccccaggagttcaggtgctgggcacggtgggcatgtgtgagttttgtcacaagatttgggctcaactttcttgtccaccttggtgttgctgggcttgtgattcacgttgcagatgtaggtctgggtgcccaagctgctggagggcacggtcaccacgctgctgagggagtagagtcctgaggactgtaggacagccgggaaggtgtgcacgccgctggtcagggcgcctgagttccacgacaccgtcaccggttcggggaagtagtccttgaccaggcagcccagggccgctgtgcccccagaggtgctcttggaggagggtgccagggggaagaccgatgggcccttggtggaggctgaggagacggtgaccagggttccttggccccaggggtccatggcgtaataatactgataaccataaccagcactaccaggatgtctggcacagtaatatacggccgtgtcctcggctctcaggctgttcatttgcagatacagcgtgttcttggaattgtctctggagatggtgaaccggcctttcgcccagctcgcgtagtatgtgtcagcattactagcaatgactccgatccactccagccccttccctggagcctggcggacccaagtgagagcctgacgactaaaagtaaatccagaagccttacaggagagtctcagggaccccccaggctgtaccaagcctcccccagactccaccagctgcacctc

SEQ ID NO: 441 - DNA, 1365 bp, synthetic construct; identical to SEQ ID NO: 410
  misc_feature 1..1365: note = 6R9B-E9
  source 1..1365: mol_type = other DNA

SEQ ID NO: 442 - DNA, 1365 bp, synthetic construct; identical to SEQ ID NO: 440
  misc_feature 1..1365: note = 6R9B-E9
  source 1..1365: mol_type = other DNA

SEQ ID NO: 443 - DNA, 1365 bp, synthetic construct
  misc_feature 1..1365: note = 6R9E-A8
  source 1..1365: mol_type = other DNA
gaggtgcagctggtggagtctgggggaggcttggtacagcctggggggtccctgagactctcctgtaaggcttctggaagtactatttctcatcaggctgtgacttgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatcctggtagtgctggttatggttatcagtattattacgccatggacccctggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 444 - DNA, 1365 bp, synthetic construct
  misc_feature 1..1365: note = 6R9E-A8
  source 1..1365: mol_type = other DNA
tttacccggagacagggagaggctcttctgcgtgtagtggttgtgcagagcctcatgcatcacggagcatgagaagacgttcccctgctgccacctgctcttgtccacggtgagcttgctgtagaggaagaaggagccgtcggagtccagcacgggaggcgtggtcttgtagttgttctccggctgcccattgctctcccactccacggcgatgtcgctgggatagaagcctttgaccaggcaggtcaggctgacctggttcttggtcagctcatcccgggatgggggcagggtgtacacctgtggttctcggggctgccctttggctttggagatggttttctcgatgggggctgggagggctttgttggagaccttgcacttgtactccttgccattcagccagtcctggtgcaggacggtgaggacgctgaccacacggtacgtgctgttgtactgctcctcccgcggctttgtcttggcattatgcacctccacgccgtccacgtaccagttgaacttgacctcagggtcttcgtggctcacgtccaccaccacgcatgtgacctcaggggtccgggagatcatgagggtgtccttgggttttggggggaagaggaagactgacggtccccccaggagttcaggtgctgggcacggtgggcatgtgtgagttttgtcacaagatttgggctcaactttcttgtccaccttggtgttgctgggcttgtgattcacgttgcagatgtaggtctgggtgcccaagctgctggagggcacggtcaccacgctgctgagggagtagagtcctgaggactgtaggacagccgggaaggtgtgcacgccgctggtcagggcgcctgagttccacgacaccgtcaccggttcggggaagtagtccttgaccaggcagcccagggccgctgtgcccccagaggtgctcttggaggagggtgccagggggaagaccgatgggcccttggtggaggctgaggagacggtgaccagggttccttggccccaggggtccatggcgtaataatactgataaccataaccagcactaccaggatgtctggcacagtaatatacggccgtgtcctcggctctcaggctgttcatttgcagatacagcgtgttcttggaattgtctctggagatggtgaaccggcctttcgcccagctcgcgtagtatgtgtcagcattactagcaatgactccgatccactccagccccttccctggagcctggcggacccaagtcacagcctgatgagaaatagtacttccagaagccttacaggagagtctcagggaccccccaggctgtaccaagcctcccccagactccaccagctgcacctc

SEQ ID NO: 445 - DNA, 642 bp, synthetic construct
  misc_feature 1..642: note = Light Chain Sequence of Clone S3R4F12
  source 1..642: mol_type = other DNA
gacatccagatgacccagtctccatcctccctgtctgcatctgtaggagacagagtcaccatcacttgcctggccagtgaggagattcattatgcgattaattggtatcagcagaaaccagggaaagcccctaagctcctgatctatgggcagagtcatcttcaggatggggtcccatcaaggttcagtggcagtggatctgggacagattacactctcaccatcagcagtctgcaacctgaagattttgcaacttactactgtcaacaaggatatgttgtgcctatactgttcggcggagggaccaaggtggagatcaaacggactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgtccgtcacaaagagcttcaacaggggagagtgt

SEQ ID NO: 446 - DNA, 642 bp, synthetic construct
  misc_feature 1..642: note = Light Chain Sequence of Clone S3R4F12
  source 1..642: mol_type = other DNA
acactctcccctgttgaagctctttgtgacggacgagctcaggccctgatgggtgacttcgcaggcgtagactttgtgtttctcgtagtctgctttgctcagcgtcagggtgctgctgaggctgtaggtgctgtccttgctgtcctgctctgtgacactctcctgggagttacccgattggagggcgttatccaccttccactgtactttggcctctctgggatagaagttattcagcaggcacacaacagaggcagttccagatttcaactgctcatcagatggcgggaagatgaagacagatggtgcagccacagtccgtttgatctccaccttggtccctccgccgaacagtataggcacaacatatccttgttgacagtagtaagttgcaaaatcttcaggttgcagactgctgatggtgagagtgtaatctgtcccagatccactgccactgaaccttgatgggaccccatcctgaagatgactctgcccatagatcaggagcttaggggctttccctggtttctgctgataccaattaatcgcataatgaatctcctcactggccaggcaagtgatggtgactctgtctcctacagatgcagacagggaggatggagactgggtcatctggatgtc

SEQ ID NO: 447 - DNA, 1365 bp, synthetic construct
  misc_feature 1..1365: note = Heavy Chain Sequence of Clone S3R4F12
  source 1..1365: mol_type = other DNA
gaggtgcagctggtggagtctgggggaggcttggtacagcctggggggtccctgagactctcctgtgaggcttctggatatagtattgctccgcaggcggtgagttgggtccgccaggctccagggaaggggctggagtggatcggagtcattgctagtaatgctgacacatactacgcgagctgggcgaaaggccggttcaccatctccagagacaattccaagaacacgctgtatctgcaaatgaacagcctgagagccgaggacacggccgtatattactgtgccagacatccgggttttgctggttatggttatggtatgtattattcgatggacccctggggccaaggaaccctggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 448 - DNA, 1365 bp, synthetic construct
  misc_feature 1..1365: note = Heavy Chain Sequence of Clone S3R4F12
  source 1..1365: mol_type = other DNA
tttacccggagacagggagaggctcttctgcgtgtagtggttgtgcagagcctcatgcatcacggagcatgagaagacgttcccctgctgccacctgctcttgtccacggtgagcttgctgtagaggaagaaggagccgtcggagtccagcacgggaggcgtggtcttgtagttgttctccggctgcccattgctctcccactccacggcgatgtcgctgggatagaagcctttgaccaggcaggtcaggctgacctggttcttggtcagctcatcccgggatgggggcagggtgtacacctgtggttctcggggctgccctttggctttggagatggttttctcgatgggggctgggagggctttgttggagaccttgcacttgtactccttgccattcagccagtcctggtgcaggacggtgaggacgctgaccacacggtacgtgctgttgtactgctcctcccgcggctttgtcttggcattatgcacctccacgccgtccacgtaccagttgaacttgacctcagggtcttcgtggctcacgtccaccaccacgcatgtgacctcaggggtccgggagatcatgagggtgtccttgggttttggggggaagaggaagactgacggtccccccaggagttcaggtgctgggcacggtgggcatgtgtgagttttgtcacaagatttgggctcaactttcttgtccaccttggtgttgctgggcttgtgattcacgttgcagatgtaggtctgggtgcccaagctgctggagggcacggtcaccacgctgctgagggagtagagtcctgaggactgtaggacagccgggaaggtgtgcacgccgctggtcagggcgcctgagttccacgacaccgtcaccggttcggggaagtagtccttgaccaggcagcccagggccgctgtgcccccagaggtgctcttggaggagggtgccagggggaagaccgatgggcccttggtggaggctgaggagacggtgaccagggttccttggccccaggggtccatcgaataatacataccataaccataaccagcaaaacccggatgtctggcacagtaatatacggccgtgtcctcggctctcaggctgttcatttgcagatacagcgtgttcttggaattgtctctggagatggtgaaccggcctttcgcccagctcgcgtagtatgtgtcagcattactagcaatgactccgatccactccagccccttccctggagcctggcggacccaactcaccgcctgcggagcaatactatatccagaagcctcacaggagagtctcagggaccccccaggctgtaccaagcctcccccagactccaccagctgcacctc

SEQ ID NO: 449 - protein, 107 aa, synthetic construct
  REGION 1..107: note = LC Interaction of 6R8C-D10 with XBB
DIQMTQSPSSLSASVGDRVTITCQASEEVHYGINWYQQKPGKAPKLLIYGANYRVDGVPSRFSGSGSGTDYTLTISSLQPEDFATYYCQQGYNVPTDFGGGTKVEIK

SEQ ID NO: 450 - protein, 125 aa, synthetic construct
  REGION 1..125: note = HC Interaction of 6R8C-D10 with XBB
EVQLVESGGGLVQPGGSLRLSCTASGYSISHQAVTWVRQAPGKGLEWIGVIASNADTYYASWAKGRFTISRDNSKNTLYLQMNSLRAEDTAVYYCARHPGSAGYGYQYYYAMDPWGQGTLVTVSS

SEQ ID NO: 451 - protein, 223 aa, synthetic construct
  REGION 1..223: note = XBB-RBD Interaction of 6R8C-D10 with XBB
RVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLFRKSKLKPFERDISTEIYQAGNKPCNGVAGSNCYSPLQSYGFRPTYGVGHQPYRVVVLSFELLHAPATVCGPKKSTNLVKNKCVNF

SEQ ID NO: 452 - protein, 107 aa, synthetic construct; identical to SEQ ID NO: 449
  REGION 1..107: note = LC Interaction of 6R8C-D10 with BA.4.6

SEQ ID NO: 453 - protein, 125 aa, synthetic construct; identical to SEQ ID NO: 450
  REGION 1..125: note = HC Interaction of 6R8C-D10 with BA.4.6

SEQ ID NO: 454 - protein, 223 aa, synthetic construct
  REGION 1..223: note = BA.4.6-RBD Interaction of 6R8C-D10 with BA.4.6
RVQPTESIVRFPNITNLCPFDEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNF

SEQ ID NO: 455 - protein, 107 aa, synthetic construct; identical to SEQ ID NO: 449
  REGION 1..107: note = LC Interaction of 6R8C-D10 with BQ.1.1

SEQ ID NO: 456 - protein, 125 aa, synthetic construct; identical to SEQ ID NO: 450
  REGION 1..125: note = HC Interaction of 6R8C-D10 with BQ.1.1

SEQ ID NO: 457 - protein, 223 aa, synthetic construct
  REGION 1..223: note = BQ.1.1-RBD Interaction of 6R8C-D10 with BQ.1.1
RVQPTESIVRFPNITNLCPFDEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSTVGGNYNYRYRLFRKSKLKPFERDISTEIYQAGNKPCNGVAGVNCYFPLQSYGFRPTYGVGHQPYRVVVLSFELLHAPATVCGPKKSTNLVKNKCVNF

SEQ ID NO: 458 - protein, 107 aa, synthetic construct; identical to SEQ ID NO: 449
  REGION 1..107: note = LC Interaction of 6R8C-D10 with BA.2.75.2

SEQ ID NO: 459 - protein, 125 aa, synthetic construct; identical to SEQ ID NO: 450
  REGION 1..125: note = HC Interaction of 6R8C-D10 with BA.2.75.2

SEQ ID NO: 460 - protein, 223 aa, synthetic construct
  REGION 1..223: note = BA.2.75.2-RBD Interaction of 6R8C-D10 with BA.2.75.2
RVQPTESIVRFPNITNLCPFHEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVSGNYNYLYRLFRKSKLKPFERDISTEIYQAGNKPCNGVAGSNCYFPLQSYGFRPTYGVGHQPYRVVVLSFELLHAPATVCGPKKSTNLVKNKCVNF

(19) United States
(12) Patent Application Publication
(10) Pub. No.: US 2026/0365719 A1
(43) Pub. Date: Nov. 19, 2026

(54) ANTIBODIES OR FRAGMENTS THEREOF FOR SARS-COV-2 AND VARIANTS THEREOF

(71) Applicant: ABWIZ BIO, INC, San Diego, CA (US)

(72) Inventors: Toshiaki MARUYAMA, La Jolla, CA (US); Shigeru CJ OKUMURA, San Diego, CA (US); Kevin ENTZMINGER, San Diego, CA (US)

(21) Appl. No.: 19/137,255

(22) PCT Filed: Dec. 11, 2023
(86) PCT No.: PCT/US2023/083390

§ 371 Date: Jun. 9, 2025

Related U.S. Application Data
(60) Provisional application No. 63/431,663, filed on Dec. 9, 2022.

(51) Int. Cl. C07K 16/104

(52) U.S. Cl. CPC C07K 16/104; C07K 2317/92

(57) ABSTRACT

This disclosure generally relates to humanized antibodies and their therapeutic use. More specifically, this disclosure is in the field of producing humanized antibodies that are optimized for biological and chemical features, including activities against SARS-CoV-2 variants, such as omicron variants.

### BACKGROUND

This disclosure generally relates to humanized antibodies and their therapeutic use. More specifically, this disclosure is in the field of producing humanized antibodies that are optimized for particular biological and chemical features, including activities against SARS-Cove-2 and variants thereof.

### SUMMARY

According to one aspect, an antibody or a fragment thereof disclosed herein inhibits binding of ACE2 protein with spike trimer protein of a SARS-CoV-2 variant selected from the group consisting of BQ.1.1, XBB, XBB.1 and combinations thereof.

According to another aspect, an antibody or a fragment thereof disclosed herein inhibits binding of ACE2-Fc and spike trimer proteins of two SARS-CoV-2 variants selected from the group consisting of BQ.1.1, XBB, and XBB.1.

According to one aspect, an antibody or a fragment thereof disclosed herein inhibits binding of ACE2-Fc and spike trimer proteins of SARS-CoV-2 variants BQ.1.1, XBB, and XBB.1.

According to one aspect, an antibody or a fragment thereof disclosed herein inhibits binding of ACE2-Fc and spike trimer protein of a SARS-CiV-2 variant better than Bebtelovimab, wherein the SARS-CiV-2 variant is selected from the group consisting of BQ.1.1, XBB, XBB.1, BA.2.3.20, and combinations thereof.

In some embodiments, the antibody has an IC50 that is less than ½ of IC50 of Bebtelovimab.

In some embodiments, the antibody has an IC50 that is less than ⅓ of IC50 of Bebtelovimab.

In some embodiments, the antibody has an IC50 that is less than ⅕ of IC50 of Bebtelovimab.

In some embodiments, the antibody has a potency against the SARS-CoV-2 variant that is at least 1.5 times that of Bebtelovimab. In some embodiments, the antibody has a potency against the SARS-CoV-2 variant that is at least 2 times that of Bebtelovimab. In some embodiments, the antibody has a potency against the SARS-CoV-2 variant that is at least 3 times that of Bebtelovimab. In some embodiments, the antibody has a potency against the SARS-CoV-2 variant that is at least 5 times that of Bebtelovimab. In some embodiments, the antibody has a potency against the SARS-CoV-2 variant that is at least 10 times that of Bebtelovimab.

In some embodiments, the antibody also inhibits binding of ACE2-Fc and spike trimer protein of a SARS-CoV-2 variant selected from B.1.1.529, BA.2, BA.5, BA.4.6, BA.2.75.2, BN.1, and combinations thereof. In some embodiments, the antibody also inhibits binding of ACE2-Fc and spike trimer protein of SARS-CoV-2 (Wuhan wild type). According to one aspect, an antibody or a fragment thereof disclosed herein inhibits the binding of ACE2 protein with spike trimer protein of SARS-CoV-2 (Wuhan wild type) and spike trimer proteins of SARS-CoV-2 variants B.1.1.529, BA.2, BA.5, BA.4.6, BA.2.75.2, BA.2.3.20, BQ.1.1, BN.1, XBB, and XBB.1.

According to one aspect, an antibody or a fragment thereof disclosed herein binds to or interacts with a SARS-CoV-2 variant XBB as in FIG. 9A. According to one aspect, an antibody or a fragment thereof disclosed herein binds to or interacts with a SARS-CoV-2 variant BA.4.6 as in FIG. 9B. According to one aspect, an antibody or a fragment thereof disclosed herein binds to or interacts with a SARS-CoV-2 variant BQ.1.1 as in FIG. 9C. According to one aspect, an antibody or a fragment thereof disclosed herein binds to or interacts with a SARS-CoV-2 variant BA.2.75.2 as in FIG. 9D.

In some embodiments, the antibody has an LCDR1 selected from SEQ ID NOs: 75-108, 287-290, and 313. The antibody or fragment of any one of claims 1-20, wherein the antibody has an LCDR2 selected from SEQ ID NOs: 109-142, 291-294, and 314. In some embodiments, the antibody has an LCDR3 selected from SEQ ID NOs: 143-176, 295-298, and 315.

In some embodiments, the antibody has an HCDR1 selected from SEQ ID NOs: 177-210, 299-302, and 316. In some embodiments, the antibody has an HCDR2 selected from SEQ ID NOs: 211-244, 303-306, and 317. In some embodiments, the antibody has an HCDR3 selected from SEQ ID NOs: 245-278, 307-310, and 318.

In some embodiments, the antibody has a light chain sequence from SEQ ID NOs: 7-40, 279-282, and 311. In some embodiments, the antibody has a heavy chain sequence from SEQ ID NOs: 41-74, 283-286, and 312.

According to one aspect, an antibody or a fragment thereof that inhibits binding of ACE2 protein with spike trimer protein of SARS-CoV-2 and/or variant(s) thereof is disclosed, wherein the antibody has an LCDR1 selected from SEQ ID NOs: 75-108, 287-290, and 313. In some embodiments, the antibody has an LCDR2 selected from SEQ ID NOs: 109-142, 291-294, and 314. In some embodiments, the antibody has an LCDR3 selected from SEQ ID NOs: 143-176, 295-298, and 315. In some embodiments, the antibody has an HCDR1 selected from SEQ ID NOs: 177-210, 299-302, and 316. In some embodiments, the antibody has an HCDR2 selected from SEQ ID NOs: 211-244, 303-306, and 317. In some embodiments, the antibody has an HCDR3 selected from SEQ ID NOs: 245-278, 307-310, and 318.

According to one aspect, an antibody or a fragment thereof that inhibits binding of ACE2 protein with spike trimer protein of SARS-CoV-2 and/or variant(s) thereof is disclosed, wherein the antibody, wherein the antibody has a light chain sequence from SEQ ID NOs: 7-40, 279-282, and 311.

According to one aspect, an antibody or a fragment thereof that inhibits binding of ACE2 protein with spike trimer protein of SARS-CoV-2 and/or variant(s) thereof is disclosed, wherein the antibody, wherein the antibody has a heavy chain sequence from SEQ ID NOs: 41-74, 283-286, and 312.

An antibody or a fragment thereof that inhibits binding of ACE2 protein with spike trimer protein of SARS-CoV-2 and/or variant(s) thereof, wherein the antibody has a light chain sequence from SEQ ID NOs: 7-40, 279-282, and 311, and has a heavy chain sequence from SEQ ID NOs: 41-74, 283-286, and 312.

One aspect of the present disclosure relates to an antibody or a fragment thereof selected from the group consisting of 3R4-H4, 5R4-H1, 5R4-F3, 5R4-E11, 6R4-H1, 6R4-A7, 6R4-G10, 6R4-G11, 6R4-E12, 6R4-E3, 6R4-H5, 6R4-H11, 6R6-A7, 6R6-E1, 6R6-E2, 6R6-E3, 6R6-E4, 6R6-F3, 6R6-F7, 6R6-F9, 6R6-F11, 6R6-F12, 3R6-D11Q, 6R6-E6Q, 6R8A-E11, 6R8A-F2, 6R8A-E1, 6R8A-A2, 6R8C-G3, 6R8C-G4, 6R8C-G8, 6R8C-D9, 6R8C-D10, 6R8C-F10, 6R9A-A2, 6R9B-B11, 6R9B-E9, 6R9E-A8, and S3R4F12.

One aspect of the present disclosure relates to an antibody or a fragment thereof selected from the group consisting of 6R8A-E11, 6R8A-F2, 6R8A-E1, 6R8A-A2, 6R8C-G8, 6R8C-D9, 6R8C-D10, 6R8C-F10, 6R9A-A2, 6R9B-B11, 6R9B-E9, and 6R9E-A8.

In some embodiments, the antibody or fragment disclosed herein are resistant to mutations at G339D/H, R346T, K356T, L368I, K444T/R, V445P, G446S, N450D, L452R/M, N460K, E484K/R, F486V/S, F490S, Q493R, G496S, T547K.

In some embodiments, the antibody or fragment disclosed herein are resistant to mutations at S371F, S373P, S375F, T376A, D405N, R408S, K417N, N440K, S477N, T478K, Q498R, N501Y, Y505H (RBD in Omicron variants).

One aspect of the present disclosure relates to an antibody or a fragment thereof that inhibits binding of ACE2 protein with spike trimer protein of a SARS-CoV-2 variant selected from the group consisting of XBB.1.31, XBB.1.16.1, EG.5, HV.1, FL.1.5.1, HK.3, BA.2.86, BA.2.86.1, JN.1, and combinations thereof.

One aspect of the present disclosure relates to an antibody or a fragment thereof that inhibits binding of ACE2-Fc and spike trimer proteins of two SARS-CoV-2 variants selected from the group consisting of HV.1, HK.3, JN.1, and combinations thereof.

One aspect of the present disclosure relates to an antibody or a fragment thereof that inhibits binding of ACE2-Fc and spike trimer proteins of SARS-CoV-2 variants HV.1.

One aspect of the present disclosure relates to an antibody or fragment that inhibits binding of ACE2-Fc and spike trimer protein of a SARS-CiV-2 variant better than Bebtelovimab, wherein the SARS-CiV-2 variant is selected from the group consisting of XBB.1.31, XBB.1.16.1, EG.5, HV.1, FL.1.5.1, HK.3, BA.2.86, BA.2.86.1, JN.1, and combinations thereof.

In some embodiments, the antibody has an IC50 that is less than ½ of IC50 of Bebtelovimab.

In some embodiments, the antibody has an IC50 that is less than ⅓ of IC50 of Bebtelovimab.

In some embodiments, the antibody has an IC50 that is less than ⅕ of IC50 of Bebtelovimab.

In some embodiments, the antibody has a potency against the SARS-CoV-2 variant that is at least 1.5 times that of Bebtelovimab. In some embodiments, the antibody has a potency against the SARS-CoV-2 variant that is at least 2 times that of Bebtelovimab. In some embodiments, the antibody has a potency against the SARS-CoV-2 variant that is at least 3 times that of Bebtelovimab. In some embodiments, the antibody has a potency against the SARS-CoV-2 variant that is at least 5 times that of Bebtelovimab. In some embodiments, the antibody has a potency against the SARS-CoV-2 variant that is at least 10 times that of Bebtelovimab.

In some embodiments, the antibody has an LCDR1 selected from SEQ ID NOs: 319, 325, 331, 337, 343, or 349. In some embodiments, the antibody has an LCDR2 selected from SEQ ID NOs: 320, 326, 332, 338, 344, or 350. In some embodiments, the antibody has an LCDR3 selected from SEQ ID NOs: 321, 327, 333, 339, 345, or 351.

In some embodiments, the antibody has an HCDR1 selected from SEQ ID NOs: 322, 328, 334, 340, 346, or 352. In some embodiments, the antibody has an HCDR2 selected from SEQ ID NOs: 323, 329, 335, 341, 347, or 353. In some embodiments, the antibody has an HCDR3 selected from SEQ ID NOs: 324, 330, 336, 342, 348, or 354.

In some embodiments, the antibody has a variable light chain comprising CDRs according to SEQ ID Nos: 319-321, and a variable heavy chain comprising CDRs according to SEQ ID Nos: 322-324. In some embodiments, the antibody has a variable light chain comprising CDRs according to SEQ ID Nos: 335-327, and a variable heavy chain comprising CDRs according to SEQ ID Nos: 328-330. In some embodiments, the antibody has a variable light chain comprising CDRs according to SEQ ID Nos: 331-333, and a variable heavy chain comprising CDRs according to SEQ ID Nos: 334-336. In some embodiments, the antibody has a variable light chain comprising CDRs according to SEQ ID Nos: 337-339, and a variable heavy chain comprising CDRs according to SEQ ID Nos: 340-342. In some embodiments, the antibody has a variable light chain comprising CDRs according to SEQ ID Nos: 343-345, and a variable heavy chain comprising CDRs according to SEQ ID Nos: 346-348. In some embodiments, the antibody has a variable light chain comprising CDRs according to SEQ ID Nos: 349-351, and a variable heavy chain comprising CDRs according to SEQ ID Nos: 352-354.

According to one aspect, an antibody or a fragment thereof disclosed herein is a humanized antibody or fragment thereof.

### BRIEF DESCRIPTION OF THE DRAWING FIGURES

FIG. 1 depicts binding of rabbit mAb C-A11 and its humanized clone hN2F tested by ELISA. Spike proteins were coated at 2 μg/mL and incubated with serially diluted IgG. The detection of bound IgGs were done with HRP-conjugated goat anti-rabbit IgG Fc and human IgG Fc specific antibodies. Both C-A11 and hN2F showed binding to spike proteins of wild type and Alpha and Beta variants.

FIG. 2 depicts binding of rabbit mAb C-A11 and its humanized clone hN2Y tested by ELISA. Spike proteins were coated at 2 μg/mL and incubated with serially diluted IgG. The detection of bound IgGs were done with HRP-conjugated goat anti-rabbit IgG Fc and human IgG Fc specific antibodies. Both C-A11 and hN2Y showed binding to spike proteins of wild type and Alpha and Beta variants.

FIG. 3 depicts a table that shows certain unique CDR sequences identified during screening selection on BA.5 and BA.2.75.2.

FIG. 4 depicts a table that shows certain unique CDR sequences identified during screening selection on BQ.1.1 and XBB Spike Trimers.

FIG. 5A-5K depicts Surrogate virus neutralization test (sVNT) using recombinant spike trimers and ACE2-Fc protein. Inhibitions of the binding of spike trimers (48 ng/mL) to ACE2-Fc (2 μg/mL) coated wells by antibodies are shown. 6R8A-E1, 6R8C-G8, 6R8A-A2, 6R9A-A2, and 6R8C-D10 showed potent neutralization of all Omicron variants and Wuhan strain (wild type) tested while Bebtelovimab did not show any significant inhibition of BQ.1.1, XBB, and XBB.1 and weak neutralization of BA.2.3.20 although it showed potent neutralization of Wuhan, BA.2, BA.5 and BA.4.6.

FIG. 6A-6O illustrate surrogate virus neutralization test (sVNT) using recombinant spike trimers and ACE2-Fc protein. Inhibitions of the binding of spike trimers (48 ng/mL) to ACE2-Fc (2 μg/mL) coated wells by antibodies are shown. Assays were performed in triplicate. 6R8A-A2, 6R8A-E1, 6R8A-E11, 6R8C-D10, 6R8C-G8, 6R9A-A2, and 6R9E-A8 showed potent neutralization of Delta and all Omicron variants, while bebtelovimab did not show any significant inhibition of BQ.1.1, XBB, and XBB.1 and weak neutralization of BA.2.3.20.

FIG. 7 depicts BLI affinity measurement shows strong binding to BQ.1.1 and XBB.1.5 for engineered IgGs. In each assay, biotinylated spike protein trimer was captured to the sensor surface and dipped into a 7-fold dilution series of IgG at a starting concentration of 10 nM. Binding was monitored for 4 minutes, followed by dissociation monitored for 30 minutes. For each variant tested, each experiment is shown with the same scale y-axis. Both the humanized lead candidate hN2Y and the light chain engineered clone LxC1-G10 lost binding activity for later SARS-CoV-2 variants, but 6R8A-A2, 6R8A-E1, 6R8A-E11, 6R8C-D10, 6R8C-G8, 6R9A-A2, and 6R9E-A8 showed strong binding to BQ.1.1 and XBB.1.5. Therapeutic mAbs tested identically showed weak or no binding to the latest Omicron variants.

FIG. 8 depicts Colabfold modeling of neutralizing antibodies (cyan and colored by CDRs) binding to spike trimer (pink). Following Colabfold simultaneous modeling and docking of antibodies and BQ.1.1 RBD, the above model was created by aligning the BQ.1.1 RBD used in our folding prediction with the cryo-EM structure (PDB ID 7A94) of the wild-type spike trimer bound to ACE2 (blue). BQ.1.1 from the folding prediction is hidden to allow visualization of epitope binding. Notably, all antibodies bind at the same location as ACE2. Nearly all antibodies showed highly similar binding epitopes, and these are overlaid above. 6R8C-D10 (SEQ ID No. 39 and SEQ ID No. 73) is shown as an example alone. 6R9B-E9 (SEQ ID No. 281 and SEQ ID No. 285) is shown separately as it showed a slightly different binding mode, but still overlapped with the ACE2 binding site.

FIGS. 9A-9D depict interactions of 6R8C-D10 (SEQ ID No. 39 and SEQ ID No. 73) with RBD of four different SARS-CoV-2 variants. Regions of RBD closest to CDRs of 6R8C-D10 are colored in GREEN. Polar contacts seen in PyMol are colored in PURPLE. Mutated amino acids different from wild type RBD are shown in BOLD. CDR H3 maintains polar contacts among all variants, and many of the contacts are predicted within a similar region of RBD, potentially supporting the experimental data showing broad neutralization of all variants.

FIG. 10 illustrates surrogate virus neutralization test (sVNT) using recombinant spike trimers and ACE2-Fc protein. Inhibitions of the binding of spike trimers (48 ng/mL) to ACE2-Fc (2 μg/mL) coated wells by antibodies are shown. Assays were performed in triplicate.

### DETAILED DESCRIPTION

In embodiments of the present disclosure, instead of back-mutating, the present disclosure analyzes all available human antibody sequences from a database that use the selected framework germline gene and dope in possible combinations of more human amino acids in each position of each CDR and make 6 single CDR libraries (LCDR1, LCDR2, LCDR3, HCDR1, HCDR2, and HCDR3) simultaneously. This lessens the chance of immunogenicity. These 6 single CDR libraries are selected against the target antigen using phage display to select the practical pool of binders in each library. Then, these pre-selected LCDR1, LCDR2, and LCDR3 libraries are combined by overlap PCR mutagenesis into a single combined light chain library. This is repeated for pre-selected HCDR1, HCDR2, and HCDR3 libraries to make a single heavy chain library. Using this novel strategy, the largest practical diversity can be covered for each CDR, and optimized CDR sequences that work cooperatively across multiple CDRs can be selected. These LCDR and HCDR combined libraries are selected on target antigen using more stringent conditions to obtain higher affinity and specificity.

In addition, the pooled phage is pre-treated with a transient heating/cooling step to remove unstable clones and retain clones that have better stability, which translates to better expression and developability. Optionally, these LCDR and HCDR libraries after selection are further combined after PCR amplification of the selected phage pool into a single LCDR/HCDR library and selected with even higher stringency to select for the best lead candidates. Stepwise combination of each library allows us to obtain more diverse antibodies with higher affinity and better function than ever before possible with conventional methods. This method of antibody optimization is flexible and can be performed in a variety of ways, including but not limited to (1) a limited number of single CDR libraries are made (e.g. LCDR3 and HCDR2 libraries) and pre-selected libraries are combined into 1 library (e.g. LCDR3/HCDR2) for further optimization, (2) only one chain is targeted for optimization (e.g., light chain) while the other is retained as wild-type, resulting in three single CDR libraries (e.g., LCDR1, LCDR2, and LCDR3) that are pre-selected and then combined into one library (e.g., LCDR), (3) an alternate method of CDR library design is used (see: Example 6 herein) to limit diversity for a particular function, but afterward pre-selected clones are combined for further selection steps. This method is also generally applicable to any recombinant protein where separate libraries are constructed targeting multiple regions of interest followed by panning and then combination of the pre-selected pools into a new library for further selection (see: Examples herein).

Comprehensively mutagenized libraries that cover entire CDRs can be enormously large such that they cannot be covered practically by the transformation of bacterial cells. Therefore, scientists tend to use an error-prone PCR, chain shuffling, CDR3 only-targeted, or CDR walking method. In a mutagenized library using NNK or trimer-phosphoramidite doping method, most of the clones do not bind to the target. Therefore, an efficient selection method is needed to capture the practical clones that show binding to the target with high affinity. Mild selection stringency can result in clones with low affinity, but if the selection is too stringent, you may not be able to recover any clones from a more focused library such as CDR3 alone, or from a CDR walking strategy that focuses only on 1 CDR at a time, or from error prone PCR or chain shuffling library. In our invention, in Stage 1, the initial libraries are made for 6 individual CDRs and selected on the target antigen in a mild condition to obtain all practical binders, even if their affinities are low. Next, in Stage 2, pre-selected light chain CDR libraries are combined and paired with the wild type heavy chain and pre-selected heavy chain CDR libraries are combined and paired with the wild type light chain. These Stage 2 libraries are separately selected against the target with higher stringency to obtain clones with higher affinity than the original antibodies. Finally, in Stage 3, these pre-selected combined light chain CDR library and pre-selected combined heavy chain CDR library are combined into 1 library and further selected with even higher stringency to obtain the clones with the highest affinity. This method ensures the widest coverage of diversity in each CDR library and captures the best combination of mutants from each library when they are combined. The purpose of Stage 1 is not selecting the best binders but rather eliminating the non-binders from each library and making the practical library size smaller e.g., 10⁴ to 10⁵ so that when multiple pre-selected CDR libraries are combined, the diversity can be covered by the transformation of the library DNA. Then, Stage 2 can select the binders with higher affinity.

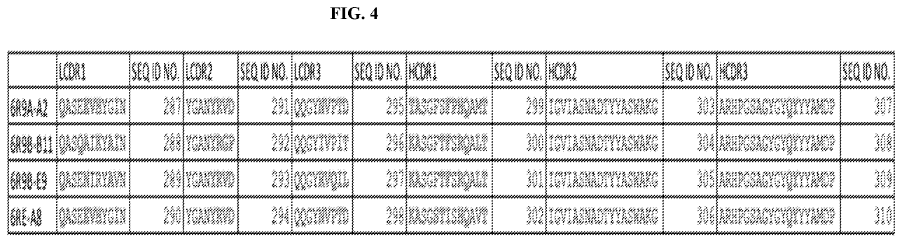

In a typical CDR walking strategy, one CDR is selected and the library is constructed and the best binder(s) are selected. Afterwards, only one or a few clones are chosen before constructing the next CDR library. That is why it is called CDR walking, as it walks through each CDR one by one. The pitfall with this strategy is that the ‘best’ mutant clone(s) from one CDR library may not be the best mutant when combined with mutants from another CDR library. The evidence from our method is that the best clones from each CDR library obtained in Stage 1 may not be the best mutations from Stage 2 library, and the mutations found in each CDR in Stage 2 library may not be the best mutant in Stage 1, vice versa. FIG. 4 shows representative sequencing results from a 3-stage affinity maturation library. Unique CDR sequences were consistently obtained in later stages of phage panning (after higher affinity selection of the combined libraries). The best candidates from single CDR libraries are often not present in later stages, indicating that only a multi-stage approach that combines entire pre-selected CDR pools can result in clones with mutations that work cooperatively across multiple CDRs. During affinity maturation, a stepwise increase from Stage 1 to 2 to 3 in the binding and affinity (off rate) is typically observed for the selected clones. These results are representative of our strategy across multiple antibodies and targets. Therefore, it is vitally important to capture the widest practical diversity in each CDR library in stage 1, and to ensure that the whole pre-selected libraries are combined in Stage 2, not just the best binder(s) in each CDR library.

In another preferred embodiment, one or more of the selected clones from screenings such as ELISA screening or flow cytometry screening can be pooled and used as a library to combine with clones from other CDR libraries or the whole library. Unlike focusing on only 1 or a few clones, it will ensure the coverage of mutants that could show better affinity when combined with mutants from another CDR. Alternatively, the heavy chains or the light chains obtained from Stage 2 and 3 could be paired with any of the light chains and heavy chains obtained from Stage 1 and 2 to increase the chances of achieving the best clones with affinity, specificity, and functions.

Current affinity maturation approaches suffer from poor developability of lead candidates. The issue with error-prone PCR is that you may get unwanted amino acid mutations in the framework that could elicit immunogenicity when the antibodies are used as a therapeutic antibody or may cause expression and stability issues even when they were made for other purposes. Chain shuffling is also currently used for affinity maturation, where one chain is paired with a random library of another chain to find the best binder. The issue with this approach is that they may show binding but there could be compatibility and stability issues, as they are artificially paired unlike from an immunized source.

The multi-stage strategy detailed herein addresses these developability problems in two important ways. First, in the design of each CDR library, the amino acid usage at each position is compared with an antibody database, and the doping strategy is carefully selected (e.g., NNK, TWT, GST, etc.) to avoid unwanted mutants that could affect the structural integrity of the antibody and to keep the library size within 10¹⁰ and cover the widest practical diversity in the library. Only desired amino acids are included in each position targeted for mutagenesis. Second, a brief heat treatment of the phage is also included to select more stable clones. This translates to obtaining good expressers and clones with longer storage life meaning better developability for a therapeutic antibody. We have observed clones with better binding and expression when the phage is treated with heat than when it is not (see: Examples herein). Additionally, further selection pressure can be incorporated into each panning stage to eliminate undesirable developability characteristics, for example by subtraction of polyreactive clones by pre-incubation on baculovirus particle (BVP) coated wells prior to transfer to antigen coated wells.

The multi-stage approach can be used to simultaneously further humanize a clone while optimizing affinity and/or function. First, the humanization of an antibody from another species (rabbit, mouse, llama, etc.) is performed by comparison to the amino acid sequences of a human germline framework. Each CDR is grafted into the selected human germline framework. Next, to perform further humanization and affinity maturation, for the design of the single CDR mutant libraries, the amino acid distribution at each position used by human antibodies of the same framework is considered. A doping strategy is determined for each position, e.g., TMT (M=A or C) when Tyr and Ser is used predominantly and NNK when there is no biased usage of amino acids. This approach maximizes the clonal diversity covered in each library and at the same time further humanizes the lead candidate instead of back mutating to the amino acid used by the animal antibodies. This approach also avoids unwanted amino acids substitutions that may affect the structural integrity and potential immunogenicity.

Since the first outbreak of COVID-19 started late 2019, it has already been 3 years but the waves of new variants do not seem to subside completely. After the Omicron variant was found, many sub-variants have emerged that the original vaccines cannot prevent symptomatic infection. Additionally, neutralizing antibodies that had been developed and FDA approved, including the first-generation antibodies such as REGEN-COV (casirivimab and imdevimab, administered together), bamlanivimab and etesevimab, administered together, and Sotrovimab, are not effective for treatment.

These first-generation neutralizing antibodies are not currently authorized in any U.S. region and may not be administered for treatment or post-exposure prevention of COVID-19 under the Emergency Use Authorization until further notice by the FDA.

Even the second-generation antibody bebtelovimab, which showed effective neutralization of the earlier phase of Omicron variants, shows no significant neutralization of BQ.1.1 and XBB. In the United States, the number of newly infected patients with BQ.1 and BQ.1.1 are rapidly increasing reaching about 67% of total infection as of Nov. 26, 2022 while BA.5 and BA.4.6 are decreasing in number 19.4% and 3.3%, respectively. XBB has reached 3.1% of total infection in the U.S. as of Nov. 26, 2022 (https://covid.cdc.gov/covid-data-tracker/#variant-proportions), and is spreading in other countries like Singapore and India.

Only few mAbs remain suitable for treatment of individuals at high risk, and only bebtelovimab shows high efficacy against multiple omicron sublineages. However, novel omicron sublineages have been detected, harboring additional S protein mutations within the epitopes of bebtelovimab and other mAbs. Novel sublineages include BA.4.6 (with increasing incidence in several countries worldwide), BA.2.75.2 (with increasing incidence in India), BJ.1 (mainly observed in India and Bangladesh; notably BJ.1 is one parental lineage of the currently increasing XBB recombinant), and BQ.1.1 (with increasing incidence in the USA and Europe).

Data reveal that emerging omicron sublineages are resistant to most (ie, BA.4.6, BA.2.75.2, and BJ.1) or all (BQ.1.1) clinically used mAbs. As a consequence, in patients at high risk, treatment with mAbs alone might not provide a therapeutic benefit in regions of the globe in which BQ.1.1 is spreading, suggesting that additional treatment options (eg, paxlovid or molnupiravir) should be considered. Furthermore, novel, broadly active mAbs are urgently needed for prophylactic or therapeutic treatment, or both, in patients at high risk.

BQ.1, BQ.1.1, BA.4.6, BF.7 and BA.2.75.2 were examined for neutralization resistance of these subvariants, as well as their ancestral BA.4/5, BA.2.75 and D614G variants, against sera from 3-dose vaccinated health care workers, hospitalized BA.1-wave patients, and BA.5-wave patients. They found enhanced neutralization resistance in all new subvariants, especially the BQ.1 and BQ.1.1 subvariants driven by a key N460K mutation, and to a lesser extent, R346T and K444T mutations, as well as the BA.2.75.2 subvariant driven largely by its F486S mutation. The BQ.1 and BQ.1.1 subvariants also exhibited enhanced fusogenicity and S processing dictated by the N460K mutation. Molecular modeling revealed the mechanisms of receptor-binding and non-receptor binding monoclonal antibody-mediated immune evasion by R346T, K444T, F486S and D1199N mutations. New antibodies that can netutralize virus variants harboring these mutations are desired.

This study established a viral amplification procedure to easily isolate Omicron strains and examined their sensitivity to 6 therapeutic monoclonal antibodies (mAbs) and to 72 sera from Pfizer BNT162b2-vaccinated individuals, with or without BA.1/BA.2 or BA.5 breakthrough infection. Ronapreve (Casirivimab and Imdevimab) and Evusheld (Cilgavimab and Tixagevimab) lost any antiviral efficacy against BA.2.75.2 and BQ.1.1, whereas Xevudy (Sotrovimab) remained weakly active. BQ.1.1 was also resistant to Bebtelovimab. Neutralizing titers in triply vaccinated individuals were low to undetectable against BQ.1.1 and BA.2.75.2, 4 months after boosting. A BA.1/BA.2 breakthrough infection increased these titers, which remained about 18-fold lower against BA.2.75.2 and BQ.1.1 than against BA.1. Reciprocally, a BA.5 breakthrough infection increased more efficiently neutralization against BA.5 and BQ.1.1 than against BA.2.75.2. Thus, the evolution trajectory of novel Omicron subvariants facilitated their spread in immunized populations and raised concerns about the efficacy of most currently available mAbs. New antibodies for these mutations are desperately needed.

Moderna and Pfizer have developed a bivalent vaccine to cover the Omicron variant. However, this could induce weaker pan-COVID-19 antibodies rather than antibodies that specifically bind and neutralize the newly emerging Omicron sub-variants. Some patients, who had been immunized with the original vaccine 3 times and were then infected with the original Omicron variant followed by a boost with the bivalent vaccine, were still susceptible to re-infection with an Omicron variant later.

Re-infection does occur, and a recent study also showed that re-infection is related to increased additional risk of death and sequelae including pulmonary, cardiovascular, hematological, diabetes, gastrointestinal, kidney, mental health, musculoskeletal and neurological disorders regardless of vaccine status (Bowe B, Xie Y, Al-Aly Z, Acute and postacute sequelae associated with SARS-CoV-2 reinfection. Nature Medicine 2022 https://www.nature.com/articles/s41591-022-02051-3). The mutations in the receptor binding domain (RBD) are seen in several hot spots such as R346, K356, K444, L452, N460K, and F486. The humoral immune response after BA.2 and BA.5 breakthrough infection caused significant reductions in the epitope diversity of neutralizing antibodies and increased proportion of non-neutralizing antibodies. Current herd immunity and BA.5 vaccine boosters may not provide good protection against infection. (Cao Y, Jian F, Wang J, et al. Imprinted SARS-CoV-2 humoral immunity induces convergent Omicron RBD evolution. bioRxiv. 2022 https://doi.org/10.1101/2022.09.15.507787).

The present disclosure recognizes the need of a new generation of neutralizing antibodies is desperately needed to treat infection by emerging Omicron variants, which are resistant to all current FDA-approved antibodies. In an aspect, a method for identifying an optimized such antibodies are utilized in the present disclosure. The method involves selecting targeted libraries against a target antigen using a first set of selection conditions to select a pool of binders in each library; combining the selected libraries into one or more libraries; and selecting the combined library against the target antigen using a second set of selection conditions to identify at least one protein having an optimized functional profile. In embodiments, the protein comprises an antibody or a fragment thereof. In embodiments, the method further involves constructing the targeted libraries. In embodiments, the second set of selection conditions is more stringent than the first set of selection conditions. In embodiments, the targeted libraries comprise complementarity determining region (CDR) libraries. In embodiments, the second set of selection conditions is more stringent than the first set of selection conditions. In embodiments, the optimized functional profile comprises a pre-defined affinity, specificity, or functional quality of the protein. In embodiments, the CDR libraries comprise light chain CDRs (LCDRs). For example, the CDR libraries may comprise LCDR1, LCDR2, and LCDR3. In embodiments, the CDR libraries comprise heavy chain CDRs (HCDRs). For example, the CDR libraries may comprise HCDR1, HCDR2, and HCDR3. In further embodiments, the CDR libraries comprise both LCDRs and HCDRs. In embodiments, the method involves combining a LCDR library and a HCDR library into a single library. In embodiments, the method involves selecting the combined LCDR/HCDR library against the target antigen using a third set of selection conditions to identify at least one protein having an optimized functional profile. In embodiments, the third set of selection conditions is more stringent than the first set of selection conditions. In embodiments, a step in the method comprises use of phage display. In embodiments, a step in the method comprises use of overlap PCR mutagenesis. In embodiments, the method includes at least one step designed to reduce identification of a protein that is polyreactive. In embodiments, the antigen comprises a viral antigen. In certain embodiments, the viral antigen comprises an antigen associated with Coronaviridae family. In certain embodiments, the viral antigen comprises an antigen associated with SARS-CoV-2 or a SARS-CoV-2 variant, including newly found Omicron variants BA.5, BA4.6, BA.2.7.5.2, BA.2.3.20, BQ.1.1, BN.1, and XBB in addition to Wuhan (wild type), B.1.1.529, and BA.2. In certain embodiments, the viral antigen comprises spike protein of SARS-CoV-2 or a SARS-CoV-2 variant. In embodiments, the viral antigen comprises RBD of SARS-CoV-2 or a SARS-CoV-2 variant.

In another aspect, a protein identified by the methods described herein is disclosed. In embodiments, the protein comprises an antibody or a fragment thereof.

According to one aspect, an antibody or a fragment thereof disclosed herein inhibits binding of ACE2 protein with spike trimer protein of a SARS-CoV-2 variant selected from the group consisting of BQ.1.1, XBB, XBB.1 and combinations thereof.

According to another aspect, an antibody or a fragment thereof disclosed herein inhibits binding of ACE2-Fc and spike trimer proteins of two SARS-CoV-2 variants selected from the group consisting of BQ.1.1, XBB, and XBB.1.

According to one aspect, an antibody or a fragment thereof disclosed herein inhibits binding of ACE2-Fc and spike trimer proteins of SARS-CoV-2 variants BQ.1.1, XBB, and XBB.1.

According to one aspect, an antibody or a fragment thereof disclosed herein inhibits binding of ACE2-Fc and spike trimer protein of a SARS-CiV-2 variant better than Bebtelovimab, wherein the SARS-CiV-2 variant is selected from the group consisting of BQ.1.1, XBB, XBB.1, BA.2.3.20, and combinations thereof.

In some embodiments, the antibody has an IC50 that is less than ½ of IC50 of Bebtelovimab.

In some embodiments, the antibody has an IC50 that is less than ⅓ of IC50 of Bebtelovimab.

In some embodiments, the antibody has an IC50 that is less than ⅕ of IC50 of Bebtelovimab.

In some embodiments, the antibody has a potency against the SARS-CoV-2 variant that is at least 2 times that of Bebtelovimab.

In some embodiments, the antibody has a potency against the SARS-CoV-2 variant that is at least 2 times that of Bebtelovimab.

In some embodiments, the antibody has a potency against the SARS-CoV-2 variant that is at least 3 times that of Bebtelovimab.

In some embodiments, the antibody has a potency against the SARS-CoV-2 variant that is at least 5 times that of Bebtelovimab.

In some embodiments, the antibody has a potency against the SARS-CoV-2 variant that is at least 10 times that of Bebtelovimab.

In some embodiments, the antibody also inhibits binding of ACE2-Fc and spike trimer protein of a SARS-CoV-2 variant selected from B.1.1.529, BA.2, BA.5, BA.4.6, BA.2.75.2, BN.1, and combinations thereof.

In some embodiments, the antibody also inhibits binding of ACE2-Fc and spike trimer protein of SARS-CoV-2 (Wuhan wild type).

According to one aspect, an antibody or a fragment thereof disclosed herein inhibits the binding of ACE2 protein with spike trimer protein of SARS-CoV-2 (Wuhan wild type) and spike trimer proteins of SARS-CoV-2 variants B.1.1.529, BA.2, BA.5, BA.4.6, BA.2.75.2, BA.2.3.20, BQ.1.1, BN.1, XBB, and XBB.1.

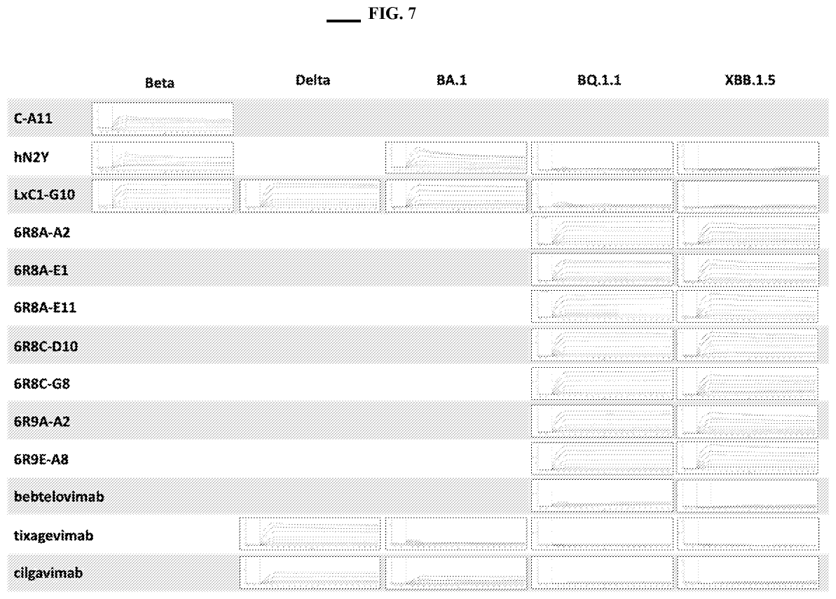

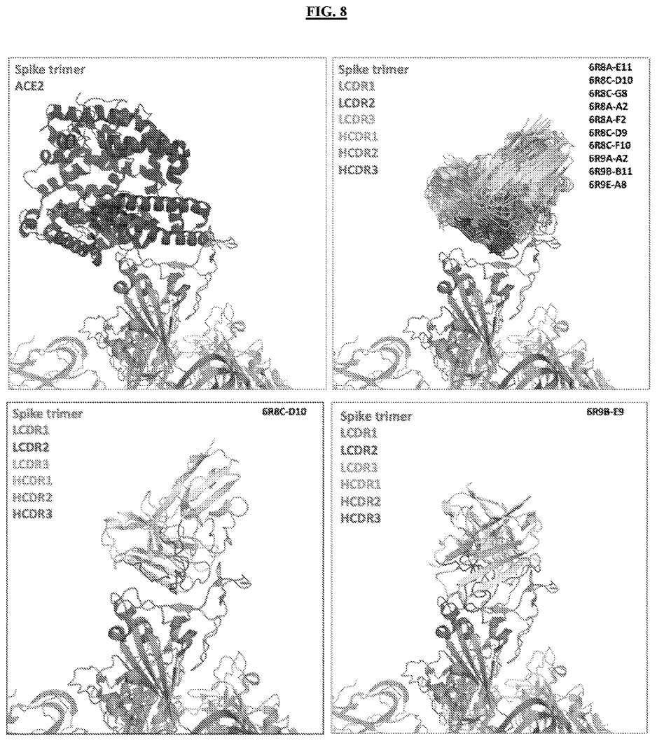

According to one aspect, an antibody or a fragment thereof disclosed herein binds to or interacts with a SARS-CoV-2 variant XBB as in FIG. 7A.

According to one aspect, an antibody or a fragment thereof disclosed herein binds to or interacts with a SARS-CoV-2 variant BA.4.6 as in FIG. 7B.

According to one aspect, an antibody or a fragment thereof disclosed herein binds to or interacts with a SARS-CoV-2 variant BQ.1.1 as in FIG. 7C.

According to one aspect, an antibody or a fragment thereof disclosed herein binds to or interacts with a SARS-CoV-2 variant BA.2.75.2 as in FIG. 7D.

In some embodiments, the antibody has an LCDR1 selected from SEQ ID NOs: 75-108 and 287-290. In some embodiments, the antibody has an LCDR2 selected from SEQ ID NOs: 109-142 and 291-294. In some embodiments, the antibody has an LCDR3 selected from SEQ ID NOs: 143-176 and 295-298.

In some embodiments, the antibody has an HCDR1 selected from SEQ ID NOs: 177-210 and 299-302. In some embodiments, the antibody has an HCDR2 selected from SEQ ID NOs: 211-244 and 303-306. In some embodiments, the antibody has an HCDR3 selected from SEQ ID NOs: 245-278 and 307-310.

In some embodiments, the antibody has a light chain sequence from SEQ ID NOs: 7-40 and 279-282. In some embodiments, the antibody has a heavy chain sequence from SEQ ID NOs: 41-74 and 283-286.

According to one aspect, an antibody or a fragment thereof disclosed herein inhibits binding of ACE2 protein with spike trimer protein of SARS-CoV-2 and/or variant(s) thereof, wherein the antibody has an LCDR1 selected from SEQ ID NOs: 75-108 and 287-290. In some embodiments, the antibody has an LCDR2 selected from SEQ ID NOs: 109-142 and 291-294. In some embodiments, the antibody has an LCDR3 selected from SEQ ID NOs: 143-176 and 295-298. In some embodiments, the antibody has an HCDR1 selected from SEQ ID NOs: 177-210 and 299-302. In some embodiments, the antibody has an HCDR2 selected from SEQ ID NOs: 211-244 and 303-306. In some embodiments, the antibody has an HCDR3 selected from SEQ ID NOs: 245-278 and 307-310.

According to one aspect, an antibody or a fragment thereof disclosed herein inhibits binding of ACE2 protein with spike trimer protein of SARS-CoV-2 and/or variant(s) thereof, wherein the antibody, wherein the antibody has a light chain sequence from SEQ ID NOs: 7-40 and 279-282.

According to one aspect, an antibody or a fragment thereof disclosed herein inhibits binding of ACE2 protein with spike trimer protein of SARS-CoV-2 and/or variant(s) thereof, wherein the antibody, wherein the antibody has a heavy chain sequence from SEQ ID NOs: 41-74 and 283-286.

According to one aspect, an antibody or a fragment thereof disclosed herein inhibits binding of ACE2 protein with spike trimer protein of SARS-CoV-2 and/or variant(s) thereof, wherein the antibody has a light chain sequence from SEQ ID NOs: 7-40 and 279-282 and has a heavy chain sequence from SEQ ID NOs: 41-74 and 283-286

According to one aspect, an antibody or a fragment thereof disclosed herein is selected from the group consisting of 3R4-H4, 5R4-H1, 5R4-F3, 5R4-E11, 6R4-H1, 6R4-A7, 6R4-G10, 6R4-G11, 6R4-E12, 6R4-E3, 6R4-H5, 6R4-H11, 6R6-A7, 6R6-E1, 6R6-E2, 6R6-E3, 6R6-E4, 6R6-F3, 6R6-F7, 6R6-F9, 6R6-F11, 6R6-F12, 3R6-D11Q, 6R6-E6Q, 6R8A-E11, 6R8A-F2, 6R8A-E1, 6R8A-A2, 6R8C-G3, 6R8C-G4, 6R8C-G8, 6R8C-D9, 6R8C-D10, 6R8C-F10, 6R9A-A2, 6R9B-B11, 6R9B-E9, and 6R9E-A8.

According to one aspect, an antibody or a fragment thereof disclosed herein is selected from the group consisting of 6R8A-E11, 6R8A-F2, 6R8A-E1, 6R8A-A2, 6R8C-G8, 6R8C-D9, 6R8C-D10, 6R8C-F10, 6R9A-A2, 6R9B-B11, 6R9B-E9, and 6R9E-A8.

According to one aspect, an antibody or a fragment thereof disclosed herein is 6R8C-D10

According to one aspect, an antibody or a fragment thereof disclosed herein are resistant to mutations at G339D/H, R346T, K356T, L368I, K444T/R, V445P, G446S, N450D, L452R/M, N460K, E484K/R, F486V/S, F490S, Q493R, G496S, T547K.

According to one aspect, an antibody or a fragment thereof disclosed herein are resistant to mutations at S371F, S373P, S375F, T376A, D405N, R408S, K417N, N440K, S477N, T478K, Q498R, N501Y, Y505H (RBD in Omicron variants).

According to one aspect, an antibody or a fragment thereof disclosed herein is a humanized antibody or fragment thereof.

Without limiting any of the present disclosure, it is specifically contemplated that reference to a sequence identify, in some embodiments, encompasses at least 80%, or at least 85%, or at least 90%, or at least 95%, or greater than 95% of sequence identity in the SEQ ID Nos. Also, reference to percentage will include percentages above. For example, reference to at least 80% will include percentages above 80%, such as 81%, 82%, 83%, 84%, 85%, 86%, 87%, 88%, 89%, 90%, 91%, 92%, 93%, 94%, 95%, 96%, 97%, 98%, 99%, and greater than 99%.

### Non-Limiting Embodiments

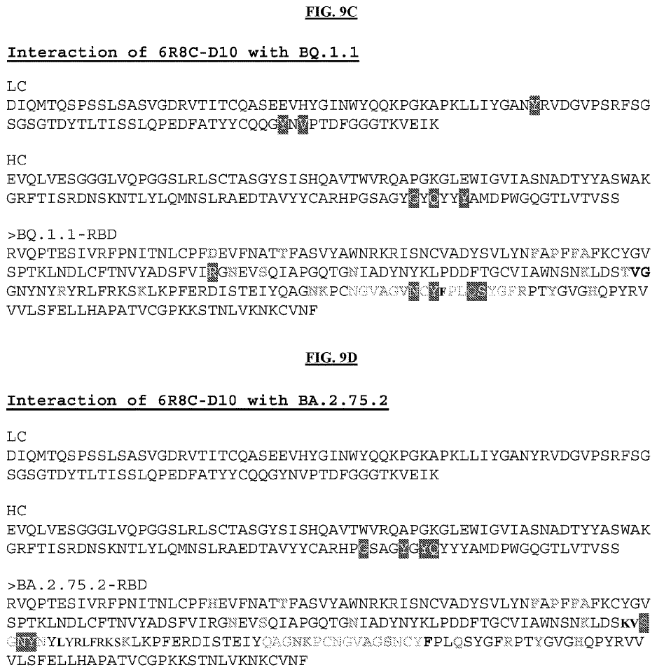

The following Embodiments are detailed by way of illustration only and are not to be construed as limiting in spirit or in scope, many modifications both in materials and in methods will be apparent to those skilled in the art.
1. An antibody or a fragment thereof that inhibits binding of ACE2 protein with spike trimer protein of a SARS-CoV-2 variant selected from the group consisting of BQ.1.1, XBB, XBB.1 and combinations thereof.
2. An antibody or a fragment thereof that inhibits binding of ACE2-Fc and spike trimer proteins of two SARS-CoV-2 variants selected from the group consisting of BQ.1.1, XBB, and XBB.1.
3. An antibody or a fragment thereof that inhibits binding of ACE2-Fc and spike trimer proteins of SARS-CoV-2 variants BQ.1.1, XBB, and XBB.1.
4. An antibody or fragment that inhibits binding of ACE2-Fc and spike trimer protein of a SARS-CiV-2 variant better than Bebtelovimab, wherein the SARS-CiV-2 variant is selected from the group consisting of BQ.1.1, XBB, XBB.1, BA.2.3.20, and combinations thereof.
5. The antibody or fragment of embodiments 1-4, wherein the antibody has an IC50 that is less than ½ of IC50 of Bebtelovimab.
6. The antibody or fragment of embodiments 1-4, wherein the antibody has an IC50 that is less than ⅓ of IC50 of Bebtelovimab.
7. The antibody or fragment of embodiments 1-4, wherein the antibody has an IC50 that is less than ⅕ of IC50 of Bebtelovimab.
8. The antibody or fragment of embodiments 1-4, wherein the antibody has a potency against the SARS-CoV-2 variant that is at least 2 times that of Bebtelovimab.
9. The antibody or fragment of embodiments 1-4, wherein the antibody has a potency against the SARS-CoV-2 variant that is at least 2 times that of Bebtelovimab.
10. The antibody or fragment of embodiments 1-4, wherein the antibody has a potency against the SARS-CoV-2 variant that is at least 3 times that of Bebtelovimab.
11. The antibody or fragment of embodiments 1-4, wherein the antibody has a potency against the SARS-CoV-2 variant that is at least 5 times that of Bebtelovimab.
12. The antibody or fragment of embodiments 1-4, wherein the antibody has a potency against the SARS-CoV-2 variant that is at least 10 times that of Bebtelovimab.
13. The antibodies or fragment of embodiments 1-12, wherein the antibody also inhibits binding of ACE2-Fc and spike trimer protein of a SARS-CoV-2 variant selected from B.1.1.529, BA.2, BA.5, BA.4.6, BA.2.75.2, BN.1, and combinations thereof.
14. The antibodies or fragment of embodiments 1-13, wherein the antibody also inhibits binding of ACE2-Fc and spike trimer protein of SARS-CoV-2 (Wuhan wild type).
15. An antibody or a fragment thereof that inhibits the binding of ACE2 protein with spike trimer protein of SARS-CoV-2 (Wuhan wild type) and spike trimer proteins of SARS-CoV-2 variants B.1.1.529, BA.2, BA.5, BA.4.6, BA.2.75.2, BA.2.3.20, BQ.1.1, BN.1, XBB, and XBB.1.
16. An antibody or a fragment thereof that binds to or interacts with a SARS-CoV-2 variant XBB at a binding or interacting site as indicated in FIG. 9A.
17. An antibody or a fragment thereof that binds to or interacts with a SARS-CoV-2 variant BA.4.6 at a binding or interacting site as indicated in FIG. 9B.
18. An antibody or a fragment thereof that binds to or interacts with a SARS-CoV-2 variant BQ.1.1 at a binding or interacting site as indicated in FIG. 9C.
19. An antibody or a fragment thereof that binds to or interacts with a SARS-CoV-2 variant BA.2.75.2 at a binding or interacting site as indicated in FIG. 9D.
20. The antibody or fragment of any one of embodiments 1-19, wherein the antibody has an LCDR1 selected from SEQ ID NOs: 75-108, 287-290, and 313.
21. The antibody or fragment of any one of embodiments 1-20, wherein the antibody has an LCDR2 selected from SEQ ID NOs: 109-142, 291-294, and 314.
22. The antibody or fragment of any one of embodiments 1-21, wherein the antibody has an LCDR3 selected from SEQ ID NOs: 143-176, 295-298, and 315.
23. The antibody or fragment of any one of embodiments 1-22, wherein the antibody has an HCDR1 selected from SEQ ID NOs: 177-210, 299-302, and 316.
24. The antibody or fragment of any one of embodiments 1-23, wherein the antibody has an HCDR2 selected from SEQ ID NOs: 211-244, 303-306, and 317.
25. The antibody or fragment of any one of embodiments 1-24, wherein the antibody has an HCDR3 selected from SEQ ID NOs: 245-278, 307-310, and 318.
26. The antibody or fragment of any one of embodiments 1-25, wherein the antibody has a light chain sequence from SEQ ID NOs: 7-40, 279-282, and 311.
27. The antibody or fragment of any one of embodiments 1-26, wherein the antibody has a heavy chain sequence from SEQ ID NOs: 41-74, 283-286, and 312.
28. An antibody or a fragment thereof that inhibits binding of ACE2 protein with spike trimer protein of SARS-CoV-2 and/or variant(s) thereof, wherein the antibody has an LCDR1 selected from SEQ ID NOs: 75-108, 287-290, and 313.
29. The antibody or a fragment thereof of any one of embodiments 28, wherein the antibody has an LCDR2 selected from SEQ ID NOs: 109-142, 291-294, and 314.
30. The antibody or fragment of any one of embodiments 28-29, wherein the antibody has an LCDR3 selected from SEQ ID NOs: 143-176, 295-298, and 315.
31. The antibody or fragment of any one of embodiments 28-30, wherein the antibody has an HCDR1 selected from SEQ ID NOs: 177-210, 299-302, and 316.
32. The antibody or fragment of any one of embodiments 28-31, wherein the antibody has an HCDR2 selected from SEQ ID NOs: 211-244, 303-306, and 317.
33. The antibody or fragment of any one of embodiments 28-32, wherein the antibody has an HCDR3 selected from SEQ ID NOs: 245-278, 307-310, and 318.
34. An antibody or a fragment thereof that inhibits binding of ACE2 protein with spike trimer protein of SARS-CoV-2 and/or variant(s) thereof, wherein the antibody, wherein the antibody has a light chain sequence from SEQ ID NOs: 7-40, 279-282, and 311.
35. An antibody or a fragment thereof that inhibits binding of ACE2 protein with spike trimer protein of SARS-CoV-2 and/or variant(s) thereof, wherein the antibody, wherein the antibody has a heavy chain sequence from SEQ ID NOs: 41-74, 283-286, and 312.
36. An antibody or a fragment thereof that inhibits binding of ACE2 protein with spike trimer protein of SARS-CoV-2 and/or variant(s) thereof, wherein the antibody has a light chain sequence from SEQ ID NOs: 7-40, 279-282, and 311, and has a heavy chain sequence from SEQ ID NOs: 41-74, 283-286, and 312.
37. An antibody or a fragment thereof selected from the group consisting of 3R4-H4, 5R4-H1, 5R4-F3, 5R4-E11, 6R4-H1, 6R4-A7, 6R4-G10, 6R4-G11, 6R4-E12, 6R4-E3, 6R4-H5, 6R4-H11, 6R6-A7, 6R6-E1, 6R6-E2, 6R6-E3, 6R6-E4, 6R6-F3, 6R6-F7, 6R6-F9, 6R6-F11, 6R6-F12, 3R6-D11Q, 6R6-E6Q, 6R8A-E11, 6R8A-F2, 6R8A-E1, 6R8A-A2, 6R8C-G3, 6R8C-G4, 6R8C-G8, 6R8C-D9, 6R8C-D10, 6R8C-F10, 6R9A-A2, 6R9B-B11, 6R9B-E9, 6R9E-A8, and S3R4F12.
38. An antibody or a fragment thereof selected from the group consisting of 6R8A-E11, 6R8A-F2, 6R8A-E1, 6R8A-A2, 6R8C-G8, 6R8C-D9, 6R8C-D10, 6R8C-F10, 6R9A-A2, 6R9B-B11, 6R9B-E9, and 6R9E-A8.
39. The antibodies or fragment of embodiment 1-38, wherein the antibody or fragment are resistant to mutations at G339D/H, R346T, K356T, L368I, K444T/R, V445P, G446S, N450D, L452R/M, N460K, E484K/R, F486V/S, F490S, Q493R, G496S, T547K.
40. The antibodies or fragment of embodiment 1-39, wherein the antibody or fragment are resistant to mutations at S371F, S373P, S375F, T376A, D405N, R408S, K417N, N440K, S477N, T478K, Q498R, N501Y, Y505H (RBD in Omicron variants).
41. An antibody or a fragment thereof that inhibits binding of ACE2 protein with spike trimer protein of a SARS-CoV-2 variant selected from the group consisting of XBB.1.31, XBB.1.16.1, EG.5, HV.1, FL.1.5.1, HK.3, BA.2.86, BA.2.86.1, JN.1, and combinations thereof.
42. An antibody or a fragment thereof that inhibits binding of ACE2-Fc and spike trimer proteins of two SARS-CoV-2 variants selected from the group consisting of HV.1, HK.3, JN.1, and combinations thereof.
43. An antibody or a fragment thereof that inhibits binding of ACE2-Fc and spike trimer proteins of SARS-CoV-2 variants HV.1.
44. An antibody or fragment that inhibits binding of ACE2-Fc and spike trimer protein of a SARS-CiV-2 variant better than Bebtelovimab, wherein the SARS-CiV-2 variant is selected from the group consisting of XBB.1.31, XBB.1.16.1, EG.5, HV.1, FL.1.5.1, HK.3, BA.2.86, BA.2.86.1, JN.1, and combinations thereof.
45. The antibody or fragment of embodiments 41-44, wherein the antibody has an IC50 that is less than ½ of IC50 of Bebtelovimab.
46. The antibody or fragment of embodiments 41-44, wherein the antibody has an IC50 that is less than ⅓ of IC50 of Bebtelovimab.
47. The antibody or fragment of embodiments 41-44, wherein the antibody has an IC50 that is less than ⅕ of IC50 of Bebtelovimab.
48. The antibody or fragment of embodiments 41-44, wherein the antibody has a potency against the SARS-CoV-2 variant that is at least 2 times that of Bebtelovimab.
49. The antibody or fragment of embodiments 41-44, wherein the antibody has a potency against the SARS-CoV-2 variant that is at least 2 times that of Bebtelovimab.
50. The antibody or fragment of embodiments 41-44, wherein the antibody has a potency against the SARS-CoV-2 variant that is at least 3 times that of Bebtelovimab.
51. The antibody or fragment of embodiments 41-44, wherein the antibody has a potency against the SARS-CoV-2 variant that is at least 5 times that of Bebtelovimab.
52. The antibody or fragment of embodiments 41-44, wherein the antibody has a potency against the SARS-CoV-2 variant that is at least 10 times that of Bebtelovimab.
53. The antibody or fragment of any one of embodiments 41-52, wherein the antibody has an LCDR1 selected from SEQ ID NOs: 319, 325, 331, 337, 343, or 349.
54. The antibody or fragment of any one of embodiments 41-53, wherein the antibody has an LCDR2 selected from SEQ ID NOs: 320, 326, 332, 338, 344, or 350.
55. The antibody or fragment of any one of embodiments 41-54, wherein the antibody has an LCDR3 selected from SEQ ID NOs: 321, 327, 333, 339, 345, or 351.
56. The antibody or fragment of any one of embodiments 41-55, wherein the antibody has an HCDR1 selected from SEQ ID NOs: 322, 328, 334, 340, 346, or 352.
57. The antibody or fragment of any one of embodiments 41-56, wherein the antibody has an HCDR2 selected from SEQ ID NOs: 323, 329, 335, 341, 347, or 353.
58. The antibody or fragment of any one of embodiments 41-57, wherein the antibody has an HCDR3 selected from SEQ ID NOs: 324, 330, 336, 342, 348, or 354.
59. The antibody or fragment of any one of embodiments 41-58, wherein the antibody has a variable light chain comprising CDRs according to SEQ ID Nos: 319-321, and a variable heavy chain comprising CDRs according to SEQ ID Nos: 322-324.
60. The antibody or fragment of any one of embodiments 41-58, wherein the antibody has a variable light chain comprising CDRs according to SEQ ID Nos: 335-327, and a variable heavy chain comprising CDRs according to SEQ ID Nos: 328-330.
61. The antibody or fragment of any one of embodiments 41-58, wherein the antibody has a variable light chain comprising CDRs according to SEQ ID Nos: 331-333, and a variable heavy chain comprising CDRs according to SEQ ID Nos: 334-336.
62. The antibody or fragment of any one of embodiments 41-58, wherein the antibody has a variable light chain comprising CDRs according to SEQ ID Nos: 337-339, and a variable heavy chain comprising CDRs according to SEQ ID Nos: 340-342.
63. The antibody or fragment of any one of embodiments 41-58, wherein the antibody has a variable light chain comprising CDRs according to SEQ ID Nos: 343-345, and a variable heavy chain comprising CDRs according to SEQ ID Nos: 346-348.
64. The antibody or fragment of any one of embodiments 41-58, wherein the antibody has a variable light chain comprising CDRs according to SEQ ID Nos: 349-351, and a variable heavy chain comprising CDRs according to SEQ ID Nos: 352-354.
65. The antibodies or fragment of embodiment 1-64, wherein the antibody is humanized antibody.

### Non-Limiting Examples

The following Examples are detailed by way of illustration only and are not to be construed as limiting in spirit or in scope, many modifications both in materials and in methods will be apparent to those skilled in the art.

### Example 1. Affinity Maturation and Specificity Engineering of a Humanized Anti-SARS-CoV-2 Neutralizing Antibodies

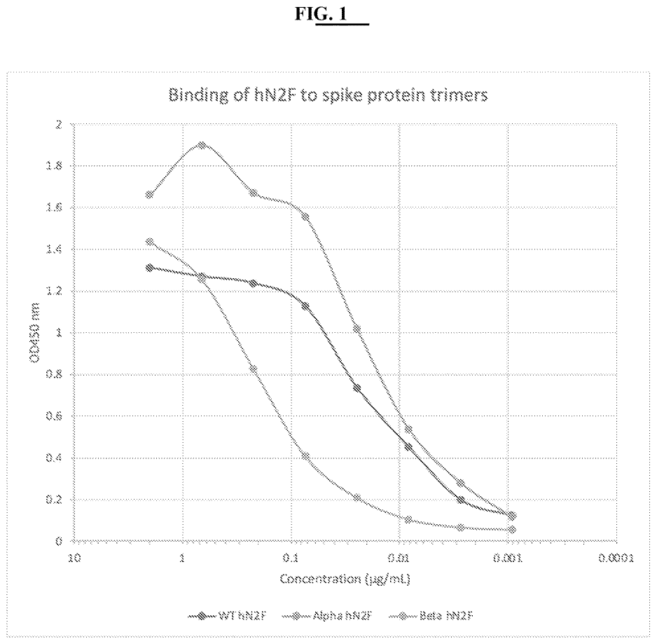

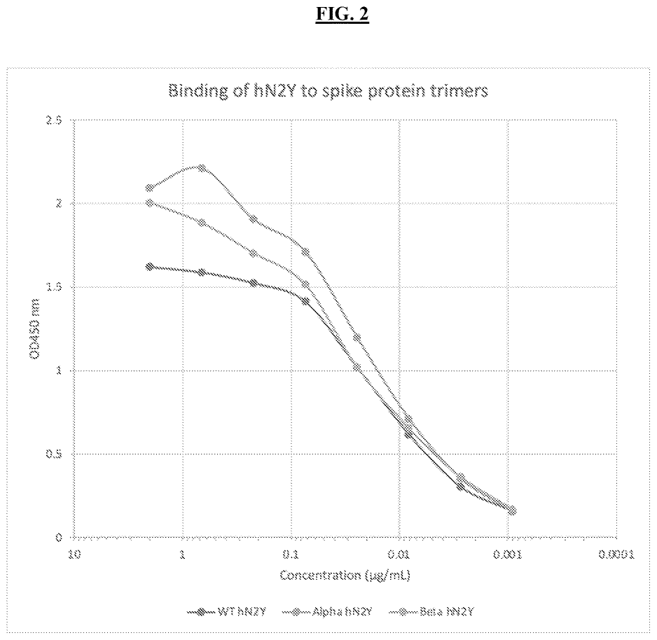

A rabbit monoclonal antibody C-A11 was developed from a rabbit immunized with a recombinant protein containing a receptor binding domain of the SARS-CoV-2 spike protein (SEQ ID No. 1, 4). The clone effectively blocked the binding of spike protein to its cell surface receptor ACE2 and was humanized by CDR grafting into selected human frameworks designated as hN2F and hN2Y (SEQ ID No. 2, 3, 5, 6). C-A11 and these humanized clones bound to the spike protein trimers of wild type (WT), Alpha, and Beta variants (FIGS. 1 and 2).

To further increase the affinity to variants of concern such as Alpha (B.1.1.7), Beta (B.1.351), Gamma (P.1), Kappa (B.1.617.1), and Epsilon (B.1.429), 6 single CDR libraries were made by careful analysis of amino acid usage by human antibodies in each CDR to cover the widest practical diversity and to further humanize CDR sequences. These libraries were panned 4 rounds on wild type spike protein trimer to eliminate non-binders in Stage 1.

The libraries were screened by ELISA and the phage from the round that gave positive signals were amplified by PCR, and pre-selected Stage 1 LCDR1, LCR2, and LCDR3 libraries were combined by an overlap PCR into a Stage 2 light chain library Lx. The same strategy was used for the heavy chain CDR libraries, and a Stage 2 heavy chain library Hx was made. These libraries were selected with higher stringency on spike protein trimers of the wild type, Alpha (B.1.1.7), Beta (B.1.351), and Epsilon (B.1.429) variants to select binders with higher affinity. The phage was treated briefly with heat before the incubation with the antigen to select for clones that are more stable. After selection, the libraries were screened by ELISA. The Stage 2 light chain library panned on the spike protein trimer of Beta (B.1.351) variant (L×C) showed a number of positive clones against the spike protein trimers of wild type and Beta (B.1.351) variant. The stage 2 heavy chain libraries panned on the spike protein trimers of wild type, Alpha (B.1.1.7), Beta (B.1.351), and Epsilon (B.1.429) (H×E, H×F, H×G, and H×H, respectively) also showed many clones with improved binding than the parent clone.

In Stage 3, the pre-selected L×C library was combined with the mixture of H×E, H×F, H×G, and H×H libraries and panned with even higher stringency on the spike protein trimer BA.5 for 6 rounds with increasing stringency followed by 2 rounds on BA.2.75.2 spike trimer to achieve the highest affinity to all variants. The phage was also pre-treated by incubation at 65 °C for 5 min to remove unstable clones and negatively selected on Baculovirus particle (BVP) coated wells to remove polyreactive clones to select clones that have favorable developability.

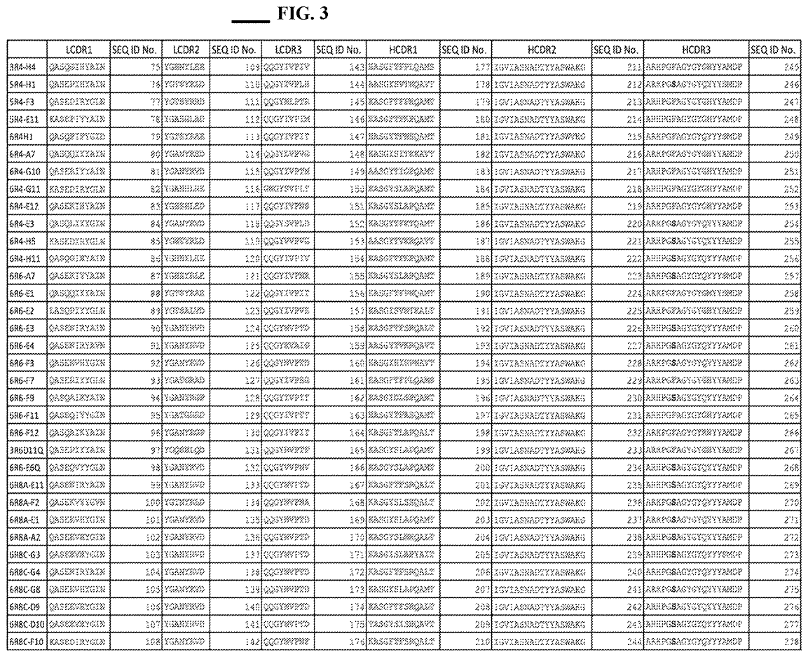

These isolated clones were converted to IgG (SEQ ID NOs: 7-40 for light chains; and SEQ ID NOs: 41-74 for heavy chains). CDRs (LCDR1, LCDR2, LCRD3, HCDR1, HCDR2, HCDR3) of those clone are show in FIG. 3 as SEQ ID NOs: 75-278.

### Further Selection on BQ.1.1 and XBB Spike Trimers

The selected phage after 6 rounds of selection on BA.5 and 2 rounds on BA.2.75.2 were further selected on BQ.1.1 and XBB spike trimers. The resultant clones 6R9B-B11 and 6R9B-E9 showed excellent inhibition of BQ.1.1 and XBB.1 spike trimer binding to ACE2 coated wells as crude Fab and were converted to IgG (SEQ ID No. 279-282 for light chains; and SEQ ID NOs: 283-286 for heavy chains). CDRs (LCDR1, LCDR2, LCRD3, HCDR1, HCDR2, HCDR3) of those clones are show in FIG. 4 as SEQ ID NOs: 287-310.

### Example 2. sVNT Against SARS-CoV-2 and SARS-CoV-2 Variants

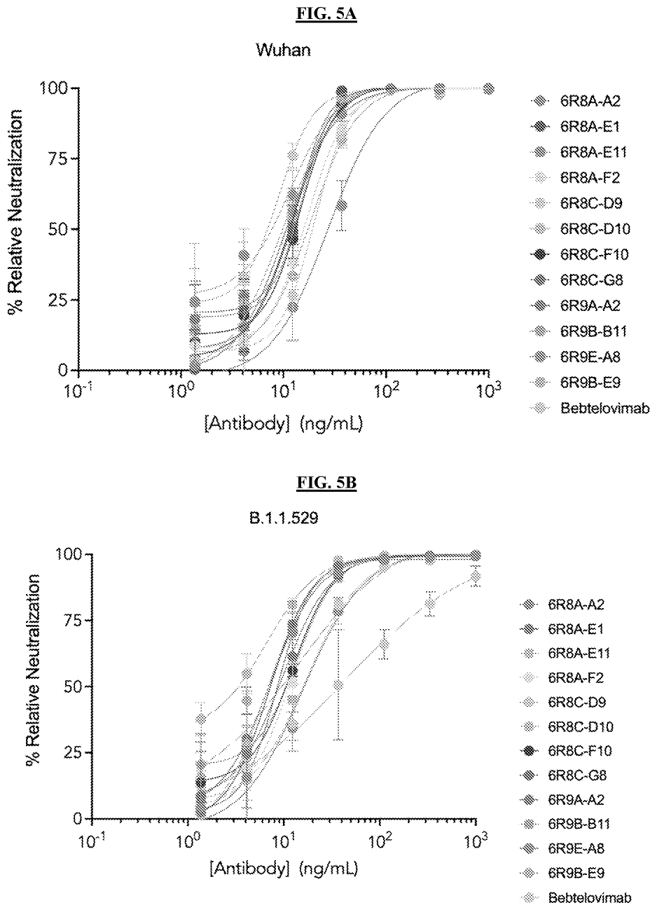

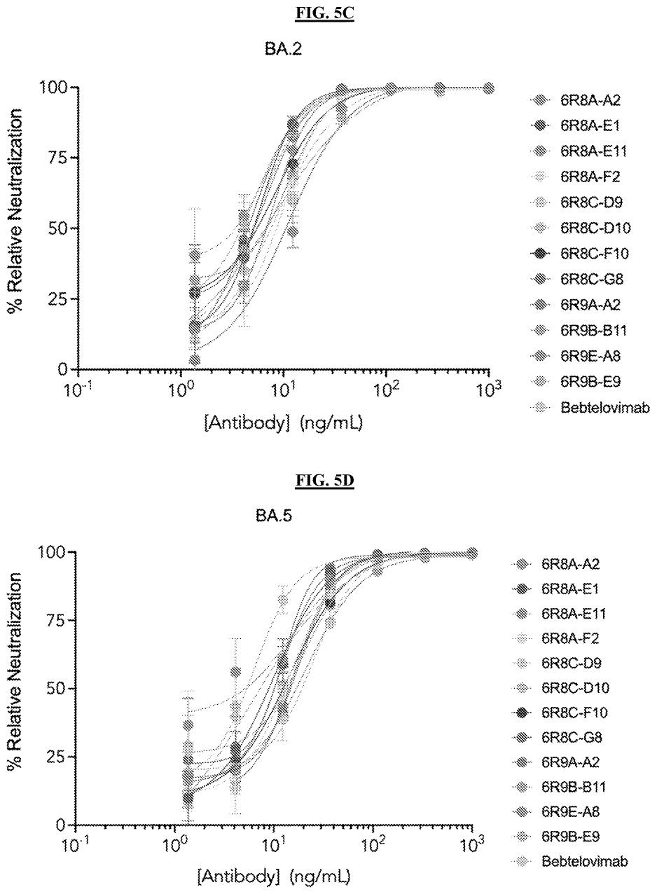

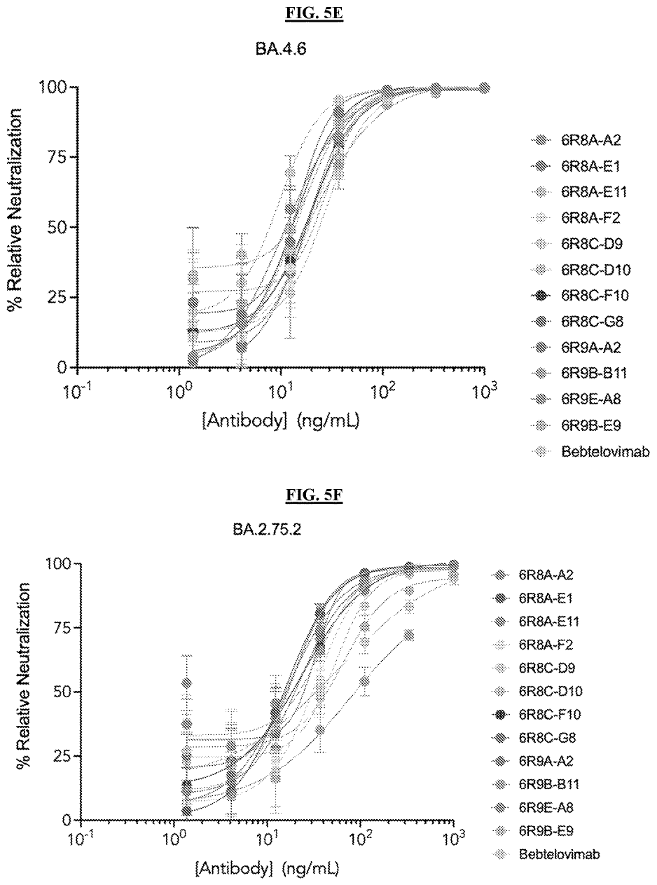

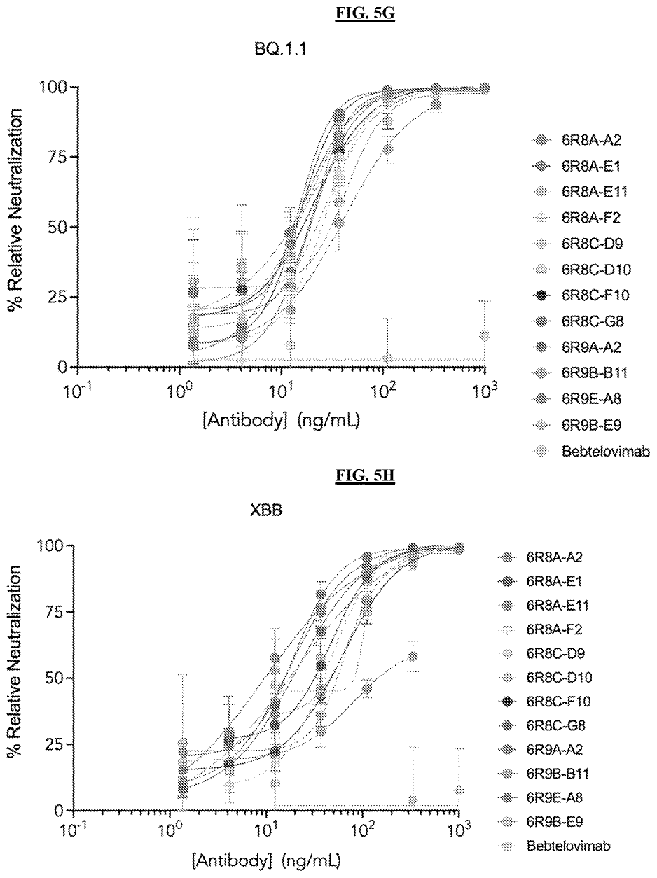

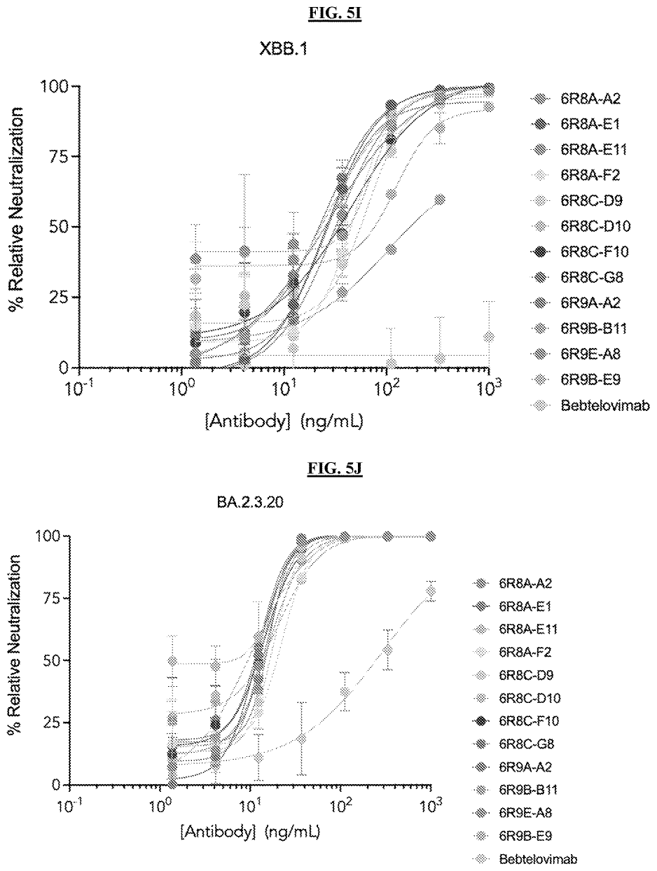

These were tested one by one in a surrogate neutralization test (Tan C W, Chia W N, Qin X, et al. 2020) using recombinant spike trimers with Histidine tag for detection. The microtiter wells were coated with recombinant ACE2-Fc protein at 2 μg/mL. The spike trimer protein (96 ng/mL) was mixed with serially diluted antibody 1:1 at RT for 1 hour and added to the wells. Bound spike trimer was detected with Horseradish peroxidase conjugated rabbit anti-His tag antibody and the inhibition of binding (IC50 ng/mL) was calculated using Prism 9 software (Table 1). The experiments were performed in triplicates. Five of these clones 6R8A-E1 (SEQ ID No. 33 and SEQ ID No. 67), 6R8C-G8 (SEQ ID No. 37 and SEQ ID No. 71), 6R8A-A2 (SEQ ID No. 34 and SEQ ID No. 68), 6R9A-A2 (SEQ ID No. 279 and SEQ ID. No 283) and 6R8C-D (SEQ ID No. 39 and SEQ ID No. 73) showed potent neutralization of Wuhan (wild type), B.1.1.529, BA.2, BA, 5, BA.4.6, BA.2.7.5.2, BQ.1.1, XBB, XBB.1, BN.1 and BA.2.3.20 (FIG. 5A-5K).

TABLE 1
IC50 (ng/mL) of clones against each spike trimer in sVNT

| | 6R8A- | 6R8A- | 6R8A- | 6R8A- | 6R8C- | 6R8C- | 6R8C- | 6R8C- | 6R9A- | 6R9B- | 6R9E- | 6R9B- | |
|---|---|---|---|---|---|---|---|---|---|---|---|---|---|
| | A2 | E1 | E11 | F2 | D9 | D10 | F10 | G8 | A2 | B11 | A8 | E9 | Bebtelovimab |
| Wuhan | 10.63 | 12.15 | 11.59 | 16.92 | 20.3 | 9.796 | 14.54 | 12.42 | 14.03 | 15.01 | 27.35 | 18.5 | 8.992 |
| B.1.1.529 | 8.645 | 7.11 | 6.61 | 11.87 | 17.26 | 6.536 | 12.3 | 7.207 | 9.626 | 13.58 | 15.8 | 13.96 | 40.3 |
| BA.2 | 7.782 | 5.832 | 6.814 | 10.22 | 8.427 | 5.849 | 8.36 | 4.876 | 7.055 | 11.09 | 11.27 | 7.766 | 5.298 |
| BA.5 | 11.82 | 9.916 | 16.06 | 14.62 | 24.78 | 5.866 | 14.42 | 12.43 | 17.8 | 20.87 | 16.11 | 20.01 | 6.346 |
| BA.4.6 | 10.83 | 13.71 | 21.28 | 21.66 | 27.73 | 14.47 | 19.85 | 15.15 | 19.14 | 18.46 | 18.24 | 28.28 | 9.649 |
| BA.2.75.2 | 16.75 | 15.36 | 36.71 | 33.84 | 53.7 | 19 | 23.75 | 20.66 | 22.04 | 78.67 | 33.25 | 65.75 | 55.87 |
| BQ.1.1 | 14.27 | 15.06 | 18.71 | 27.4 | 26.66 | 18.75 | 21.12 | 15.89 | 22.03 | 41.83 | 19.39 | 43.42 | >1,000 |
| XBB | 9.624 | 46.92 | 77.86 | 43.68 | 106.6 | 18.82 | 61.01 | 20.78 | 20.24 | 79.34 | 17.82 | 65.27 | >1,000 |
| XBB.1 | 26.45 | 25.58 | 72.88 | 48.8 | 64.33 | 24.89 | 45.18 | 25.12 | 26.06 | 103.4 | 31.09 | 124.2 | >1,000 |
| BA.2.3.20 | 13.59 | 11.93 | 18.13 | 22.48 | 18.12 | 10.13 | 14.3 | 13.05 | 15.48 | 16.05 | 16.24 | 20.49 | 297.6 |
| BN.1 | 8.917 | 7.877 | 18.53 | 19.96 | 16.27 | 9.924 | 16.01 | 9.288 | 10.28 | 17.52 | 23.03 | 31.24 | 35.53 |

*Bebtelovimab was purchased through ProteoGenix (Schiltigheim, France).
ND: Not done.

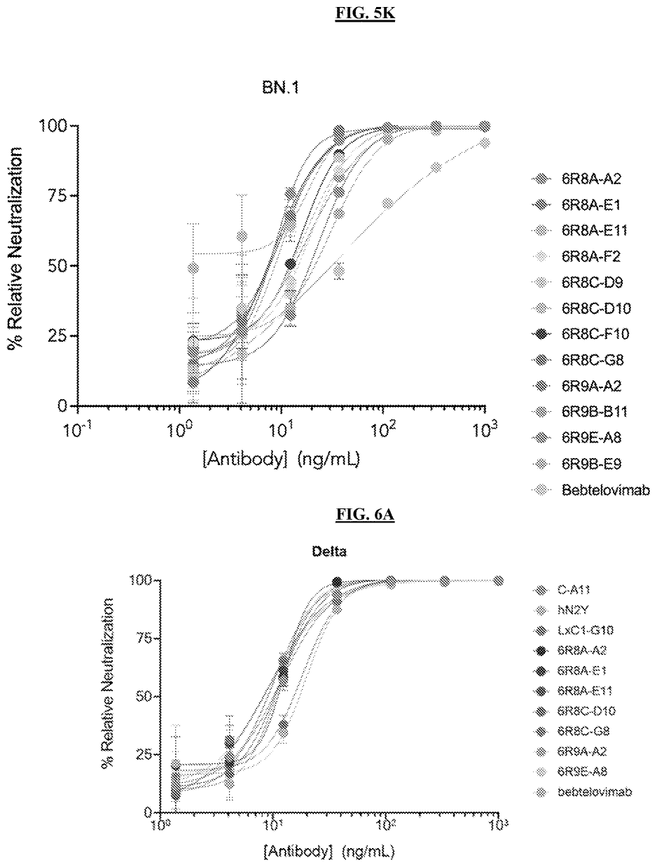

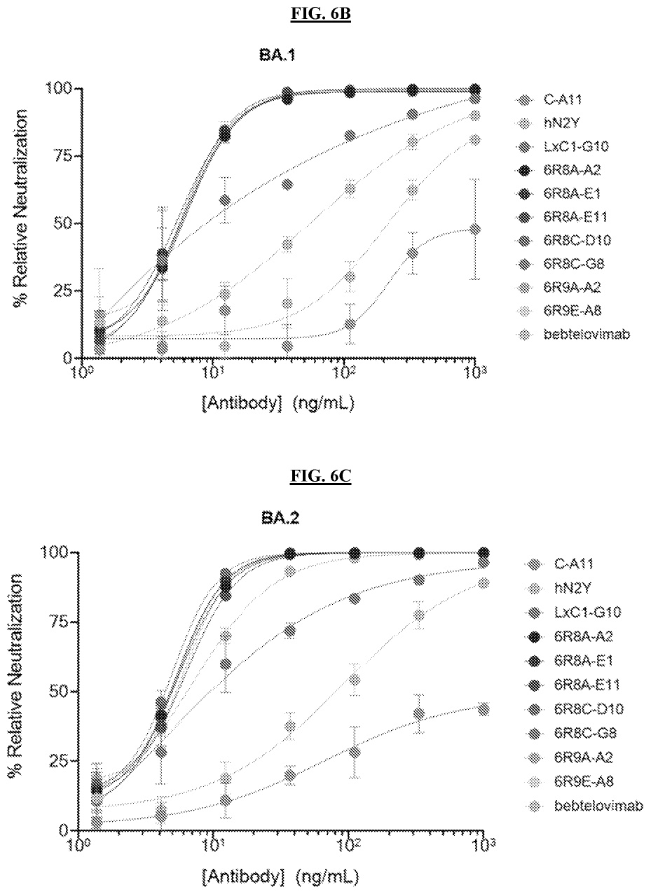

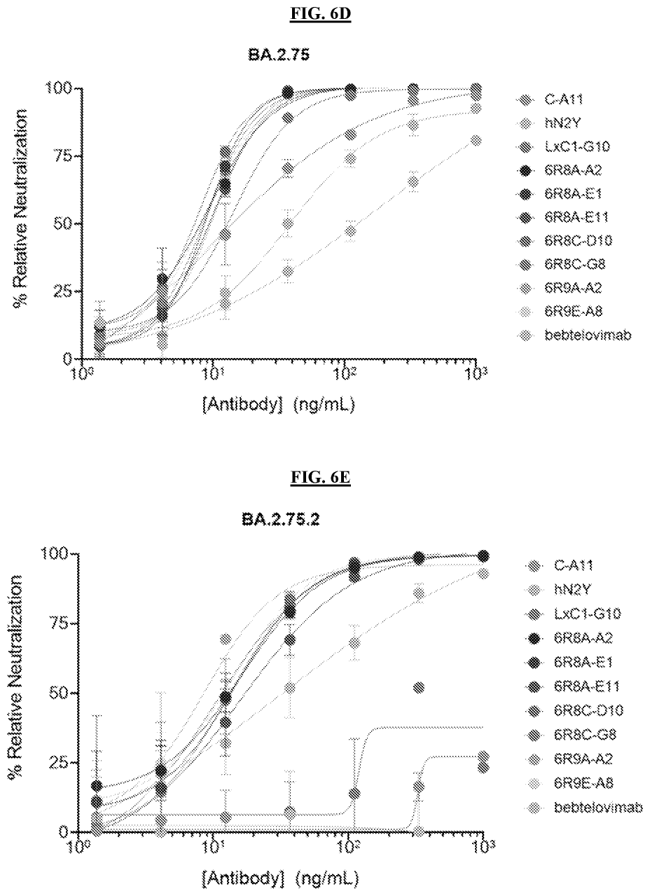

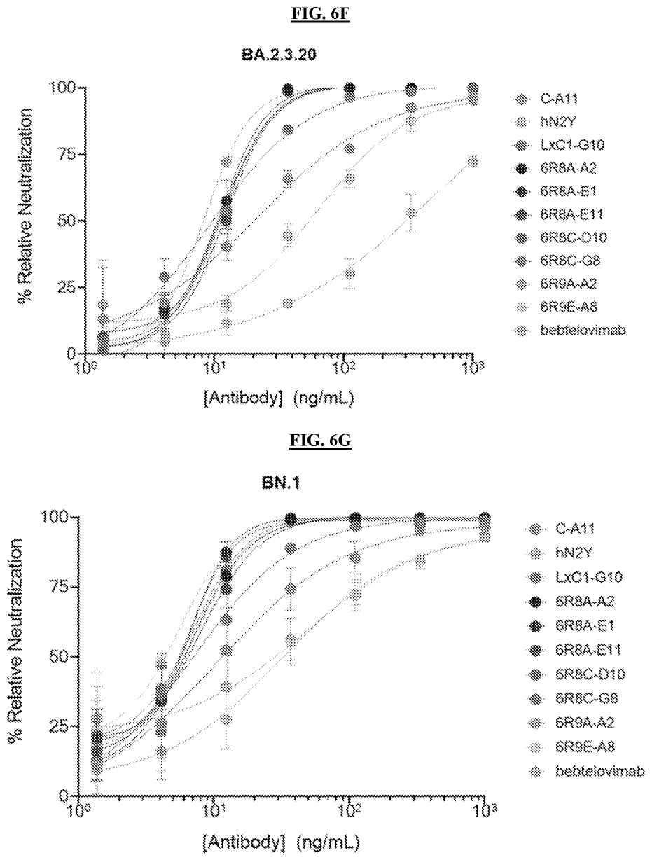

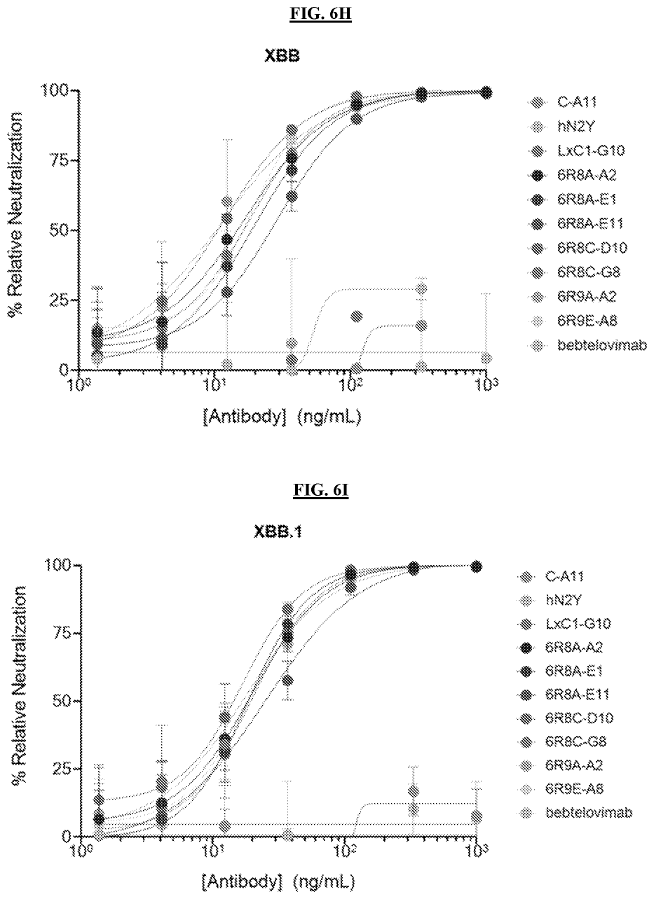

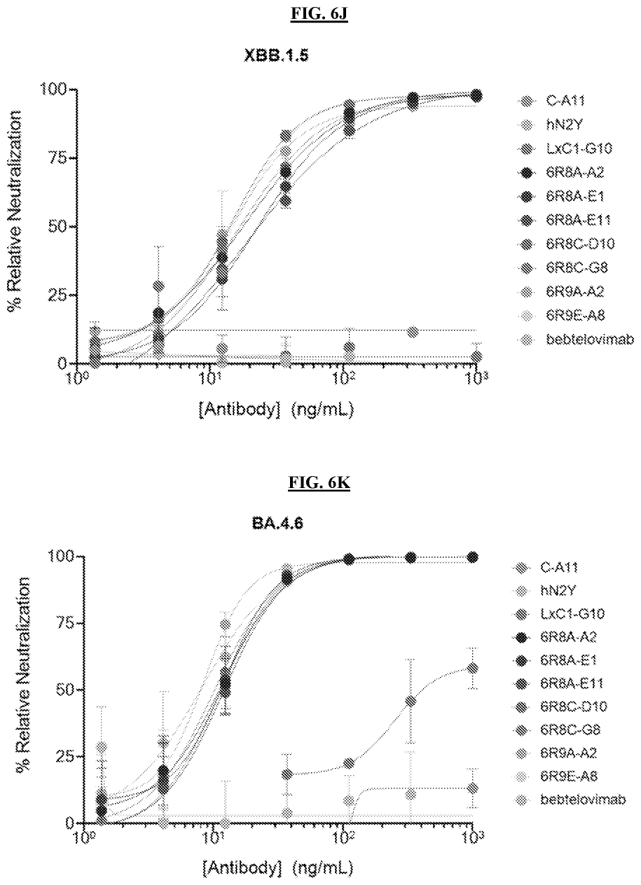

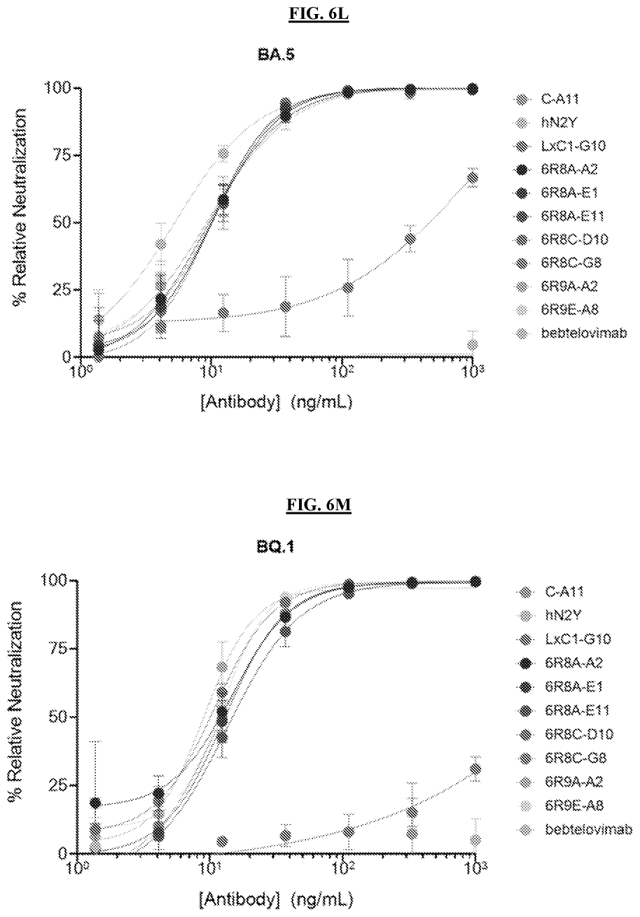

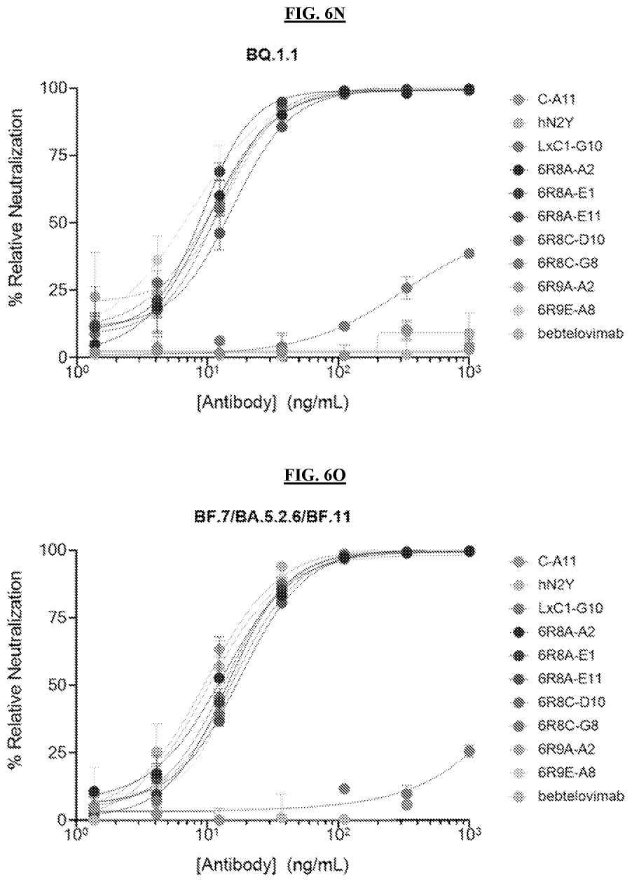

Addition sVNT testing were conducted through surrogate virus neutralization test (sVNT) using recombinant spike trimers and ACE2-Fc protein. As shown in FIG. 6A-6O, inhibitions of the binding of spike trimers (48 ng/mL) to ACE2-Fc (2 μg/mL) coated wells by antibodies are shown. Assays were performed in triplicate. 6R8A-A2, 6R8A-E1, 6R8A-E11, 6R8C-D10, 6R8C-G8, 6R9A-A2, and 6R9E-A8 showed potent neutralization of Delta and all Omicron variants, while bebtelovimab did not show any significant inhibition of BQ.1.1, XBB, and XBB.1 and weak neutralization of BA.2.3.20.

Table 2 shows calculated IC50 values derived from IgG sVNT data. 6R8A-A2, 6R8A-E1, 6R8A-E11, 6R8C-D10, 6R8C-G8, 6R9A-A2, 6R9E-A8, and S3R4F12 showed low ng/mL IC50 values for all variants tested, while bebtelovimab did not neutralize the latest Omicron variants including XBB, XBB.1, XBB.1.5, BQ.1, or BQ.1.1. NA, non-neutralizing (>1000 ng/mL IC50); UD, undefined; nd, not done.

TABLE 2

| | C-A11 | | hN2Y | | LxC1-G10 | | 6R8A-A2 | | 6R8A-E1 | | 6R8A-E11 | |
|---|---|---|---|---|---|---|---|---|---|---|---|---|
| Wuhan | 9.7 | [6.1-13.3] | 19.4 | [16.9-22.4] | 6.7 | [4.0-9.1] | 11.7 | [9.9-13.3] | 11.6 | [9.6-13.5] | 11.1 | [9.3-12.8] |
| Delta | 11.6 | [10.3-12.8] | 19.8 | [16.0-24.5] | 18.3 | [14.1-23.1] | 12.7 | [11.2-14.4] | 8.9 | [6.0-11.9] | 11.5 | [9.3-13.5] |
| BA.1 | [symbol] | | 211.5 | [118.8-6215] | 5.7 | [UD-17.9] | 6.2 | [5.8-6.7] | 5.4 | [3.8-6.9] | 5.7 | [4.7-6.9] |
| BA.2 | 66.9 | [25.1-UD] | 91.9 | [57.9-232.7] | 4.6 | [UD-11.8] | 5.5 | [5.1-6.0] | 5.2 | [4.8-5.6] | 6.2 | [5.4-7.2] |
| BA.2.75 | 10.2 | [4.9-14.7] | 38.5 | [29.6-51.0] | 14.4 | [11.0-17.9] | 9.2 | [7.0-11.4] | 8.9 | [8.2-9.8] | 9.8 | [8.7-10.9] |
| BA.2.75.2 | [symbol] | | [symbol] | | [symbol] | | 16.5 | [12.1-21.7] | 14.9 | [6.8-24.8] | 15.9 | [3.2-27.6] |
| BA.2.3.20 | 21.5 | [6.0-38.7] | 63.6 | [41.4-110.6] | 9.1 | [4.5-13.1] | 11.5 | [10.5-12.4] | 11.2 | [9.6-12.7] | 12.2 | [10.5-13.6] |
| BN.1 | 10.4 | [0.02-19.5] | 32.8 | [20.0-53.5] | 7.1 | [2.5-10.8] | 6.7 | [5.4-8.3] | 6.9 | [5.8-8.4] | 7.6 | [5.6-9.8] |

| XBB | [symbol] | [symbol] | [symbol] | | 17.1 | [9.7-26.6] | 19.4 | [12.8-28.1] | 28.9 | [20.9-39.3] |
|---|---|---|---|---|---|---|---|---|---|---|
| XBB.1 | [symbol] | [symbol] | [symbol] | | 20.0 | [13.3-28.6] | 18.5 | [12.7-26.0] | 25.1 | [14.2-39.1] |
| XBB.1.5 | [symbol] | [symbol] | [symbol] | | 18.4 | [13.2-24.0] | 22.5 | [19.0-26.4] | 18.1 | [13.8-22.5] |
| BA.4.6 | [symbol] | [symbol] | 252.1 | [UD] | 11.8 | [8.1-15.5] | 12.4 | [9.4-15.8] | 11.9 | [8.4-15.4] |
| BA.5 | [symbol] | [symbol] | 788.6 | [187.2-UD] | 9.4 | [6.8-11.9] | 10.3 | [8.0-12.6] | 10.3 | [7.9-12.8] |

| | | | | | | | | | |
|---|---|---|---|---|---|---|---|---|---|
| BQ.1 | [symbol] | [symbol] | [symbol] | 14.7 | [10.5-20.2] | 12.9 | [10.5-15.6] | 13.5 | [11.1-16.2] |
| BQ.1.1 | [symbol] | [symbol] | [symbol] | 9.9 | [7.9-11.9] | 9.3 | [8.5-10.3] | 15.4 | [12.4-19.0] |
| BF.7/ | [symbol] | [symbol] | [symbol] | 13.1 | [10.3-16.3] | 14.3 | [13.2-15.5] | 17.8 | [15.6-20.3] |
| BA.5.2.6/ | | | | | | | | | |
| BF.11 | | | | | | | | | |

| | 6R8C-D10 | | 6R8C-G8 | | 6R9A-A2 | | 6R9E-A8 | | S3R4F12 | | bebtelovimab | |
|---|---|---|---|---|---|---|---|---|---|---|---|---|
| Wuhan | 12.5 | [9.7-14.9] | 11.9 | [10.6-13.1] | 9.8 | [8.1-11.6] | 13.1 | [11.0-15.0] | 9.6 | [6.8-12.4] | 8.1 | [6.3-10.1] |
| Delta | 11.6 | [9.7-13.1] | 9.7 | [8.4-11.0] | 10.5 | [9.8-11.2] | 12.6 | [11.3-13.8] | 7.4 | [2.0-11.9] | 9.5 | [8.0-11.1] |
| BA.1 | 5.8 | [5.4-6.2] | 6.0 | [5.5-6.5] | 6.7 | [6.1-7.4] | 5.6 | [5.1-6.1] | nd | | 54.9 | [37.4-96.3] |
| BA.2 | 5.0 | [4.6-5.4] | 5.6 | [5.3-5.9] | 6.2 | [5.9-6.6] | 5.6 | [5.4-5.7] | 6.1 | [5.4-6.9] | 7.2 | [6.5-7.8] |
| BA.2.75 | 8.1 | [6.9-9.4] | 8.8 | [8.0-9.6] | 10.5 | [9.8-11.2] | 8.9 | [8.0-9.8] | 8.1 | [7.0-9.3] | 180.8 | [63.3-UD] |
| BA.2.75.2 | 10.6 | [1.9-17.8] | 13.0 | [9.3-17.1] | 8.6 | [4.6-11.9] | 13.9 | [9.6-18.3] | 9.0 | [7.1-11.0] | 30 | [UD] |
| BA.2.3.20 | 11.8 | [10.0-13.4] | 10.5 | [8.6-12.4] | 8.4 | [7.8-9.1] | 13.2 | [11.3-15.0] | nd | | 536.2 | [144.3-UD] |
| BN.1 | 6.6 | [5.0-9.1] | 7.2 | [5.5-9.1] | 7.3 | [6.0-8.8] | 5.6 | [5.0-6.3] | 5.5 | [2.4-7.9] | 48.4 | [15.9-UD] |
| XBB | 11.6 | [7.5-15.8] | 18.6 | [12.4-26.8] | 11.2 | [2.4-23.0] | 19.0 | [14.7-24.8] | 10.9 | [6.1-15.6] | [symbol] | |
| XBB.1 | 16.9 | [13.1-21.6] | 20.0 | [13.3-28.3] | 17.8 | [11.7-25.8] | 19.3 | [15.7-23.5] | nd | | [symbol] | |
| XBB.1.5 | 14.9 | [13.4-16.6] | 15.7 | [12.9-18.8] | 14.7 | [10.8-19.9] | 15.9 | [14.2-17.7] | 15.2 | [13.9-16.6] | [symbol] | |
| BA.4.6 | 11.1 | [7.0-15.2] | 10.2 | [7.3-13.1] | 8.6 | [6.6-UD] | 12.5 | [9.2-15.8] | nd | | 8.3 | [3.0-12.9] |
| BA.5 | 10.0 | [8.5-11.4] | 10.7 | [7.5-14.1] | 9.4 | [7.1-11.7] | 9.7 | [6.2-13.0] | 7.6 | [6.7-8.7] | 5.3 | [2.8-7.1] |

| | | | | | | | | | | | |
|---|---|---|---|---|---|---|---|---|---|---|---|
| BQ.1 | 10.9 | [9.2-12.7] | 11.5 | [9.7-13.3] | 9.1 | [7.4-10.8] | 12.5 | [11.0-14.0] | 9.3 | [7.0-11.8] | [symbol] |
| BQ.1.1 | 12.0 | [10.9-13.1] | 11.1 | [9.3-12.9] | 14.1 | [11.4-17.6] | 7.4 | [4.6-9.9] | 9.8 | [8.9-10.8] | [symbol] |

| | | | | | | | | | | | |
|---|---|---|---|---|---|---|---|---|---|---|---|
| BF.7/ | 14.0 | [12.0-16.1] | 16.2 | [14.3-18.2] | 10.0 | [8.2-11.9] | 16.5 | [14.6-18.7] | nd | 9.9 | [7.5-12.3] |
| BA.5.2.6/ | | | | | | | | | | | |
| BF.11 | | | | | | | | | | | |

[symbol]

indicates data missing or illegible when filed

### Example 3. BLI Affinity Measurement for Engineered IgGs

BLI affinity measurement of binding to BQ.1.1 and XBB.1.5 for engineered IgGs were conducted. In each assay, biotinylated spike protein trimer was captured to the sensor surface and dipped into a 7-fold dilution series of IgG at a starting concentration of 10 nM. Binding was monitored for 4 minutes, followed by dissociation monitored for 30 minutes. For each variant tested, each experiment is shown with the same scale y-axis. As illustrated in FIG. 7, both the humanized lead candidate hN2Y and the light chain engineered clone L×CI-G10 lost binding activity for later SARS-CoV-2 variants, but 6R8A-A2, 6R8A-E1, 6R8A-E11, 6R8C-D10, 6R8C-G8, 6R9A-A2, and 6R9E-A8 showed strong binding to BQ.1.1 and XBB.1.5. Therapeutic mAbs tested identically showed weak or no binding to the latest Omicron variants.

BLI traces were globally fit to a 1:1 binding model to derive affinity and kinetic values. As show in Table 3 below, some IgG affinities were below the detection limit of the assay (<1 pM) due to exceptionally slow off-rates even when measured for 30 minutes. For some weakly binding clones, affinity and/or kinetic values could not be determined. N.B., non-binding. 6R8A-A2, 6R8A-E1, 6R8A-E11, 6R8C-D10, 6R8C-G8, 6R9A-A2, and 6R9E-A8 showed high affinity to XBB.1.5 and BQ.1.1 variants.

TABLE 3

| | Beta | Delta | BA.1 | BQ.1.1 | XBB.1.5 |
|---|---|---|---|---|---|
| | pM K_{D} | | | | |
| C-A11 | 40 ± 1 | | | | |
| hN2Y | 190 ± 20 | | 120 ± 1 | 32000 ± 3800 | N.B. |
| LxC1-G10 | <1.0 | 21 ± 1 | 24 ± 1 | 2100 ± 25 | N.B. |
| 6R8A-A2 | | | | 6.9 ± 1 | 36 ± 1 |
| 6R8A-E1 | | | | 1.2 ± 1 | 52 ± 1 |
| 6R8A-E11 | | | | <1 | 66 ± 1 |
| 6R8C-D10 | | | | 5.2 ± 1 | 39 ± 1 |
| 6R8C-G8 | | | | <1 | 18 ± 1 |
| 6R9A-A2 | | | | 4.8 ± 1 | 67 ± 1 |
| 6R9E-A8 | | | | <1 | 42 ± 1 |
| bebtelovimab | | | | 3800 ± 49 | N.B. |
| tixagevimab | | 27.6 ± 1 | 1700000 | N.B. | N.B. |
| cilgavimab | | <1 | 1700 | N.B. | N.B. |
| | kₐ, ×10⁶M⁻¹*s⁻¹ | | | | |
| C-A11 | 2.61 ± 0.01 | | | | |
| hN2Y | 0.41 ± 0.04 | | 2.32 ± 0.009 | 0.21 ± 0.02 | N.B. |
| LxC1-G10 | 2.14 ± 0.003 | 1.14 ± 0.002 | 1.67 ± 0.003 | 2.03 ± 0.02 | N.B. |
| 6R8A-A2 | | | | 1.41 ± 0.003 | 1.47 ± 0.003 |
| 6R8A-E1 | | | | 1.83 ± 0.002 | 1.97 ± 0.005 |
| 6R8A-E11 | | | | 1.53 ± 0.002 | 1.39 ± 0.003 |
| 6R8C-D10 | | | | 1.70 ± 0.002 | 1.42 ± 0.004 |
| 6R8C-G8 | | | | 0.98 ± 0.002 | 1.39 ± 0.003 |
| 6R9A-A2 | | | | 1.46 ± 0.002 | 1.47 ± 0.003 |
| 6R9E-A8 | | | | 1.47 ± 0.001 | 1.82 ± 0.003 |
| bebtelovimab | | | | 2.08 ± 0.02 | N.B. |
| tixagevimab | | 1.77 ± 0.003 | n.d. | N.B. | N.B. |
| cilgavimab | | 31.0 ± 0.002 | n.d. | N.B. | N.B. |
| | kₐ, ×10⁶ s⁻¹ | | | | |
| C-A11 | 106 ± 0.5 | | | | |
| hN2Y | 78.3 ± 0.7 | | 286 ± 0.5 | 6680 ± 120 | N.B. |
| LxC1-G10 | <0.1 | 24.1 ± 0.1 | 39.9 ± 0.2 | 4310 ± 22 | N.B. |
| 6R8A-A2 | | | | 9.75 ± 0.2 | 52.7 ± 0.2 |
| 6R8A-E1 | | | | 21.8 ± 0.1 | 103 ± 0.2 |
| 6R8A-E11 | | | | <0.1 | 92.4 ± 0.2 |
| 6R8C-D10 | | | | 8.94 ± 0.1 | 55.1 ± 0.2 |
| 6R8C-G8 | | | | <0.1 | 25.1 ± 0.2 |
| 6R9A-A2 | | | | 6.94 ± 0.1 | 98.6 ± 0.2 |
| 6R9E-A8 | | | | 1.29 ± 0.1 | 76.3 ± 0.2 |
| bebtelovimab | | | | 7900 ± 56 | N.B. |
| tixagevimab | | 49.0 ± 0.2 | n.d. | N.B. | N.B. |
| cilgavimab | | <0.1 | n.d. | N.B. | N.B. |

### Example 4. Epitop Binding Prediction Using ColabFold Model

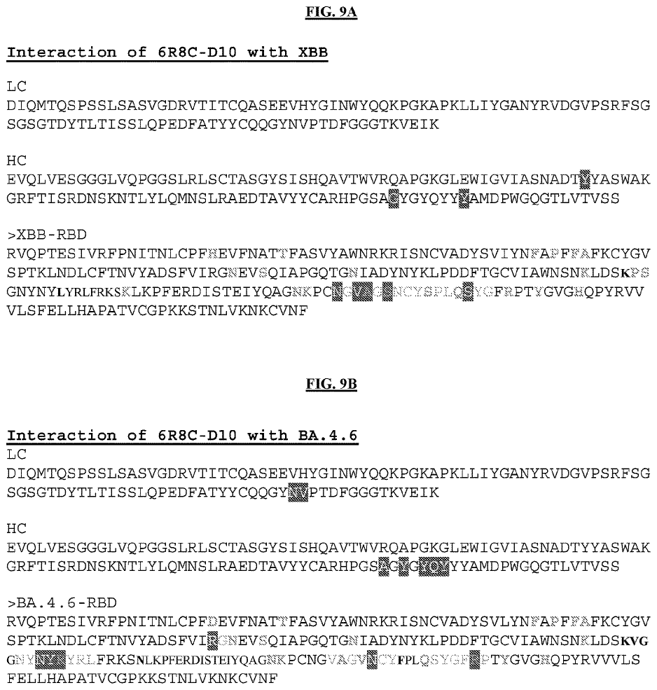

The structure of antibodies 6R8A-A2 (SEQ ID No. 34 and SEQ ID No. 68), 6R8A-E1 (SEQ ID No. 33 and SEQ ID No. 67), 6R8A-E11 (SEQ ID No. 31 and SEQ ID No. 65), 6R8A-F2 (SEQ ID No. 32 and SEQ ID No. 66), 6R8C-D9 (SEQ ID No. 38 and SEQ ID No. 72), 6R8C-D10 (SEQ ID No. 39 and SEQ ID No. 73), 6R8C-F10 (SEQ ID No. 40 and SEQ ID No. 74), 6R8C-G8 (SEQ ID No. 37 and SEQ ID No. 71), 6R9A-A2 (SEQ ID No. 279 and SEQ ID No. 283), 6R9B-B11 (SEQ ID No. 280 and SEQ ID No. 284), 6R9B-E9 (SEQ ID No. 281 and SEQ ID No. 285), and 6R9E-A8 (SEQ ID No. 282 and SEQ ID No. 286) were separately predicted and modeled using ColabFold. Both Fvs and SARS-CoV-2 BQ.1.1 variant RBD sequences were simultaneously modeled, and docked structures were predicted. The prediction was compared to an existing cryo-EM structure of spike protein-ACE2 complex (FIG. 8). The BQ.1.1 RBD of each predicted docked structure was aligned to the RBD of the cryo-EM structure to show the binding epitope of the antibodies. Notably, all antibodies are predicted to bind within the established neutralizing epitope RBD2, defined by the Coronavirus Immunotherapy Consortium, overlapping with the ACE2 binding site.

Similarly, 6R8C-D10 (SEQ ID No. 39 and SEQ ID No. 73) was modeled and docked with RBD variants XBB, BA.4.6, BQ.1.1, and BA.2.75.2 (FIG. 9A-9D). Residues involved in polar contacts are highlighted and show a potential mechanism of broad neutralization of the variants where CDRs L3 and H3 recognize a similar region of RBD among the variants.

### Example 5. S3R4F12

To create the clone S3R4F12, single CDR libraries were first created based on the humanized lead candidate hN2Y. These single CDR libraries were selected on wild-type spike trimer to create a pool of functional CDR sequences. Pre-selected CDR pools were then amplified from phage and combined by overlap PCR to create separate combined light and combined heavy chain libraries. These libraries were then selected on BA.5 spike trimer to enrich for clones that bind to this variant. Next, these pre-selected light and heavy chain libraries were then paired to create a single combined library. The single combined library was selected on late-stage Omicron variants BQ.1.1, XBB, and XBB.1 to increase neutralization potency. Fab screening identified the strong neutralizing clone S3R4F12, which was converted to IgG for further characterization. sVNT revealed strong neutralization potency for Wuhan, Delta, BA.2, BA.2.75, BA.2.75.2, BN.1, XBB, XBB.1.5, BA.5, BQ.1, and BQ.1.1.

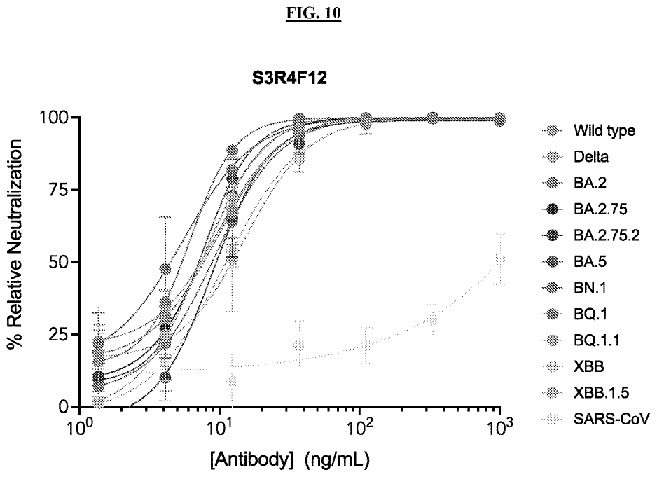

FIG. 10 illustrates surrogate virus neutralization test (sVNT) using recombinant spike trimers and ACE2-Fc protein. Inhibitions of the binding of spike trimers (48 ng/mL) to ACE2-Fc (2 μg/mL) coated wells by antibodies are shown. Assays were performed in triplicate. S3R4F12 shows strong neutralization of all SARS-CoV-2 variants tested and moderate neutralization of SARS-CoV B039.

### Example 6. Additional Clones for Recent Omicron Variants

After obtaining broadly neutralizing 6R8/6R9 series mAbs, extensively characterized above, new Stage A1 libraries targeting all six CDRs were designed and constructed, using 6R8C-D10 and 6R9C-G8 as templates. These six CDR libraries were selected alternating rounds using directly coated XBB.1.16 and XBB.1.31 spike protein trimers for four rounds and using pH 5.8 elution to obtain mild pH sensitive clones with increased antibody recycling function in vivo. Following Fab expression and screening, new libraries were prepared for Stage A2 by amplifying pre-selected CDR pools from the final round of selected phage. CDR pools from Stage A1 selected CDRs L1, L2, and L3 were combined by overlap PCR to create a combined light chain library, paired with the wild-type 6R8C-D10/6R9C-G8 heavy chain, and CDR pools from Stage A1 selected CDRs H1, H2, and H3 were combined by overlap PCR to create a combined heavy chain library, paired with the wild-type 6R8C-D10/6R9C-G8 light chain. Stage A2 selection was performed by selecting on directly coated EG.5 spike protein trimer for four rounds, using pH 5.8 elution. Fab screening identified clone H×B10 from the combined heavy chain Stage A2 panned library. One Stage B library was created by amplifying light or heavy chains from the final round selected phage from Stage A2 panning and sequentially cloning into a new Fab-phage vector (Stage B library A and G). Stage B library B was created by cloning H×B10 heavy chain and pairing this with the naive Stage A2 combined light chain library. Stage B library C used the same library from which the 6R8/6R9 series mAb clones were obtained. During Stage B selection of libraries, His-tagged spike trimer was captured by anti-His mAb for selection, and pH 5.8 buffer was used for elution. For libraries A, B, and C, alternating variants were used each round for selection including EG.5 in round 1, FL.1.5.1 in round 2, BA.2.86 in round 3, BA.2.86.1 in round 4, and HK.3 in rounds 5-6. For library G, HK.3 was used for four rounds of selection. Fab screening following the final round of selection identified AR6-B9 from library A, BR6-C10 from library B, CR6-C8 from library C, and GR4-B12 and GR4-E11 from library G.

These clones were tested one by one in a surrogate neutralization test (Tan C W, Chia W N, Qin X, et al. 2020) using recombinant spike trimers with Histidine tag for detection. The microtiter wells were coated with recombinant ACE2-Fc protein at 2 μg/mL. The spike trimer protein (96 ng/mL) was mixed with serially diluted antibody 1:1 at RT for 1 hour and added to the wells. Bound spike trimer was detected with Horseradish peroxidase conjugated rabbit anti-His tag antibody and the inhibition of binding (IC50 ng/mL) was calculated using Prism 9 software (Table 4).

TABLE 4
sVNT IC₅₀ values (ng/mL) for additional clones against SARS-CoV-2 Omicron subvariants

| Clone ID | XBB.1.31 | XBB.1.16.1 | EG.5 | HV.1 | FL.1.5.1 | HK.3 | BA.2.86 | BA.2.86.1 | JN.1 |
|---|---|---|---|---|---|---|---|---|---|
| HxB10 | 22 | 15 | 15 | 31 | 21 | >1,000 | 10 | 13 | 81 |
| AR6-B9 | N.D. | N.D. | N.D. | 26 | N.D. | 676 | N.D. | N.D. | 80 |
| BR6-C10 | N.D. | N.D. | N.D. | 42 | N.D. | >1,000 | N.D. | N.D. | 41 |
| CR6-C8 | N.D. | N.D. | N.D. | 32 | N.D. | 38 | N.D. | N.D. | >1,000 |
| GR4-B12 | N.D. | N.D. | N.D. | 27 | N.D. | 150 | N.D. | N.D. | 32 |
| GR4-E11 | N.D. | N.D. | N.D. | 31 | N.D. | 60 | N.D. | N.D. | 26 |

N.D.: not done.

### SEQUENCE LISTING

The following nucleotide and amino acid sequences are referred to herein:

### Rabbit and Humanized Antibodies

Light Chain Sequences of Rabbit (C-A11) and Humanized (hN2F and hN2Y) Antibodies

| C-A11 | | |
|---|---|---|
| SEQ ID No. 1 | | |
| 1: | AlaIleGluMetThrGlnSerProSerSerLeuAlaAlaSerValGlyAspThrValThr | |
| | GCCATCGAGATGACCCAGTCTCCATCCTCCCTGGCTGCATCTGTGGGAGACACAGTCACC | |
| 1 | ---------!---------!---------!---------!---------!---------! | 60 |
| 1: | IleThrCysLysAlaSerGluAspIleArgTyrGlyLeuAsnTrpTyrGlnGlnLysLeu | |
| | ATCACTTGTAAGGCCAGTGAGGACATTCGTTATGGGTTAAACTGGTATCAGCAGAAACTA | |
| 61 | ---------!---------!---------!---------!---------!---------! | 120 |
| 1: | GlyIleAlaProLysLeuLeuIleTyrGlyAlaAsnThrLeuGluAlaGlyValProSer | |
| | GGGATAGCTCCTAAGCTCCTGATCTATGGGGCAAACACTTTAGAGGCTGGGGTCCCATCG | |
| 121 | ---------!---------!---------!---------!---------!---------! | 180 |
| 1: | ArgPheSerGlySerGlySerGluThrAspTyrThrLeuThrIleSerSerValGlnAla | |
| | AGGTTCAGTGGCAGCGGATCAGAGACCGATTACACCCTCACCATCAGCAGCGTGCAGGCT | |
| 181 | ---------!---------!---------!---------!---------!---------! | 240 |
| 1: | GluAspAlaGlyIleTyrTyrCysGlnGlnGlyTyrSerThrProLeuThrPheGlyAla | |
| | GAAGATGCAGGAATTTATTACTGTCAGCAAGGATATAGTACCCCTTTGACTTTTGGAGCT | |
| 241 | ---------!---------!---------!---------!---------!---------! | 300 |
| 1: | GlyThrLysValGluIleLys | |
| | GGCACCAAGGTGGAAATCAAA | |
| 301 | ---------!---------!- | 321 |
| hN2F | | |
| SEQ ID No. 2 | | |
| 1: | AspIleGlnMetThrGlnSerProSerSerLeuSerAlaSerValGlyAspArgValThr | |
| | GACATCCAGATGACCCAGTCTCCATCCTCCCTGTCTGCATCTGTAGGAGACAGAGTCACC | |
| 1 | ---------!---------!---------!---------!---------!---------! | 60 |
| 1: | IleThrCysLysAlaSerGluAspIleArgTyrGlyLeuAsnTrpTyrGlnGlnLysPro | |
| | ATCACTTGCAAGGCCAGTGAGGACATTCGTTATGGGTTAAACTGGTATCAGCAGAAACCA | |
| 61 | ---------!---------!---------!---------!---------!---------! | 120 |
| 1: | GlyLysAlaProLysLeuLeuIleTyrGlyAlaAsnThrLeuGluAlaGlyValProSer | |
| | GGGAAAGCCCCTAAGCTCCTGATCTATGGGGCAAACACTTTAGAGGCTGGGGTCCCATCA | |
| 121 | ---------!---------!---------!---------!---------!---------! | 180 |
| 1: | ArgPheSerGlySerGlySerGlyThrAspPheThrLeuThrIleSerSerLeuGlnPro | |
| | AGGTTCAGTGGCAGTGGATCTGGGACAGATTTCACTCTCACCATCAGCAGTCTGCAACCT | |
| 181 | ---------!---------!---------!---------!---------!---------! | 240 |
| 1: | GluAspPheAlaThrTyrTyrCysGlnGlnGlyTyrSerThrProLeuThrPheGlyGly | |
| | GAAGATTTTGCAACTTACTACTGTCAGCAAGGATATAGTACCCCTTTGACTTTCGGCGGA | |
| 241 | ---------!---------!---------!---------!---------!---------! | 300 |
| 1: | GlyThrLysValGluIleLys | |
| | GGGACCAAGGTGGAGATCAAA | |
| 301 | ---------!---------!- | 321 |
| hN2Y | | |
| SEQ ID No. 3 | | |
| 1: | AspIleGlnMetThrGlnSerProSerSerLeuSerAlaSerValGlyAspArgValThr | |
| | GACATCCAGATGACCCAGTCTCCATCCTCCCTGTCTGCATCTGTAGGAGACAGAGTCACC | |
| 1 | ---------!---------!---------!---------!---------!---------! | 60 |
| 1: | IleThrCysLysAlaSerGluAspIleArgTyrGlyLeuAsnTrpTyrGlnGlnLysPro | |
| | ATCACTTGCAAGGCCAGTGAGGACATTCGTTATGGGTTAAACTGGTATCAGCAGAAACCA | |
| 61 | ---------!---------!---------!---------!---------!---------! | 120 |
| 1: | GlyLysAlaProLysLeuLeuIleTyrGlyAlaAsnThrLeuGluAlaGlyValProSer | |
| | GGGAAAGCCCCTAAGCTCCTGATCTATGGGGCAAACACTTTAGAGGCTGGGGTCCCATCA | |
| 121 | ---------!---------!---------!---------!---------!---------! | 180 |
| 1: | ArgPheSerGlySerGlySerGlyThrAspTyrThrLeuThrIleSerSerLeuGlnPro | |
| | AGGTTCAGTGGCAGTGGATCTGGGACAGATTACACTCTCACCATCAGCAGTCTGCAACCT | |
| 181 | ---------!---------!---------!---------!---------!---------! | 240 |
| 1: | GluAspPheAlaThrTyrTyrCysGlnGlnGlyTyrSerThrProLeuThrPheGlyGly | |
| | GAAGATTTTGCAACTTACTACTGTCAGCAAGGATATAGTACCCCTTTGACTTTCGGCGGA | |
| 241 | ---------!---------!---------!---------!---------!---------! | 300 |
| 1: | GlyThrLysValGluIleLys | |
| | GGGACCAAGGTGGAGATCAAA | |
| 301 | ---------!---------!- | 321 |

Heavy Chain Sequences of Rabbit (C-A11) and Humanized (hN2F and hN2Y) Antibodies

| C-A11 | | |
|---|---|---|
| SEQ ID No. 4 | | |
| 1: | GlnSerLeuGluGluSerGlyGlyArgLeuValThrProGlyThrProLeuThrLeuThr | |
| | CAGTCGCTGGAGGAGTCCGGGGGTCGCCTGGTCACGCCTGGGACACCCCTGACACTCACC | |
| 1 | ---------!---------!---------!---------!---------!---------! | 60 |
| 1: | CysThrValSerGlyPheSerLeuSerThrTyrAlaMetSerTrpValArgGlnAlaPro | |
| | TGCACAGTCTCTGGATTCTCCCTCAGTACCTATGCAATGAGCTGGGTCCGCCAGGCTCCA | |
| 61 | ---------!---------!---------!---------!---------!---------! | 120 |
| 1: | GlyLysGlyLeuGluTrpIleGlyValIleAlaSerAsnAlaAspThrTyrTyrAlaSer | |
| | GGGAAGGGACTGGAATGGATCGGAGTCATTGCTAGTAATGCTGACACATACTACGCGAGC | |
| 121 | ---------!---------!---------!---------!---------!---------! | 180 |
| 1: | TrpAlaLysGlyArgIleThrIleSerLysThrSerThrThrValAspLeuLysIleThr | |
| | TGGGCGAAAGGCCGAATCACCATCTCCAAAACCTCGACCACGGTGGATCTGAAGATCACC | |
| 181 | ---------!---------!---------!---------!---------!---------! | 240 |
| 1: | SerProThrThrGluAspThrAlaThrTyrPheCysAlaArgHisProGlySerAlaGly | |
| | AGTCCGACGACCGAGGACACGGCCACCTATTTCTGTGCCAGACATCCTGGTAGTGCTGGT | |
| 241 | ---------!---------!---------!---------!---------!---------! | 300 |
| 1: | TyrGlyTyrAlaValTyrTyrAlaMetAspProTrpGlyProGlyThrLeuValThrVal | |
| | TATGGTTATGCTGTTTATTACGCCATGGACCCCTGGGGCCCAGGGACCCTCGTCACCGTC | |
| 301 | ---------!---------!---------!---------!---------!---------! | 360 |
| 1: | SerSer | |
| | TCTTCA | |
| 361 | ------ | 366 |
| hN2F | | |
| SEQ ID No. 5 | | |
| 1: | GluValGlnLeuValGluSerGlyGlyGlyLeuValGlnProGlyGlySerLeuArgLeu | |
| | GAGGTGCAGCTGGTGGAGTCTGGGGGAGGCTTGGTACAGCCTGGGGGGTCCCTGAGACTC | |
| 1 | ---------!---------!---------!---------!---------!---------! | 60 |
| 1: | SerCysThrValSerGlyPheSerLeuSerThrTyrAlaMetSerTrpValArgGlnAla | |
| | TCCTGTACAGTCTCTGGATTCTCCCTCAGTACCTATGCAATGAGCTGGGTCCGCCAGGCT | |
| 61 | ---------!---------!---------!---------!---------!---------! | 120 |
| 1: | ProGlyLysGlyLeuGluTrpIleGlyValIleAlaSerAsnAlaAspThrTyrTyrAla | |
| | CCAGGGAAGGGGCTGGAGTGGATCGGAGTCATTGCTAGTAATGCTGACACATACTACGCG | |
| 121 | ---------!---------!---------!---------!---------!---------! | 180 |
| 1: | SerTrpAlaLysGlyArgPheThrIleSerArgAspAsnSerLysAsnThrLeuTyrLeu | |
| | AGCTGGGCGAAAGGCCGGTTCACCATCTCCAGAGACAATTCCAAGAACACGCTGTATCTG | |
| 181 | ---------!---------!---------!---------!---------!---------! | 240 |
| 1: | GlnMetAsnSerLeuArgAlaGluAspThrAlaValTyrTyrCysAlaArgHisProGly | |
| | CAAATGAACAGCCTGAGAGCCGAGGACACGGCCGTATATTACTGTGCCAGACATCCTGGT | |
| 241 | ---------!---------!---------!---------!---------!---------! | 300 |
| 1: | SerAlaGlyTyrGlyTyrAlaValTyrTyrAlaMetAspProTrpGlyGlnGlyThrLeu | |
| | AGTGCTGGTTATGGTTATGCTGTTTATTACGCCATGGACCCCTGGGGCCAAGGAACCCTG | |
| 301 | ---------!---------!---------!---------!---------!---------! | 360 |
| 1: | ValThrValSerSer | |
| | GTCACCGTCTCCTCA | |
| 361 | ---------!----- | 375 |
| hN2Y | | |
| SEQ ID No. 6 | | |
| 1: | GluValGlnLeuValGluSerGlyGlyGlyLeuValGlnProGlyGlySerLeuArgLeu | |
| | GAGGTGCAGCTGGTGGAGTCTGGGGGAGGCTTGGTACAGCCTGGGGGGTCCCTGAGACTC | |
| 1 | ---------!---------!---------!---------!---------!---------! | 60 |
| 1: | SerCysThrValSerGlyPheSerLeuSerThrTyrAlaMetSerTrpValArgGlnAla | |
| | TCCTGTACAGTCTCTGGATTCTCCCTCAGTACCTATGCAATGAGCTGGGTCCGCCAGGCT | |
| 61 | ---------!---------!---------!---------!---------!---------! | 120 |
| 1: | ProGlyLysGlyLeuGluTrpIleGlyValIleAlaSerAsnAlaAspThrTyrTyrAla | |
| | CCAGGGAAGGGGCTGGAGTGGATCGGAGTCATTGCTAGTAATGCTGACACATACTACGCG | |
| 121 | ---------!---------!---------!---------!---------!---------! | 180 |
| 1: | SerTrpAlaLysGlyArgPheThrIleSerArgAspAsnSerLysAsnThrLeuTyrLeu | |
| | AGCTGGGCGAAAGGCCGGTTCACCATCTCCAGAGACAATTCCAAGAACACGCTGTATCTG | |
| 181 | ---------!---------!---------!---------!---------!---------! | 240 |
| 1: | GlnMetAsnSerLeuArgAlaGluAspThrAlaValTyrTyrCysAlaArgHisProGly | |
| | CAAATGAACAGCCTGAGAGCCGAGGACACGGCCGTATATTACTGTGCCAGACATCCTGGT | |
| 241 | ---------!---------!---------!---------!---------!---------! | 300 |
| 1: | SerAlaGlyTyrGlyTyrAlaValTyrTyrAlaMetAspProTrpGlyGlnGlyThrLeu | |
| | AGTGCTGGTTATGGTTATGCTGTTTATTACGCCATGGACCCCTGGGGCCAAGGAACCCTG | |
| 301 | ---------!---------!---------!---------!---------!---------! | 360 |
| 1: | ValThrValSerSer | |
| | GTCACCGTCTCCTCA | |
| 361 | ---------!----- | 375 |

### Clones Selected on BA.5 and BA.2.75.2

### Light Chain Sequences of Clones Selected on BA.5 and BA.2.75.2

| 3R4-H4 | | |
|---|---|---|
| SEQ ID No. 7 | | |
| 1: | AspIleGlnMetThrGlnSerProSerSerLeuSerAlaSerValGlyAspArgValThrIleThrCysGlnAlaSerGlnGlyIleHisTyrAlaIleA | |
| | GACATCCAGATGACCCAGTCTCCATCCTCCCTGTCTGCATCTGTAGGAGACAGAGTCACCATCACTTGCCAGGCCAGTCAGGGGATTCATTATGCGATTA | |
| 1 | ---------!---------!---------!---------!---------!---------!---------!---------!---------!---------! | 100 |
| 1: | snTrpTyrGlnGlnLysProGlyLysAlaProLysLeuLeuIleTyrGlyHisAsnTyrLeuGluGluGlyValProSerArgPheSerGlySerGlySe | |
| | ATTGGTATCAGCAGAAACCAGGGAAAGCCCCTAAGCTCCTGATCTATGGGCATAATTATCTTGAGGAGGGGGTCCCATCAAGGTTCAGTGGCAGTGGATC | |
| 101 | ---------!---------!---------!---------!---------!---------!---------!---------!---------!---------! | 200 |
| 1: | rGlyThrAspTyrThrLeuThrIleSerSerLeuGlnProGluAspPheAlaThrTyrTyrCysGlnGlnGlyTyrIleValProIleValPheGlyGly | |
| | TGGGACAGACTACACTCTCACCATCAGCAGTCTGCAACCTGAAGATTTTGCAACTTACTACTGTCAACAAGGATATATTGTGCCGATTGTTTTCGGCGGA | |
| 201 | ---------!---------!---------!---------!---------!---------!---------!---------!---------!---------! | 300 |
| 1: | GlyThrLysValGluIleLysArgThrValAlaAlaProSerValPheIlePheProProSerAspGluGlnLeuLysSerGlyThrAlaSerValValC | |
| | GGGACCAAGGTGGAGATCAAACGGACTGTGGCTGCACCATCTGTCTTCATCTTCCCGCCATCTGATGAGCAGTTGAAATCTGGAACTGCCTCTGTTGTGT | |
| 301 | ---------!---------!---------!---------!---------!---------!---------!---------!---------!---------! | 400 |
| 1: | ysLeuLeuAsnAsnPheTyrProArgGluAlaLysValGlnTrpLysValAspAsnAlaLeuGlnSerGlyAsnSerGlnGluSerValThrGluGlnAs | |
| | GCCTGCTGAATAACTTCTATCCCAGAGAGGCCAAAGTACAGTGGAAGGTGGATAACGCCCTCCAATCGGGTAACTCCCAGGAGAGTGTCACAGAGCAGGA | |
| 401 | ---------!---------!---------!---------!---------!---------!---------!---------!---------!---------! | 500 |
| 1: | pSerLysAspSerThrTyrSerLeuSerSerThrLeuThrLeuSerLysAlaAspTyrGluLysHisLysValTyrAlaCysGluValThrHisGlnGly | |
| | CAGCAAGGACAGCACCTACAGCCTCAGCAGCACCCTGACGCTGAGCAAAGCAGACTACGAGAAACACAAAGTCTACGCCTGCGAAGTCACCCATCAGGGC | |
| 501 | ---------!---------!---------!---------!---------!---------!---------!---------!---------!---------! | 600 |
| 1: | LeuSerSerProValThrLysSerPheAsnArgGlyGluCys | |
| | CTGAGCTCGCCCGTCACAAAGAGCTTCAACAGGGGAGAGTGT | |
| 601 | ---------!---------!---------!---------!-- | 642 |
| 5R4-H1 | | |
| SEQ ID No. 8 | | |
| 1: | AspIleGlnMetThrGlnSerProSerSerLeuSerAlaSerValGlyAspArgValThrIleThrCysGlnAlaSerGluProIleHisTyrAlaIleA | |
| | GACATCCAGATGACCCAGTCTCCATCCTCCCTGTCTGCATCTGTAGGAGACAGAGTCACCATCACTTGCCAGGCCAGTGAGCCGATTCATTATGCTATTA | |
| 1 | ---------!---------!---------!---------!---------!---------!---------!---------!---------!---------! | 100 |
| 1: | snTrpTyrGlnGlnLysProGlyLysAlaProLysLeuLeuIleTyrGlyThrSerTyrArgLeuAspGlyValProSerArgPheSerGlySerGlySe | |
| | ATTGGTATCAGCAGAAACCAGGGAAAGCCCCTAAGCTCCTGATCTATGGGACTAGTTATCGTTTGGATGGGGTCCCATCAAGGTTCAGTGGCAGTGGATC | |
| 101 | ---------!---------!---------!---------!---------!---------!---------!---------!---------!---------! | 200 |
| 1: | rGlyThrAspPheThrLeuThrIleSerSerLeuGlnProGluAspPheAlaThrTyrTyrCysGlnGlnGlyTyrThrValProLeuHisPheGlyGly | |
| | TGGGACAGATTTCACTCTCACCATCAGCAGTCTGCAACCTGAAGATTTTGCAACTTACTACTGTCAACAAGGATATACTGTGCCTTTGCATTTCGGCGGA | |
| 201 | ---------!---------!---------!---------!---------!---------!---------!---------!---------!---------! | 300 |
| 1: | GlyThrLysValGluIleLysArgThrValAlaAlaProSerValPheIlePheProProSerAspGluGlnLeuLysSerGlyThrAlaSerValValC | |
| | GGGACCAAGGTGGAGATCAAACGGACTGTGGCTGCACCATCTGTCTTCATCTTCCCGCCATCTGATGAGCAGTTGAAATCTGGAACTGCCTCTGTTGTGT | |
| 301 | ---------!---------!---------!---------!---------!---------!---------!---------!---------!---------! | 400 |
| 1: | ysLeuLeuAsnAsnPheTyrProArgGluAlaLysValGlnTrpLysValAspAsnAlaLeuGlnSerGlyAsnSerGlnGluSerValThrGluGlnAs | |
| | GCCTGCTGAATAACTTCTATCCCAGAGAGGCCAAAGTACAGTGGAAGGTGGATAACGCCCTCCAATCGGGTAACTCCCAGGAGAGTGTCACAGAGCAGGA | |
| 401 | ---------!---------!---------!---------!---------!---------!---------!---------!---------!---------! | 500 |
| 1: | pSerLysAspSerThrTyrSerLeuSerSerThrLeuThrLeuSerLysAlaAspTyrGluLysHisLysValTyrAlaCysGluValThrHisGlnGly | |
| | CAGCAAGGACAGCACCTACAGCCTCAGCAGCACCCTGACGCTGAGCAAAGCAGACTACGAGAAACACAAAGTCTACGCCTGCGAAGTCACCCATCAGGGC | |
| 501 | ---------!---------!---------!---------!---------!---------!---------!---------!---------!---------! | 600 |
| 1: | LeuSerSerProValThrLysSerPheAsnArgGlyGluCys | |
| | CTGAGCTCGCCCGTCACAAAGAGCTTCAACAGGGGAGAGTGT | |
| 601 | ---------!---------!---------!---------!-- | 642 |
| 5R4-F3 | | |
| SEQ ID No. 9 | | |
| 1: | AspIleGlnMetThrGlnSerProSerSerLeuSerAlaSerValGlyAspArgValThrIleThrCysGlnAlaSerGluAspIleArgTyrGlyLeuA | |
| | GACATCCAGATGACCCAGTCTCCATCCTCCCTGTCTGCATCTGTAGGAGACAGAGTCACCATCACTTGCCAGGCCAGTGAGGACATTCGTTATGGGTTAA | |
| 1 | ---------!---------!---------!---------!---------!---------!---------!---------!---------!---------! | 100 |
| 1: | snTrpTyrGlnGlnLysProGlyLysAlaProLysLeuLeuIleTyrGlyThrSerTyrArgArgAspGlyValProSerArgPheSerGlySerGlySe | |
| | ACTGGTATCAGCAGAAACCAGGGAAAGCCCCTAAGCTCCTGATCTATGGGACTAGTTATCGTCGTGATGGGGTCCCATCAAGGTTCAGTGGCAGTGGATC | |
| 101 | ---------!---------!---------!---------!---------!---------!---------!---------!---------!---------! | 200 |
| 1: | rGlyThrAspTyrThrLeuThrIleSerSerLeuGlnProGluAspPheAlaThrTyrTyrCysGlnGlnGlyTyrAsnLeuProThrArgPheGlyGly | |
| | TGGGACAGATTACACTCTCACCATCAGCAGTCTGCAACCTGAAGATTTTGCAACTTACTACTGTCAACAAGGATATAATCTTCCGACTAGGTTCGGCGGA | |
| 201 | ---------!---------!---------!---------!---------!---------!---------!---------!---------!---------! | 300 |
| 1: | GlyThrLysValGluIleLysArgThrValAlaAlaProSerValPheIlePheProProSerAspGluGlnLeuLysSerGlyThrAlaSerValValC | |
| | GGGACCAAGGTGGAGATCAAACGGACTGTGGCTGCACCATCTGTCTTCATCTTCCCGCCATCTGATGAGCAGTTGAAATCTGGAACTGCCTCTGTTGTGT | |
| 301 | ---------!---------!---------!---------!---------!---------!---------!---------!---------!---------! | 400 |
| 1: | ysLeuLeuAsnAsnPheTyrProArgGluAlaLysValGlnTrpLysValAspAsnAlaLeuGlnSerGlyAsnSerGlnGluSerValThrGluGlnAs | |
| | GCCTGCTGAATAACTTCTATCCCAGAGAGGCCAAAGTACAGTGGAAGGTGGATAACGCCCTCCAATCGGGTAACTCCCAGGAGAGTGTCACAGAGCAGGA | |
| 401 | ---------!---------!---------!---------!---------!---------!---------!---------!---------!---------! | 500 |
| 1: | pSerLysAspSerThrTyrSerLeuSerSerThrLeuThrLeuSerLysAlaAspTyrGluLysHisLysValTyrAlaCysGluValThrHisGlnGly | |
| | CAGCAAGGACAGCACCTACAGCCTCAGCAGCACCCTGACGCTGAGCAAAGCAGACTACGAGAAACACAAAGTCTACGCCTGCGAAGTCACCCATCAGGGC | |
| 501 | ---------!---------!---------!---------!---------!---------!---------!---------!---------!---------! | 600 |
| 1: | LeuSerSerProValThrLysSerPheAsnArgGlyGluCys | |
| | CTGAGCTCGCCCGTCACAAAGAGCTTCAACAGGGGAGAGTGT | |
| 5R4-E11 | | |
| SEQ ID No. 10 | | |
| 1: | AspIleGlnMetThrGlnSerProSerSerLeuSerAlaSerValGlyAspArgValThrIleThrCysLysAlaSerGluProIleTyrTyrAlaIleA | |
| | GACATCCAGATGACCCAGTCTCCATCCTCCCTGTCTGCATCTGTAGGAGACAGAGTCACCATCACTTGCAAAGCCAGTGAGCCTATTTATTATGCGATTA | |
| 1 | ---------!---------!---------!---------!---------!---------!---------!---------!---------!---------! | 100 |
| 1: | snTrpTyrGlnGlnLysProGlyLysAlaProLysLeuLeuIleTyrGlyAlaSerGlyLeuAlaAspGlyValProSerArgPheSerGlySerGlySe | |
| | ATTGGTATCAGCAGAAACCAGGGAAAGCCCCTAAGCTCCTGATCTATGGTGCGAGTGGTCTTGCGGATGGGGTCCCATCAAGGTTCAGTGGCAGTGGATC | |
| 101 | ---------!---------!---------!---------!---------!---------!---------!---------!---------!---------! | 200 |
| 1: | rGlyThrAspTyrThrLeuThrIleSerSerLeuGlnProGluAspPheAlaThrTyrTyrCysGlnGlnGlyTyrIleValProIleMetPheGlyGly | |
| | TGGGACAGATTACACTCTCACCATCAGCAGTCTGCAACCTGAAGATTTTGCAACTTACTACTGTCAACAAGGATATATTGTGCCTATTATGTTCGGCGGA | |
| 201 | ---------!---------!---------!---------!---------!---------!---------!---------!---------!---------! | 300 |
| 1: | GlyThrLysValGluIleLysArgThrValAlaAlaProSerValSerIlePheProProSerAspGluGlnLeuLysSerGlyThrAlaSerValValC | |
| | GGGACCAAGGTGGAGATCAAACGGACTGTGGCTGCACCATCTGTCTCCATCTTCCCGCCATCTGATGAGCAGTTGAAATCTGGAACTGCCTCTGTTGTGT | |
| 301 | ---------!---------!---------!---------!---------!---------!---------!---------!---------!---------! | 400 |
| 1: | ysLeuLeuAsnAsnPheTyrProArgGluAlaLysValGlnTrpLysValAspAsnAlaLeuGlnSerGlyAsnSerGlnGluSerValThrGluGlnAs | |
| | GCCTGCTGAATAACTTCTATCCCAGAGAGGCCAAAGTACAGTGGAAGGTGGATAACGCCCTCCAATCGGGTAACTCCCAGGAGAGTGTCACAGAGCAGGA | |
| 401 | ---------!---------!---------!---------!---------!---------!---------!---------!---------!---------! | 500 |
| 1: | pSerLysAspSerThrTyrSerLeuSerSerThrLeuThrLeuSerLysAlaAspTyrGluLysHisLysValTyrAlaCysGluValThrHisGlnGly | |
| | CAGCAAGGACAGCACCTACAGCCTCAGCAGCACCCTGACGCTGAGCAAAGCAGACTACGAGAAACACAAAGTCTACGCCTGCGAAGTCACCCATCAGGGC | |
| 501 | ---------!---------!---------!---------!---------!---------!---------!---------!---------!---------! | 600 |
| 1: | LeuSerSerProValThrLysSerPheAsnArgGlyGluCys | |
| | CTGAGCTCGCCCGTCACAAAGAGCTTCAACAGGGGAGAGTGT | |
| 601 | ---------!---------!---------!---------!-- | 642 |
| 6R4-H1 | | |
| SEQ ID No. 11 | | |
| 1: | AspIleGlnMetThrGlnSerProSerSerLeuSerAlaSerValGlyAspArgValThrIleThrCysGlnAlaSerGlnProIlePheTyrGlyIleA | |
| | GACATCCAGATGACCCAGTCTCCATCCTCCCTGTCTGCATCTGTAGGAGACAGAGTCACCATCACTTGCCAGGCCAGTCAGCCTATTTTTTATGGTATTG | |
| 1 | ---------!---------!---------!---------!---------!---------!---------!---------!---------!---------! | 100 |
| 1: | spTrpTyrGlnGlnLysProGlyLysAlaProLysLeuLeuIleTyrGlyThrSerTyrArgAlaGluGlyValProSerArgPheSerGlySerGlySe | |
| | ATTGGTATCAGCAGAAACCAGGGAAAGCCCCTAAGCTCCTGATCTATGGGACTAGTTATCGTGCGGAGGGGGTCCCATCAAGGTTCAGTGGCAGTGGATC | |
| 101 | ---------!---------!---------!---------!---------!---------!---------!---------!---------!---------! | 200 |
| 1: | rGlyThrAspTyrThrLeuThrIleSerSerLeuGlnProGluAspPheAlaThrTyrTyrCysGlnGlnGlyTyrIleValProIleThrPheGlyGly | |
| | TGGGACAGATTACACTCTCACCATCAGCAGTCTGCAACCTGAAGATTTTGCAACTTACTACTGTCAACAAGGATATATTGTGCCTATTACGTTCGGCGGA | |
| 201 | ---------!---------!---------!---------!---------!---------!---------!---------!---------!---------! | 300 |
| 1: | GlyThrLysValGluIleLysArgThrValAlaAlaProSerValPheIlePheProProSerAspGluGlnLeuLysSerGlyThrAlaSerValValC | |
| | GGGACCAAGGTGGAGATCAAACGGACTGTGGCTGCACCATCTGTCTTCATCTTCCCGCCATCTGATGAGCAGTTGAAATCTGGAACTGCCTCTGTTGTGT | |
| 301 | ---------!---------!---------!---------!---------!---------!---------!---------!---------!---------! | 400 |
| 1: | ysLeuLeuAsnAsnPheTyrProArgGluAlaLysValGlnTrpLysValAspAsnAlaLeuGlnSerGlyAsnSerGlnGluSerValThrGluGlnAs | |
| | GCCTGCTGAATAACTTCTATCCCAGAGAGGCCAAAGTACAGTGGAAGGTGGATAACGCCCTCCAATCGGGTAACTCCCAGGAGAGTGTCACGGAGCAGGA | |
| 401 | ---------!---------!---------!---------!---------!---------!---------!---------!---------!---------! | 500 |
| 1: | pSerLysAspSerThrTyrSerLeuSerSerThrLeuThrLeuSerLysAlaAspTyrGluLysHisLysValTyrAlaCysGluValThrHisGlnGly | |
| | CAGCAAGGACAGCACCTACAGCCTCAGCAGCACCCTGACGCTGAGCAAAGCAGACTACGAGAAACACAAAGTCTACGCCTGCGAAGTCACCCATCAGGGC | |
| 501 | ---------!---------!---------!---------!---------!---------!---------!---------!---------!---------! | 600 |
| 1: | LeuSerSerProValThrLysSerPheAsnArgGlyGluCys | |
| | CTGAGCTCGCCCGTCACAAAGAGCTTCAACAGGGGAGAGTGT | |
| 601 | ---------!---------!---------!---------!-- | 642 |
| 6R4-A7 | | |
| SEQ ID No. 12 | | |
| 1: | AspIleGlnMetThrGlnSerProSerSerLeuSerAlaSerValGlyAspArgValThrIleThrCysGlnAlaSerGlnGlnIleTyrTyrAlaIleA | |
| | GACATCCAGATGACCCAGTCTCCATCCTCCCTGTCTGCATCTGTAGGAGACAGAGTCACCATCACTTGCCAGGCCAGTCAGCAGATTTATTATGCTATTA | |
| 1 | ---------!---------!---------!---------!---------!---------!---------!---------!---------!---------! | 100 |
| 1: | snTrpTyrGlnGlnLysProGlyLysAlaProLysLeuLeuIleTyrGlyAlaAsnTyrArgGluAspGlyValProSerArgPheSerGlySerGlySe | |
| | ATTGGTATCAGCAGAAACCAGGGAAAGCCCCTAAGCTCCTGATCTATGGTGCGAATTATCGTGAGGATGGGGTCCCATCAAGGTTCAGTGGCAGTGGATC | |
| 101 | ---------!---------!---------!---------!---------!---------!---------!---------!---------!---------! | 200 |
| 1: | rGlyThrAspTyrThrLeuThrIleSerSerLeuGlnProGluAspPheAlaThrTyrTyrCysGlnGlnGlyTyrLeuValProValGlyPheGlyGly | |
| | TGGGACAGATTACACTCTCACCATCAGCAGTCTGCAACCTGAAGATTTTGCAACTTACTACTGTCAACAAGGATATCTGGTTCCTGTTGGTTTCGGCGGA | |
| 201 | ---------!---------!---------!---------!---------!---------!---------!---------!---------!---------! | 300 |
| 1: | GlyThrLysValGluIleLysArgThrValAlaAlaProSerValPheIlePheProProSerAspGluGlnLeuLysSerGlyThrAlaSerValValC | |
| | GGGACCAAGGTGGAGATCAAACGGACTGTGGCCGCACCATCTGTCTTCATCTTCCCGCCATCTGATGAGCAGTTGAAATCTGGAACTGCCTCTGTTGTGT | |
| 301 | ---------!---------!---------!---------!---------!---------!---------!---------!---------!---------! | 400 |
| 1: | ysLeuLeuAsnAsnPheTyrProArgGluAlaLysValGlnTrpLysValAspAsnAlaLeuGlnSerGlyAsnSerGlnGluSerValThrGluGlnAs | |
| | GCCTGCTGAATAACTTCTATCCCAGAGAGGCCAAAGTACAGTGGAAGGTGGATAACGCCCTCCAATCGGGTAACTCCCAGGAGAGTGTCACAGAGCAGGA | |
| 401 | ---------!---------!---------!---------!---------!---------!---------!---------!---------!---------! | 500 |
| 1: | pSerLysAspSerThrTyrSerLeuSerSerThrLeuThrLeuSerLysAlaAspTyrGluLysHisLysValTyrAlaCysGluValThrHisGlnGly | |
| | CAGCAAGGACAGCACCTACAGCCTCAGCAGCACCCTGACGCTGAGCAAAGCGGACTACGAGAAACACAAAGTCTACGCCTGCGAAGTCACCCATCAGGGC | |
| 501 | ---------!---------!---------!---------!---------!---------!---------!---------!---------!---------! | 600 |
| 1: | LeuSerSerProValThrLysSerPheAsnArgGlyGluCys | |
| | CTGAGCTCGCCCGTCACAAAGAGCTTCAACAGGGGAGAGTGT | |
| 601 | ---------!---------!---------!---------!-- | 642 |
| 6R4-G10 | | |
| SEQ ID No. 13 | | |
| 1: | AspIleGlnMetThrGlnSerProSerSerLeuSerAlaSerValGlyAspArgValThrIleThrCysGlnAlaSerGluArgIleTyrTyrAlaIleA | |
| | GACATCCAGATGACCCAGTCTCCATCCTCCCTGTCTGCATCTGTAGGAGACAGAGTCACCATCACTTGCCAGGCCAGTGAGCGTATTTATTATGCGATTA | |
| 1 | ---------!---------!---------!---------!---------!---------!---------!---------!---------!---------! | 100 |
| 1: | snTrpTyrGlnGlnLysProGlyLysAlaProLysLeuLeuIleTyrGlyAlaAsnTyrArgValAspGlyValProSerArgPheSerGlySerGlySe | |
| | ATTGGTATCAGCAGAAACCAGGGAAAGCCCCTAAGCTCCTGATCTATGGTGCGAATTATCGTGTGGATGGGGTCCCATCAAGGTTCAGTGGCAGTGGATC | |
| 101 | ---------!---------!---------!---------!---------!---------!---------!---------!---------!---------! | 200 |
| 1: | rGlyThrAspTyrThrLeuThrIleSerSerLeuGlnProGluAspPheAlaThrTyrTyrCysGlnGlnGlyTyrIleValProThrAsnPheGlyGly | |
| | TGGGACAGATTACACTCTCACCATCAGCAGTCTGCAACCTGAAGATTTTGCAACTTACTACTGTCAACAAGGATATATTGTTCCGACGAATTTCGGCGGA | |
| 201 | ---------!---------!---------!---------!---------!---------!---------!---------!---------!---------! | 300 |
| 1: | GlyThrLysValGluIleLysArgThrValAlaAlaProSerValPheIlePheProProSerAspGluGlnLeuLysSerGlyThrAlaSerValValC | |
| | GGGACCAAGGTGGAGATCAAACGGACTGTGGCTGCACCATCTGTCTTCATCTTCCCGCCATCTGATGAGCAGTTGAAATCTGGAACTGCCTCTGTTGTGT | |
| 301 | ---------!---------!---------!---------!---------!---------!---------!---------!---------!---------! | 400 |
| 1: | ysLeuLeuAsnAsnPheTyrProArgGluAlaLysValGlnTrpLysValAspAsnAlaLeuGlnSerGlyAsnSerGlnGluSerValAlaGluGlnAs | |
| | GCCTGCTGAATAACTTCTATCCCAGAGAGGCCAAAGTACAGTGGAAGGTGGATAACGCCCTCCAATCGGGTAACTCCCAGGAGAGTGTCGCAGAGCAGGA | |
| 401 | ---------!---------!---------!---------!---------!---------!---------!---------!---------!---------! | 500 |
| 1: | pSerLysAspSerThrTyrSerLeuSerSerThrLeuThrLeuSerLysAlaAspTyrGluLysHisLysValTyrAlaCysGluValThrHisGlnGly | |
| | CAGCAAGGACAGCACCTACAGCCTCAGCAGCACCCTGACGCTGAGCAAAGCAGACTACGAGAAACACAAAGTCTACGCCTGCGAAGTCACCCATCAGGGC | |
| 501 | ---------!---------!---------!---------!---------!---------!---------!---------!---------!---------! | 600 |
| 1: | LeuSerSerProValThrLysSerPheAsnArgGlyGluCys | |
| | CTGAGCTCGCCCGTCACAAAGAGCTTCAACAGGGGAGAGTGT | |
| 601 | ---------!---------!---------!---------!-- | 642 |
| 6R4-G11 | | |
| SEQ ID No. 14 | | |
| 1: | AspIleGlnMetThrGlnSerProSerSerLeuSerAlaSerValGlyAspArgValThrIleThrCysLysAlaSerGluAspIleArgTyrGlyLeuA | |
| | GACATCCAGATGACCCAGTCTCCATCCTCCCTGTCTGCATCTGTAGGAGACAGAGTCACCATCACTTGCAAGGCCAGTGAGGACATTCGTTATGGGTTAA | |
| 1 | ---------!---------!---------!---------!---------!---------!---------!---------!---------!---------! | 100 |
| 1: | snTrpTyrGlnGlnLysProGlyLysAlaProLysLeuLeuIleTyrGlyAlaAsnHisLeuArgGluGlyValProSerArgPheSerGlySerGlySe | |
| | ACTGGTATCAGCAGAAACCAGGGAAAGCCCCTAAGCTCCTGATCTATGGGGCTAATCATCTTAGGGAGGGGGTCCCATCAAGGTTCAGTGGCAGTGGATC | |
| 101 | ---------!---------!---------!---------!---------!---------!---------!---------!---------!---------! | 200 |
| 1: | rGlyThrAspTyrThrLeuThrIleSerSerLeuGlnProGluAspPheAlaThrTyrTyrCysGlyTrpGlyTyrSerValProLeuThrPheGlyGly | |
| | TGGGACAGATTACACTCTCACCATCAGCAGTCTGCAACCTGAAGATTTTGCAACTTACTACTGTGGTTGGGGGTATAGTGTTCCTTTGACTTTCGGCGGA | |
| 201 | ---------!---------!---------!---------!---------!---------!---------!---------!---------!---------! | 300 |
| 1: | GlyThrLysValGluIleLysArgThrValAlaAlaProSerValPheIlePheProProSerAspGluGlnLeuLysSerGlyThrAlaSerValValC | |
| | GGGACCAAGGTGGAGATCAAACGGACTGTGGCTGCACCATCTGTCTTCATCTTCCCGCCATCTGATGAGCAGTTGAAATCTGGAACTGCCTCTGTTGTGT | |
| 301 | ---------!---------!---------!---------!---------!---------!---------!---------!---------!---------! | 400 |
| 1: | ysLeuLeuAsnAsnPheTyrProArgGluAlaLysValGlnTrpLysValAspAsnAlaLeuGlnSerGlyAsnSerGlnGluSerValThrGluGlnAs | |
| | GCCTGCTGAATAACTTCTATCCCAGAGAGGCCAAAGTACAGTGGAAGGTGGATAACGCCCTCCAATCGGGTAACTCCCAGGAGAGTGTCACAGAGCAGGA | |
| 401 | ---------!---------!---------!---------!---------!---------!---------!---------!---------!---------! | 500 |
| 1: | pSerLysAspSerThrTyrSerLeuSerSerThrLeuThrLeuGlyArgAlaAspTyrGluLysHisLysValTyrAlaCysGluValThrHisGlnGly | |
| | CAGCAAGGACAGCACCTACAGCCTCAGCAGCACCCTGACGCTGGGCAGAGCAGACTACGAGAAACACAAAGTCTACGCCTGCGAAGTCACCCATCAGGGC | |
| 501 | ---------!---------!---------!---------!---------!---------!---------!---------!---------!---------! | 600 |
| 1: | LeuSerSerProValThrLysSerPheAsnArgGlyGluCys | |
| | CTGAGCTCGCCCGTCACAAAGAGCTTCAACAGGGGAGAGTGT | |
| 601 | ---------!---------!---------!---------!-- | 642 |
| 6R4-E12 | | |
| SEQ ID No. 15 | | |
| 1: | AspIleGlnMetThrGlnSerProSerSerLeuSerAlaSerValGlyAspArgValThrIleThrCysGlnAlaSerGluLysIleHisTyrAlaIleA | |
| | GACATCCAGATGACCCAGTCTCCATCCTCCCTGTCTGCATCTGTAGGAGACAGAGTCACCATCACTTGCCAGGCCAGTGAGAAGATTCATTATGCGATTA | |
| 1 | ---------!---------!---------!---------!---------!---------!---------!---------!---------!---------! | 100 |
| 1: | snTrpTyrGlnGlnLysProGlyLysAlaProLysLeuLeuIleTyrGlyHisSerHisLeuGluAspGlyValProSerArgPheSerGlySerGlySe | |
| | ATTGGTATCAGCAGAAACCAGGGAAAGCCCCTAAGCTCCTGATCTATGGGCATAGTCATCTTGAGGATGGGGTCCCATCAAGGTTCAGTGGCAGTGGATC | |
| 101 | ---------!---------!---------!---------!---------!---------!---------!---------!---------!---------! | 200 |
| 1: | rGlyThrAspTyrThrLeuThrIleSerSerLeuGlnProGluAspPheAlaThrTyrTyrCysGlnGlnGlyTyrIleValProAsnSerPheGlyGly | |
| | TGGGACAGATTACACTCTCACCATCAGCAGTCTGCAACCTGAAGATTTTGCAACTTACTACTGTCAACAAGGATATATTGTTCCGAATTCTTTCGGCGGA | |
| 201 | ---------!---------!---------!---------!---------!---------!---------!---------!---------!---------! | 300 |
| 1: | GlyThrLysValGluIleLysArgThrValAlaAlaProSerValPheIlePheProProSerAspGluGlnLeuLysSerGlyThrAlaSerValValC | |
| | GGGACCAAGGTGGAGATCAAACGGACTGTGGCTGCACCATCTGTCTTCATCTTCCCGCCATCTGATGAGCAGTTGAAATCTGGAACTGCCTCTGTTGTGT | |
| 301 | ---------!---------!---------!---------!---------!---------!---------!---------!---------!---------! | 400 |
| 1: | ysLeuLeuAsnAsnPheTyrProArgGluAlaLysValGlnTrpLysValAspAsnAlaLeuGlnSerGlyAsnSerGlnGluSerValThrGluGlnAs | |
| | GCCTGCTGAATAACTTCTATCCCAGAGAGGCCAAAGTACAGTGGAAGGTGGATAACGCCCTCCAATCGGGTAACTCCCAGGAGAGTGTCACAGAGCAGGA | |
| 401 | ---------!---------!---------!---------!---------!---------!---------!---------!---------!---------! | 500 |
| 1: | pSerLysAspSerThrTyrSerLeuSerSerThrLeuThrLeuSerLysAlaAspTyrGluLysHisLysValTyrAlaCysGluValThrHisGlnGly | |
| | CAGCAAGGACAGCACCTACAGCCTCAGCAGCACCCTGACGCTGAGCAAAGCAGACTACGAGAAACACAAAGTCTACGCCTGCGAAGTCACCCATCAGGGC | |
| 501 | ---------!---------!---------!---------!---------!---------!---------!---------!---------!---------! | 600 |
| 1: | LeuSerSerProValThrLysSerPheAsnArgGlyGluCys | |
| | CTGAGCTCGCCCGTCACAAAGAGCTTCAACAGGGGAGAGTGT | |
| 601 | ---------!---------!---------!---------!-- | 642 |
| 6R4-E3 | | |
| SEQ ID No. 16 | | |
| 1: | AspIleGlnMetThrGlnSerProSerSerLeuSerAlaSerValGlyAspArgValThrIleThrCysGlnAlaSerGlnLeuIleTyrTyrGlyIleA | |
| | GACATCCAGATGACCCAGTCTCCATCCTCCCTGTCTGCATCTGTAGGAGACAGAGTCACCATCACTTGCCAGGCCAGTCAGCTGATTTATTATGGTATTA | |
| 1 | ---------!---------!---------!---------!---------!---------!---------!---------!---------!---------! | 100 |
| 1: | snTrpTyrGlnGlnLysProGlyLysAlaProLysLeuLeuIleTyrGlyAlaAsnTyrArgValAspGlyValProSerArgPheSerGlySerGlySe | |
| | ATTGGTATCAGCAGAAACCAGGGAAAGCCCCTAAGCTCCTGATCTATGGTGCGAATTATCGTGTGGATGGGGTCCCATCAAGGTTCAGTGGCAGTGGATC | |
| 101 | ---------!---------!---------!---------!---------!---------!---------!---------!---------!---------! | 200 |
| 1: | rGlyThrAspTyrThrLeuThrIleSerSerLeuGlnProGluAspPheAlaThrTyrTyrCysGlnGlnGlyTyrSerValProLeuAspPheGlyGly | |
| | TGGGACAGATTACACTCTCACCATCAGCAGTCTGCAACCTGAAGATTTTGCAACTTACTACTGTCAACAAGGATATAGTGTTCCGCTTGATTTCGGCGGA | |
| 201 | ---------!---------!---------!---------!---------!---------!---------!---------!---------!---------! | 300 |
| 1: | GlyThrLysValGluIleLysArgThrValAlaAlaProSerValPheIlePheProProSerAspGluGlnLeuLysSerGlyThrAlaSerValValC | |
| | GGGACCAAGGTGGAGATCAAACGGACTGTGGCTGCACCATCTGTCTTCATCTTCCCGCCATCTGATGAGCAGTTGAAATCTGGAACTGCCTCTGTTGTGT | |
| 301 | ---------!---------!---------!---------!---------!---------!---------!---------!---------!---------! | 400 |
| 1: | ysLeuLeuAsnAsnPheTyrProArgGluAlaLysValGlnTrpLysValAspAsnAlaLeuGlnSerGlyAsnSerGlnGluSerValThrGluGlnAs | |
| | GCCTGCTGAATAACTTCTATCCCAGAGAGGCCAAAGTACAGTGGAAGGTGGATAACGCCCTCCAATCGGGTAACTCCCAGGAGAGTGTCACAGAGCAGGA | |
| 401 | ---------!---------!---------!---------!---------!---------!---------!---------!---------!---------! | 500 |
| 1: | pSerLysAspSerThrTyrSerLeuSerSerThrLeuThrLeuSerLysAlaAspTyrGluLysHisLysValTyrAlaCysGluValThrHisGlnGly | |
| | CAGCAAGGACAGCACCTACAGCCTCAGCAGCACCCTGACGCTGAGCAAAGCAGACTACGAGAAACACAAAGTCTACGCCTGCGAAGTCACCCATCAGGGC | |
| 501 | ---------!---------!---------!---------!---------!---------!---------!---------!---------!---------! | 600 |
| 1: | LeuSerSerProValThrLysSerPheAsnArgGlyGluCys | |
| | CTGAGCTCGCCCGTCACAAAGAGCTTCAACAGGGGAGAGTGT | |
| 601 | ---------!---------!---------!---------!-- | 642 |
| 6R4-H5 | | |
| SEQ ID No. 17 | | |
| 1: | AspIleGlnMetThrGlnSerProSerSerLeuSerAlaSerValGlyAspArgValThrIleThrCysLysAlaSerGluAspIleArgTyrGlyLeuA | |
| | GACATCCAGATGACCCAGTCTCCATCCTCCCTGTCTGCATCTGTAGGAGACAGAGTCACCATCACTTGCAAGGCCAGTGAGGACATTCGTTATGGGTTAA | |
| 1 | ---------!---------!---------!---------!---------!---------!---------!---------!---------!---------! | 100 |
| 1: | snTrpTyrGlnGlnLysProGlyLysAlaProLysLeuLeuIleTyrGlyHisThrTyrArgLeuAspGlyValProSerArgPheSerGlySerGlySe | |
| | ACTGGTATCAGCAGAAACCAGGGAAAGCCCCTAAGCTCCTGATCTATGGGCATACTTATCGTTTGGATGGGGTCCCATCAAGGTTCAGTGGCAGTGGATC | |
| 101 | ---------!---------!---------!---------!---------!---------!---------!---------!---------!---------! | 200 |
| 1: | rGlyThrAspTyrThrLeuThrIleSerSerLeuGlnProGluAspPheAlaThrTyrTyrCysGlnGlnGlyTyrValValProValGlyPheGlyGly | |
| | TGGGACAGATTACACTCTCACCATCAGCAGTCTGCAACCTGAAGATTTTGCAACTTACTACTGTCAACAAGGATATGTGGTGCCGGTGGGGTTCGGCGGA | |
| 201 | ---------!---------!---------!---------!---------!---------!---------!---------!---------!---------! | 300 |
| 1: | GlyThrLysValGluIleLysArgThrValAlaAlaProSerValPheIlePheProProSerAspGluGlnLeuLysSerGlyThrAlaSerValValC | |
| | GGGACCAAGGTGGAGATCAAACGGACTGTGGCTGCACCATCTGTCTTCATCTTCCCGCCATCTGATGAGCAGTTGAAATCTGGAACTGCCTCTGTTGTGT | |
| 301 | ---------!---------!---------!---------!---------!---------!---------!---------!---------!---------! | 400 |
| 1: | ysLeuLeuAsnAsnPheTyrProArgGluAlaLysValGlnTrpLysValAspAsnAlaLeuGlnSerGlyAsnSerGlnGluSerValThrGluGlnAs | |
| | GCCTGCTGAATAACTTCTATCCCAGAGAGGCCAAAGTACAGTGGAAGGTGGATAACGCCCTCCAATCGGGTAACTCCCAGGAGAGTGTCACAGAGCAGGA | |
| 401 | ---------!---------!---------!---------!---------!---------!---------!---------!---------!---------! | 500 |
| 1: | pSerLysAspSerThrTyrSerLeuSerSerThrLeuThrLeuSerLysAlaAspTyrGluLysHisLysValTyrAlaCysGluValThrHisGlnGly | |
| | CAGCAAGGACAGCACCTACAGCCTCAGCAGCACCCTGACGCTGAGCAAAGCAGACTACGAGAAACACAAAGTCTACGCCTGCGAAGTCACCCATCAGGGC | |
| 501 | ---------!---------!---------!---------!---------!---------!---------!---------!---------!---------! | 600 |
| 1: | LeuSerSerProValThrLysSerPheAsnArgGlyGluCys | |
| | CTGAGCTCGCCCGTCACAAAGAGCTTCAACAGGGGAGAGTGT | |
| 601 | ---------!---------!---------!---------!-- | 642 |
| 6R4-H11 | | |
| SEQ ID No. 18 | | |
| 1: | AspIleGlnMetThrGlnSerProSerSerLeuSerAlaSerValGlyAspArgValThrIleThrCysGlnAlaSerGlnGlyIleHisTyrAlaIleA | |
| | GACATCCAGATGACCCAGTCTCCATCCTCCCTGTCTGCATCTGTAGGAGACAGAGTCACCATCACTTGCCAGGCCAGTCAGGGGATTCATTATGCGATTA | |
| 1 | ---------!---------!---------!---------!---------!---------!---------!---------!---------!---------! | 100 |
| 1: | snTrpTyrGlnGlnLysProGlyLysAlaProLysLeuLeuIleTyrGlyHisAsnTyrLeuGluGluGlyValProSerArgPheSerGlySerGlySe | |
| | ATTGGTATCAGCAGAAACCAGGGAAAGCCCCTAAGCTCCTGATCTATGGGCATAATTATCTTGAGGAGGGGGTCCCATCAAGGTTCAGTGGCAGTGGATC | |
| 101 | ---------!---------!---------!---------!---------!---------!---------!---------!---------!---------! | 200 |
| 1: | rGlyThrAspTyrThrLeuThrIleSerSerLeuGlnProGluAspPheAlaThrTyrTyrCysGlnGlnGlyTyrIleValProIleValPheGlyGly | |
| | TGGGACAGACTACACTCTCACCATCAGCAGTCTGCAACCTGAAGATTTTGCAACTTACTACTGTCAACAAGGATATATTGTGCCGATTGTTTTCGGCGGA | |
| 201 | ---------!---------!---------!---------!---------!---------!---------!---------!---------!---------! | 300 |
| 1: | GlyThrLysValGluIleLysArgThrValAlaAlaProSerValPheIlePheProProSerAspGluGlnLeuLysSerGlyThrAlaSerValValC | |
| | GGGACCAAGGTGGAGATCAAACGGACTGTGGCTGCACCATCTGTCTTCATCTTCCCGCCATCTGATGAGCAGTTGAAATCTGGAACTGCCTCTGTTGTGT | |
| 301 | ---------!---------!---------!---------!---------!---------!---------!---------!---------!---------! | 400 |
| 1: | ysLeuLeuAsnAsnPheTyrProArgGluAlaLysValGlnTrpLysValAspAsnAlaLeuGlnSerGlyAsnSerGlnGluSerValThrGluGlnAs | |
| | GCCTGCTGAATAACTTCTATCCCAGAGAGGCCAAAGTACAGTGGAAGGTGGATAACGCCCTCCAATCGGGTAACTCCCAGGAGAGTGTCACAGAGCAGGA | |
| 401 | ---------!---------!---------!---------!---------!---------!---------!---------!---------!---------! | 500 |
| 1: | pSerLysAspSerThrTyrSerLeuSerSerThrLeuThrLeuSerLysAlaAspTyrGluLysHisLysValTyrAlaCysGluValThrHisGlnGly | |
| | CAGCAAGGACAGCACCTACAGCCTCAGCAGCACCCTGACGCTGAGCAAAGCAGACTACGAGAAACACAAAGTCTACGCCTGCGAAGTCACCCATCAGGGC | |
| 501 | ---------!---------!---------!---------!---------!---------!---------!---------!---------!---------! | 600 |
| 1: | LeuSerSerProValThrLysSerPheAsnArgGlyGluCys | |
| | CTGAGCTCGCCCGTCACAAAGAGCTTCAACAGGGGAGAGTGT | |
| 601 | ---------!---------!---------!---------!-- | 642 |
| 6R6-A7 | | |
| SEQ ID No. 19 | | |
| 1: | AspIleGlnMetThrGlnSerProSerSerLeuSerAlaSerValGlyAspArgValThrIleThrCysGlnAlaSerGluLysIleTyrTyrAlaIleA | |
| | GACATCCAGATGACCCAGTCTCCATCCTCCCTGTCTGCATCTGTAGGAGACAGAGTCACCATCACTTGCCAGGCCAGTGAGAAGATTTATTATGCGATTA | |
| 1 | ---------!---------!---------!---------!---------!---------!---------!---------!---------!---------! | 100 |
| 1: | snTrpTyrGlnGlnLysProGlyLysAlaProLysLeuLeuIleTyrGlyHisSerTyrArgLeuGluGlyValProSerArgPheSerGlySerGlySe | |
| | ATTGGTATCAGCAGAAACCAGGGAAAGCCCCTAAGCTCCTGATCTATGGGCATAGTTATCGTTTGGAGGGGGTCCCATCAAGGTTCAGTGGCAGTGGATC | |
| 101 | ---------!---------!---------!---------!---------!---------!---------!---------!---------!---------! | 200 |
| 1: | rGlyThrAspTyrThrLeuThrIleSerSerLeuGlnProGluAspPheAlaThrTyrTyrCysGlnGlnGlyTyrIleValProAsnArgPheGlyGly | |
| | TGGGACAGATTACACTCTCACCATCAGCAGTCTGCAACCTGAAGATTTTGCAACTTACTACTGTCAACAAGGATATATTGTGCCTAATCGTTTCGGCGGA | |
| 201 | ---------!---------!---------!---------!---------!---------!---------!---------!---------!---------! | 300 |
| 1: | GlyThrLysValGluIleLysArgThrValAlaAlaProSerValPheIlePheProProSerAspGluGlnLeuLysSerGlyThrAlaSerValValC | |
| | GGGACCAAGGTGGAGATCAAACGGACTGTGGCTGCACCATCTGTCTTCATCTTCCCGCCATCTGATGAGCAGTTGAAATCTGGAACTGCCTCTGTTGTGT | |
| 301 | ---------!---------!---------!---------!---------!---------!---------!---------!---------!---------! | 400 |
| 1: | ysLeuLeuAsnAsnPheTyrProArgGluAlaLysValGlnTrpLysValAspAsnAlaLeuGlnSerGlyAsnSerGlnGluSerValThrGluGlnAs | |
| | GCCTGCTGAATAACTTCTATCCCAGAGAGGCCAAAGTACAGTGGAAGGTGGATAACGCCCTCCAATCGGGTAACTCCCAGGAGAGTGTCACAGAGCAGGA | |
| 401 | ---------!---------!---------!---------!---------!---------!---------!---------!---------!---------! | 500 |
| 1: | pSerLysAspSerThrTyrSerLeuSerSerThrLeuThrLeuSerLysAlaAspTyrGluLysHisLysValTyrAlaCysGluValThrHisGlnGly | |
| | CAGCAAGGACAGCACCTACAGCCTCAGCAGCACCCTGACGCTGAGCAAAGCAGACTACGAGAAACACAAAGTCTACGCCTGCGAAGTCACCCATCAGGGC | |
| 501 | ---------!---------!---------!---------!---------!---------!---------!---------!---------!---------! | 600 |
| 1: | LeuSerSerProValThrLysSerPheAsnArgGlyGluCys | |
| | CTGAGCTCGCCCGTCACAAAGAGCTTCAACAGGGGAGAGTGT | |
| 601 | ---------!---------!---------!---------!-- | 642 |
| 6R6-E1 | | |
| SEQ ID No. 20 | | |
| 1: | AspIleGlnMetThrGlnSerProSerSerLeuSerAlaSerValGlyAspArgValThrIleThrCysGlnAlaSerGluLysIleTyrTyrAlaIleA | |
| | GACATCCAGATGACCCAGTCTCCATCCTCCCTGTCTGCATCTGTAGGAGACAGAGTCACCATCACTTGCCAGGCCAGTGAGAAGATTTATTATGCGATTA | |
| 1 | ---------!---------!---------!---------!---------!---------!---------!---------!---------!---------! | 100 |
| 1: | snTrpTyrGlnGlnLysProGlyLysAlaProLysLeuLeuIleTyrGlyHisSerTyrArgLeuGluGlyValProSerArgPheSerGlySerGlySe | |
| | ATTGGTATCAGCAGAAACCAGGGAAAGCCCCTAAGCTCCTGATCTATGGGCATAGTTATCGTTTGGAGGGGGTCCCATCAAGGTTCAGTGGCAGTGGATC | |
| 101 | ---------!---------!---------!---------!---------!---------!---------!---------!---------!---------! | 200 |
| 1: | rGlyThrAspTyrThrLeuThrIleSerSerLeuGlnProGluAspPheAlaThrTyrTyrCysGlnGlnGlyTyrIleValProAsnArgPheGlyGly | |
| | TGGGACAGATTACACTCTCACCATCAGCAGTCTGCAACCTGAAGATTTTGCAACTTACTACTGTCAACAAGGATATATTGTGCCTAATCGTTTCGGCGGA | |
| 201 | ---------!---------!---------!---------!---------!---------!---------!---------!---------!---------! | 300 |
| 1: | GlyThrLysValGluIleLysArgThrValAlaAlaProSerValPheIlePheProProSerAspGluGlnLeuLysSerGlyThrAlaSerValValC | |
| | GGGACCAAGGTGGAGATCAAACGGACTGTGGCTGCACCATCTGTCTTCATCTTCCCGCCATCTGATGAGCAGTTGAAATCTGGAACTGCCTCTGTTGTGT | |
| 301 | ---------!---------!---------!---------!---------!---------!---------!---------!---------!---------! | 400 |
| 1: | ysLeuLeuAsnAsnPheTyrProArgGluAlaLysValGlnTrpLysValAspAsnAlaLeuGlnSerGlyAsnSerGlnGluSerValThrGluGlnAs | |
| | GCCTGCTGAATAACTTCTATCCCAGAGAGGCCAAAGTACAGTGGAAGGTGGATAACGCCCTCCAATCGGGTAACTCCCAGGAGAGTGTCACAGAGCAGGA | |
| 401 | ---------!---------!---------!---------!---------!---------!---------!---------!---------!---------! | 500 |
| 1: | pSerLysAspSerThrTyrSerLeuSerSerThrLeuThrLeuSerLysAlaAspTyrGluLysHisLysValTyrAlaCysGluValThrHisGlnGly | |
| | CAGCAAGGACAGCACCTACAGCCTCAGCAGCACCCTGACGCTGAGCAAAGCAGACTACGAGAAACACAAAGTCTACGCCTGCGAAGTCACCCATCAGGGC | |
| 501 | ---------!---------!---------!---------!---------!---------!---------!---------!---------!---------! | 600 |
| 1: | LeuSerSerProValThrLysSerPheAsnArgGlyGluCys | |
| | CTGAGCTCGCCCGTCACAAAGAGCTTCAACAGGGGAGAGTGT | |
| 601 | ---------!---------!---------!---------!-- | 642 |
| 6R6-E2 | | |
| SEQ ID No. 21 | | |
| 1: | AspIleGlnMetThrGlnSerProSerSerLeuSerAlaSerValGlyAspArgValThrIleThrCysLeuAlaSerGlnProIleTyrTyrGlyLeuA | |
| | GACATCCAGATGACCCAGTCTCCATCCTCCCTGTCTGCATCTGTAGGAGACAGAGTCACCATCACTTGCCTGGCCAGTCAGCCTATTTATTATGGTCTTA | |
| 1 | ---------!---------!---------!---------!---------!---------!---------!---------!---------!---------! | 100 |
| 1: | snTrpTyrGlnGlnLysProGlyLysAlaProLysLeuLeuIleTyrGlyThrSerAlaLeuValAspGlyValProSerArgPheSerGlySerGlySe | |
| | ATTGGTATCAGCAGAAACCAGGGAAAGCCCCTAAGCTCCTGATCTATGGGACGAGTGCGCTTGTGGATGGGGTCCCATCAAGGTTCAGTGGCAGTGGATC | |
| 101 | ---------!---------!---------!---------!---------!---------!---------!---------!---------!---------! | 200 |
| 1: | rGlyThrAspTyrThrLeuThrIleSerSerLeuGlnProGluAspPheAlaThrTyrTyrCysGlnGlnGlyTyrIleValProValSerPheGlyGly | |
| | TGGGACAGATTACACTCTCACCATCAGCAGTCTGCAACCTGAAGATTTTGCAACTTACTACTGTCAACAGGGATATATTGTGCCTGTGTCGTTCGGCGGA | |
| 201 | ---------!---------!---------!---------!---------!---------!---------!---------!---------!---------! | 300 |
| 1: | GlyThrLysValGluIleLysArgThrValAlaAlaProSerValPheIlePheProProSerAspGluGlnLeuLysSerGlyThrAlaSerValValC | |
| | GGGACCAAGGTGGAGATCAAACGGACTGTGGCTGCACCATCTGTCTTCATCTTCCCGCCATCTGATGAGCAGTTGAAATCTGGAACTGCCTCTGTTGTGT | |
| 301 | ---------!---------!---------!---------!---------!---------!---------!---------!---------!---------! | 400 |
| 1: | ysLeuLeuAsnAsnPheTyrProArgGluAlaLysValGlnTrpLysValAspAsnAlaLeuGlnSerGlyAsnSerGlnGluSerValThrGluGlnAs | |
| | GCCTGCTGAATAACTTCTATCCCAGAGAGGCCAAAGTACAGTGGAAGGTGGATAACGCCCTCCAATCGGGTAACTCCCAGGAGAGTGTCACAGAGCAGGA | |
| 401 | ---------!---------!---------!---------!---------!---------!---------!---------!---------!---------! | 500 |
| 1: | pSerLysAspSerThrTyrSerLeuSerSerThrLeuThrLeuSerLysAlaAspTyrGluLysHisLysValTyrAlaCysGluValThrHisGlnGly | |
| | CAGCAAGGACAGCACCTACAGCCTCAGCAGCACCCTGACGCTGAGCAAAGCAGACTACGAGAAACACAAAGTCTACGCCTGCGAAGTCACCCATCAGGGC | |
| 501 | ---------!---------!---------!---------!---------!---------!---------!---------!---------!---------! | 600 |
| 1: | LeuSerSerProValThrLysSerPheAsnArgGlyGluCys | |
| | CTGAGTTCGCCCGTCACAAAGAGCTTCAACAGGGGAGAGTGT | |
| 601 | ---------!---------!---------!---------!-- | 642 |
| 6R6-E3 | | |
| SEQ ID No. 22 | | |
| 1: | AspIleGlnMetThrGlnSerProSerSerLeuSerAlaSerValGlyAspArgValThrIleThrCysGlnAlaSerGluAsnIleArgTyrAlaIleA | |
| | GACATCCAGATGACCCAGTCTCCATCCTCCCTGTCTGCATCTGTAGGAGACAGAGTCACCATCACTTGCCAGGCCAGTGAGAATATTAGGTATGCTATTA | |
| 1 | ---------!---------!---------!---------!---------!---------!---------!---------!---------!---------! | 100 |
| 1: | snTrpTyrGlnGlnLysProGlyLysAlaProLysLeuLeuIleTyrGlyAlaAsnTyrArgValAspGlyValProSerArgPheSerGlySerGlySe | |
| | ATTGGTATCAGCAGAAACCAGGGAAAGCCCCTAAGCTCCTGATCTATGGTGCGAATTATCGTGTGGATGGGGTCCCATCAAGGTTCAGTGGCAGTGGATC | |
| 101 | ---------!---------!---------!---------!---------!---------!---------!---------!---------!---------! | 200 |
| 1: | rGlyThrAspTyrThrLeuThrIleSerSerLeuGlnProGluAspPheAlaThrTyrTyrCysGlnGlnGlyTyrAsnValProThrAspPheGlyGly | |
| | TGGGACAGATTACACTCTCACCATCAGCAGTCTGCAACCTGAAGATTTTGCAACTTACTACTGTCAACAAGGATATAATGTGCCTACTGATTTCGGCGGA | |
| 201 | ---------!---------!---------!---------!---------!---------!---------!---------!---------!---------! | 300 |
| 1: | GlyThrLysValGluIleLysArgThrValAlaAlaProSerValPheIlePheProProSerAspGluGlnLeuLysSerGlyThrAlaSerValValC | |
| | GGGACCAAGGTGGAGATCAAACGGACTGTGGCTGCACCATCTGTCTTCATCTTCCCGCCATCTGATGAGCAGTTGAAATCTGGAACTGCCTCTGTTGTGT | |
| 301 | ---------!---------!---------!---------!---------!---------!---------!---------!---------!---------! | 400 |
| 1: | ysLeuLeuAsnAsnPheTyrProArgGluAlaLysValGlnTrpLysValAspAsnAlaLeuGlnSerGlyAsnSerGlnGluSerValThrGluGlnAs | |
| | GCCTGCTGAATAACTTCTATCCCAGAGAGGCCAAAGTACAGTGGAAGGTGGATAACGCCCTCCAATCGGGTAACTCCCAGGAGAGTGTCACAGAGCAGGA | |
| 401 | ---------!---------!---------!---------!---------!---------!---------!---------!---------!---------! | 500 |
| 1: | pSerLysAspGlyThrTyrSerLeuSerSerThrLeuThrLeuSerLysAlaAspTyrGluLysHisLysValTyrAlaCysGluValThrHisGlnGly | |
| | CAGCAAGGACGGCACCTACAGCCTCAGCAGCACCCTGACGCTGAGCAAAGCAGACTACGAGAAACACAAAGTCTACGCCTGCGAAGTCACCCATCAGGGC | |
| 501 | ---------!---------!---------!---------!---------!---------!---------!---------!---------!---------! | 600 |
| 1: | LeuSerSerProValThrLysSerPheAsnArgGlyGluCys | |
| | CTGAGCTCGCCCGTCACAAAGAGCTTCAACAGGGGAGAGTGT | |
| 601 | ---------!---------!---------!---------!-- | 642 |
| 6R6-E4 | | |
| SEQ ID No. 23 | | |
| 1: | AspIleGlnMetThrGlnSerProSerSerLeuSerAlaSerValGlyAspArgValThrIleThrCysGlnAlaSerGluAsnIleArgTyrAlaValA | |
| | GACATCCAGATGACCCAGTCTCCATCCTCCCTGTCTGCATCTGTAGGAGACAGAGTCACCATCACTTGCCAGGCCAGTGAGAATATTAGGTATGCTGTTA | |
| 1 | ---------!---------!---------!---------!---------!---------!---------!---------!---------!---------! | 100 |
| 1: | snTrpTyrGlnGlnLysProGlyLysAlaProLysLeuLeuIleTyrGlyAlaAsnTyrArgValAspGlyValProSerArgPheSerGlySerGlySe | |
| | ATTGGTATCAGCAGAAACCAGGGAAAGCCCCTAAGCTCCTGATCTATGGTGCGAATTATCGTGTGGATGGGGTCCCATCAAGGTTCAGTGGCAGTGGATC | |
| 101 | ---------!---------!---------!---------!---------!---------!---------!---------!---------!---------! | 200 |
| 1: | rGlyThrAspTyrThrLeuThrIleSerSerLeuGlnProGluAspPheAlaThrTyrTyrCysGlnGlnGlyTyrArgValAlaIleGlyPheGlyGly | |
| | TGGGACAGATTACACTCTCACCATCAGCAGTCTGCAACCTGAAGATTTTGCAACTTACTACTGTCAACAAGGATATCGTGTGGCTATTGGGTTCGGCGGA | |
| 201 | ---------!---------!---------!---------!---------!---------!---------!---------!---------!---------! | 300 |
| 1: | GlyThrLysValGluIleLysArgThrValAlaAlaProSerValPheIlePheProProSerAspGluGlnLeuLysSerGlyThrAlaSerValValC | |
| | GGGACCAAGGTGGAGATCAAACGGACTGTGGCTGCACCATCTGTCTTCATCTTCCCGCCATCTGATGAGCAGTTGAAATCTGGAACTGCCTCTGTTGTGT | |
| 301 | ---------!---------!---------!---------!---------!---------!---------!---------!---------!---------! | 400 |
| 1: | ysLeuLeuAsnAsnPheTyrProArgGluAlaLysValGlnTrpLysValAspAsnAlaLeuGlnSerGlyAsnSerGlnGluSerValThrGluGlnAs | |
| | GCCTGCTGAATAACTTCTATCCCAGAGAGGCCAAAGTACAGTGGAAGGTGGATAACGCCCTCCAATCGGGTAACTCCCAGGAGAGTGTCACAGAGCAGGA | |
| 401 | ---------!---------!---------!---------!---------!---------!---------!---------!---------!---------! | 500 |
| 1: | pSerLysAspSerThrTyrSerLeuSerSerThrLeuThrLeuSerLysAlaAspTyrGluLysHisLysValTyrAlaCysGluValThrHisGlnGly | |
| | CAGCAAGGACAGTACCTACAGCCTCAGCAGCACCCTGACGCTGAGCAAAGCAGACTACGAGAAACACAAAGTCTACGCCTGCGAAGTCACCCATCAGGGC | |
| 501 | ---------!---------!---------!---------!---------!---------!---------!---------!---------!---------! | 600 |
| 1: | LeuSerSerProValThrLysSerPheAsnArgGlyGluCys | |
| | CTGAGCTCGCCCGTCACAAAGAGCTTCAACAGGGGAGAGTGT | |
| 601 | ---------!---------!---------!---------!-- | 642 |
| 6R6-F3 | | |
| SEQ ID No. 24 | | |
| 1: | AspIleGlnMetThrGlnSerProSerSerLeuSerAlaSerValGlyAspArgValThrIleThrCysGlnAlaSerGluGluValHisTyrGlyIleA | |
| | GACATCCAGATGACCCAGTCTCCATCCTCCCTGTCTGCATCTGTAGGAGACAGAGTCACCATCACTTGCCAGGCCAGTGAGGAGGTTCATTATGGGATTA | |
| 1 | ---------!---------!---------!---------!---------!---------!---------!---------!---------!---------! | 100 |
| 1: | snTrpTyrGlnGlnLysProGlyLysAlaProLysLeuLeuIleTyrGlyAlaAsnTyrArgValAspGlyValProSerArgPheSerGlySerGlySe | |
| | ATTGGTATCAGCAGAAACCAGGGAAAGCCCCTAAGCTCCTGATCTATGGTGCGAATTATCGTGTGGATGGGGTCCCATCAAGGTTCAGTGGCAGTGGATC | |
| 101 | ---------!---------!---------!---------!---------!---------!---------!---------!---------!---------! | 200 |
| 1: | rGlyThrAspTyrThrLeuThrIleSerSerLeuGlnProGluAspPheAlaThrTyrTyrCysGlnGlnGlyTyrAsnValProThrAspPheGlyGly | |
| | TGGGACAGATTACACTCTCACCATCAGCAGTCTGCAACCTGAAGATTTTGCAACTTACTACTGTCAACAAGGATATAATGTGCCTACTGATTTCGGCGGA | |
| 201 | ---------!---------!---------!---------!---------!---------!---------!---------!---------!---------! | 300 |
| 1: | GlyThrLysValGluIleLysArgThrValAlaAlaProSerValPheIlePheProProSerAspGluGlnLeuLysSerGlyThrAlaSerValValC | |
| | GGGACCAAGGTGGAGATCAAACGGACTGTGGCTGCACCATCTGTCTTCATCTTCCCGCCATCTGATGAGCAGTTGAAATCTGGAACTGCCTCTGTTGTGT | |
| 301 | ---------!---------!---------!---------!---------!---------!---------!---------!---------!---------! | 400 |
| 1: | ysLeuLeuAsnAsnPheTyrProArgGluAlaLysValGlnTrpLysValAspAsnAlaLeuGlnSerGlyAsnSerGlnGluSerValThrGluGlnAs | |
| | GCCTGCTGAATAACTTCTATCCCAGAGAGGCCAAAGTACAGTGGAAGGTGGATAACGCCCTCCAATCGGGTAACTCCCAGGAGAGTGTCACAGAGCAGGA | |
| 401 | ---------!---------!---------!---------!---------!---------!---------!---------!---------!---------! | 500 |
| 1: | pSerLysAspSerThrTyrSerLeuSerSerThrLeuThrLeuSerLysAlaAspTyrGluLysHisLysValTyrAlaCysGluValThrHisGlnGly | |
| | CAGCAAGGACAGCACCTACAGCCTCAGCAGCACCCTGACGCTGAGCAAAGCAGACTACGAGAAACACAAAGTCTACGCCTGCGAAGTCACCCATCAGGGC | |
| 501 | ---------!---------!---------!---------!---------!---------!---------!---------!---------!---------! | 600 |
| 1: | LeuSerSerProValThrLysSerPheAsnArgGlyGluCys | |
| | CTGAGCTCGCCCGTCACAAAGAGCTTCAACAGGGGAGAGTGT | |
| 601 | ---------!---------!---------!---------!-- | 642 |
| 6R6-F7 | | |
| SEQ ID No. 25 | | |
| 1: | AspIleGlnMetThrGlnSerProSerSerLeuSerAlaSerValGlyAspArgValThrIleThrCysGlnAlaSerGluArgIleTyrTyrGlyLeuA | |
| | GACATCCAGATGACCCAGTCTCCATCCTCCCTGTCCGCATCTGTAGGAGACAGAGTCACCATCACTTGCCAGGCCAGTGAGAGGATTTATTATGGGCTTA | |
| 1 | ---------!---------!---------!---------!---------!---------!---------!---------!---------!---------! | 100 |
| 1: | snTrpTyrGlnGlnLysProGlyLysAlaProLysLeuLeuIleTyrGlyAlaThrGlyArgAlaAspGlyValProSerArgPheSerGlySerGlySe | |
| | ATTGGTATCAGCAGAAACCAGGGAAAGCCCCTAAGCTCCTGATCTATGGGGCTACTGGTCGTGCGGATGGGGTCCCATCAAGGTTCAGTGGCAGTGGATC | |
| 101 | ---------!---------!---------!---------!---------!---------!---------!---------!---------!---------! | 200 |
| 1: | rGlyThrAspTyrThrLeuThrIleSerSerLeuGlnProGluAspPheAlaThrTyrTyrCysGlnGlnGlyTyrIleValProSerGlyPheGlyGly | |
| | TGGGACAGATTACACTCTCACCATCAGCAGTCTGCAACCTGAAGATTTTGCAACTTACTACTGTCAACAAGGATATATTGTGCCGTCGGGGTTCGGCGGA | |
| 201 | ---------!---------!---------!---------!---------!---------!---------!---------!---------!---------! | 300 |
| 1: | GlyThrLysValGluIleLysArgThrValAlaAlaProSerValPheIlePheProProSerAspGluGlnLeuLysSerGlyThrAlaSerValValC | |
| | GGGACCAAGGTGGAGATCAAACGGACTGTGGCTGCACCATCTGTCTTCATCTTCCCGCCATCTGATGAGCAGTTGAAATCTGGAACTGCCTCTGTTGTGT | |
| 301 | ---------!---------!---------!---------!---------!---------!---------!---------!---------!---------! | 400 |
| 1: | ysLeuLeuAsnAsnPheTyrProArgGluAlaLysValGlnTrpLysValAspAsnAlaLeuGlnSerGlyAsnSerGlnGluSerValThrGluGlnAs | |
| | GCCTGCTGAATAACTTCTATCCCAGAGAGGCCAAAGTACAGTGGAAGGTGGATAACGCCCTCCAATCGGGTAACTCCCAGGAGAGTGTCACAGAGCAGGA | |
| 401 | ---------!---------!---------!---------!---------!---------!---------!---------!---------!---------! | 500 |
| 1: | pSerLysAspSerThrTyrSerLeuSerSerThrLeuThrLeuSerLysAlaAspTyrGluLysHisLysValTyrAlaCysGluValThrHisGlnGly | |
| | CAGCAAGGACAGCACCTACAGCCTCAGCAGCACCCTGACGCTGAGCAAAGCAGACTACGAGAAACACAAAGTCTACGCCTGCGAAGTCACCCATCAGGGC | |
| 501 | ---------!---------!---------!---------!---------!---------!---------!---------!---------!---------! | 600 |
| 1: | LeuSerSerProValThrLysSerPheAsnArgGlyGluCys | |
| | CTGAGCTCGCCCGTCACAAAGAGCTTCAACAGGGGAGAGTGT | |
| 601 | ---------!---------!---------!---------!-- | 642 |
| 6R6-F9 | | |
| SEQ ID No. 26 | | |
| 1: | AspIleGlnMetThrGlnSerProSerSerLeuSerAlaSerValGlyAspArgValThrIleThrCysLysAlaSerGluAspIleArgTyrGlyLeuA | |
| | GACATCCAGATGACCCAGTCTCCATCCTCCCTGTCTGCATCTGTAGGAGACAGAGTCACCATCACTTGCAAGGCCAGTGAGGACATTCGTTATGGGTTAA | |
| 1 | ---------!---------!---------!---------!---------!---------!---------!---------!---------!---------! | 100 |
| 1: | snTrpTyrGlnGlnLysProGlyLysAlaProLysLeuLeuIleTyrGlyHisThrTyrArgLeuAspGlyValProSerArgPheSerGlySerGlySe | |
| | ACTGGTATCAGCAGAAACCAGGGAAAGCCCCTAAGCTCCTGATCTATGGGCATACTTATCGTTTGGATGGGGTCCCATCAAGGTTCAGTGGCAGTGGATC | |
| 101 | ---------!---------!---------!---------!---------!---------!---------!---------!---------!---------! | 200 |
| 1: | rGlyThrAspTyrThrLeuThrIleSerSerLeuGlnProGluAspPheAlaThrTyrTyrCysGlnGlnGlyTyrIleValProAsnMetPheGlyGly | |
| | TGGGACAGATTACACTCTCACCATCAGCAGTCTGCAACCTGAAGATTTTGCAACTTACTACTGTCAACAAGGATATATTGTGCCTAATATGTTCGGCGGA | |
| 201 | ---------!---------!---------!---------!---------!---------!---------!---------!---------!---------! | 300 |
| 1: | GlyThrLysValGluIleLysArgThrValAlaAlaProSerValPheIlePheProProSerAspGluGlnLeuLysSerGlyThrAlaSerValValC | |
| | GGGACCAAGGTGGAGATCAAACGGACTGTGGCTGCACCATCTGTCTTCATCTTCCCGCCATCTGATGAGCAGTTGAAATCTGGAACTGCCTCTGTTGTGT | |
| 301 | ---------!---------!---------!---------!---------!---------!---------!---------!---------!---------! | 400 |
| 1: | ysLeuLeuAsnAsnPheTyrProArgGluAlaLysValGlnTrpLysValAspAsnAlaLeuGlnSerGlyAsnSerGlnGluSerValThrGluGlnAs | |
| | GCCTGCTGAATAACTTCTATCCCAGAGAGGCCAAAGTACAGTGGAAGGTGGATAACGCCCTCCAATCGGGTAACTCCCAGGAGAGTGTCACAGAGCAGGA | |
| 401 | ---------!---------!---------!---------!---------!---------!---------!---------!---------!---------! | 500 |
| 1: | pSerLysAspSerThrTyrSerLeuSerSerThrLeuThrLeuSerLysAlaAspTyrGluLysHisLysValTyrAlaCysGluValThrHisGlnGly | |
| | CAGCAAGGACAGCACCTACAGCCTCAGCAGCACCCTGACGCTGAGCAAAGCAGACTACGAGAAACACAAAGTCTACGCCTGCGAAGTCACCCATCAGGGC | |
| 501 | ---------!---------!---------!---------!---------!---------!---------!---------!---------!---------! | 600 |
| 1: | LeuSerSerProValThrLysSerPheAsnArgGlyGluCys | |
| | CTGAGCTCGCCCGTCACAAAGAGCTTCAACAGGGGAGAGTGT | |
| 601 | ---------!---------!---------!---------!-- | 642 |
| 6R6-F11 | | |
| SEQ ID No. 27 | | |
| 1: | AspIleGlnMetThrGlnSerProSerSerLeuSerAlaSerValGlyAspArgValThrIleThrCysGlnAlaSerGluGlnIleTyrTyrGlyIleA | |
| | GACATCCAGATGACCCAGTCTCCATCCTCCCTGTCTGCATCTGTAGGAGACAGAGTCACCATCACTTGCCAGGCCAGTGAGCAGATTTATTATGGTATTA | |
| 1 | ---------!---------!---------!---------!---------!---------!---------!---------!---------!---------! | 100 |
| 1: | snTrpTyrGlnGlnLysProGlyLysAlaProLysLeuLeuIleTyrGlyAlaThrGlyArgArgAspGlyValProSerArgPheSerGlySerGlySe | |
| | ATTGGTATCAGCAGAAACCAGGGAAAGCCCCTAAGCTCCTGATCTATGGTGCGACTGGTCGTAGGGATGGGGTCCCATCAAGGTTCAGTGGCAGTGGATC | |
| 101 | ---------!---------!---------!---------!---------!---------!---------!---------!---------!---------! | 200 |
| 1: | rGlyThrAspTyrThrLeuThrIleSerSerLeuGlnProGluAspPheAlaThrTyrTyrCysGlnGlnGlyTyrIleValProThrThrPheGlyGly | |
| | TGGGACAGATTACACTCTCACCATCAGCAGTCTGCAACCTGAAGATTTTGCAACTTACTACTGTCAACAAGGATATATTGTGCCTACGACGTTCGGCGGA | |
| 201 | ---------!---------!---------!---------!---------!---------!---------!---------!---------!---------! | 300 |
| 1: | GlyThrLysValGluIleLysArgThrValAlaAlaProSerValPheIlePheProProSerAspGluGlnLeuLysSerGlyThrAlaSerValValC | |
| | GGGACCAAGGTGGAGATCAAACGGACTGTGGCTGCACCATCTGTCTTCATCTTCCCGCCATCTGATGAGCAGTTGAAATCTGGAACTGCCTCTGTTGTGT | |
| 301 | ---------!---------!---------!---------!---------!---------!---------!---------!---------!---------! | 400 |
| 1: | ysLeuLeuAsnAsnPheTyrProArgGluAlaLysValGlnTrpLysValAspAsnAlaLeuGlnSerGlyAsnSerGlnGluSerValThrGluGlnAs | |
| | GCCTGCTGAATAACTTCTATCCCAGAGAGGCCAAAGTACAGTGGAAGGTGGATAACGCCCTCCAATCGGGTAACTCCCAGGAGAGTGTCACAGAGCAGGA | |
| 401 | ---------!---------!---------!---------!---------!---------!---------!---------!---------!---------! | 500 |
| 1: | pSerLysAspSerThrTyrSerLeuSerSerThrLeuThrLeuSerLysAlaAspTyrGluLysHisLysValTyrAlaCysGluValThrHisGlnGly | |
| | CAGCAAGGACAGCACCTACAGCCTCAGCAGCACCCTGACGCTGAGCAAAGCAGACTACGAGAAACACAAAGTCTACGCCTGCGAAGTCACCCATCAGGGC | |
| 501 | ---------!---------!---------!---------!---------!---------!---------!---------!---------!---------! | 600 |
| 1: | LeuSerSerProValThrLysSerPheAsnArgGlyGluCys | |
| | CTGAGCTCGCCCGTCACAAAGAGCTTCAACAGGGGAGAGTGT | |
| 601 | ---------!---------!---------!---------!-- | 642 |
| 6R6-F12 | | |
| SEQ ID No. 28 | | |
| 1: | AspIleGlnMetThrGlnSerProSerSerLeuSerAlaSerValGlyAspArgValThrIleThrCysGlnAlaSerGlnAlaIleLysTyrAlaIleA | |
| | GACATCCAGATGACCCAGTCTCCATCCTCCCTGTCTGCATCTGTAGGAGACAGAGTCACCATCACTTGCCAGGCCAGTCAGGCGATTAAGTATGCGATTA | |
| 1 | ---------!---------!---------!---------!---------!---------!---------!---------!---------!---------! | 100 |
| 1: | snTrpTyrGlnGlnLysProGlyLysAlaProLysLeuLeuIleTyrGlyAlaAsnTyrArgGlyProGlyValProSerArgPheSerGlySerGlySe | |
| | ATTGGTATCAGCAGAAACCAGGGAAAGCCCCTAAGCTCCTGATCTATGGTGCTAATTATCGTGGGCCGGGGGTCCCATCAAGGTTCAGTGGCAGTGGATC | |
| 101 | ---------!---------!---------!---------!---------!---------!---------!---------!---------!---------! | 200 |
| 1: | rGlyThrAspTyrThrLeuThrIleSerSerLeuGlnProGluAspPheAlaThrTyrTyrCysGlnGlnGlyTyrIleValProIleThrPheGlyGly | |
| | TGGGACAGATTACACTCTCACCATCAGCAGTCTGCAACCTGAAGATTTTGCAACTTACTACTGTCAACAAGGATATATTGTGCCTATTACGTTCGGCGGA | |
| 201 | ---------!---------!---------!---------!---------!---------!---------!---------!---------!---------! | 300 |
| 1: | GlyThrLysValGluIleLysArgThrValAlaAlaProSerValPheIlePheProProSerAspGluGlnLeuLysSerGlyThrAlaSerValValC | |
| | GGGACCAAGGTGGAGATCAAACGGACTGTGGCTGCACCATCTGTCTTCATCTTCCCGCCATCTGATGAGCAGTTGAAATCTGGAACTGCCTCTGTTGTGT | |
| 301 | ---------!---------!---------!---------!---------!---------!---------!---------!---------!---------! | 400 |
| 1: | ysLeuLeuAsnAsnPheTyrProArgGluAlaLysValGlnTrpLysValAspAsnAlaLeuGlnSerGlyAsnSerGlnGluSerValThrGluGlnAs | |
| | GCCTGCTGAATAACTTCTATCCCAGAGAGGCCAAAGTACAGTGGAAGGTGGATAACGCCCTCCAATCGGGTAACTCCCAGGAGAGTGTCACAGAGCAGGA | |
| 401 | ---------!---------!---------!---------!---------!---------!---------!---------!---------!---------! | 500 |
| 1: | pSerLysAspSerThrTyrSerLeuSerSerThrLeuThrLeuSerLysAlaAspTyrGluLysHisLysValTyrAlaCysGluValThrHisGlnGly | |
| | CAGCAAGGACAGCACCTACAGCCTCAGCAGCACCCTGACGCTGAGCAAAGCAGACTACGAGAAACACAAAGTCTACGCCTGCGAAGTCACCCATCAGGGC | |
| 501 | ---------!---------!---------!---------!---------!---------!---------!---------!---------!---------! | 600 |
| 1: | LeuSerSerProValThrLysSerPheAsnArgGlyGluCys | |
| | CTGAGCTCGCCCGTCACAAAGAGCTTCAACAGGGGAGAGTGT | |
| 601 | ---------!---------!---------!---------!-- | 642 |
| 3R6D11Q | | |
| SEQ ID No. 29 | | |
| 1: | AspIleGlnMetThrGlnSerProSerSerLeuSerAlaSerValGlyAspArgValThrIleThrCysGlnAlaSerGluProIleTyrTyrAlaIleA | |
| | GACATCCAGATGACCCAGTCTCCATCCTCCCTGTCTGCATCTGTAGGAGACAGAGTCACCATCACTTGCCAGGCCAGTGAGCCGATTTATTATGCTATTA | |
| 1 | ---------!---------!---------!---------!---------!---------!---------!---------!---------!---------! | 100 |
| 1: | snTrpTyrGlnGlnLysProGlyLysAlaProLysLeuLeuIleTyrGlyGlnSerHisLeuGlnAspGlyValProSerArgPheSerGlySerGlySe | |
| | ATTGGTATCAGCAGAAACCAGGGAAAGCCCCTAAGCTCCTGATCTATGGGCAGAGTCATCTTCAGGATGGGGTCCCATCAAGGTTCAGTGGCAGTGGATC | |
| 101 | ---------!---------!---------!---------!---------!---------!---------!---------!---------!---------! | 200 |
| 1: | rGlyThrAspTyrThrLeuThrIleSerSerLeuGlnProGluAspPheAlaThrTyrTyrCysGlnGlnGlyTyrArgValProThrPhePheGlyGly | |
| | TGGGACAGATTACACTCTCACCATCAGCAGTCTGCAACCTGAAGATTTTGCAACTTACTACTGTCAACAAGGATATCGGGTGCCTACTTTTTTCGGCGGA | |
| 201 | ---------!---------!---------!---------!---------!---------!---------!---------!---------!---------! | 300 |
| 1: | GlyThrLysValGluIleLysArgThrValAlaAlaProSerValPheIlePheProProSerAspGluGlnLeuLysSerGlyThrAlaSerValValC | |
| | GGGACCAAGGTGGAGATCAAACGGACTGTGGCTGCACCATCTGTCTTCATCTTCCCGCCATCTGATGAGCAGTTGAAATCTGGAACTGCCTCTGTTGTGT | |
| 301 | ---------!---------!---------!---------!---------!---------!---------!---------!---------!---------! | 400 |
| 1: | ysLeuLeuAsnAsnPheTyrProArgGluAlaLysValGlnTrpLysValAspAsnAlaLeuGlnSerGlyAsnSerGlnGluSerValThrGluGlnAs | |
| | GCCTGCTGAATAACTTCTATCCCAGAGAGGCCAAAGTACAGTGGAAGGTGGATAACGCCCTCCAATCGGGTAACTCCCAGGAGAGTGTCACAGAGCAGGA | |
| 401 | ---------!---------!---------!---------!---------!---------!---------!---------!---------!---------! | 500 |
| 1: | pSerLysAspSerThrTyrSerLeuSerSerThrLeuThrLeuSerLysAlaAspTyrGluLysHisLysValTyrAlaCysGluValThrHisGlnGly | |
| | CAGCAAGGACAGCACCTACAGCCTCAGCAGCACCCTGACGCTGAGCAAAGCAGACTACGAGAAACACAAAGTCTACGCCTGCGAAGTCACCCATCAGGGC | |
| 501 | ---------!---------!---------!---------!---------!---------!---------!---------!---------!---------! | 600 |
| 1: | LeuSerSerProValThrLysSerPheAsnArgGlyGluCys | |
| | CTGAGCTCGCCCGTCACAAAGAGCTTCAACAGGGGAGAGTGT | |
| 601 | ---------!---------!---------!---------!-- | 642 |
| 6R6-E6Q | | |
| SEQ ID No. 30 | | |
| 1: | AspIleGlnMetThrGlnSerProSerSerLeuSerAlaSerValGlyAspArgValThrIleThrCysGlnAlaSerGluGlnValTyrTyrGlyLeuA | |
| | GACATCCAGATGACCCAGTCTCCATCCTCCCTGTCTGCATCTGTAGGAGACAGAGTCACCATCACTTGCCAGGCCAGTGAGCAGGTTTATTATGGTCTTA | |
| 1 | ---------!---------!---------!---------!---------!---------!---------!---------!---------!---------! | 100 |
| 1: | snTrpTyrGlnGlnLysProGlyLysAlaProLysLeuLeuIleTyrGlyAlaAsnTyrArgValAspGlyValProSerArgPheSerGlySerGlySe | |
| | ATTGGTATCAGCAGAAACCAGGGAAAGCCCCTAAGCTCCTGATCTATGGTGCGAATTATCGTGTGGATGGGGTCCCATCAAGGTTCAGTGGCAGTGGATC | |
| 101 | ---------!---------!---------!---------!---------!---------!---------!---------!---------!---------! | 200 |
| 1: | rGlyThrAspTyrThrLeuThrIleSerSerLeuGlnProGluAspPheAlaThrTyrTyrCysGlnGlnGlyTyrValValProAsnValPheGlyGly | |
| | TGGGACAGATTACACTCTCACCATCAGCAGTCTGCAACCTGAAGATTTTGCAACTTACTACTGTCAACAAGGATATGTTGTTCCGAATGTGTTCGGCGGA | |
| 201 | ---------!---------!---------!---------!---------!---------!---------!---------!---------!---------! | 300 |
| 1: | GlyThrLysValGluIleLysArgThrValAlaAlaProSerValPheIlePheProProSerAspGluGlnLeuLysSerGlyThrAlaSerValValC | |
| | GGGACCAAGGTGGAGATCAAACGGACTGTGGCTGCACCATCTGTCTTCATCTTCCCGCCATCTGATGAGCAGTTGAAATCTGGAACTGCCTCTGTTGTGT | |
| 301 | ---------!---------!---------!---------!---------!---------!---------!---------!---------!---------! | 400 |
| 1: | ysLeuLeuAsnAsnPheTyrProArgGluAlaLysValGlnTrpLysValAspAsnAlaLeuGlnSerGlyAsnSerGlnGluSerValThrGluGlnAs | |
| | GCCTGCTGAATAACTTCTATCCCAGAGAGGCCAAAGTACAGTGGAAGGTGGATAACGCCCTCCAATCGGGTAACTCCCAGGAGAGTGTCACAGAGCAGGA | |
| 401 | ---------!---------!---------!---------!---------!---------!---------!---------!---------!---------! | 500 |
| 1: | pSerLysAspSerThrTyrSerLeuSerSerThrLeuThrLeuSerLysAlaAspTyrGluLysHisLysValTyrAlaCysGluValThrHisGlnGly | |
| | CAGCAAGGACAGCACCTACAGCCTCAGCAGCACCCTGACGCTGAGCAAAGCAGACTACGAGAAACACAAAGTCTACGCCTGCGAAGTCACCCATCAGGGC | |
| 501 | ---------!---------!---------!---------!---------!---------!---------!---------!---------!---------! | 600 |
| 1: | LeuSerSerProValThrLysSerPheAsnArgGlyGluCys | |
| | CTGAGCTCGCCCGTCACAAAGAGCTTCAACAGGGGAGAGTGT | |
| 601 | ---------!---------!---------!---------!-- | 642 |
| 6R8A-E11 | | |
| SEQ ID No. 31 | | |
| 1: | AspIleGlnMetThrGlnSerProSerSerLeuSerAlaSerValGlyAspArgValThrIleThrCysGlnAlaSerGluAsnIleArgTyrAlaIleA | |
| | GACATCCAGATGACCCAGTCTCCATCCTCCCTGTCTGCATCTGTAGGAGACAGAGTCACCATCACTTGCCAGGCCAGTGAGAATATTAGGTATGCTATTA | |
| 1 | ---------!---------!---------!---------!---------!---------!---------!---------!---------!---------! | 100 |
| 1: | snTrpTyrGlnGlnLysProGlyLysAlaProLysLeuLeuIleTyrGlyAlaAsnTyrArgValAspGlyValProSerArgPheSerGlySerGlySe | |
| | ATTGGTATCAGCAGAAACCAGGGAAAGCCCCTAAGCTCCTGATCTATGGTGCGAATTATCGTGTGGATGGGGTCCCATCAAGGTTCAGTGGCAGTGGATC | |
| 101 | ---------!---------!---------!---------!---------!---------!---------!---------!---------!---------! | 200 |
| 1: | rGlyThrAspTyrThrLeuThrIleSerSerLeuGlnProGluAspPheAlaThrTyrTyrCysGlnGlnGlyTyrAsnValProThrAspPheGlyGly | |
| | TGGGACAGATTACACTCTCACCATCAGCAGTCTGCAACCTGAAGATTTTGCAACTTACTACTGTCAACAAGGATATAATGTGCCTACTGATTTCGGCGGA | |
| 201 | ---------!---------!---------!---------!---------!---------!---------!---------!---------!---------! | 300 |
| 1: | GlyThrLysValGluIleLysArgThrValAlaAlaProSerValPheIlePheProProSerAspGluGlnLeuLysSerGlyThrAlaSerValValC | |
| | GGGACCAAGGTGGAGATCAAACGGACTGTGGCTGCACCATCTGTCTTCATCTTCCCGCCATCTGATGAGCAGTTGAAATCTGGAACTGCCTCTGTTGTGT | |
| 301 | ---------!---------!---------!---------!---------!---------!---------!---------!---------!---------! | 400 |
| 1: | ysLeuLeuAsnAsnPheTyrProArgGluAlaLysValGlnTrpLysValAspAsnAlaLeuGlnSerGlyAsnSerGlnGluSerValThrGluGlnAs | |
| | GCCTGCTGAATAACTTCTATCCCAGAGAGGCCAAAGTACAGTGGAAGGTGGATAACGCCCTCCAATCGGGTAACTCCCAGGAGAGTGTCACAGAGCAGGA | |
| 401 | ---------!---------!---------!---------!---------!---------!---------!---------!---------!---------! | 500 |
| 1: | pSerLysAspGlyThrTyrSerLeuSerSerThrLeuThrLeuSerLysAlaAspTyrGluLysHisLysValTyrAlaCysGluValThrHisGlnGly | |
| | CAGCAAGGACGGCACCTACAGCCTCAGCAGCACCCTGACGCTGAGCAAAGCAGACTACGAGAAACACAAAGTCTACGCCTGCGAAGTCACCCATCAGGGC | |
| 501 | ---------!---------!---------!---------!---------!---------!---------!---------!---------!---------! | 600 |
| 1: | LeuSerSerProValThrLysSerPheAsnArgGlyGluCys | |
| | CTGAGCTCGCCCGTCACAAAGAGCTTCAACAGGGGAGAGTGT | |
| 601 | ---------!---------!---------!---------!-- | 642 |
| 6R8A-F2 | | |
| SEQ ID No. 32 | | |
| 1: | AspIleGlnMetThrGlnSerProSerSerLeuSerAlaSerValGlyAspArgValThrIleThrCysGlnAlaSerGluLysValTyrTyrGlyValA | |
| | GACATCCAGATGACCCAGTCTCCATCCTCCCTGTCTGCATCTGTAGGAGACAGAGTCACCATCACTTGCCAGGCCAGTGAGAAGGTTTATTATGGGGTTA | |
| 1 | ---------!---------!---------!---------!---------!---------!---------!---------!---------!---------! | 100 |
| 1: | snTrpTyrGlnGlnLysProGlyLysAlaProLysLeuLeuIleTyrGlyThrAsnTyrArgLeuAspGlyValProSerArgPheSerGlySerGlySe | |
| | ATTGGTATCAGCAGAAACCAGGGAAAGCCCCTAAGCTCCTGATCTATGGTACGAATTATCGTCTGGATGGGGTCCCATCAAGGTTCAGTGGCAGTGGATC | |
| 101 | ---------!---------!---------!---------!---------!---------!---------!---------!---------!---------! | 200 |
| 1: | rGlyThrAspSerThrLeuThrIleSerSerLeuGlnProGluAspPheAlaThrTyrTyrCysGlnGlnGlyTyrAsnValProAsnAlaPheGlyGly | |
| | TGGGACAGATTCCACTCTCACCATCAGCAGTCTGCAACCTGAAGATTTTGCAACTTACTACTGTCAACAAGGATATAATGTTCCGAATGCGTTCGGCGGA | |
| 201 | ---------!---------!---------!---------!---------!---------!---------!---------!---------!---------! | 300 |
| 1: | GlyThrLysValGluIleLysArgThrValAlaAlaProSerValPheIlePheProProSerAspGluGlnLeuLysSerGlyThrAlaSerValValC | |
| | GGGACCAAGGTGGAGATCAAACGGACTGTGGCTGCACCATCTGTCTTCATCTTCCCGCCATCTGATGAGCAGTTGAAATCTGGAACTGCCTCTGTTGTGT | |
| 301 | ---------!---------!---------!---------!---------!---------!---------!---------!---------!---------! | 400 |
| 1: | ysLeuLeuAsnAsnPheTyrProArgGluAlaLysValGlnTrpLysValAspAsnAlaLeuGlnSerGlyAsnSerGlnGluSerValThrGluGlnAs | |
| | GCCTGCTGAATAACTTCTATCCCAGAGAGGCCAAAGTACAGTGGAAGGTGGATAACGCCCTCCAATCGGGTAACTCCCAGGAGAGTGTCACAGAGCAGGA | |
| 401 | ---------!---------!---------!---------!---------!---------!---------!---------!---------!---------! | 500 |
| 1: | pSerLysAspSerThrTyrSerLeuSerSerThrLeuThrLeuSerLysAlaAspTyrGluLysHisLysValTyrAlaCysGluValThrHisGlnGly | |
| | CAGCAAGGACAGCACCTACAGCCTCAGCAGCACCCTGACGCTGAGCAAAGCAGACTACGAGAAACACAAAGTCTACGCCTGCGAAGTCACCCATCAGGGC | |
| 501 | ---------!---------!---------!---------!---------!---------!---------!---------!---------!---------! | 600 |
| 1: | LeuSerSerProValThrLysSerPheAsnArgGlyGluCys | |
| | CTGAGCTCGCCCGTCACAAAGAGCTTCAACAGGGGAGAGTGT | |
| 601 | ---------!---------!---------!---------!-- | 642 |
| 6R8A-E1 | | |
| SEQ ID No. 33 | | |
| 1: | AspIleGlnMetThrGlnSerProSerSerLeuSerAlaSerValGlyAspArgValThrIleThrCysGlnAlaSerGluAsnIleArgTyrAlaIleA | |
| | GACATCCAGATGACCCAGTCTCCATCCTCCCTGTCTGCATCTGTAGGAGACAGAGTCACCATCACTTGCCAGGCCAGTGAGAATATTAGGTATGCTATTA | |
| 1 | ---------!---------!---------!---------!---------!---------!---------!---------!---------!---------! | 100 |
| 1: | snTrpTyrGlnGlnLysProGlyLysAlaProLysLeuLeuIleTyrGlyAlaAsnTyrArgValAspGlyValProSerArgPheSerGlySerGlySe | |
| | ATTGGTATCAGCAGAAACCAGGGAAAGCCCCTAAGCTCCTGATCTATGGTGCGAATTATCGTGTGGATGGGGTCCCATCAAGGTTCAGTGGCAGTGGATC | |
| 101 | ---------!---------!---------!---------!---------!---------!---------!---------!---------!---------! | 200 |
| 1: | rGlyThrAspTyrThrLeuThrIleSerSerLeuGlnProGluAspPheAlaThrTyrTyrCysGlnGlnGlyTyrAsnValProThrAspPheGlyGly | |
| | TGGGACAGATTACACTCTCACCATCAGCAGTCTGCAACCTGAAGATTTTGCAACTTACTACTGTCAACAAGGATATAATGTGCCTACTGATTTCGGCGGA | |
| 201 | ---------!---------!---------!---------!---------!---------!---------!---------!---------!---------! | 300 |
| 1: | GlyThrLysValGluIleLysArgThrValAlaAlaProSerValPheIlePheProProSerAspGluGlnLeuLysSerGlyThrAlaSerValValC | |
| | GGGACCAAGGTGGAGATCAAACGGACTGTGGCTGCACCATCTGTCTTCATCTTCCCGCCATCTGATGAGCAGTTGAAATCTGGAACTGCCTCTGTTGTGT | |
| 301 | ---------!---------!---------!---------!---------!---------!---------!---------!---------!---------! | 400 |
| 1: | ysLeuLeuAsnAsnPheTyrProArgGluAlaLysValGlnTrpLysValAspAsnAlaLeuGlnSerGlyAsnSerGlnGluSerValThrGluGlnAs | |
| | GCCTGCTGAATAACTTCTATCCCAGAGAGGCCAAAGTACAGTGGAAGGTGGATAACGCCCTCCAATCGGGTAACTCCCAGGAGAGTGTCACAGAGCAGGA | |
| 401 | ---------!---------!---------!---------!---------!---------!---------!---------!---------!---------! | 500 |
| 1: | pSerLysAspGlyThrTyrSerLeuSerSerThrLeuThrLeuSerLysAlaAspTyrGluLysHisLysValTyrAlaCysGluValThrHisGlnGly | |
| | CAGCAAGGACGGCACCTACAGCCTCAGCAGCACCCTGACGCTGAGCAAAGCAGACTACGAGAAACACAAAGTCTACGCCTGCGAAGTCACCCATCAGGGC | |
| 501 | ---------!---------!---------!---------!---------!---------!---------!---------!---------!---------! | 600 |
| 1: | LeuSerSerProValThrLysSerPheAsnArgGlyGluCys | |
| | CTGAGCTCGCCCGTCACAAAGAGCTTCAACAGGGGAGAGTGT | |
| 601 | ---------!---------!---------!---------!-- | 642 |
| 6R8A-A2 | | |
| SEQ ID No. 34 | | |
| 1: | AspIleGlnMetThrGlnSerProSerSerLeuSerAlaSerValGlyAspArgValThrIleThrCysGlnAlaSerGluGluValHisTyrGlyIleA | |
| | GACATCCAGATGACCCAGTCTCCATCCTCCCTGTCTGCATCTGTAGGAGACAGAGTCACCATCACTTGCCAGGCCAGTGAGGAGGTTCATTATGGGATTA | |
| 1 | ---------!---------!---------!---------!---------!---------!---------!---------!---------!---------! | 100 |
| 1: | snTrpTyrGlnGlnLysProGlyLysAlaProLysLeuLeuIleTyrGlyAlaAsnTyrArgValAspGlyValProSerArgPheSerGlySerGlySe | |
| | ATTGGTATCAGCAGAAACCAGGGAAAGCCCCTAAGCTCCTGATCTATGGTGCGAATTATCGTGTGGATGGGGTCCCATCAAGGTTCAGTGGCAGTGGATC | |
| 101 | ---------!---------!---------!---------!---------!---------!---------!---------!---------!---------! | 200 |
| 1: | rGlyThrAspTyrThrLeuThrIleSerSerLeuGlnProGluAspPheAlaThrTyrTyrCysGlnGlnGlyTyrAsnValProThrAspPheGlyGly | |
| | TGGGACAGATTACACTCTCACCATCAGCAGTCTGCAACCTGAAGATTTTGCAACTTACTACTGTCAACAAGGATATAATGTGCCTACTGATTTCGGCGGA | |
| 201 | ---------!---------!---------!---------!---------!---------!---------!---------!---------!---------! | 300 |
| 1: | GlyThrLysValGluIleLysArgThrValAlaAlaProSerValPheIlePheProProSerAspGluGlnLeuLysSerGlyThrAlaSerValValC | |
| | GGGACCAAGGTGGAGATCAAACGGACTGTGGCTGCACCATCTGTCTTCATCTTCCCGCCATCTGATGAGCAGTTGAAATCTGGAACTGCCTCTGTTGTGT | |
| 301 | ---------!---------!---------!---------!---------!---------!---------!---------!---------!---------! | 400 |
| 1: | ysLeuLeuAsnAsnPheTyrProArgGluAlaLysValGlnTrpLysValAspAsnAlaLeuGlnSerGlyAsnSerGlnGluSerValThrGluGlnAs | |
| | GCCTGCTGAATAACTTCTATCCCAGAGAGGCCAAAGTACAGTGGAAGGTGGATAACGCCCTCCAATCGGGTAACTCCCAGGAGAGTGTCACAGAGCAGGA | |
| 401 | ---------!---------!---------!---------!---------!---------!---------!---------!---------!---------! | 500 |
| 1: | pSerLysAspSerThrTyrSerLeuSerSerThrLeuThrLeuSerLysAlaAspTyrGluLysHisLysValTyrAlaCysGluValThrHisGlnGly | |
| | CAGCAAGGACAGCACCTACAGCCTCAGCAGCACCCTGACGCTGAGCAAAGCAGACTACGAGAAACACAAAGTCTACGCCTGCGAAGTCACCCATCAGGGC | |
| 501 | ---------!---------!---------!---------!---------!---------!---------!---------!---------!---------! | 600 |
| 1: | LeuSerSerProValThrLysSerPheAsnArgGlyGluCys | |
| | CTGAGCTCGCCCGTCACAAAGAGCTTCAACAGGGGAGAGTGT | |
| 601 | ---------!---------!---------!---------!-- | 642 |
| 6R8C-G3 | | |
| SEQ ID No. 35 | | |
| 1: | AspIleGlnMetThrGlnSerProSerSerLeuSerAlaSerValGlyAspArgValThrIleThrCysGlnAlaSerGluGluValHisTyrGlyIleA | |
| | GACATCCAGATGACCCAGTCTCCATCCTCCCTGTCTGCATCTGTAGGAGACAGAGTCACCATCACTTGCCAGGCCAGTGAGGAGGTTCATTATGGGATTA | |
| 1 | ---------!---------!---------!---------!---------!---------!---------!---------!---------!---------! | 100 |
| 1: | snTrpTyrGlnGlnLysProGlyLysAlaProLysLeuLeuIleTyrGlyAlaAsnTyrArgValAspGlyValProSerArgPheSerGlySerGlySe | |
| | ATTGGTATCAGCAGAAACCAGGGAAAGCCCCTAAGCTCCTGATCTATGGTGCGAATTATCGTGTGGATGGGGTCCCATCAAGGTTCAGTGGCAGTGGATC | |
| 101 | ---------!---------!---------!---------!---------!---------!---------!---------!---------!---------! | 200 |
| 1: | rGlyThrAspTyrThrLeuThrIleSerSerLeuGlnProGluAspPheAlaThrTyrTyrCysGlnGlnGlyTyrAsnValProThrAspPheGlyGly | |
| | TGGGACAGATTACACTCTCACCATCAGCAGTCTGCAACCTGAAGATTTTGCAACTTACTACTGTCAACAAGGATATAATGTGCCTACTGATTTCGGCGGA | |
| 201 | ---------!---------!---------!---------!---------!---------!---------!---------!---------!---------! | 300 |
| 1: | GlyThrLysValGluIleLysArgThrValAlaAlaProSerValPheIlePheProProSerAspGluGlnLeuLysSerGlyThrAlaSerValValC | |
| | GGGACCAAGGTGGAGATCAAACGGACTGTGGCTGCACCATCTGTCTTCATCTTCCCGCCATCTGATGAGCAGTTGAAATCTGGAACTGCCTCTGTTGTGT | |
| 301 | ---------!---------!---------!---------!---------!---------!---------!---------!---------!---------! | 400 |
| 1: | ysLeuLeuAsnAsnPheTyrProArgGluAlaLysValGlnTrpLysValAspAsnAlaLeuGlnSerGlyAsnSerGlnGluSerValThrGluGlnAs | |
| | GCCTGCTGAATAACTTCTATCCCAGAGAGGCCAAAGTACAGTGGAAGGTGGATAACGCCCTCCAATCGGGTAACTCCCAGGAGAGTGTCACAGAGCAGGA | |
| 401 | ---------!---------!---------!---------!---------!---------!---------!---------!---------!---------! | 500 |
| 1: | pSerLysAspSerThrTyrSerLeuSerSerThrLeuThrLeuSerLysAlaAspTyrGluLysHisLysValTyrAlaCysGluValThrHisGlnGly | |
| | CAGCAAGGACAGCACCTACAGCCTCAGCAGCACCCTGACGCTGAGCAAAGCAGACTACGAGAAACACAAAGTCTACGCCTGCGAAGTCACCCATCAGGGC | |
| 501 | ---------!---------!---------!---------!---------!---------!---------!---------!---------!---------! | 600 |
| 1: | LeuSerSerProValThrLysSerPheAsnArgGlyGluCys | |
| | CTGAGCTCGCCCGTCACAAAGAGCTTCAACAGGGGAGAGTGT | |
| 601 | ---------!---------!---------!---------!-- | 642 |
| 6R8C-G4 | | |
| SEQ ID No. 36 | | |
| 1: | AspIleGlnMetThrGlnSerProSerSerLeuSerAlaSerValGlyAspArgValThrIleThrCysGlnAlaSerGluAsnIleArgTyrAlaIleA | |
| | GACATCCAGATGACCCAGTCTCCATCCTCCCTGTCTGCATCTGTAGGAGACAGAGTCACCATCACTTGCCAGGCCAGTGAGAATATTAGGTATGCTATTA | |
| 1 | ---------!---------!---------!---------!---------!---------!---------!---------!---------!---------! | 100 |
| 1: | snTrpTyrGlnGlnLysProGlyLysAlaProLysLeuLeuIleTyrGlyAlaAsnTyrArgValAspGlyValProSerArgPheSerGlySerGlySe | |
| | ATTGGTATCAGCAGAAACCAGGGAAAGCCCCTAAGCTCCTGATCTATGGTGCGAATTATCGTGTGGATGGGGTCCCATCAAGGTTCAGTGGCAGTGGATC | |
| 101 | ---------!---------!---------!---------!---------!---------!---------!---------!---------!---------! | 200 |
| 1: | rGlyThrAspTyrThrLeuThrIleSerSerLeuGlnProGluAspPheAlaThrTyrTyrCysGlnGlnGlyTyrAsnValProThrAspPheGlyGly | |
| | TGGGACAGATTACACTCTCACCATCAGCAGTCTGCAACCTGAAGATTTTGCAACTTACTACTGTCAACAAGGATATAATGTGCCTACTGATTTCGGCGGA | |
| 201 | ---------!---------!---------!---------!---------!---------!---------!---------!---------!---------! | 300 |
| 1: | GlyThrLysValGluIleLysArgThrValAlaAlaProSerValPheIlePheProProSerAspGluGlnLeuLysSerGlyThrAlaSerValValC | |
| | GGGACCAAGGTGGAGATCAAACGGACTGTGGCTGCACCATCTGTCTTCATCTTCCCGCCATCTGATGAGCAGTTGAAATCTGGAACTGCCTCTGTTGTGT | |
| 301 | ---------!---------!---------!---------!---------!---------!---------!---------!---------!---------! | 400 |
| 1: | ysLeuLeuAsnAsnPheTyrProArgGluAlaLysValGlnTrpLysValAspAsnAlaLeuGlnSerGlyAsnSerGlnGluSerValThrGluGlnAs | |
| | GCCTGCTGAATAACTTCTATCCCAGAGAGGCCAAAGTACAGTGGAAGGTGGATAACGCCCTCCAATCGGGTAACTCCCAGGAGAGTGTCACAGAGCAGGA | |
| 401 | ---------!---------!---------!---------!---------!---------!---------!---------!---------!---------! | 500 |
| 1: | pSerLysAspGlyThrTyrSerLeuSerSerThrLeuThrLeuSerLysAlaAspTyrGluLysHisLysValTyrAlaCysGluValThrHisGlnGly | |
| | CAGCAAGGACGGCACCTACAGCCTCAGCAGCACCCTGACGCTGAGCAAAGCAGACTACGAGAAACACAAAGTCTACGCCTGCGAAGTCACCCATCAGGGC | |
| 501 | ---------!---------!---------!---------!---------!---------!---------!---------!---------!---------! | 600 |
| 1: | LeuSerSerProValThrLysSerPheAsnArgGlyGluCys | |
| | CTGAGCTCGCCCGTCACAAAGAGCTTCAACAGGGGAGAGTGT | |
| 601 | ---------!---------!---------!---------!-- | 642 |
| 6R8C-G8 | | |
| SEQ ID No. 37 | | |
| 1: | AspIleGlnMetThrGlnSerProSerSerLeuSerAlaSerValGlyAspArgValThrIleThrCysGlnAlaSerGluGluValHisTyrGlyIleA | |
| | GACATCCAGATGACCCAGTCTCCATCCTCCCTGTCTGCATCTGTAGGAGACAGAGTCACCATCACTTGCCAGGCCAGTGAGGAGGTTCATTATGGGATTA | |
| 1 | ---------!---------!---------!---------!---------!---------!---------!---------!---------!---------! | 100 |
| 1: | snTrpTyrGlnGlnLysProGlyLysAlaProLysLeuLeuIleTyrGlyAlaAsnTyrArgValAspGlyValProSerArgPheSerGlySerGlySe | |
| | ATTGGTATCAGCAGAAACCAGGGAAAGCCCCTAAGCTCCTGATCTATGGTGCGAATTATCGTGTGGATGGGGTCCCATCAAGGTTCAGTGGCAGTGGATC | |
| 101 | ---------!---------!---------!---------!---------!---------!---------!---------!---------!---------! | 200 |
| 1: | rGlyThrAspTyrThrLeuThrIleSerSerLeuGlnProGluAspPheAlaThrTyrTyrCysGlnGlnGlyTyrAsnValProThrAspPheGlyGly | |
| | TGGGACAGATTACACTCTCACCATCAGCAGTCTGCAACCTGAAGATTTTGCAACTTACTACTGTCAACAAGGATATAATGTGCCTACTGATTTCGGCGGA | |
| 201 | ---------!---------!---------!---------!---------!---------!---------!---------!---------!---------! | 300 |
| 1: | GlyThrLysValGluIleLysArgThrValAlaAlaProSerValPheIlePheProProSerAspGluGlnLeuLysSerGlyThrAlaSerValValC | |
| | GGGACCAAGGTGGAGATCAAACGGACTGTGGCTGCACCATCTGTCTTCATCTTCCCGCCATCTGATGAGCAGTTGAAATCTGGAACTGCCTCTGTTGTGT | |
| 301 | ---------!---------!---------!---------!---------!---------!---------!---------!---------!---------! | 400 |
| 1: | ysLeuLeuAsnAsnPheTyrProArgGluAlaLysValGlnTrpLysValAspAsnAlaLeuGlnSerGlyAsnSerGlnGluSerValThrGluGlnAs | |
| | GCCTGCTGAATAACTTCTATCCCAGAGAGGCCAAAGTACAGTGGAAGGTGGATAACGCCCTCCAATCGGGTAACTCCCAGGAGAGTGTCACAGAGCAGGA | |
| 401 | ---------!---------!---------!---------!---------!---------!---------!---------!---------!---------! | 500 |
| 1: | pSerLysAspSerThrTyrSerLeuSerSerThrLeuThrLeuSerLysAlaAspTyrGluLysHisLysValTyrAlaCysGluValThrHisGlnGly | |
| | CAGCAAGGACAGCACCTACAGCCTCAGCAGCACCCTGACGCTGAGCAAAGCAGACTACGAGAAACACAAAGTCTACGCCTGCGAAGTCACCCATCAGGGC | |
| 501 | ---------!---------!---------!---------!---------!---------!---------!---------!---------!---------! | 600 |
| 1: | LeuSerSerProValThrLysSerPheAsnArgGlyGluCys | |
| | CTGAGCTCGCCCGTCACAAAGAGCTTCAACAGGGGAGAGTGT | |
| 601 | ---------!---------!---------!---------!-- | 642 |
| 6R8C-D9 | | |
| SEQ ID No. 38 | | |
| 1: | AspIleGlnMetThrGlnSerProSerSerLeuSerAlaSerValGlyAspArgValThrIleThrCysGlnAlaSerGluGluValHisTyrGlyIleA | |
| | GACATCCAGATGACCCAGTCTCCATCCTCCCTGTCTGCATCTGTAGGAGACAGAGTCACCATCACTTGCCAGGCCAGTGAGGAGGTTCATTATGGGATTA | |
| 1 | ---------!---------!---------!---------!---------!---------!---------!---------!---------!---------! | 100 |
| 1: | snTrpTyrGlnGlnLysProGlyLysAlaProLysLeuLeuIleTyrGlyAlaAsnTyrArgValAspGlyValProSerArgPheSerGlySerGlySe | |
| | ATTGGTATCAGCAGAAACCAGGGAAAGCCCCTAAGCTCCTGATCTATGGTGCGAATTATCGTGTGGATGGGGTCCCATCAAGGTTCAGTGGCAGTGGATC | |
| 101 | ---------!---------!---------!---------!---------!---------!---------!---------!---------!---------! | 200 |
| 1: | rGlyThrAspTyrThrLeuThrIleSerSerLeuGlnProGluAspPheAlaThrTyrTyrCysGlnGlnGlyTyrAsnValProThrAspPheGlyGly | |
| | TGGGACAGATTACACTCTCACCATCAGCAGTCTGCAACCTGAAGATTTTGCAACTTACTACTGTCAACAAGGATATAATGTGCCTACTGATTTCGGCGGA | |
| 201 | ---------!---------!---------!---------!---------!---------!---------!---------!---------!---------! | 300 |
| 1: | GlyThrLysValGluIleLysArgThrValAlaAlaProSerValPheIlePheProProSerAspGluGlnLeuLysSerGlyThrAlaSerValValC | |
| | GGGACCAAGGTGGAGATCAAACGGACTGTGGCTGCACCATCTGTCTTCATCTTCCCGCCATCTGATGAGCAGTTGAAATCTGGAACTGCCTCTGTTGTGT | |
| 301 | ---------!---------!---------!---------!---------!---------!---------!---------!---------!---------! | 400 |
| 1: | ysLeuLeuAsnAsnPheTyrProArgGluAlaLysValGlnTrpLysValAspAsnAlaLeuGlnSerGlyAsnSerGlnGluSerValThrGluGlnAs | |
| | GCCTGCTGAATAACTTCTATCCCAGAGAGGCCAAAGTACAGTGGAAGGTGGATAACGCCCTCCAATCGGGTAACTCCCAGGAGAGTGTCACAGAGCAGGA | |
| 401 | ---------!---------!---------!---------!---------!---------!---------!---------!---------!---------! | 500 |
| 1: | pSerLysAspSerThrTyrSerLeuSerSerThrLeuThrLeuSerLysAlaAspTyrGluLysHisLysValTyrAlaCysGluValThrHisGlnGly | |
| | CAGCAAGGACAGCACCTACAGCCTCAGCAGCACCCTGACGCTGAGCAAAGCAGACTACGAGAAACACAAAGTCTACGCCTGCGAAGTCACCCATCAGGGC | |
| 501 | ---------!---------!---------!---------!---------!---------!---------!---------!---------!---------! | 600 |
| 1: | LeuSerSerProValThrLysSerPheAsnArgGlyGluCys | |
| | CTGAGCTCGCCCGTCACAAAGAGCTTCAACAGGGGAGAGTGT | |
| 601 | ---------!---------!---------!---------!-- | 642 |
| 6R8C-D10 | | |
| SEQ ID No. 39 | | |
| 1: | AspIleGlnMetThrGlnSerProSerSerLeuSerAlaSerValGlyAspArgValThrIleThrCysGlnAlaSerGluGluValHisTyrGlyIleA | |
| | GACATCCAGATGACCCAGTCTCCATCCTCCCTGTCTGCATCTGTAGGAGACAGAGTCACCATCACTTGCCAGGCCAGTGAGGAGGTTCATTATGGGATTA | |
| 1 | ---------!---------!---------!---------!---------!---------!---------!---------!---------!---------! | 100 |
| 1: | snTrpTyrGlnGlnLysProGlyLysAlaProLysLeuLeuIleTyrGlyAlaAsnTyrArgValAspGlyValProSerArgPheSerGlySerGlySe | |
| | ATTGGTATCAGCAGAAACCAGGGAAAGCCCCTAAGCTCCTGATCTATGGTGCGAATTATCGTGTGGATGGGGTCCCATCAAGGTTCAGTGGCAGTGGATC | |
| 101 | ---------!---------!---------!---------!---------!---------!---------!---------!---------!---------! | 200 |
| 1: | rGlyThrAspTyrThrLeuThrIleSerSerLeuGlnProGluAspPheAlaThrTyrTyrCysGlnGlnGlyTyrAsnValProThrAspPheGlyGly | |
| | TGGGACAGATTACACTCTCACCATCAGCAGTCTGCAACCTGAAGATTTTGCAACTTACTACTGTCAACAAGGATATAATGTGCCTACTGATTTCGGCGGA | |
| 201 | ---------!---------!---------!---------!---------!---------!---------!---------!---------!---------! | 300 |
| 1: | GlyThrLysValGluIleLysArgThrValAlaAlaProSerValPheIlePheProProSerAspGluGlnLeuLysSerGlyThrAlaSerValValC | |
| | GGGACCAAGGTGGAGATCAAACGGACTGTGGCTGCACCATCTGTCTTCATCTTCCCGCCATCTGATGAGCAGTTGAAATCTGGAACTGCCTCTGTTGTGT | |
| 301 | ---------!---------!---------!---------!---------!---------!---------!---------!---------!---------! | 400 |
| 1: | ysLeuLeuAsnAsnPheTyrProArgGluAlaLysValGlnTrpLysValAspAsnAlaLeuGlnSerGlyAsnSerGlnGluSerValThrGluGlnAs | |
| | GCCTGCTGAATAACTTCTATCCCAGAGAGGCCAAAGTACAGTGGAAGGTGGATAACGCCCTCCAATCGGGTAACTCCCAGGAGAGTGTCACAGAGCAGGA | |
| 401 | ---------!---------!---------!---------!---------!---------!---------!---------!---------!---------! | 500 |
| 1: | pSerLysAspSerThrTyrSerLeuSerSerThrLeuThrLeuSerLysAlaAspTyrGluLysHisLysValTyrAlaCysGluValThrHisGlnGly | |
| | CAGCAAGGACAGCACCTACAGCCTCAGCAGCACCCTGACGCTGAGCAAAGCAGACTACGAGAAACACAAAGTCTACGCCTGCGAAGTCACCCATCAGGGC | |
| 501 | ---------!---------!---------!---------!---------!---------!---------!---------!---------!---------! | 600 |
| 1: | LeuSerSerProValThrLysSerPheAsnArgGlyGluCys | |
| | CTGAGCTCGCCCGTCACAAAGAGCTTCAACAGGGGAGAGTGT | |
| 601 | ---------!---------!---------!---------!-- | 642 |
| 6R8C-F10 | | |
| SEQ ID No. 40 | | |
| 1: | AspIleGlnMetThrGlnSerProSerSerLeuSerAlaSerValGlyAspArgValThrIleThrCysLysAlaSerGluAspIleArgTyrGlyLeuA | |
| | GACATCCAGATGACCCAGTCTCCATCCTCCCTGTCCGCATCTGTAGGAGACAGAGTCACCATCACTTGCAAGGCCAGTGAGGACATTCGTTATGGGTTAA | |
| 1 | ---------!---------!---------!---------!---------!---------!---------!---------!---------!---------! | 100 |
| 1: | snTrpTyrGlnGlnLysProGlyLysAlaProLysLeuLeuIleTyrGlyAlaAsnTyrArgValAspGlyValProSerArgPheSerGlySerGlySe | |
| | ACTGGTATCAGCAGAAACCAGGGAAAGCCCCTAAGCTCCTGATCTATGGTGCGAATTATCGTGTGGATGGGGTCCCATCAAGGTTCAGTGGCAGTGGATC | |
| 101 | ---------!---------!---------!---------!---------!---------!---------!---------!---------!---------! | 200 |
| 1: | rGlyThrAspTyrThrLeuThrIleSerSerLeuGlnProGluAspPheAlaThrTyrTyrCysGlnGlnGlyTyrAsnValProAsnPhePheGlyGly | |
| | TGGGACAGATTACACTCTCACCATCAGCAGTCTGCAACCTGAAGATTTTGCAACTTACTACTGTCAACAAGGATATAATGTGCCTAATTTTTTCGGCGGA | |
| 201 | ---------!---------!---------!---------!---------!---------!---------!---------!---------!---------! | 300 |
| 1: | GlyThrLysValGluIleLysArgThrValAlaAlaProSerValPheIlePheProProSerAspGluGlnLeuLysSerGlyThrAlaSerValValC | |
| | GGGACCAAGGTGGAGATCAAACGGACTGTGGCTGCACCATCTGTCTTCATCTTCCCGCCATCTGATGAGCAGTTGAAATCTGGAACTGCCTCTGTTGTGT | |
| 301 | ---------!---------!---------!---------!---------!---------!---------!---------!---------!---------! | 400 |
| 1: | ysLeuLeuAsnAsnPheTyrProArgGluAlaLysValGlnTrpLysValAspAsnAlaLeuGlnSerGlyAsnSerGlnGluSerValThrGluGlnAs | |
| | GCCTGCTGAATAACTTCTATCCCAGAGAGGCCAAAGTACAGTGGAAGGTGGATAACGCCCTCCAATCGGGTAACTCCCAGGAGAGTGTCACAGAGCAGGA | |
| 401 | ---------!---------!---------!---------!---------!---------!---------!---------!---------!---------! | 500 |
| 1: | pSerLysAspSerThrTyrSerLeuSerSerThrLeuThrLeuSerLysAlaAspTyrGluLysHisLysValTyrAlaCysGluValThrHisGlnGly | |
| | CAGCAAGGACAGCACCTACAGCCTCAGCAGCACCCTGACGCTGAGCAAAGCAGACTACGAGAAACACAAAGTCTACGCCTGCGAAGTCACCCATCAGGGC | |
| 501 | ---------!---------!---------!---------!---------!---------!---------!---------!---------!---------! | 600 |
| 1: | LeuSerSerProValThrLysSerPheAsnArgGlyGluCys | |
| | CTGAGCTCGCCCGTCACAAAGAGCTTCAACAGGGGAGAGTGT | |
| 601 | ---------!---------!---------!---------!-- | 642 |

### Heavy Chain Sequences of Clones Selected on BA.5 and BA.2.75.2

3R4H4 SEQ ID No. 41

| | | |
|---|---|---|
| 1: | GluValGlnLeuValGluSerGlyGlyGlyLeuValGlnProGlyGlySerLeuArgLeuSerCysGluAlaSerGlyPheThrPheProLeuGlnAlaM | |
| | GAGGTGCAGCTGGTGGAGTCTGGGGGAGGCTTGGTACAGCCTGGGGGGTCCCTGAGACTCTCCTGTGAGGCTTCTGGATTTACTTTTCCTCTTCAGGCTA | |
| 1 | ---------!---------!---------!---------!---------!---------!---------!---------!---------!---------! | 100 |
| 1: | etSerTrpValArgGlnAlaProGlyLysGlyLeuGluTrpIleGlyValIleAlaSerAsnAlaAspThrTyrTyrAlaSerTrpAlaLysGlyArgPh | |
| | TGAGTTGGGTCCGCCAGGCTCCAGGGAAGGGGCTGGAGTGGATCGGAGTCATTGCTAGTAATGCTGACACATACTACGCGAGCTGGGCGAAAGGCCGGTT | |
| 101 | ---------!---------!---------!---------!---------!---------!---------!---------!---------!---------! | 200 |
| 1: | eThrIleSerArgAspAsnSerLysAsnThrLeuTyrLeuGlnMetAsnSerLeuArgAlaGluAspThrAlaValTyrTyrCysAlaArgHisProGly | |
| | CACCATCTCCAGAGACAATTCCAAGAACACGCTGTATCTGCAAATGAACAGCCTGAGAGCCGAGGACACGGCCGTATATTACTGTGCCAGACATCCGGGT | |
| 201 | ---------!---------!---------!---------!---------!---------!---------!---------!---------!---------! | 300 |
| 1: | PheAlaGlyTyrGlyTyrGlyHisTyrTyrAlaMetAspProTrpGlyGlnGlyThrLeuValThrValSerSerAlaSerThrLysGlyProSerValP | |
| | TTTGCTGGTTATGGTTATGGGCATTATTACGCCATGGACCCCTGGGGCCAAGGAACCCTGGTCACCGTCTCCTCAGCCTCCACCAAGGGCCCATCGGTCT | |
| 301 | ---------!---------!---------!---------!---------!---------!---------!---------!---------!---------! | 400 |
| 1: | heProLeuAlaProSerSerLysSerThrSerGlyGlyThrAlaAlaLeuGlyCysLeuValLysAspTyrPheProGluProValThrValSerTrpAs | |
| | TCCCCCTGGCACCCTCCTCCAAGAGCACCTCTGGGGGCACAGCGGCCCTGGGCTGCCTGGTCAAGGACTACTTCCCCGAACCGGTGACGGTGTCGTGGAA | |
| 401 | ---------!---------!---------!---------!---------!---------!---------!---------!---------!---------! | 500 |
| 1: | nSerGlyAlaLeuThrSerGlyValHisThrPheProAlaValLeuGlnSerSerGlyLeuTyrSerLeuSerSerValValThrValProSerSerSer | |
| | CTCAGGCGCCCTGACCAGCGGCGTGCACACCTTCCCGGCTGTCCTACAGTCCTCAGGACTCTACTCCCTCAGCAGCGTGGTGACCGTGCCCTCCAGCAGC | |
| 501 | ---------!---------!---------!---------!---------!---------!---------!---------!---------!---------! | 600 |
| 1: | LeuGlyThrGlnThrTyrIleCysAsnValAsnHisLysProSerAsnThrLysValAspLysLysValGluProLysSerCysAspLysThrHisThrC | |
| | TTGGGCACCCAGACCTACATCTGCAACGTGAATCACAAGCCCAGCAACACCAAGGTGGACAAGAAAGTTGAGCCCAAATCTTGTGACAAAACTCACACAT | |
| 601 | ---------!---------!---------!---------!---------!---------!---------!---------!---------!---------! | 700 |
| 1: | ysProProCysProAlaProGluLeuLeuGlyGlyProSerValPheLeuPheProProLysProLysAspThrLeuMetIleSerArgThrProGluVa | |
| | GCCCACCGTGCCCAGCACCTGAACTCCTGGGGGGACCGTCAGTCTTCCTCTTCCCCCCAAAACCCAAGGACACCCTCATGATCTCCCGGACCCCTGAGGT | |
| 701 | ---------!---------!---------!---------!---------!---------!---------!---------!---------!---------! | 800 |
| 1: | lThrCysValValValAspValSerHisGluAspProGluValLysPheAsnTrpTyrValAspGlyValGluValHisAsnAlaLysThrLysProArg | |
| | CACATGCGTGGTGGTGGACGTGAGCCACGAAGACCCTGAGGTCAAGTTCAACTGGTACGTGGACGGCGTGGAGGTGCATAATGCCAAGACAAAGCCGCGG | |
| 801 | ---------!---------!---------!---------!---------!---------!---------!---------!---------!---------! | 900 |
| 1: | GluGluGlnTyrAsnSerThrTyrArgValValSerValLeuThrValLeuHisGlnAspTrpLeuAsnGlyLysGluTyrLysCysLysValSerAsnL | |
| | GAGGAGCAGTACAACAGCACGTACCGTGTGGTCAGCGTCCTCACCGTCCTGCACCAGGACTGGCTGAATGGCAAGGAGTACAAGTGCAAGGTCTCCAACA | |
| 901 | ---------!---------!---------!---------!---------!---------!---------!---------!---------!---------! | 1000 |
| 1: | ysAlaLeuProAlaProIleGluLysThrIleSerLysAlaLysGlyGlnProArgGluProGlnValTyrThrLeuProProSerArgAspGluLeuTh | |
| | AAGCCCTCCCAGCCCCCATCGAGAAAACCATCTCCAAAGCCAAAGGGCAGCCCCGAGAACCACAGGTGTACACCCTGCCCCCATCCCGGGATGAGCTGAC | |
| 1001 | ---------!---------!---------!---------!---------!---------!---------!---------!---------!---------! | 1100 |
| 1: | rLysAsnGlnValSerLeuThrCysLeuValLysGlyPheTyrProSerAspIleAlaValGluTrpGluSerAsnGlyGlnProGluAsnAsnTyrLys | |
| | CAAGAACCAGGTCAGCCTGACCTGCCTGGTCAAAGGCTTCTATCCCAGCGACATCGCCGTGGAGTGGGAGAGCAATGGGCAGCCGGAGAACAACTACAAG | |
| 1101 | ---------!---------!---------!---------!---------!---------!---------!---------!---------!---------! | 1200 |
| 1: | ThrThrProProValLeuAspSerAspGlySerPhePheLeuTyrSerLysLeuThrValAspLysSerArgTrpGlnGlnGlyAsnValPheSerCysS | |
| | ACCACGCCTCCCGTGCTGGACTCCGACGGCTCCTTCTTCCTCTACAGCAAGCTCACCGTGGACAAGAGCAGGTGGCAGCAGGGGAACGTCTTCTCATGCT | |
| 1201 | ---------!---------!---------!---------!---------!---------!---------!---------!---------!---------! | 1300 |
| 1: | erValMetHisGluAlaLeuHisAsnHisTyrThrGlnLysSerLeuSerLeuSerProGlyLys | |
| | CCGTGATGCATGAGGCTCTGCACAACCACTACACGCAGAAGAGCCTCTCCCTGTCTCCGGGTAAA | |
| 1301 | ---------!---------!---------!---------!---------!---------!----- | 1365 |

5R4H1 SEQ ID No. 42

| | | |
|---|---|---|
| 1: | GluValGlnLeuValGluSerGlyGlyGlyLeuValGlnProGlyGlySerLeuArgLeuSerCysAlaAlaSerGlyTyrSerValThrHisGlnAlaV | |
| | GAGGTGCAGCTGGTGGAGTCTGGGGGAGGCTTGGTACAGCCTGGGGGGTCCCTGAGACTCTCCTGTGCGGCTTCTGGATATAGTGTTACGCATCAGGCTG | |
| 1 | ---------!---------!---------!---------!---------!---------!---------!---------!---------!---------! | 100 |
| 1: | alThrTrpValArgGlnAlaProGlyLysGlyLeuGluTrpIleGlyValIleAlaSerAsnAlaAspThrTyrTyrAlaSerTrpAlaLysGlyArgPh | |
| | TGACTTGGGTCCGCCAGGCTCCAGGGAAGGGGCTGGAGTGGATCGGAGTCATTGCTAGTAATGCTGACACATACTACGCGAGCTGGGCGAAAGGCCGGTT | |
| 101 | ---------!---------!---------!---------!---------!---------!---------!---------!---------!---------! | 200 |
| 1: | eThrIleSerArgAspAsnSerLysAsnThrLeuTyrLeuGlnMetAsnSerLeuArgAlaGluAspThrAlaValTyrTyrCysAlaArgHisProGly | |
| | CACCATCTCCAGAGACAATTCCAAGAACACGCTGTATCTGCAAATGAACAGCCTGAGAGCCGAGGACACGGCCGTATATTACTGTGCCAGACATCCTGGT | |
| 201 | ---------!---------!---------!---------!---------!---------!---------!---------!---------!---------! | 300 |
| 1: | SerAlaGlyTyrGlyTyrGlnTyrTyrTyrSerMetAspProTrpGlyGlnGlyThrLeuValThrValSerSerAlaSerThrLysGlyProSerValP | |
| | AGTGCTGGTTATGGTTATCAGTATTATTATAGTATGGACCCCTGGGGCCAAGGAACCCTGGTCACCGTCTCCTCAGCCTCCACCAAGGGCCCATCGGTCT | |
| 301 | ---------!---------!---------!---------!---------!---------!---------!---------!---------!---------! | 400 |
| 1: | heProLeuAlaProSerSerLysSerThrSerGlyGlyThrAlaAlaLeuGlyCysLeuValLysAspTyrPheProGluProValThrValSerTrpAs | |
| | TCCCCCTGGCACCCTCCTCCAAGAGCACCTCTGGGGGCACAGCGGCCCTGGGCTGCCTGGTCAAGGACTACTTCCCCGAACCGGTGACGGTGTCGTGGAA | |
| 401 | ---------!---------!---------!---------!---------!---------!---------!---------!---------!---------! | 500 |
| 1: | nSerGlyAlaLeuThrSerGlyValHisThrPheProAlaValLeuGlnSerSerGlyLeuTyrSerLeuSerSerValValThrValProSerSerSer | |
| | CTCAGGCGCCCTGACCAGCGGCGTGCACACCTTCCCGGCTGTCCTACAGTCCTCAGGACTCTACTCCCTCAGCAGCGTGGTGACCGTGCCCTCCAGCAGC | |
| 501 | ---------!---------!---------!---------!---------!---------!---------!---------!---------!---------! | 600 |
| 1: | LeuGlyThrGlnThrTyrIleCysAsnValAsnHisLysProSerAsnThrLysValAspLysLysValGluProLysSerCysAspLysThrHisThrC | |
| | TTGGGCACCCAGACCTACATCTGCAACGTGAATCACAAGCCCAGCAACACCAAGGTGGACAAGAAAGTTGAGCCCAAATCTTGTGACAAAACTCACACAT | |
| 601 | ---------!---------!---------!---------!---------!---------!---------!---------!---------!---------! | 700 |
| 1: | ysProProCysProAlaProGluLeuLeuGlyGlyProSerValPheLeuPheProProLysProLysAspThrLeuMetIleSerArgThrProGluVa | |
| | GCCCACCGTGCCCAGCACCTGAACTCCTGGGGGGACCGTCAGTCTTCCTCTTCCCCCCAAAACCCAAGGACACCCTCATGATCTCCCGGACCCCTGAGGT | |
| 701 | ---------!---------!---------!---------!---------!---------!---------!---------!---------!---------! | 800 |
| 1: | lThrCysValValValAspValSerHisGluAspProGluValLysPheAsnTrpTyrValAspGlyValGluValHisAsnAlaLysThrLysProArg | |
| | CACATGCGTGGTGGTGGACGTGAGCCACGAAGACCCTGAGGTCAAGTTCAACTGGTACGTGGACGGCGTGGAGGTGCATAATGCCAAGACAAAGCCGCGG | |
| 801 | ---------!---------!---------!---------!---------!---------!---------!---------!---------!---------! | 900 |
| 1: | GluGluGlnTyrAsnSerThrTyrArgValValSerValLeuThrValLeuHisGlnAspTrpLeuAsnGlyLysGluTyrLysCysLysValSerAsnL | |
| | GAGGAGCAGTACAACAGCACGTACCGTGTGGTCAGCGTCCTCACCGTCCTGCACCAGGACTGGCTGAATGGCAAGGAGTACAAGTGCAAGGTCTCCAACA | |
| 901 | ---------!---------!---------!---------!---------!---------!---------!---------!---------!---------! | 1000 |
| 1: | ysAlaLeuProAlaProIleGluLysThrIleSerLysAlaLysGlyGlnProArgGluProGlnValTyrThrLeuProProSerArgAspGluLeuTh | |
| | AAGCCCTCCCAGCCCCCATCGAGAAAACCATCTCCAAAGCCAAAGGGCAGCCCCGAGAACCACAGGTGTACACCCTGCCCCCATCCCGGGATGAGCTGAC | |
| 1001 | ---------!---------!---------!---------!---------!---------!---------!---------!---------!---------! | 1100 |
| 1: | rLysAsnGlnValSerLeuThrCysLeuValLysGlyPheTyrProSerAspIleAlaValGluTrpGluSerAsnGlyGlnProGluAsnAsnTyrLys | |
| | CAAGAACCAGGTCAGCCTGACCTGCCTGGTCAAAGGCTTCTATCCCAGCGACATCGCCGTGGAGTGGGAGAGCAATGGGCAGCCGGAGAACAACTACAAG | |
| 1101 | ---------!---------!---------!---------!---------!---------!---------!---------!---------!---------! | 1200 |
| 1: | ThrThrProProValLeuAspSerAspGlySerPhePheLeuTyrSerLysLeuThrValAspLysSerArgTrpGlnGlnGlyAsnValPheSerCysS | |
| | ACCACGCCTCCCGTGCTGGACTCCGACGGCTCCTTCTTCCTCTACAGCAAGCTCACCGTGGACAAGAGCAGGTGGCAGCAGGGGAACGTCTTCTCATGCT | |
| 1201 | | 1300 |
| 1: | erValMetHisGluAlaLeuHisAsnHisTyrThrGlnLysSerLeuSerLeuSerProGlyLys | |
| | CCGTGATGCATGAGGCTCTGCACAACCACTACACGCAGAAGAGCCTCTCCCTGTCTCCGGGTAAA | |
| 1301 | ---------!---------!---------!---------!---------!---------!----- | 1365 |

5R4F3 SEQ ID No. 43

| | | |
|---|---|---|
| 1: | GluValGlnLeuValGluSerGlyGlyGlyLeuValGlnProGlyGlySerLeuArgLeuSerCysLysAlaSerGlyPheThrPheProLysGlnAlaM | |
| | GAGGTGCAGCTGGTGGAGTCTGGGGGAGGCTTGGTACAGCCTGGGGGGTCCCTGAGACTCTCCTGTAAGGCTTCTGGATTTACTTTTCCTAAGCAGGCTA | |
| 1 | ---------!---------!---------!---------!---------!---------!---------!---------!---------!---------! | 100 |
| 1: | etThrTrpValArgGlnAlaProGlyLysGlyLeuGluTrpIleGlyValIleAlaSerAsnAlaAspThrTyrTyrAlaSerTrpAlaLysGlyArgPh | |
| | TGACTTGGGTCCGCCAGGCTCCAGGGAAGGGGCTGGAGTGGATCGGAGTCATTGCTAGTAATGCTGACACATACTACGCGAGCTGGGCGAAAGGCCGGTT | |
| 101 | ---------!---------!---------!---------!---------!---------!---------!---------!---------!---------! | 200 |
| 1: | eThrIleSerArgAspAsnSerLysAsnThrLeuTyrLeuGlnMetAsnSerLeuArgAlaGluAspThrAlaValTyrTyrCysAlaArgHisProGly | |
| | CACCATCTCCAGAGACAATTCCAAGAACACGCTGTATCTGCAAATGAACAGCCTGAGAGCCGAGGACACGGCCGTATATTACTGTGCCAGACATCCGGGT | |
| 201 | ---------!---------!---------!---------!---------!---------!---------!---------!---------!---------! | 300 |
| 1: | PheAlaGlyTyrGlyTyrGlyHisTyrTyrAlaMetAspProTrpGlyGlnGlyThrLeuValThrValSerSerAlaSerThrLysGlyProSerValP | |
| | TTTGCTGGTTATGGTTATGGGCATTATTACGCCATGGACCCCTGGGGCCAAGGAACCCTGGTCACCGTCTCCTCAGCCTCCACCAAGGGCCCATCGGTCT | |
| 301 | ---------!---------!---------!---------!---------!---------!---------!---------!---------!---------! | 400 |
| 1: | heProLeuAlaProSerSerLysSerThrSerGlyGlyThrAlaAlaLeuGlyCysLeuValLysAspTyrPheProGluProValThrValSerTrpAs | |
| | TCCCCCTGGCACCCTCCTCCAAGAGCACCTCTGGGGGCACAGCGGCCCTGGGCTGCCTGGTCAAGGACTACTTCCCCGAACCGGTGACGGTGTCGTGGAA | |
| 401 | ---------!---------!---------!---------!---------!---------!---------!---------!---------!---------! | 500 |
| 1: | nSerGlyAlaLeuThrSerGlyValHisThrPheProAlaValLeuGlnSerSerGlyLeuTyrSerLeuSerSerValValThrValProSerSerSer | |
| | CTCAGGCGCCCTGACCAGCGGCGTGCACACCTTCCCGGCTGTCCTACAGTCCTCAGGACTCTACTCCCTCAGCAGCGTGGTGACCGTGCCCTCCAGCAGC | |
| 501 | ---------!---------!---------!---------!---------!---------!---------!---------!---------!---------! | 600 |
| 1: | LeuGlyThrGlnThrTyrIleCysAsnValAsnHisLysProSerAsnThrLysValAspLysLysValGluProLysSerCysAspLysThrHisThrC | |
| | TTGGGCACCCAGACCTACATCTGCAACGTGAATCACAAGCCCAGCAACACCAAGGTGGACAAGAAAGTTGAGCCCAAATCTTGTGACAAAACTCACACAT | |
| 601 | ---------!---------!---------!---------!---------!---------!---------!---------!---------!---------! | 700 |
| 1: | ysProProCysProAlaProGluLeuLeuGlyGlyProSerValPheLeuPheProProLysProLysAspThrLeuMetIleSerArgThrProGluVa | |
| | GCCCACCGTGCCCAGCACCTGAACTCCTGGGGGGACCGTCAGTCTTCCTCTTCCCCCCAAAACCCAAGGACACCCTCATGATCTCCCGGACCCCTGAGGT | |
| 701 | ---------!---------!---------!---------!---------!---------!---------!---------!---------!---------! | 800 |
| 1: | lThrCysValValValAspValSerHisGluAspProGluValLysPheAsnTrpTyrValAspGlyValGluValHisAsnAlaLysThrLysProArg | |
| | CACATGCGTGGTGGTGGACGTGAGCCACGAAGACCCTGAGGTCAAGTTCAACTGGTACGTGGACGGCGTGGAGGTGCATAATGCCAAGACAAAGCCGCGG | |
| 801 | ---------!---------!---------!---------!---------!---------!---------!---------!---------!---------! | 900 |
| 1: | GluGluGlnTyrAsnSerThrTyrArgValValSerValLeuThrValLeuHisGlnAspTrpLeuAsnGlyLysGluTyrLysCysLysValSerAsnL | |
| | GAGGAGCAGTACAACAGCACGTACCGTGTGGTCAGCGTCCTCACCGTCCTGCACCAGGACTGGCTGAATGGCAAGGAGTACAAGTGCAAGGTCTCCAACA | |
| 901 | ---------!---------!---------!---------!---------!---------!---------!---------!---------!---------! | 1000 |
| 1: | ysAlaLeuProAlaProIleGluLysThrIleSerLysAlaLysGlyGlnProArgGluProGlnValTyrThrLeuProProSerArgAspGluLeuTh | |
| | AAGCCCTCCCAGCCCCCATCGAGAAAACCATCTCCAAAGCCAAAGGGCAGCCCCGAGAACCACAGGTGTACACCCTGCCCCCATCCCGGGATGAGCTGAC | |
| 1001 | ---------!---------!---------!---------!---------!---------!---------!---------!---------!---------! | 1100 |
| 1: | rLysAsnGlnValSerLeuThrCysLeuValLysGlyPheTyrProSerAspIleAlaValGluTrpGluSerAsnGlyGlnProGluAsnAsnTyrLys | |
| | CAAGAACCAGGTCAGCCTGACCTGCCTGGTCAAAGGCTTCTATCCCAGCGACATCGCCGTGGAGTGGGAGAGCAATGGGCAGCCGGAGAACAACTACAAG | |
| 1101 | ---------!---------!---------!---------!---------!---------!---------!---------!---------!---------! | 1200 |
| 1: | ThrThrProProValLeuAspSerAspGlySerPhePheLeuTyrSerLysLeuThrValAspLysSerArgTrpGlnGlnGlyAsnValPheSerCysS | |
| | ACCACGCCTCCCGTGCTGGACTCCGACGGCTCCTTCTTCCTCTACAGCAAGCTCACCGTGGACAAGAGCAGGTGGCAGCAGGGGAACGTCTTCTCATGCT | |
| 1201 | ---------!---------!---------!---------!---------!---------!---------!---------!---------!---------! | 1300 |
| 1: | erValMetHisGluAlaLeuHisAsnHisTyrThrGlnLysSerLeuSerLeuSerProGlyLys | |
| | CCGTGATGCATGAGGCTCTGCACAACCACTACACGCAGAAGAGCCTCTCCCTGTCTCCGGGTAAA | |
| 1301 | ---------!---------!---------!---------!---------!---------!----- | 1365 |

5R4E11 SEQ ID No. 44

| | | |
|---|---|---|
| 1: | GluValGlnLeuValGluSerGlyGlyGlyLeuValGlnProGlyGlySerLeuArgLeuSerCysLysAlaSerGlyPheThrPheLysProGlnAlaM | |
| | GAGGTGCAGCTGGTGGAGTCTGGGGGAGGCTTGGTACAGCCTGGGGGGTCCCTGAGACTCTCCTGTAAGGCTTCTGGATTTACTTTTAAGCCGCAGGCTA | |
| 1 | ---------!---------!---------!---------!---------!---------!---------!---------!---------!---------! | 100 |
| 1: | etThrTrpValArgGlnAlaProGlyLysGlyLeuGluTrpIleGlyValIleAlaSerAsnAlaAspThrTyrTyrAlaSerTrpAlaLysGlyArgPh | |
| | TGACATGGGTCCGCCAGGCTCCAGGGAAGGGGCTGGAGTGGATCGGAGTCATTGCTAGTAATGCTGACACATACTACGCGAGCTGGGCGAAAGGCCGATT | |
| 101 | ---------!---------!---------!---------!---------!---------!---------!---------!---------!---------! | 200 |
| 1: | eThrIleSerArgAspAsnSerLysAsnThrLeuTyrLeuGlnMetAsnSerLeuArgAlaGluAspThrAlaValTyrTyrCysAlaArgHisProGly | |
| | CACCATCTCCAGAGACAATTCCAAGAACACGCTGTATCTGCAAATGAACAGCCTGAGAGCCGAGGACACGGCCGTATATTACTGTGCCAGACATCCGGGT | |
| 201 | ---------!---------!---------!---------!---------!---------!---------!---------!---------!---------! | 300 |
| 1: | PheAlaGlyTyrGlyTyrGlyHisTyrTyrAlaMetAspProTrpGlyGlnGlyThrLeuValThrValSerSerAlaSerThrLysGlyProSerValP | |
| | TTTGCTGGTTATGGTTATGGGCATTATTACGCCATGGACCCCTGGGGCCAAGGAACCCTGGTCACCGTCTCCTCAGCCTCCACCAAGGGCCCATCGGTCT | |
| 301 | ---------!---------!---------!---------!---------!---------!---------!---------!---------!---------! | 400 |
| 1: | heProLeuAlaProSerSerLysSerThrSerGlyGlyThrAlaAlaLeuGlyCysLeuValLysAspTyrPheProGluProValThrValSerTrpAs | |
| | TCCCCCTGGCACCCTCCTCCAAGAGCACCTCTGGGGGCACAGCGGCCCTGGGCTGCCTGGTCAAGGACTACTTCCCCGAACCGGTGACGGTGTCGTGGAA | |
| 401 | ---------!---------!---------!---------!---------!---------!---------!---------!---------!---------! | 500 |
| 1: | nSerGlyAlaLeuThrSerGlyValHisThrPheProAlaValLeuGlnSerSerGlyLeuTyrSerLeuSerSerValValThrValProSerSerSer | |
| | CTCAGGCGCCCTGACCAGCGGCGTGCACACCTTCCCGGCTGTCCTACAGTCCTCAGGACTCTACTCCCTCAGCAGCGTGGTGACCGTGCCCTCCAGCAGC | |
| 501 | ---------!---------!---------!---------!---------!---------!---------!---------!---------!---------! | 600 |
| 1: | LeuGlyThrGlnThrTyrIleCysAsnValAsnHisLysProSerAsnThrLysValAspLysLysValGluProLysSerCysAspLysThrHisThrC | |
| | TTGGGCACCCAGACCTACATCTGCAACGTGAATCACAAGCCCAGCAACACCAAGGTGGACAAGAAAGTTGAGCCCAAATCTTGTGACAAAACTCACACAT | |
| 601 | ---------!---------!---------!---------!---------!---------!---------!---------!---------!---------! | 700 |
| 1: | ysProProCysProAlaProGluLeuLeuGlyGlyProSerValPheLeuPheProProLysProLysAspThrLeuMetIleSerArgThrProGluVa | |
| | GCCCACCGTGCCCAGCACCTGAACTCCTGGGGGGACCGTCAGTCTTCCTCTTCCCCCCAAAACCCAAGGACACCCTCATGATCTCCCGGACCCCTGAGGT | |
| 701 | ---------!---------!---------!---------!---------!---------!---------!---------!---------!---------! | 800 |
| 1: | lThrCysValValValAspValSerHisGluAspProGluValLysPheAsnTrpTyrValAspGlyValGluValHisAsnAlaLysThrLysProArg | |
| | CACATGCGTGGTGGTGGACGTGAGCCACGAAGACCCTGAGGTCAAGTTCAACTGGTACGTGGACGGCGTGGAGGTGCATAATGCCAAGACAAAGCCGCGG | |
| 801 | ---------!---------!---------!---------!---------!---------!---------!---------!---------!---------! | 900 |
| 1: | GluGluGlnTyrAsnSerThrTyrArgValValSerValLeuThrValLeuHisGlnAspTrpLeuAsnGlyLysGluTyrLysCysLysValSerAsnL | |
| | GAGGAGCAGTACAACAGCACGTACCGTGTGGTCAGCGTCCTCACCGTCCTGCACCAGGACTGGCTGAATGGCAAGGAGTACAAGTGCAAGGTCTCCAACA | |
| 901 | ---------!---------!---------!---------!---------!---------!---------!---------!---------!---------! | 1000 |
| 1: | ysAlaLeuProAlaProIleGluLysThrIleSerLysAlaLysGlyGlnProArgGluProGlnValTyrThrLeuProProSerArgAspGluLeuTh | |
| | AAGCCCTCCCAGCCCCCATCGAGAAAACCATCTCCAAAGCCAAAGGGCAGCCCCGAGAACCACAGGTGTACACCCTGCCCCCATCCCGGGATGAGCTGAC | |
| 1001 | ---------!---------!---------!---------!---------!---------!---------!---------!---------!---------! | 1100 |
| 1: | rLysAsnGlnValSerLeuThrCysLeuValLysGlyPheTyrProSerAspIleAlaValGluTrpGluSerAsnGlyGlnProGluAsnAsnTyrLys | |
| | CAAGAACCAGGTCAGCCTGACCTGCCTGGTCAAAGGCTTCTATCCCAGCGACATCGCCGTGGAGTGGGAGAGCAATGGGCAGCCGGAGAACAACTACAAG | |
| 1101 | ---------!---------!---------!---------!---------!---------!---------!---------!---------!---------! | 1200 |
| 1: | ThrThrProProValLeuAspSerAspGlySerPhePheLeuTyrSerLysLeuThrValAspLysSerArgTrpGlnGlnGlyAsnValPheSerCysS | |
| | ACCACGCCTCCCGTGCTGGACTCCGACGGCTCCTTCTTCCTCTACAGCAAGCTCACCGTGGACAAGAGCAGGTGGCAGCAGGGGAACGTCTTCTCATGCT | |
| 1201 | ---------!---------!---------!---------!---------!---------!---------!---------!---------!---------! | 1300 |
| 1: | erValMetHisGluAlaLeuHisAsnHisTyrThrGlnLysSerLeuSerLeuSerProGlyLys | |
| | CCGTGATGCATGAGGCTCTGCACAACCACTACACGCAGAAGAGCCTCTCCCTGTCTCCGGGTAAA | |
| 1301 | ---------!---------!---------!---------!---------!---------!----- | 1365 |

6R4H1 SEQ ID No. 45

| | | |
|---|---|---|
| 1: | GluValGlnLeuValGluSerGlyGlyGlyLeuValGlnProGlyGlySerLeuArgLeuSerCysGluAlaSerGlyTyrThrPheTrpSerGlnAlaM | |
| | GAGGTGCAGCTGGTGGAGTCTGGGGGAGGCTTGGTACAGCCTGGGGGGTCCCTGAGACTCTCCTGTGAGGCTTCTGGATATACTTTTTGGTCGCAGGCTA | |
| 1 | ---------!---------!---------!---------!---------!---------!---------!---------!---------!---------! | 100 |
| 1: | etThrTrpValArgGlnAlaProGlyLysGlyLeuGluTrpIleGlyValIleAlaSerAsnAlaAspThrTyrTyrAlaSerTrpValLysGlyArgPh | |
| | TGACTTGGGTCCGCCAGGCTCCAGGGAAGGGGCTGGAGTGGATCGGAGTCATTGCTAGTAATGCTGACACATACTACGCGAGCTGGGTGAAAGGCCGGTT | |
| 101 | ---------!---------!---------!---------!---------!---------!---------!---------!---------!---------! | 200 |
| 1: | eThrIleSerArgAspAsnSerLysAsnThrLeuTyrLeuGlnMetAsnSerLeuArgAlaGluAspThrAlaValTyrTyrCysAlaArgHisProGly | |
| | CACCATCTCCAGAGACAATTCCAAGAACACGCTGTATCTGCAAATGAACAGCCTGAGAGCCGAGGACACGGCCGTATATTACTGTGCCAGACATCCGGGT | |
| 201 | ---------!---------!---------!---------!---------!---------!---------!---------!---------!---------! | 300 |
| 1: | PheAlaGlyTyrGlyTyrGlnTyrTyrTyrSerMetAspProTrpGlyGlnGlyThrLeuValThrValSerSerAlaSerThrLysGlyProSerValP | |
| | TTTGCTGGTTATGGTTATCAGTATTATTATAGTATGGACCCCTGGGGCCAAGGAACCCTGGTCACCGTCTCCTCAGCCTCCACCAAGGGCCCATCGGTCT | |
| 301 | ---------!---------!---------!---------!---------!---------!---------!---------!---------!---------! | 400 |
| 1: | heProLeuAlaProSerSerLysSerThrSerGlyGlyThrAlaAlaLeuGlyCysLeuValLysAspTyrPheProGluProValThrValSerTrpAs | |
| | TCCCCCTGGCACCCTCCTCCAAGAGCACCTCTGGGGGCACAGCGGCCCTGGGCTGCCTGGTCAAGGACTACTTCCCCGAACCGGTGACGGTGTCGTGGAA | |
| 401 | ---------!---------!---------!---------!---------!---------!---------!---------!---------!---------! | 500 |
| 1: | nSerGlyAlaLeuThrSerGlyValHisThrPheProAlaValLeuGlnSerSerGlyLeuTyrSerLeuSerSerValValThrValProSerSerSer | |
| | CTCAGGCGCCCTGACCAGCGGCGTGCACACCTTCCCGGCTGTCCTACAGTCCTCAGGACTCTACTCCCTCAGCAGCGTGGTGACCGTGCCCTCCAGCAGC | |
| 501 | ---------!---------!---------!---------!---------!---------!---------!---------!---------!---------! | 600 |
| 1: | LeuGlyThrGlnThrTyrIleCysAsnValAsnHisLysProSerAsnThrLysValAspLysLysValGluProLysSerCysAspLysThrHisThrC | |
| | TTGGGCACCCAGACCTACATCTGCAACGTGAATCACAAGCCCAGCAACACCAAGGTGGACAAGAAAGTTGAGCCCAAATCTTGTGACAAAACTCACACAT | |
| 601 | ---------!---------!---------!---------!---------!---------!---------!---------!---------!---------! | 700 |
| 1: | ysProProCysProAlaProGluLeuLeuGlyGlyProSerValPheLeuPheProProLysProLysAspThrLeuMetIleSerArgThrProGluVa | |
| | GCCCACCGTGCCCAGCACCTGAACTCCTGGGGGGACCGTCAGTCTTCCTCTTCCCCCCAAAACCCAAGGACACCCTCATGATCTCCCGGACCCCTGAGGT | |
| 701 | ---------!---------!---------!---------!---------!---------!---------!---------!---------!---------! | 800 |
| 1: | lThrCysValValValAspValSerHisGluAspProGluValLysPheAsnTrpTyrValAspGlyValGluValHisAsnAlaLysThrLysProArg | |
| | CACATGCGTGGTGGTGGACGTGAGCCACGAAGACCCTGAGGTCAAGTTCAACTGGTACGTGGACGGCGTGGAGGTGCATAATGCCAAGACAAAGCCGCGG | |
| 801 | ---------!---------!---------!---------!---------!---------!---------!---------!---------!---------! | 900 |
| 1: | GluGluGlnTyrAsnSerThrTyrArgValValSerValLeuThrValLeuHisGlnAspTrpLeuAsnGlyLysGluTyrLysCysLysValSerAsnL | |
| | GAGGAGCAGTACAACAGCACGTACCGTGTGGTCAGCGTCCTCACCGTCCTGCACCAGGACTGGCTGAATGGCAAGGAGTACAAGTGCAAGGTCTCCAACA | |
| 901 | ---------!---------!---------!---------!---------!---------!---------!---------!---------!---------! | 1000 |
| 1: | ysAlaLeuProAlaProIleGluLysThrIleSerLysAlaLysGlyGlnProArgGluProGlnValTyrThrLeuProProSerArgAspGluLeuTh | |
| | AAGCCCTCCCAGCCCCCATCGAGAAAACCATCTCCAAAGCCAAAGGGCAGCCCCGAGAACCACAGGTGTACACCCTGCCCCCATCCCGGGATGAGCTGAC | |
| 1001 | ---------!---------!---------!---------!---------!---------!---------!---------!---------!---------! | 1100 |
| 1: | rLysAsnGlnValSerLeuThrCysLeuValLysGlyPheTyrProSerAspIleAlaValGluTrpGluSerAsnGlyGlnProGluAsnAsnTyrLys | |
| | CAAGAACCAGGTCAGCCTGACCTGCCTGGTCAAAGGCTTCTATCCCAGCGACATCGCCGTGGAGTGGGAGAGCAATGGGCAGCCGGAGAACAACTACAAG | |
| 1101 | ---------!---------!---------!---------!---------!---------!---------!---------!---------!---------! | 1200 |
| 1: | ThrThrProProValLeuAspSerAspGlySerPhePheLeuTyrSerLysLeuThrValAspLysSerArgTrpGlnGlnGlyAsnValPheSerCysS | |
| | ACCACGCCTCCCGTGCTGGACTCCGACGGCTCCTTCTTCCTCTACAGCAAGCTCACCGTGGACAAGAGCAGGTGGCAGCAGGGGAACGTCTTCTCATGCT | |
| 1201 | ---------!---------!---------!---------!---------!---------!---------!---------!---------!---------! | 1300 |
| 1: | erValMetHisGluAlaLeuHisAsnHisTyrThrGlnLysSerLeuSerLeuSerProGlyLys | |
| | CCGTGATGCATGAGGCTCTGCACAACCACTACACGCAGAAGAGCCTCTCCCTGTCTCCGGGTAAA | |
| 1301 | ---------!---------!---------!---------!---------!---------!----- | 1365 |

6R4A7 SEQ ID No. 46

| | | |
|---|---|---|
| 1: | GluValGlnLeuValGluSerGlyGlyGlyLeuValGlnProGlyGlySerLeuArgLeuSerCysLysAlaSerGlyIleSerIleThrGluLysAlaV | |
| | GAGGTGCAGCTGGTGGAGTCTGGGGGAGGCTTGGTACAGCCTGGGGGGTCCCTGAGACTCTCCTGTAAGGCTTCTGGAATTAGTATTACGGAGAAGGCTG | |
| 1 | ---------!---------!---------!---------!---------!---------!---------!---------!---------!---------! | 100 |
| 1: | alThrTrpValArgGlnAlaProGlyLysGlyLeuGluTrpIleGlyValIleAlaSerAsnAlaAspThrTyrTyrAlaSerTrpAlaLysGlyArgPh | |
| | TGACTTGGGTCCGCCAGGCTCCAGGGAAGGGGCTGGAGTGGATCGGAGTCATTGCTAGTAATGCTGACACATACTACGCGAGCTGGGCGAAAGGCCGGTT | |
| 101 | ---------!---------!---------!---------!---------!---------!---------!---------!---------!---------! | 200 |
| 1: | eThrIleSerArgAspAsnSerLysAsnThrLeuTyrLeuGlnMetAsnSerLeuArgAlaGluAspThrAlaValTyrTyrCysAlaArgHisProGly | |
| | CACCATCTCCAGAGACAATTCCAAGAACACGCTGTATCTGCAAATGAACAGCCTGAGAGCCGAGGACACGGCCGTATATTACTGTGCCAGACATCCGGGT | |
| 201 | ---------!---------!---------!---------!---------!---------!---------!---------!---------!---------! | 300 |
| 1: | PheAlaGlyTyrGlyTyrGlyHisTyrTyrAlaMetAspProTrpGlyGlnGlyThrLeuValThrValSerSerAlaSerThrLysGlyProSerValP | |
| | TTTGCTGGTTATGGTTATGGGCATTATTACGCCATGGACCCCTGGGGCCAAGGAACCCTGGTCACCGTCTCCTCAGCCTCCACCAAGGGCCCATCGGTCT | |
| 301 | ---------!---------!---------!---------!---------!---------!---------!---------!---------!---------! | 400 |
| 1: | heProLeuAlaProSerSerLysSerThrSerGlyGlyThrAlaAlaLeuGlyCysLeuValLysAspTyrPheProGluProValThrValSerTrpAs | |
| | TCCCCCTGGCACCCTCCTCCAAGAGCACCTCTGGGGGCACAGCGGCCCTGGGCTGCCTGGTCAAGGACTACTTCCCCGAACCGGTGACGGTGTCGTGGAA | |
| 401 | ---------!---------!---------!---------!---------!---------!---------!---------!---------!---------! | 500 |
| 1: | nSerGlyAlaLeuThrSerGlyValHisThrPheProAlaValLeuGlnSerSerGlyLeuTyrSerLeuSerSerValValThrValProSerSerSer | |
| | CTCAGGCGCCCTGACCAGCGGCGTGCACACCTTCCCGGCTGTCCTACAGTCCTCAGGACTCTACTCCCTCAGCAGCGTGGTGACCGTGCCCTCCAGCAGC | |
| 501 | ---------!---------!---------!---------!---------!---------!---------!---------!---------!---------! | 600 |
| 1: | LeuGlyThrGlnThrTyrIleCysAsnValAsnHisLysProSerAsnThrLysValAspLysLysValGluProLysSerCysAspLysThrHisThrC | |
| | TTGGGCACCCAGACCTACATCTGCAACGTGAATCACAAGCCCAGCAACACCAAGGTGGACAAGAAAGTTGAGCCCAAATCTTGTGACAAAACTCACACAT | |
| 601 | ---------!---------!---------!---------!---------!---------!---------!---------!---------!---------! | 700 |
| 1: | ysProProCysProAlaProGluLeuLeuGlyGlyProSerValPheLeuPheProProLysProLysAspThrLeuMetIleSerArgThrProGluVa | |
| | GCCCACCGTGCCCAGCACCTGAACTCCTGGGGGGACCGTCAGTCTTCCTCTTCCCCCCAAAACCCAAGGACACCCTCATGATCTCCCGGACCCCTGAGGT | |
| 701 | ---------!---------!---------!---------!---------!---------!---------!---------!---------!---------! | 800 |
| 1: | lThrCysValValValAspValSerHisGluAspProGluValLysPheAsnTrpTyrValAspGlyValGluValHisAsnAlaLysThrLysProArg | |
| | CACATGCGTGGTGGTGGACGTGAGCCACGAAGACCCTGAGGTCAAGTTCAACTGGTACGTGGACGGCGTGGAGGTGCATAATGCCAAGACAAAGCCGCGG | |
| 801 | ---------!---------!---------!---------!---------!---------!---------!---------!---------!---------! | 900 |
| 1: | GluGluGlnTyrAsnSerThrTyrArgValValSerValLeuThrValLeuHisGlnAspTrpLeuAsnGlyLysGluTyrLysCysLysValSerAsnI | |
| | GAGGAGCAGTACAACAGCACGTACCGTGTGGTCAGCGTCCTCACCGTCCTGCACCAGGACTGGCTGAATGGCAAGGAGTACAAGTGCAAGGTCTCCAACA | |
| 901 | ---------!---------!---------!---------!---------!---------!---------!---------!---------!---------! | 1000 |
| 1: | ysAlaLeuProAlaProIleGluLysThrIleSerLysAlaLysGlyGlnProArgGluProGlnValTyrThrLeuProProSerArgAspGluLeuTh | |
| | AAGCCCTCCCAGCCCCCATCGAGAAAACCATCTCCAAAGCCAAAGGGCAGCCCCGAGAACCACAGGTGTACACCCTGCCCCCATCCCGGGATGAGCTGAC | |
| 1001 | ---------!---------!---------!---------!---------!---------!---------!---------!---------!---------! | 1100 |
| 1: | rLysAsnGlnValSerLeuThrCysLeuValLysGlyPheTyrProSerAspIleAlaValGluTrpGluSerAsnGlyGlnProGluAsnAsnTyrLys | |
| | CAAGAACCAGGTCAGCCTGACCTGCCTGGTCAAAGGCTTCTATCCCAGCGACATCGCCGTGGAGTGGGAGAGCAATGGGCAGCCGGAGAACAACTACAAG | |
| 1101 | ---------!---------!---------!---------!---------!---------!---------!---------!---------!---------! | 1200 |
| 1: | ThrThrProProValLeuAspSerAspGlySerPhePheLeuTyrSerLysLeuThrValAspLysSerArgTrpGlnGlnGlyAsnValPheSerCysS | |
| | ACCACGCCTCCCGTGCTGGACTCCGACGGCTCCTTCTTCCTCTACAGCAAGCTCACCGTGGACAAGAGCAGGTGGCAGCAGGGGAACGTCTTCTCATGCT | |
| 1201 | ---------!---------!---------!---------!---------!---------!---------!---------!---------!---------! | 1300 |
| 1: | erValMetHisGluAlaLeuHisAsnHisTyrThrGlnLysSerLeuSerLeuSerProGlyLys | |
| | CCGTGATGCATGAGGCTCTGCACAACCACTACACGCAGAAGAGCCTCTCCCTGTCTCCGGGTAAA | |
| 1301 | ---------!---------!---------!---------!---------!---------!----- | 1365 |

6R4G10 SEQ ID No. 47

| | | |
|---|---|---|
| 1: | GluValGlnLeuValGluSerGlyGlyGlyLeuValGlnProGlyArgSerLeuArgLeuSerCysAlaAlaSerGlyTyrThrIleGlyProGlnAlaM | |
| | GAGGTGCAGCTGGTGGAGTCTGGGGGAGGCTTGGTACAGCCTGGGCGGTCCCTGAGACTCTCCTGTGCGGCTTCTGGATATACTATTGGGCCTCAGGCGA | |
| 1 | ---------!---------!---------!---------!---------!---------!---------!---------!---------!---------! | 100 |
| 1: | etThrTrpValArgGlnAlaProGlyLysGlyLeuGluTrpIleGlyValIleAlaSerAsnAlaAspThrTyrTyrAlaSerTrpAlaLysGlyArgPh | |
| | TGACTTGGGTCCGCCAGGCTCCAGGGAAGGGGCTGGAGTGGATCGGAGTCATTGCTAGTAATGCTGACACATACTACGCGAGCTGGGCGAAAGGCCGATT | |
| 101 | ---------!---------!---------!---------!---------!---------!---------!---------!---------!---------! | 200 |
| 1: | eThrIleSerArgAspAsnSerLysAsnThrLeuTyrLeuGlnMetAsnSerLeuArgAlaGluAspThrAlaValTyrTyrCysAlaArgHisProGly | |
| | CACCATCTCCAGAGACAATTCCAAGAACACGCTGTATCTGCAAATGAACAGCCTGAGAGCCGAGGACACGGCCGTATATTACTGTGCCAGACATCCGGGT | |
| 201 | ---------!---------!---------!---------!---------!---------!---------!---------!---------!---------! | 300 |
| 1: | PheAlaGlyTyrGlyTyrGlyHisTyrTyrAlaMetAspProTrpGlyGlnGlyThrLeuValThrValSerSerAlaSerThrLysGlyProSerValP | |
| | TTTGCTGGTTATGGTTATGGGCATTATTACGCCATGGACCCCTGGGGCCAAGGAACCCTGGTCACCGTCTCCTCAGCCTCCACCAAGGGCCCATCGGTCT | |
| 301 | ---------!---------!---------!---------!---------!---------!---------!---------!---------!---------! | 400 |
| 1: | heProLeuAlaProSerSerLysSerThrSerGlyGlyThrAlaAlaLeuGlyCysLeuValLysAspTyrPheProGluProValThrValSerTrpAs | |
| | TCCCCCTGGCACCCTCCTCCAAGAGCACCTCTGGGGGCACAGCGGCCCTGGGCTGCCTGGTCAAGGACTACTTCCCCGAACCGGTGACGGTGTCGTGGAA | |
| 401 | ---------!---------!---------!---------!---------!---------!---------!---------!---------!---------! | 500 |
| 1: | nSerGlyAlaLeuThrSerGlyValHisThrPheProAlaValLeuGlnSerSerGlyLeuTyrSerLeuSerSerValValThrValProSerSerSer | |
| | CTCAGGCGCCCTGACCAGCGGCGTGCACACCTTCCCGGCTGTCCTACAGTCCTCAGGACTCTACTCCCTCAGCAGCGTGGTGACCGTGCCCTCCAGCAGC | |
| 501 | ---------!---------!---------!---------!---------!---------!---------!---------!---------!---------! | 600 |
| 1: | LeuGlyThrGlnThrTyrIleCysAsnValAsnHisLysProSerAsnThrLysValAspLysLysValGluProLysSerCysAspLysThrHisThrC | |
| | TTGGGCACCCAGACCTACATCTGCAACGTGAATCACAAGCCCAGCAACACCAAGGTGGACAAGAAAGTTGAGCCCAAATCTTGTGACAAAACTCACACAT | |
| 601 | ---------!---------!---------!---------!---------!---------!---------!---------!---------!---------! | 700 |
| 1: | ysProProCysProAlaProGluLeuLeuGlyGlyProSerValPheLeuPheProProLysProLysAspThrLeuMetIleSerArgThrProGluVa | |
| | GCCCACCGTGCCCAGCACCTGAACTCCTGGGGGGACCGTCAGTCTTCCTCTTCCCCCCAAAACCCAAGGACACCCTCATGATCTCCCGGACCCCTGAGGT | |
| 701 | ---------!---------!---------!---------!---------!---------!---------!---------!---------!---------! | 800 |
| 1: | lThrCysValValValAspValSerHisGluAspProGluValLysPheAsnTrpTyrValAspGlyValGluValHisAsnAlaLysThrLysProArg | |
| | CACATGCGTGGTGGTGGACGTGAGCCACGAAGACCCTGAGGTCAAGTTCAACTGGTACGTGGACGGCGTGGAGGTGCATAATGCCAAGACAAAGCCGCGG | |
| 801 | ---------!---------!---------!---------!---------!---------!---------!---------!---------!---------! | 900 |
| 1: | GluGluGlnTyrAsnSerThrTyrArgValValSerValLeuThrValLeuHisGlnAspTrpLeuAsnGlyLysGluTyrLysCysLysValSerAsnL | |
| | GAGGAGCAGTACAACAGCACGTACCGTGTGGTCAGCGTCCTCACCGTCCTGCACCAGGACTGGCTGAATGGCAAGGAGTACAAGTGCAAGGTCTCCAACA | |
| 901 | ---------!---------!---------!---------!---------!---------!---------!---------!---------!---------! | 1000 |
| 1: | ysAlaLeuProAlaProIleGluLysThrIleSerLysAlaLysGlyGlnProArgGluProGlnValTyrThrLeuProProSerArgAspGluLeuTh | |
| | AAGCCCTCCCAGCCCCCATCGAGAAAACCATCTCCAAAGCCAAAGGGCAGCCCCGAGAACCACAGGTGTACACCCTGCCCCCATCCCGGGATGAGCTGAC | |
| 1001 | ---------!---------!---------!---------!---------!---------!---------!---------!---------!---------! | 1100 |
| 1: | rLysAsnGlnValSerLeuThrCysLeuValLysGlyPheTyrProSerAspIleAlaValGluTrpGluSerAsnGlyGlnProGluAsnAsnTyrLys | |
| | CAAGAACCAGGTCAGCCTGACCTGCCTGGTCAAAGGCTTCTATCCCAGCGACATCGCCGTGGAGTGGGAGAGCAATGGGCAGCCGGAGAACAACTACAAG | |
| 1101 | ---------!---------!---------!---------!---------!---------!---------!---------!---------!---------! | 1200 |
| 1: | ThrThrProProValLeuAspSerAspGlySerPhePheLeuTyrSerLysLeuThrValAspLysSerArgTrpGlnGlnGlyAsnValPheSerCysS | |
| | ACCACGCCTCCCGTGCTGGACTCCGACGGCTCCTTCTTCCTCTACAGCAAGCTCACCGTGGACAAGAGCAGGTGGCAGCAGGGGAACGTCTTCTCATGCT | |
| 1201 | ---------!---------!---------!---------!---------!---------!---------!---------!---------!---------! | 1300 |
| 1: | erValMetHisGluAlaLeuHisAsnHisTyrThrGlnLysSerLeuSerLeuSerProGlyLys | |
| | CCGTGATGCATGAGGCTCTGCACAACCACTACACGCAGAAGAGCCTCTCCCTGTCTCCGGGTAAA | |
| 1301 | ---------!---------!---------!---------!---------!---------!----- | 1365 |

6R4G11 SEQ ID No. 48

| | | |
|---|---|---|
| 1: | GluValGlnLeuValGluSerGlyGlyGlyLeuValGlnProGlyGlySerLeuArgLeuSerCysLysAlaSerGlyTyrSerLeuAlaProGlnAlaM | |
| | GAGGTGCAGCTGGTGGAGTCTGGGGGAGGCTTGGTACAGCCTGGGGGGTCCCTGAGACTCTCCTGTAAGGCTTCTGGATATAGTCTTGCGCCGCAGGCGA | |
| 1 | ---------!---------!---------!---------!---------!---------!---------!---------!---------!---------! | 100 |
| 1: | etThrTrpValArgGlnAlaProGlyLysGlyLeuGluTrpIleGlyValIleAlaSerAsnAlaAspThrTyrTyrAlaSerTrpAlaLysGlyArgPh | |
| | TGACTTGGGTCCGCCAGGCTCCAGGGAAGGGGCTGGAGTGGATCGGAGTCATTGCTAGTAATGCTGACACATACTACGCGAGCTGGGCGAAAGGCCGATT | |
| 101 | ---------!---------!---------!---------!---------!---------!---------!---------!---------!---------! | 200 |
| 1: | eThrIleSerArgAspAsnSerLysAsnThrLeuTyrLeuGlnMetAsnSerLeuArgAlaGluAspThrAlaValTyrTyrCysAlaArgHisProGly | |
| | CACCATCTCCAGAGACAATTCCAAGAACACGCTGTATCTGCAAATGAACAGCCTGAGAGCCGAGGACACGGCCGTATATTACTGTGCCAGACATCCGGGT | |
| 201 | ---------!---------!---------!---------!---------!---------!---------!---------!---------!---------! | 300 |
| 1: | PheAlaGlyTyrGlyTyrGlyHisTyrTyrAlaMetAspProTrpGlyGlnGlyThrLeuValThrValSerSerAlaSerThrLysGlyProSerValP | |
| | TTTGCTGGTTATGGTTATGGGCATTATTACGCCATGGACCCCTGGGGCCAAGGAACCCTGGTCACCGTCTCCTCAGCCTCCACCAAGGGCCCATCGGTCT | |
| 301 | ---------!---------!---------!---------!---------!---------!---------!---------!---------!---------! | 400 |
| 1: | heProLeuAlaProSerSerLysSerThrSerGlyGlyThrAlaAlaLeuGlyCysLeuValLysAspTyrPheProGluProValThrValSerTrpAs | |
| | TCCCCCTGGCACCCTCCTCCAAGAGCACCTCTGGGGGCACAGCGGCCCTGGGCTGCCTGGTCAAGGACTACTTCCCCGAACCGGTGACGGTGTCGTGGAA | |
| 401 | ---------!---------!---------!---------!---------!---------!---------!---------!---------!---------! | 500 |
| 1: | nSerGlyAlaLeuThrSerGlyValHisThrPheProAlaValLeuGlnSerSerGlyLeuTyrSerLeuSerSerValValThrValProSerSerSer | |
| | CTCAGGCGCCCTGACCAGCGGCGTGCACACCTTCCCGGCTGTCCTACAGTCCTCAGGACTCTACTCCCTCAGCAGCGTGGTGACCGTGCCCTCCAGCAGC | |
| 501 | ---------!---------!---------!---------!---------!---------!---------!---------!---------!---------! | 600 |
| 1: | LeuGlyThrGlnThrTyrIleCysAsnValAsnHisLysProSerAsnThrLysValAspLysLysValGluProLysSerCysAspLysThrHisThrC | |
| | TTGGGCACCCAGACCTACATCTGCAACGTGAATCACAAGCCCAGCAACACCAAGGTGGACAAGAAAGTTGAGCCCAAATCTTGTGACAAAACTCACACAT | |
| 601 | ---------!---------!---------!---------!---------!---------!---------!---------!---------!---------! | 700 |
| 1: | ysProProCysProAlaProGluLeuLeuGlyGlyProSerValPheLeuPheProProLysProLysAspThrLeuMetIleSerArgThrProGluVa | |
| | GCCCACCGTGCCCAGCACCTGAACTCCTGGGGGGACCGTCAGTCTTCCTCTTCCCCCCAAAACCCAAGGACACCCTCATGATCTCCCGGACCCCTGAGGT | |
| 701 | ---------!---------!---------!---------!---------!---------!---------!---------!---------!---------! | 800 |
| 1: | lThrCysValValValAspValSerHisGluAspProGluValLysPheAsnTrpTyrValAspGlyValGluValHisAsnAlaLysThrLysProArg | |
| | CACATGCGTGGTGGTGGACGTGAGCCACGAAGACCCTGAGGTCAAGTTCAACTGGTACGTGGACGGCGTGGAGGTGCATAATGCCAAGACAAAGCCGCGG | |
| 801 | ---------!---------!---------!---------!---------!---------!---------!---------!---------!---------! | 900 |
| 1: | GluGluGlnTyrAsnSerThrTyrArgValValSerValLeuThrValLeuHisGlnAspTrpLeuAsnGlyLysGluTyrLysCysLysValSerAsnL | |
| | GAGGAGCAGTACAACAGCACGTACCGTGTGGTCAGCGTCCTCACCGTCCTGCACCAGGACTGGCTGAATGGCAAGGAGTACAAGTGCAAGGTCTCCAACA | |
| 901 | ---------!---------!---------!---------!---------!---------!---------!---------!---------!---------! | 1000 |
| 1: | ysAlaLeuProAlaProIleGluLysThrIleSerLysAlaLysGlyGlnProArgGluProGlnValTyrThrLeuProProSerArgAspGluLeuTh | |
| | AAGCCCTCCCAGCCCCCATCGAGAAAACCATCTCCAAAGCCAAAGGGCAGCCCCGAGAACCACAGGTGTACACCCTGCCCCCATCCCGGGATGAGCTGAC | |
| 1001 | ---------!---------!---------!---------!---------!---------!---------!---------!---------!---------! | 1100 |
| 1: | rLysAsnGlnValSerLeuThrCysLeuValLysGlyPheTyrProSerAspIleAlaValGluTrpGluSerAsnGlyGlnProGluAsnAsnTyrLys | |
| | CAAGAACCAGGTCAGCCTGACCTGCCTGGTCAAAGGCTTCTATCCCAGCGACATCGCCGTGGAGTGGGAGAGCAATGGGCAGCCGGAGAACAACTACAAG | |
| 1101 | ---------!---------!---------!---------!---------!---------!---------!---------!---------!---------! | 1200 |
| 1: | ThrThrProProValLeuAspSerAspGlySerPhePheLeuTyrSerLysLeuThrValAspLysSerArgTrpGlnGlnGlyAsnValPheSerCysS | |
| | ACCACGCCTCCCGTGCTGGACTCCGACGGCTCCTTCTTCCTCTACAGCAAGCTCACCGTGGACAAGAGCAGGTGGCAGCAGGGGAACGTCTTCTCATGCT | |
| 1201 | ---------!---------!---------!---------!---------!---------!---------!---------!---------!---------! | 1300 |
| 1: | erValMetHisGluAlaLeuHisAsnHisTyrThrGlnLysSerLeuSerLeuSerProGlyLys | |
| | CCGTGATGCATGAGGCTCTGCACAACCACTACACGCAGAAGAGCCTCTCCCTGTCTCCGGGTAAA | |
| 1301 | ---------!---------!---------!---------!---------!---------!----- | 1365 |

6R4E12 SEQ ID No. 49

| | | |
|---|---|---|
| 1: | GluValGlnLeuValGluSerGlyGlyGlyLeuValGlnLeuGlyGlySerLeuArgLeuSerCysLysAlaSerGlyTyrSerLeuAlaProGlnAlaM | |
| | GAGGTGCAGCTGGTGGAGTCTGGGGGAGGCTTGGTACAGCTTGGGGGGTCCCTGAGACTCTCCTGTAAGGCTTCTGGATATAGTCTTGCGCCGCAGGCGA | |
| 1 | ---------!---------!---------!---------!---------!---------!---------!---------!---------!---------! | 100 |
| 1: | etThrTrpValArgGlnAlaProGlyLysGlyLeuGluTrpIleGlyValIleAlaSerAsnAlaAspThrTyrTyrAlaSerTrpAlaLysGlyArgPh | |
| | TGACTTGGGTCCGCCAGGCTCCAGGGAAGGGGCTGGAGTGGATCGGAGTCATTGCTAGTAATGCTGACACATACTACGCGAGCTGGGCGAAAGGCCGATT | |
| 101 | ---------!---------!---------!---------!---------!---------!---------!---------!---------!---------! | 200 |
| 1: | eThrIleSerArgAspAsnSerLysAsnThrLeuTyrLeuGlnMetAsnSerLeuArgAlaGluAspThrAlaValTyrTyrCysAlaArgHisProGly | |
| | CACCATCTCCAGAGACAATTCCAAGAACACGCTGTATCTGCAAATGAACAGCCTGAGAGCCGAGGACACGGCCGTATATTACTGTGCCAGACATCCGGGT | |
| 201 | ---------!---------!---------!---------!---------!---------!---------!---------!---------!---------! | 300 |
| 1: | PheAlaGlyTyrGlyTyrGlyHisTyrTyrAlaMetAspProTrpGlyGlnGlyThrLeuValThrValSerSerAlaSerThrLysGlyProSerValP | |
| | TTTGCTGGTTATGGTTATGGGCATTATTACGCCATGGACCCCTGGGGCCAAGGAACCCTGGTCACCGTCTCCTCAGCCTCCACCAAGGGCCCATCGGTCT | |
| 301 | ---------!---------!---------!---------!---------!---------!---------!---------!---------!---------! | 400 |
| 1: | heProLeuAlaProSerSerLysSerThrSerGlyGlyThrAlaAlaLeuGlyCysLeuValLysAspTyrPheProGluProValThrValSerTrpAs | |
| | TCCCCCTGGCACCCTCCTCCAAGAGCACCTCTGGGGGCACAGCGGCCCTGGGCTGCCTGGTCAAGGACTACTTCCCCGAACCGGTGACGGTGTCGTGGAA | |
| 401 | ---------!---------!---------!---------!---------!---------!---------!---------!---------!---------! | 500 |
| 1: | nSerGlyAlaLeuThrSerGlyValHisThrPheProAlaValLeuGlnSerSerGlyLeuTyrSerLeuSerSerValValThrValProSerSerSer | |
| | CTCAGGCGCCCTGACCAGCGGCGTGCACACCTTCCCGGCTGTCCTACAGTCCTCAGGACTCTACTCCCTCAGCAGCGTGGTGACCGTGCCCTCCAGCAGC | |
| 501 | ---------!---------!---------!---------!---------!---------!---------!---------!---------!---------! | 600 |
| 1: | LeuGlyThrGlnThrTyrIleCysAsnValAsnHisLysProSerAsnThrLysValAspLysLysValGluProLysSerCysAspLysThrHisThrC | |
| | TTGGGCACCCAGACCTACATCTGCAACGTGAATCACAAGCCCAGCAACACCAAGGTGGACAAGAAAGTTGAGCCCAAATCTTGTGACAAAACTCACACAT | |
| 601 | ---------!---------!---------!---------!---------!---------!---------!---------!---------!---------! | 700 |
| 1: | ysProProCysProAlaProGluLeuLeuGlyGlyProSerValPheLeuPheProProLysProLysAspThrLeuMetIleSerArgThrProGluVa | |
| | GCCCACCGTGCCCAGCACCTGAACTCCTGGGGGGACCGTCAGTCTTCCTCTTCCCCCCAAAACCCAAGGACACCCTCATGATCTCCCGGACCCCTGAGGT | |
| 701 | ---------!---------!---------!---------!---------!---------!---------!---------!---------!---------! | 800 |
| 1: | lThrCysValValValAspValSerHisGluAspProGluValLysPheAsnTrpTyrValAspGlyValGluValHisAsnAlaLysThrLysProArg | |
| | CACATGCGTGGTGGTGGACGTGAGCCACGAAGACCCTGAGGTCAAGTTCAACTGGTACGTGGACGGCGTGGAGGTGCATAATGCCAAGACAAAGCCGCGG | |
| 801 | ---------!---------!---------!---------!---------!---------!---------!---------!---------!---------! | 900 |
| 1: | GluGluGlnTyrAsnSerThrTyrArgValValSerValLeuThrValLeuHisGlnAspTrpLeuAsnGlyLysGluTyrLysCysLysValSerAsnL | |
| | GAGGAGCAGTACAACAGCACGTACCGTGTGGTCAGCGTCCTCACCGTCCTGCACCAGGACTGGCTGAATGGCAAGGAGTACAAGTGCAAGGTCTCCAACA | |
| 901 | ---------!---------!---------!---------!---------!---------!---------!---------!---------!---------! | 1000 |
| 1: | ysAlaLeuProAlaProIleGluLysThrIleSerLysAlaLysGlyGlnProArgGluProGlnValTyrThrLeuProProSerArgAspGluLeuTh | |
| | AAGCCCTCCCAGCCCCCATCGAGAAAACCATCTCCAAAGCCAAAGGGCAGCCCCGAGAACCACAGGTGTACACCCTGCCCCCATCCCGGGATGAGCTGAC | |
| 1001 | ---------!---------!---------!---------!---------!---------!---------!---------!---------!---------! | 1100 |
| 1: | rLysAsnGlnValSerLeuThrCysLeuValLysGlyPheTyrProSerAspIleAlaValGluTrpGluSerAsnGlyGlnProGluAsnAsnTyrLys | |
| | CAAGAACCAGGTCAGCCTGACCTGCCTGGTCAAAGGCTTCTATCCCAGCGACATCGCCGTGGAGTGGGAGAGCAATGGGCAGCCGGAGAACAACTACAAG | |
| 1101 | ---------!---------!---------!---------!---------!---------!---------!---------!---------!---------! | 1200 |
| 1: | ThrThrProProValLeuAspSerAspGlySerPhePheLeuTyrSerLysLeuThrValAspLysSerArgTrpGlnGlnGlyAsnValPheSerCysS | |
| | ACCACGCCTCCCGTGCTGGACTCCGACGGCTCCTTCTTCCTCTACAGCAAGCTCACCGTGGACAAGAGCAGGTGGCAGCAGGGGAACGTCTTCTCATGCT | |
| 1201 | ---------!---------!---------!---------!---------!---------!---------!---------!---------!---------! | 1300 |
| 1: | erValMetHisGluAlaLeuHisAsnHisTyrThrGlnLysSerLeuSerLeuSerProGlyLys | |
| | CCGTGATGCATGAGGCTCTGCACAACCACTACACGCAGAAGAGCCTCTCCCTGTCTCCGGGTAAA | |
| 1301 | ---------!---------!---------!---------!---------!---------!----- | 1365 |

6R4E3 SEQ ID No. 50

| | | |
|---|---|---|
| 1: | GluValGlnLeuValGluSerGlyGlyGlyLeuValGlnProGlyGlySerLeuArgLeuSerCysLysAlaSerGlyTyrThrPheGluThrGlnAlaM | |
| | GAGGTGCAGCTGGTGGAGTCTGGGGGAGGCTTGGTACAGCCTGGGGGGTCCCTGAGACTCTCCTGTAAGGCTTCTGGATATACTTTTGAGACTCAGGCGA | |
| 1 | ---------!---------!---------!---------!---------!---------!---------!---------!---------!---------! | 100 |
| 1: | etThrTrpValArgGlnAlaProGlyLysGlyLeuGluTrpIleGlyValIleAlaSerAsnAlaAspThrTyrTyrAlaSerTrpAlaLysGlyArgPh | |
| | TGACTTGGGTCCGCCAGGCTCCAGGGAAGGGGCTGGAGTGGATCGGAGTCATTGCTAGTAATGCTGACACATACTACGCGAGCTGGGCGAAAGGCCGGTT | |
| 101 | ---------!---------!---------!---------!---------!---------!---------!---------!---------!---------! | 200 |
| 1: | eThrIleSerArgAspAsnSerLysAsnThrLeuTyrLeuGlnMetAsnSerLeuArgAlaGluAspThrAlaValTyrTyrCysAlaArgHisProGly | |
| | CACCATCTCCAGAGACAATTCCAAGAACACGCTGTATCTGCAAATGAACAGCCTGAGAGCCGAGGACACGGCCGTATATTACTGTGCCAGACATCCTGGT | |
| 201 | ---------!---------!---------!---------!---------!---------!---------!---------!---------!---------! | 300 |
| 1: | SerAlaGlyTyrGlyTyrGlnTyrTyrTyrAlaMetAspProTrpGlyGlnGlyThrLeuValThrValSerSerAlaSerThrLysGlyProSerValP | |
| | AGTGCTGGTTATGGTTATCAGTATTATTACGCCATGGACCCCTGGGGCCAAGGAACCCTGGTCACCGTCTCCTCAGCCTCCACCAAGGGCCCATCGGTCT | |
| 301 | ---------!---------!---------!---------!---------!---------!---------!---------!---------!---------! | 400 |
| 1: | heProLeuAlaProSerSerLysSerThrSerGlyGlyThrAlaAlaLeuGlyCysLeuValLysAspTyrPheProGluProValThrValSerTrpAs | |
| | TCCCCCTGGCACCCTCCTCCAAGAGCACCTCTGGGGGCACAGCGGCCCTGGGCTGCCTGGTCAAGGACTACTTCCCCGAACCGGTGACGGTGTCGTGGAA | |
| 401 | ---------!---------!---------!---------!---------!---------!---------!---------!---------!---------! | 500 |
| 1: | nSerGlyAlaLeuThrSerGlyValHisThrPheProAlaValLeuGlnSerSerGlyLeuTyrSerLeuSerSerValValThrValProSerSerSer | |
| | CTCAGGCGCCCTGACCAGCGGCGTGCACACCTTCCCGGCTGTCCTACAGTCCTCAGGACTCTACTCCCTCAGCAGCGTGGTGACCGTGCCCTCCAGCAGC | |
| 501 | ---------!---------!---------!---------!---------!---------!---------!---------!---------!---------! | 600 |
| 1: | LeuGlyThrGlnThrTyrIleCysAsnValAsnHisLysProSerAsnThrLysValAspLysLysValGluProLysSerCysAspLysThrHisThrC | |
| | TTGGGCACCCAGACCTACATCTGCAACGTGAATCACAAGCCCAGCAACACCAAGGTGGACAAGAAAGTTGAGCCCAAATCTTGTGACAAAACTCACACAT | |
| 601 | ---------!---------!---------!---------!---------!---------!---------!---------!---------!---------! | 700 |
| 1: | ysProProCysProAlaProGluLeuLeuGlyGlyProSerValPheLeuPheProProLysProLysAspThrLeuMetIleSerArgThrProGluVa | |
| | GCCCACCGTGCCCAGCACCTGAACTCCTGGGGGGACCGTCAGTCTTCCTCTTCCCCCCAAAACCCAAGGACACCCTCATGATCTCCCGGACCCCTGAGGT | |
| 701 | ---------!---------!---------!---------!---------!---------!---------!---------!---------!---------! | 800 |
| 1: | lThrCysValValValAspValSerHisGluAspProGluValLysPheAsnTrpTyrValAspGlyValGluValHisAsnAlaLysThrLysProArg | |
| | CACATGCGTGGTGGTGGACGTGAGCCACGAAGACCCTGAGGTCAAGTTCAACTGGTACGTGGACGGCGTGGAGGTGCATAATGCCAAGACAAAGCCGCGG | |
| 801 | ---------!---------!---------!---------!---------!---------!---------!---------!---------!---------! | 900 |
| 1: | GluGluGlnTyrAsnSerThrTyrArgValValSerValLeuThrValLeuHisGlnAspTrpLeuAsnGlyLysGluTyrLysCysLysValSerAsnL | |
| | GAGGAGCAGTACAACAGCACGTACCGTGTGGTCAGCGTCCTCACCGTCCTGCACCAGGACTGGCTGAATGGCAAGGAGTACAAGTGCAAGGTCTCCAACA | |
| 901 | ---------!---------!---------!---------!---------!---------!---------!---------!---------!---------! | 1000 |
| 1: | ysAlaLeuProAlaProIleGluLysThrIleSerLysAlaLysGlyGlnProArgGluProGlnValTyrThrLeuProProSerArgAspGluLeuTh | |
| | AAGCCCTCCCAGCCCCCATCGAGAAAACCATCTCCAAAGCCAAAGGGCAGCCCCGAGAACCACAGGTGTACACCCTGCCCCCATCCCGGGATGAGCTGAC | |
| 1001 | ---------!---------!---------!---------!---------!---------!---------!---------!---------!---------! | 1100 |
| 1: | rLysAsnGlnValSerLeuThrCysLeuValLysGlyPheTyrProSerAspIleAlaValGluTrpGluSerAsnGlyGlnProGluAsnAsnTyrLys | |
| | CAAGAACCAGGTCAGCCTGACCTGCCTGGTCAAAGGCTTCTATCCCAGCGACATCGCCGTGGAGTGGGAGAGCAATGGGCAGCCGGAGAACAACTACAAG | |
| 1101 | ---------!---------!---------!---------!---------!---------!---------!---------!---------!---------! | 1200 |
| 1: | ThrThrProProValLeuAspSerAspGlySerPhePheLeuTyrSerLysLeuThrValAspLysSerArgTrpGlnGlnGlyAsnValPheSerCysS | |
| | ACCACGCCTCCCGTGCTGGACTCCGACGGCTCCTTCTTCCTCTACAGCAAGCTCACCGTGGACAAGAGCAGGTGGCAGCAGGGGAACGTCTTCTCATGCT | |
| 1201 | ---------!---------!---------!---------!---------!---------!---------!---------!---------!---------! | 1300 |
| 1: | erValMetHisGluAlaLeuHisAsnHisTyrThrGlnLysSerLeuSerLeuSerProGlyLys | |
| | CCGTGATGCATGAGGCTCTGCACAACCACTACACGCAGAAGAGCCTCTCCCTGTCTCCGGGTAAA | |
| 1301 | ---------!---------!---------!---------!---------!---------!----- | 1365 |

R4H5 SEQ ID No. 51

| | | |
|---|---|---|
| 1: | GluValGlnLeuValGluSerGlyGlyGlyLeuValGlnProGlyGlySerLeuArgLeuSerCysAlaAlaSerGlyTyrThrValLysArgGlnAlaV | |
| | GAGGTGCAGCTGGTGGAGTCTGGGGGAGGCTTGGTACAGCCTGGGGGGTCCCTGAGACTCTCCTGTGCGGCTTCTGGATATACTGTTAAGAGGCAGGCTG | |
| 1 | ---------!---------!---------!---------!---------!---------!---------!---------!---------!---------! | 100 |
| 1: | alThrTrpValArgGlnAlaProGlyLysGlyLeuGluTrpIleGlyValIleAlaSerAsnAlaAspThrTyrTyrAlaSerTrpAlaLysGlyArgPh | |
| | TCACTTGGGTCCGCCAGGCTCCAGGGAAGGGGCTGGAGTGGATCGGAGTCATTGCTAGTAATGCTGACACATACTACGCGAGCTGGGCGAAAGGCCGGTT | |
| 101 | ---------!---------!---------!---------!---------!---------!---------!---------!---------!---------! | 200 |
| 1: | eThrIleSerArgAspAsnSerLysAsnThrLeuTyrLeuGlnMetAsnSerLeuArgAlaGluAspThrAlaValTyrTyrCysAlaArgHisProGly | |
| | CACCATCTCCAGAGACAATTCCAAGAACACGCTGTATCTGCAAATGAACAGCCTGAGAGCCGAGGACACGGCCGTATATTACTGTGCCAGACATCCTGGT | |
| 201 | ---------!---------!---------!---------!---------!---------!---------!---------!---------!---------! | 300 |
| 1: | SerAlaGlyTyrGlyTyrGlnTyrTyrTyrAlaMetAspProTrpGlyGlnGlyThrLeuValThrValSerSerAlaSerThrLysGlyProSerValP | |
| | AGTGCTGGTTATGGTTATCAGTATTATTACGCCATGGACCCCTGGGGCCAAGGAACCCTGGTCACCGTCTCCTCAGCCTCCACCAAGGGCCCATCGGTCT | |
| 301 | ---------!---------!---------!---------!---------!---------!---------!---------!---------!---------! | 400 |
| 1: | heProLeuAlaProSerSerLysSerThrSerGlyGlyThrAlaAlaLeuGlyCysLeuValLysAspTyrPheProGluProValThrValSerTrpAs | |
| | TCCCCCTGGCACCCTCCTCCAAGAGCACCTCTGGGGGCACAGCGGCCCTGGGCTGCCTGGTCAAGGACTACTTCCCCGAACCGGTGACGGTGTCGTGGAA | |
| 401 | ---------!---------!---------!---------!---------!---------!---------!---------!---------!---------! | 500 |
| 1: | nSerGlyAlaLeuThrSerGlyValHisThrPheProAlaValLeuGlnSerSerGlyLeuTyrSerLeuSerSerValValThrValProSerSerSer | |
| | CTCAGGCGCCCTGACCAGCGGCGTGCACACCTTCCCGGCTGTCCTACAGTCCTCAGGACTCTACTCCCTCAGCAGCGTGGTGACCGTGCCCTCCAGCAGC | |
| 501 | ---------!---------!---------!---------!---------!---------!---------!---------!---------!---------! | 600 |
| 1: | LeuGlyThrGlnThrTyrIleCysAsnValAsnHisLysProSerAsnThrLysValAspLysLysValGluProLysSerCysAspLysThrHisThrC | |
| | TTGGGCACCCAGACCTACATCTGCAACGTGAATCACAAGCCCAGCAACACCAAGGTGGACAAGAAAGTTGAGCCCAAATCTTGTGACAAAACTCACACAT | |
| 601 | ---------!---------!---------!---------!---------!---------!---------!---------!---------!---------! | 700 |
| 1: | ysProProCysProAlaProGluLeuLeuGlyGlyProSerValPheLeuPheProProLysProLysAspThrLeuMetIleSerArgThrProGluVa | |
| | GCCCACCGTGCCCAGCACCTGAACTCCTGGGGGGACCGTCAGTCTTCCTCTTCCCCCCAAAACCCAAGGACACCCTCATGATCTCCCGGACCCCTGAGGT | |
| 701 | ---------!---------!---------!---------!---------!---------!---------!---------!---------!---------! | 800 |
| 1: | lThrCysValValValAspValSerHisGluAspProGluValLysPheAsnTrpTyrValAspGlyValGluValHisAsnAlaLysThrLysProArg | |
| | CACATGCGTGGTGGTGGACGTGAGCCACGAAGACCCTGAGGTCAAGTTCAACTGGTACGTGGACGGCGTGGAGGTGCATAATGCCAAGACAAAGCCGCGG | |
| 801 | ---------!---------!---------!---------!---------!---------!---------!---------!---------!---------! | 900 |
| 1: | GluGluGlnTyrAsnSerThrTyrArgValValSerValLeuThrValLeuHisGlnAspTrpLeuAsnGlyLysGluTyrLysCysLysValSerAsnL | |
| | GAGGAGCAGTACAACAGCACGTACCGTGTGGTCAGCGTCCTCACCGTCCTGCACCAGGACTGGCTGAATGGCAAGGAGTACAAGTGCAAGGTCTCCAACA | |
| 901 | ---------!---------!---------!---------!---------!---------!---------!---------!---------!---------! | 1000 |
| 1: | ysAlaLeuProAlaProIleGluLysThrIleSerLysAlaLysGlyGlnProArgGluProGlnValTyrThrLeuProProSerArgAspGluLeuTh | |
| | AAGCCCTCCCAGCCCCCATCGAGAAAACCATCTCCAAAGCCAAAGGGCAGCCCCGAGAACCACAGGTGTACACCCTGCCCCCATCCCGGGATGAGCTGAC | |
| 1001 | ---------!---------!---------!---------!---------!---------!---------!---------!---------!---------! | 1100 |
| 1: | rLysAsnGlnValSerLeuThrCysLeuValLysGlyPheTyrProSerAspIleAlaValGluTrpGluSerAsnGlyGlnProGluAsnAsnTyrLys | |
| | CAAGAACCAGGTCAGCCTGACCTGCCTGGTCAAAGGCTTCTATCCCAGCGACATCGCCGTGGAGTGGGAGAGCAATGGGCAGCCGGAGAACAACTACAAG | |
| 1101 | ---------!---------!---------!---------!---------!---------!---------!---------!---------!---------! | 1200 |
| 1: | ThrThrProProValLeuAspSerAspGlySerPhePheLeuTyrSerLysLeuThrValAspLysSerArgTrpGlnGlnGlyAsnValPheSerCysS | |
| | ACCACGCCTCCCGTGCTGGACTCCGACGGCTCCTTCTTCCTCTACAGCAAGCTCACCGTGGACAAGAGCAGGTGGCAGCAGGGGAACGTCTTCTCATGCT | |
| 1201 | ---------!---------!---------!---------!---------!---------!---------!---------!---------!---------! | 1300 |
| 1: | erValMetHisGluAlaLeuHisAsnHisTyrThrGlnLysSerLeuSerLeuSerProGlyLys | |
| | CCGTGATGCATGAGGCTCTGCACAACCACTACACGCAGAAGAGCCTCTCCCTGTCTCCGGGTAAA | |
| 1301 | ---------!---------!---------!---------!---------!---------!----- | 1365 |

6R4H11 SEQ ID No. 52

| | | |
|---|---|---|
| 1: | GluValGlnLeuValGluSerGlyGlyGlyLeuValGlnProGlyGlySerLeuArgLeuSerCysLysAlaSerGlyPheThrPheLysProGlnAlaM | |
| | GAGGTGCAGCTGGTGGAGTCTGGGGGAGGCTTGGTACAGCCTGGGGGGTCCCTGAGACTCTCCTGTAAGGCTTCTGGATTTACTTTTAAGCCGCAGGCTA | |
| 1 | ---------!---------!---------!---------!---------!---------!---------!---------!---------!---------! | 100 |
| 1: | etThrTrpValArgGlnAlaProGlyLysGlyLeuGluTrpIleGlyValIleAlaSerAsnAlaAspThrTyrTyrAlaSerTrpAlaLysGlyArgPh | |
| | TGACATGGGTCCGCCAGGCTCCAGGGAAGGGGCTGGAGTGGATCGGAGTCATTGCTAGTAATGCTGACACATACTACGCGAGCTGGGCGAAAGGCCGGTT | |
| 101 | ---------!---------!---------!---------!---------!---------!---------!---------!---------!---------! | 200 |
| 1: | eThrIleSerArgAspAsnSerLysAsnThrLeuTyrLeuGlnMetAsnSerLeuArgAlaGluAspThrAlaValTyrTyrCysAlaArgHisProGly | |
| | CACCATCTCCAGAGACAATTCCAAGAACACGCTGTATCTGCAAATGAACAGCCTGAGAGCCGAGGACACGGCCGTATATTACTGTGCCAGACATCCTGGT | |
| 201 | ---------!---------!---------!---------!---------!---------!---------!---------!---------!---------! | 300 |
| 1: | SerAlaGlyTyrGlyTyrGlnTyrTyrTyrAlaMetAspProTrpGlyGlnGlyThrLeuValThrValSerSerAlaSerThrLysGlyProSerValP | |
| | AGTGCTGGTTATGGTTATCAGTATTATTACGCCATGGACCCCTGGGGCCAAGGAACCCTGGTCACCGTCTCCTCAGCCTCCACCAAGGGCCCATCGGTCT | |
| 301 | ---------!---------!---------!---------!---------!---------!---------!---------!---------!---------! | 400 |
| 1: | heProLeuAlaProSerSerLysSerThrSerGlyGlyThrAlaAlaLeuGlyCysLeuValLysAspTyrPheProGluProValThrValSerTrpAs | |
| | TCCCCCTGGCACCCTCCTCCAAGAGCACCTCTGGGGGCACAGCGGCCCTGGGCTGCCTGGTCAAGGACTACTTCCCCGAACCGGTGACGGTGTCGTGGAA | |
| 401 | ---------!---------!---------!---------!---------!---------!---------!---------!---------!---------! | 500 |
| 1: | nSerGlyAlaLeuThrSerGlyValHisThrPheProAlaValLeuGlnSerSerGlyLeuTyrSerLeuSerSerValValThrValProSerSerSer | |
| | CTCAGGCGCCCTGACCAGCGGCGTGCACACCTTCCCGGCTGTCCTACAGTCCTCAGGACTCTACTCCCTCAGCAGCGTGGTGACCGTGCCCTCCAGCAGC | |
| 501 | ---------!---------!---------!---------!---------!---------!---------!---------!---------!---------! | 600 |
| 1: | LeuGlyThrGlnThrTyrIleCysAsnValAsnHisLysProSerAsnThrLysValAspLysLysValGluProLysSerCysAspLysThrHisThrC | |
| | TTGGGCACCCAGACCTACATCTGCAACGTGAATCACAAGCCCAGCAACACCAAGGTGGACAAGAAAGTTGAGCCCAAATCTTGTGACAAAACTCACACAT | |
| 601 | ---------!---------!---------!---------!---------!---------!---------!---------!---------!---------! | 700 |
| 1: | ysProProCysProAlaProGluLeuLeuGlyGlyProSerValPheLeuPheProProLysProLysAspThrLeuMetIleSerArgThrProGluVa | |
| | GCCCACCGTGCCCAGCACCTGAACTCCTGGGGGGACCGTCAGTCTTCCTCTTCCCCCCAAAACCCAAGGACACCCTCATGATCTCCCGGACCCCTGAGGT | |
| 701 | ---------!---------!---------!---------!---------!---------!---------!---------!---------!---------! | 800 |
| 1: | lThrCysValValValAspValSerHisGluAspProGluValLysPheAsnTrpTyrValAspGlyValGluValHisAsnAlaLysThrLysProArg | |
| | CACATGCGTGGTGGTGGACGTGAGCCACGAAGACCCTGAGGTCAAGTTCAACTGGTACGTGGACGGCGTGGAGGTGCATAATGCCAAGACAAAGCCGCGG | |
| 801 | ---------!---------!---------!---------!---------!---------!---------!---------!---------!---------! | 900 |
| 1: | GluGluGlnTyrAsnSerThrTyrArgValValSerValLeuThrValLeuHisGlnAspTrpLeuAsnGlyLysGluTyrLysCysLysValSerAsnL | |
| | GAGGAGCAGTACAACAGCACGTACCGTGTGGTCAGCGTCCTCACCGTCCTGCACCAGGACTGGCTGAATGGCAAGGAGTACAAGTGCAAGGTCTCCAACA | |
| 901 | ---------!---------!---------!---------!---------!---------!---------!---------!---------!---------! | 1000 |
| 1: | ysAlaLeuProAlaProIleGluLysThrIleSerLysAlaLysGlyGlnProArgGluProGlnValTyrThrLeuProProSerArgAspGluLeuTh | |
| | AAGCCCTCCCAGCCCCCATCGAGAAAACCATCTCCAAAGCCAAAGGGCAGCCCCGAGAACCACAGGTGTACACCCTGCCCCCATCCCGGGATGAGCTGAC | |
| 1001 | ---------!---------!---------!---------!---------!---------!---------!---------!---------!---------! | 1100 |
| 1: | rLysAsnGlnValSerLeuThrCysLeuValLysGlyPheTyrProSerAspIleAlaValGluTrpGluSerAsnGlyGlnProGluAsnAsnTyrLys | |
| | CAAGAACCAGGTCAGCCTGACCTGCCTGGTCAAAGGCTTCTATCCCAGCGACATCGCCGTGGAGTGGGAGAGCAATGGGCAGCCGGAGAACAACTACAAG | |
| 1101 | ---------!---------!---------!---------!---------!---------!---------!---------!---------!---------! | 1200 |
| 1: | ThrThrProProValLeuAspSerAspGlySerPhePheLeuTyrSerLysLeuThrValAspLysSerArgTrpGlnGlnGlyAsnValPheSerCysS | |
| | ACCACGCCTCCCGTGCTGGACTCCGACGGCTCCTTCTTCCTCTACAGCAAGCTCACCGTGGACAAGAGCAGGTGGCAGCAGGGGAACGTCTTCTCATGCT | |
| 1201 | ---------!---------!---------!---------!---------!---------!---------!---------!---------!---------! | 1300 |
| 1: | erValMetHisGluAlaLeuHisAsnHisTyrThrGlnLysSerLeuSerLeuSerProGlyLys | |
| | CCGTGATGCATGAGGCTCTGCACAACCACTACACGCAGAAGAGCCTCTCCCTGTCTCCGGGTAAA | |
| 1301 | ---------!---------!---------!---------!---------!---------!----- | 1365 |

6R6A7 SEQ ID No. 53

| | | |
|---|---|---|
| 1: | GluValGlnLeuValGluSerGlyGlyGlyLeuValGlnLeuGlyGlySerLeuArgLeuSerCysLysAlaSerGlyTyrSerLeuAlaProGlnAlaM | |
| | GAGGTGCAGCTGGTGGAGTCTGGGGGAGGCTTGGTACAGCTTGGGGGGTCCCTGAGACTCTCCTGTAAGGCTTCTGGATATAGTCTTGCGCCGCAGGCGA | |
| 1 | ---------!---------!---------!---------!---------!---------!---------!---------!---------!---------! | 100 |
| 1: | etThrTrpValArgGlnAlaProGlyLysGlyLeuGluTrpIleGlyValIleAlaSerAsnAlaAspThrTyrTyrAlaSerTrpAlaLysGlyArgPh | |
| | TGACTTGGGTCCGCCAGGCTCCAGGGAAGGGGCTGGAGTGGATCGGAGTCATTGCTAGTAATGCTGACACATACTACGCGAGCTGGGCGAAAGGCCGGTT | |
| 101 | ---------!---------!---------!---------!---------!---------!---------!---------!---------!---------! | 200 |
| 1: | eThrIleSerArgAspAsnSerLysAsnThrLeuTyrLeuGlnMetAsnSerLeuArgAlaGluAspThrAlaValTyrTyrCysAlaArgHisProGly | |
| | CACCATCTCCAGAGACAATTCCAAGAACACGCTGTATCTGCAAATGAACAGCCTGAGAGCCGAGGACACGGCCGTATATTACTGTGCCAGACATCCTGGT | |
| 201 | ---------!---------!---------!---------!---------!---------!---------!---------!---------!---------! | 300 |
| 1: | SerAlaGlyTyrGlyTyrGlnTyrTyrTyrSerMetAspProTrpGlyGlnGlyThrLeuValThrValSerSerAlaSerThrLysGlyProSerValP | |
| | AGTGCTGGTTATGGTTATCAGTATTATTATAGTATGGACCCCTGGGGCCAAGGAACCCTGGTCACCGTCTCCTCAGCCTCCACCAAGGGCCCATCGGTCT | |
| 301 | ---------!---------!---------!---------!---------!---------!---------!---------!---------!---------! | 400 |
| 1: | heProLeuAlaProSerSerLysSerThrSerGlyGlyThrAlaAlaLeuGlyCysLeuValLysAspTyrPheProGluProValThrValSerTrpAs | |
| | TCCCCCTGGCACCCTCCTCCAAGAGCACCTCTGGGGGCACAGCGGCCCTGGGCTGCCTGGTCAAGGACTACTTCCCCGAACCGGTGACGGTGTCGTGGAA | |
| 401 | ---------!---------!---------!---------!---------!---------!---------!---------!---------!---------! | 500 |
| 1: | nSerGlyAlaLeuThrSerGlyValHisThrPheProAlaValLeuGlnSerSerGlyLeuTyrSerLeuSerSerValValThrValProSerSerSer | |
| | CTCAGGCGCCCTGACCAGCGGCGTGCACACCTTCCCGGCTGTCCTACAGTCCTCAGGACTCTACTCCCTCAGCAGCGTGGTGACCGTGCCCTCCAGCAGC | |
| 501 | ---------!---------!---------!---------!---------!---------!---------!---------!---------!---------! | 600 |
| 1: | LeuGlyThrGlnThrTyrIleCysAsnValAsnHisLysProSerAsnThrLysValAspLysLysValGluProLysSerCysAspLysThrHisThrC | |
| | TTGGGCACCCAGACCTACATCTGCAACGTGAATCACAAGCCCAGCAACACCAAGGTGGACAAGAAAGTTGAGCCCAAATCTTGTGACAAAACTCACACAT | |
| 601 | ---------!---------!---------!---------!---------!---------!---------!---------!---------!---------! | 700 |
| 1: | ysProProCysProAlaProGluLeuLeuGlyGlyProSerValPheLeuPheProProLysProLysAspThrLeuMetIleSerArgThrProGluVa | |
| | GCCCACCGTGCCCAGCACCTGAACTCCTGGGGGGACCGTCAGTCTTCCTCTTCCCCCCAAAACCCAAGGACACCCTCATGATCTCCCGGACCCCTGAGGT | |
| 701 | ---------!---------!---------!---------!---------!---------!---------!---------!---------!---------! | 800 |
| 1: | lThrCysValValValAspValSerHisGluAspProGluValLysPheAsnTrpTyrValAspGlyValGluValHisAsnAlaLysThrLysProArg | |
| | CACATGCGTGGTGGTGGACGTGAGCCACGAAGACCCTGAGGTCAAGTTCAACTGGTACGTGGACGGCGTGGAGGTGCATAATGCCAAGACAAAGCCGCGG | |
| 801 | ---------!---------!---------!---------!---------!---------!---------!---------!---------!---------! | 900 |
| 1: | GluGluGlnTyrAsnSerThrTyrArgValValSerValLeuThrValLeuHisGlnAspTrpLeuAsnGlyLysGluTyrLysCysLysValSerAsnL | |
| | GAGGAGCAGTACAACAGCACGTACCGTGTGGTCAGCGTCCTCACCGTCCTGCACCAGGACTGGCTGAATGGCAAGGAGTACAAGTGCAAGGTCTCCAACA | |
| 901 | ---------!---------!---------!---------!---------!---------!---------!---------!---------!---------! | 1000 |
| 1: | ysAlaLeuProAlaProIleGluLysThrIleSerLysAlaLysGlyGlnProArgGluProGlnValTyrThrLeuProProSerArgAspGluLeuTh | |
| | AAGCCCTCCCAGCCCCCATCGAGAAAACCATCTCCAAAGCCAAAGGGCAGCCCCGAGAACCACAGGTGTACACCCTGCCCCCATCCCGGGATGAGCTGAC | |
| 1001 | ---------!---------!---------!---------!---------!---------!---------!---------!---------!---------! | 1100 |
| 1: | rLysAsnGlnValSerLeuThrCysLeuValLysGlyPheTyrProSerAspIleAlaValGluTrpGluSerAsnGlyGlnProGluAsnAsnTyrLys | |
| | CAAGAACCAGGTCAGCCTGACCTGCCTGGTCAAAGGCTTCTATCCCAGCGACATCGCCGTGGAGTGGGAGAGCAATGGGCAGCCGGAGAACAACTACAAG | |
| 1101 | ---------!---------!---------!---------!---------!---------!---------!---------!---------!---------! | 1200 |
| 1: | ThrThrProProValLeuAspSerAspGlySerPhePheLeuTyrSerLysLeuThrValAspLysSerArgTrpGlnGlnGlyAsnValPheSerCysS | |
| | ACCACGCCTCCCGTGCTGGACTCCGACGGCTCCTTCTTCCTCTACAGCAAGCTCACCGTGGACAAGAGCAGGTGGCAGCAGGGGAACGTCTTCTCATGCT | |
| 1201 | ---------!---------!---------!---------!---------!---------!---------!---------!---------!---------! | 1300 |
| 1: | erValMetHisGluAlaLeuHisAsnHisTyrThrGlnLysSerLeuSerLeuSerProGlyLys | |
| | CCGTGATGCATGAGGCTCTGCACAACCACTACACGCAGAAGAGCCTCTCCCTGTCTCCGGGTAAA | |
| 1301 | ---------!---------!---------!---------!---------!---------!----- | 1365 |

6R6E1 SEQ ID No. 54

| | | |
|---|---|---|
| 1: | GluValGlnLeuValGluSerGlyGlyGlyLeuValGlnProGlyGlySerLeuArgLeuSerCysGluAlaSerGlyPheThrPheProAsnGlnAlaM | |
| | GAGGTGCAGCTGGTGGAGTCTGGGGGAGGCTTGGTACAGCCTGGGGGGTCCCTGAGACTCTCCTGTGAGGCTTCTGGATTTACTTTTCCGAATCAGGCTA | |
| 1 | ---------!---------!---------!---------!---------!---------!---------!---------!---------!---------! | 100 |
| 1: | etThrTrpValArgGlnAlaProGlyLysGlyLeuGluTrpIleGlyValIleAlaSerAsnAlaAspThrTyrTyrAlaSerTrpAlaLysGlyArgPh | |
| | TGACTTGGGTCCGCCAGGCTCCAGGGAAGGGGCTGGAGTGGATCGGAGTCATTGCTAGTAATGCTGACACATACTACGCGAGCTGGGCGAAAGGCCGGTT | |
| 101 | ---------!---------!---------!---------!---------!---------!---------!---------!---------!---------! | 200 |
| 1: | eThrIleSerArgAspAsnSerLysAsnThrLeuTyrLeuGlnMetAsnSerLeuArgAlaGluAspThrAlaValTyrTyrCysAlaArgHisProGly | |
| | CACCATCTCCAGAGACAATTCCAAGAACACGCTGTATCTGCAAATGAACAGCCTGAGAGCCGAGGACACGGCCGTATATTACTGTGCCAGACATCCGGGT | |
| 201 | ---------!---------!---------!---------!---------!---------!---------!---------!---------!---------! | 300 |
| 1: | PheAlaGlyTyrGlyTyrGlyMetTyrTyrSerMetAspProTrpGlyGlnGlyThrLeuValThrValSerSerAlaSerThrLysGlyProSerValP | |
| | TTTGCTGGTTATGGTTATGGTATGTATTATTCGATGGACCCCTGGGGCCAAGGAACCCTGGTCACCGTCTCCTCAGCCTCCACCAAGGGCCCATCGGTCT | |
| 301 | ---------!---------!---------!---------!---------!---------!---------!---------!---------!---------! | 400 |
| 1: | heProLeuAlaProSerSerLysSerThrSerGlyGlyThrAlaAlaLeuGlyCysLeuValLysAspTyrPheProGluProValThrValSerTrpAs | |
| | TCCCCCTGGCACCCTCCTCCAAGAGCACCTCTGGGGGCACAGCGGCCCTGGGCTGCCTGGTCAAGGACTACTTCCCCGAACCGGTGACGGTGTCGTGGAA | |
| 401 | ---------!---------!---------!---------!---------!---------!---------!---------!---------!---------! | 500 |
| 1: | nSerGlyAlaLeuThrSerGlyValHisThrPheProAlaValLeuGlnSerSerGlyLeuTyrSerLeuSerSerValValThrValProSerSerSer | |
| | CTCAGGCGCCCTGACCAGCGGCGTGCACACCTTCCCGGCTGTCCTACAGTCCTCAGGACTCTACTCCCTCAGCAGCGTGGTGACCGTGCCCTCCAGCAGC | |
| 501 | ---------!---------!---------!---------!---------!---------!---------!---------!---------!---------! | 600 |
| 1: | LeuGlyThrGlnThrTyrIleCysAsnValAsnHisLysProSerAsnThrLysValAspLysLysValGluProLysSerCysAspLysThrHisThrC | |
| | TTGGGCACCCAGACCTACATCTGCAACGTGAATCACAAGCCCAGCAACACCAAGGTGGACAAGAAAGTTGAGCCCAAATCTTGTGACAAAACTCACACAT | |
| 601 | ---------!---------!---------!---------!---------!---------!---------!---------!---------!---------! | 700 |
| 1: | ysProProCysProAlaProGluLeuLeuGlyGlyProSerValPheLeuPheProProLysProLysAspThrLeuMetIleSerArgThrProGluVa | |
| | GCCCACCGTGCCCAGCACCTGAACTCCTGGGGGGACCGTCAGTCTTCCTCTTCCCCCCAAAACCCAAGGACACCCTCATGATCTCCCGGACCCCTGAGGT | |
| 701 | ---------!---------!---------!---------!---------!---------!---------!---------!---------!---------! | 800 |
| 1: | lThrCysValValValAspValSerHisGluAspProGluValLysPheAsnTrpTyrValAspGlyValGluValHisAsnAlaLysThrLysProArg | |
| | CACATGCGTGGTGGTGGACGTGAGCCACGAAGACCCTGAGGTCAAGTTCAACTGGTACGTGGACGGCGTGGAGGTGCATAATGCCAAGACAAAGCCGCGG | |
| 801 | ---------!---------!---------!---------!---------!---------!---------!---------!---------!---------! | 900 |
| 1: | GluGluGlnTyrAsnSerThrTyrArgValValSerValLeuThrValLeuHisGlnAspTrpLeuAsnGlyLysGluTyrLysCysLysValSerAsnL | |
| | GAGGAGCAGTACAACAGCACGTACCGTGTGGTCAGCGTCCTCACCGTCCTGCACCAGGACTGGCTGAATGGCAAGGAGTACAAGTGCAAGGTCTCCAACA | |
| 901 | ---------!---------!---------!---------!---------!---------!---------!---------!---------!---------! | 1000 |
| 1: | ysAlaLeuProAlaProIleGluLysThrIleSerLysAlaLysGlyGlnProArgGluProGlnValTyrThrLeuProProSerArgAspGluLeuTh | |
| | AAGCCCTCCCAGCCCCCATCGAGAAAACCATCTCCAAAGCCAAAGGGCAGCCCCGAGAACCACAGGTGTACACCCTGCCCCCATCCCGGGATGAGCTGAC | |
| 1001 | ---------!---------!---------!---------!---------!---------!---------!---------!---------!---------! | 1100 |
| 1: | rLysAsnGlnValSerLeuThrCysLeuValLysGlyPheTyrProSerAspIleAlaValGluTrpGluSerAsnGlyGlnProGluAsnAsnTyrLys | |
| | CAAGAACCAGGTCAGCCTGACCTGCCTGGTCAAAGGCTTCTATCCCAGCGACATCGCCGTGGAGTGGGAGAGCAATGGGCAGCCGGAGAACAACTACAAG | |
| 1101 | ---------!---------!---------!---------!---------!---------!---------!---------!---------!---------! | 1200 |
| 1: | ThrThrProProValLeuAspSerAspGlySerPhePheLeuTyrSerLysLeuThrValAspLysSerArgTrpGlnGlnGlyAsnValPheSerCysS | |
| | ACCACGCCTCCCGTGCTGGACTCCGACGGCTCCTTCTTCCTCTACAGCAAGCTCACCGTGGACAAGAGCAGGTGGCAGCAGGGGAACGTCTTCTCATGCT | |
| 1201 | ---------!---------!---------!---------!---------!---------!---------!---------!---------!---------! | 1300 |
| 1: | erValMetHisGluAlaLeuHisAsnHisTyrThrGlnLysSerLeuSerLeuSerProGlyLys | |
| | CCGTGATGCATGAGGCTCTGCACAACCACTACACGCAGAAGAGCCTCTCCCTGTCTCCGGGTAAA | |
| 1301 | ---------!---------!---------!---------!---------!---------!----- | 1365 |

6R6E2 SEQ ID No. 55

| | | |
|---|---|---|
| 1: | GluValGlnLeuValGluSerGlyGlyGlyLeuValGlnProGlyGlySerLeuArgLeuSerCysLysAlaSerGlyIleSerValAsnThrLysAlaL | 100 |
| | GAGGTGCAGCTGGTGGAGTCTGGGGGAGGCTTGGTACAGCCTGGGGGGTCCCTGAGACTCTCCTGTAAGGCTTCTGGAATTAGTGTTAATACTAAGGCGC | |
| 1 | ---------!---------!---------!---------!---------!---------!---------!---------!---------!---------! | |
| 1: | euThrTrpValArgGlnAlaProGlyLysGlyLeuGluTrpIleGlyValIleAlaSerAsnAlaAspThrTyrTyrAlaSerTrpAlaLysGlyArgPh | |
| | TCACTTGGGTCCGCCAGGCTCCAGGGAAGGGGCTGGAGTGGATCGGAGTCATTGCTAGTAATGCTGACACATACTACGCGAGCTGGGCGAAAGGCCGATT | |
| 101 | ---------!---------!---------!---------!---------!---------!---------!---------!---------!---------! | 200 |
| 1: | eThrIleSerArgAspAsnSerLysAsnThrLeuTyrLeuGlnMetAsnSerLeuArgAlaGluAspThrAlaValTyrTyrCysAlaArgHisProGly | |
| | CACCATCTCCAGAGACAATTCCAAGAACACGCTGTATCTGCAAATGAACAGCCTGAGAGCCGAGGACACGGCCGTATATTACTGTGCCAGACATCCGGGT | |
| 201 | ---------!---------!---------!---------!---------!---------!---------!---------!---------!---------! | 300 |
| 1: | PheAlaGlyTyrGlyTyrGlyHisTyrTyrAlaMetAspProTrpGlyGlnGlyThrLeuValThrValSerSerAlaSerThrLysGlyProSerValP | |
| | TTTGCTGGTTATGGTTATGGGCATTATTACGCCATGGACCCCTGGGGCCAAGGAACCCTGGTCACCGTCTCCTCAGCCTCCACCAAGGGCCCATCGGTCT | |
| 301 | ---------!---------!---------!---------!---------!---------!---------!---------!---------!---------! | 400 |
| 1: | heProLeuAlaProSerSerLysSerThrSerGlyGlyThrAlaAlaLeuGlyCysLeuValLysAspTyrPheProGluProValThrValSerTrpAs | |
| | TCCCCCTGGCACCCTCCTCCAAGAGCACCTCTGGGGGCACAGCGGCCCTGGGCTGCCTGGTCAAGGACTACTTCCCCGAACCGGTGACGGTGTCGTGGAA | |
| 401 | ---------!---------!---------!---------!---------!---------!---------!---------!---------!---------! | 500 |
| 1: | nSerGlyAlaLeuThrSerGlyValHisThrPheProAlaValLeuGlnSerSerGlyLeuTyrSerLeuSerSerValValThrValProSerSerSer | |
| | CTCAGGCGCCCTGACCAGCGGCGTGCACACCTTCCCGGCTGTCCTACAGTCCTCAGGACTCTACTCCCTCAGCAGCGTGGTGACCGTGCCCTCCAGCAGC | |
| 501 | ---------!---------!---------!---------!---------!---------!---------!---------!---------!---------! | 600 |
| 1: | LeuGlyThrGlnThrTyrIleCysAsnValAsnHisLysProSerAsnThrLysValAspLysLysValGluProLysSerCysAspLysThrHisThrC | |
| | TTGGGCACCCAGACCTACATCTGCAACGTGAATCACAAGCCCAGCAACACCAAGGTGGACAAGAAAGTTGAGCCCAAATCTTGTGACAAAACTCACACAT | |
| 601 | ---------!---------!---------!---------!---------!---------!---------!---------!---------!---------! | 700 |
| 1: | ysProProCysProAlaProGluLeuLeuGlyGlyProSerValPheLeuPheProProLysProLysAspThrLeuMetIleSerArgThrProGluVa | |
| | GCCCACCGTGCCCAGCACCTGAACTCCTGGGGGGACCGTCAGTCTTCCTCTTCCCCCCAAAACCCAAGGACACCCTCATGATCTCCCGGACCCCTGAGGT | |
| 701 | ---------!---------!---------!---------!---------!---------!---------!---------!---------!---------! | 800 |
| 1: | lThrCysValValValAspValSerHisGluAspProGluValLysPheAsnTrpTyrValAspGlyValGluValHisAsnAlaLysThrLysProArg | |
| | CACATGCGTGGTGGTGGACGTGAGCCACGAAGACCCTGAGGTCAAGTTCAACTGGTACGTGGACGGCGTGGAGGTGCATAATGCCAAGACAAAGCCGCGG | |
| 801 | ---------!---------!---------!---------!---------!---------!---------!---------!---------!---------! | 900 |
| 1: | GluGluGlnTyrAsnSerThrTyrArgValValSerValLeuThrValLeuHisGlnAspTrpLeuAsnGlyLysGluTyrLysCysLysValSerAsnL | |
| | GAGGAGCAGTACAACAGCACGTACCGTGTGGTCAGCGTCCTCACCGTCCTGCACCAGGACTGGCTGAATGGCAAGGAGTACAAGTGCAAGGTCTCCAACA | |
| 901 | ---------!---------!---------!---------!---------!---------!---------!---------!---------!---------! | 1000 |
| 1: | ysAlaLeuProAlaProIleGluLysThrIleSerLysAlaLysGlyGlnProArgGluProGlnValTyrThrLeuProProSerArgAspGluLeuTh | |
| | AAGCCCTCCCAGCCCCCATCGAGAAAACCATCTCCAAAGCCAAAGGGCAGCCCCGAGAACCACAGGTGTACACCCTGCCCCCATCCCGGGATGAGCTGAC | |
| 1001 | ---------!---------!---------!---------!---------!---------!---------!---------!---------!---------! | 1100 |
| 1: | rLysAsnGlnValSerLeuThrCysLeuValLysGlyPheTyrProSerAspIleAlaValGluTrpGluSerAsnGlyGlnProGluAsnAsnTyrLys | |
| | CAAGAACCAGGTCAGCCTGACCTGCCTGGTCAAAGGCTTCTATCCCAGCGACATCGCCGTGGAGTGGGAGAGCAATGGGCAGCCGGAGAACAACTACAAG | |
| 1101 | ---------!---------!---------!---------!---------!---------!---------!---------!---------!---------! | 1200 |
| 1: | ThrThrProProValLeuAspSerAspGlySerPhePheLeuTyrSerLysLeuThrValAspLysSerArgTrpGlnGlnGlyAsnValPheSerCysS | |
| | ACCACGCCTCCCGTGCTGGACTCCGACGGCTCCTTCTTCCTCTACAGCAAGCTCACCGTGGACAAGAGCAGGTGGCAGCAGGGGAACGTCTTCTCATGCT | |
| 1201 | | 1300 |
| 1: | erValMetHisGluAlaLeuHisAsnHisTyrThrGlnLysSerLeuSerLeuSerProGlyLys | |
| | CCGTGATGCATGAGGCTCTGCACAACCACTACACGCAGAAGAGCCTCTCCCTGTCTCCGGGTAAA | |
| 1301 | ---------!---------!---------!---------!---------!---------!----- | 1365 |

6R6E3 SEQ ID No. 56

| | | |
|---|---|---|
| 1: | GluValGlnLeuValGluSerGlyGlyGlyLeuValGlnProGlyGlySerLeuArgLeuSerCysLysAlaSerGlyPheThrPheSerArgGlnAlaL | |
| | GAGGTGCAGCTGGTGGAGTCTGGGGGAGGCTTGGTACAGCCTGGGGGGTCCCTGAGACTCTCCTGTAAGGCTTCTGGATTTACTTTTAGTCGTCAGGCTC | |
| 1 | ---------!---------!---------!---------!---------!---------!---------!---------!---------!---------! | 100 |
| 1: | euThrTrpValArgGlnAlaProGlyLysGlyLeuGluTrpIleGlyValIleAlaSerAsnAlaAspThrTyrTyrAlaSerTrpAlaLysGlyArgPh | |
| | TCACTTGGGTCCGCCAGGCTCCAGGGAAGGGGCTGGAGTGGATCGGAGTCATTGCTAGTAATGCTGACACATACTACGCGAGCTGGGCGAAAGGCCGGTT | |
| 101 | ---------!---------!---------!---------!---------!---------!---------!---------!---------!---------! | 200 |
| 1: | eThrIleSerArgAspAsnSerLysAsnThrLeuTyrLeuGlnMetAsnSerLeuArgAlaGluAspThrAlaValTyrTyrCysAlaArgHisProGly | |
| | CACCATCTCCAGAGACAATTCCAAGAACACGCTGTATCTGCAAATGAACAGCCTGAGAGCCGAGGACACGGCCGTATATTACTGTGCCAGACATCCTGGT | |
| 201 | ---------!---------!---------!---------!---------!---------!---------!---------!---------!---------! | 300 |
| 1: | SerAlaGlyTyrGlyTyrGlnTyrTyrTyrAlaMetAspProTrpGlyGlnGlyThrLeuValThrValSerSerAlaSerThrLysGlyProSerValP | |
| | AGTGCTGGTTATGGTTATCAGTATTATTACGCCATGGACCCCTGGGGCCAAGGAACCCTGGTCACCGTCTCCTCAGCCTCCACCAAGGGCCCATCGGTCT | |
| 301 | ---------!---------!---------!---------!---------!---------!---------!---------!---------!---------! | 400 |
| 1: | heProLeuAlaProSerSerLysSerThrSerGlyGlyThrAlaAlaLeuGlyCysLeuValLysAspTyrPheProGluProValThrValSerTrpAs | |
| | TCCCCCTGGCACCCTCCTCCAAGAGCACCTCTGGGGGCACAGCGGCCCTGGGCTGCCTGGTCAAGGACTACTTCCCCGAACCGGTGACGGTGTCGTGGAA | |
| 401 | ---------!---------!---------!---------!---------!---------!---------!---------!---------!---------! | 500 |
| 1: | nSerGlyAlaLeuThrSerGlyValHisThrPheProAlaValLeuGlnSerSerGlyLeuTyrSerLeuSerSerValValThrValProSerSerSer | |
| | CTCAGGCGCCCTGACCAGCGGCGTGCACACCTTCCCGGCTGTCCTACAGTCCTCAGGACTCTACTCCCTCAGCAGCGTGGTGACCGTGCCCTCCAGCAGC | |
| 501 | ---------!---------!---------!---------!---------!---------!---------!---------!---------!---------! | 600 |
| 1: | LeuGlyThrGlnThrTyrIleCysAsnValAsnHisLysProSerAsnThrLysValAspLysLysValGluProLysSerCysAspLysThrHisThrC | |
| | TTGGGCACCCAGACCTACATCTGCAACGTGAATCACAAGCCCAGCAACACCAAGGTGGACAAGAAAGTTGAGCCCAAATCTTGTGACAAAACTCACACAT | |
| 601 | ---------!---------!---------!---------!---------!---------!---------!---------!---------!---------! | 700 |
| 1: | ysProProCysProAlaProGluLeuLeuGlyGlyProSerValPheLeuPheProProLysProLysAspThrLeuMetIleSerArgThrProGluVa | |
| | GCCCACCGTGCCCAGCACCTGAACTCCTGGGGGGACCGTCAGTCTTCCTCTTCCCCCCAAAACCCAAGGACACCCTCATGATCTCCCGGACCCCTGAGGT | |
| 701 | ---------!---------!---------!---------!---------!---------!---------!---------!---------!---------! | 800 |
| 1: | lThrCysValValValAspValSerHisGluAspProGluValLysPheAsnTrpTyrValAspGlyValGluValHisAsnAlaLysThrLysProArg | |
| | CACATGCGTGGTGGTGGACGTGAGCCACGAAGACCCTGAGGTCAAGTTCAACTGGTACGTGGACGGCGTGGAGGTGCATAATGCCAAGACAAAGCCGCGG | |
| 801 | ---------!---------!---------!---------!---------!---------!---------!---------!---------!---------! | 900 |
| 1: | GluGluGlnTyrAsnSerThrTyrArgValValSerValLeuThrValLeuHisGlnAspTrpLeuAsnGlyLysGluTyrLysCysLysValSerAsnL | |
| | GAGGAGCAGTACAACAGCACGTACCGTGTGGTCAGCGTCCTCACCGTCCTGCACCAGGACTGGCTGAATGGCAAGGAGTACAAGTGCAAGGTCTCCAACA | |
| 901 | ---------!---------!---------!---------!---------!---------!---------!---------!---------!---------! | 1000 |
| 1: | ysAlaLeuProAlaProIleGluLysThrIleSerLysAlaLysGlyGlnProArgGluProGlnValTyrThrLeuProProSerArgAspGluLeuTh | |
| | AAGCCCTCCCAGCCCCCATCGAGAAAACCATCTCCAAAGCCAAAGGGCAGCCCCGAGAACCACAGGTGTACACCCTGCCCCCATCCCGGGATGAGCTGAC | |
| 1001 | ---------!---------!---------!---------!---------!---------!---------!---------!---------!---------! | 1100 |
| 1: | rLysAsnGlnValSerLeuThrCysLeuValLysGlyPheTyrProSerAspIleAlaValGluTrpGluSerAsnGlyGlnProGluAsnAsnTyrLys | |
| | CAAGAACCAGGTCAGCCTGACCTGCCTGGTCAAAGGCTTCTATCCCAGCGACATCGCCGTGGAGTGGGAGAGCAATGGGCAGCCGGAGAACAACTACAAG | |
| 1101 | ---------!---------!---------!---------!---------!---------!---------!---------!---------!---------! | 1200 |
| 1: | ThrThrProProValLeuAspSerAspGlySerPhePheLeuTyrSerLysLeuThrValAspLysSerArgTrpGlnGlnGlyAsnValPheSerCysS | |
| | ACCACGCCTCCCGTGCTGGACTCCGACGGCTCCTTCTTCCTCTACAGCAAGCTCACCGTGGACAAGAGCAGGTGGCAGCAGGGGAACGTCTTCTCATGCT | |
| 1201 | ---------!---------!---------!---------!---------!---------!---------!---------!---------!---------! | 1300 |
| 1: | erValMetHisGluAlaLeuHisAsnHisTyrThrGlnLysSerLeuSerLeuSerProGlyLys | |
| | CCGTGATGCATGAGGCTCTGCACAACCACTACACGCAGAAGAGCCTCTCCCTGTCTCCGGGTAAA | |
| 1301 | ---------!---------!---------!---------!---------!---------!----- | 1365 |

6R6E4 SEQ ID No. 57

| | | |
|---|---|---|
| 1: | GluValGlnLeuValGluSerGlyGlyGlyLeuValGlnProGlyGlySerLeuArgLeuSerCysAlaAlaSerGlyTyrThrValLysArgGlnAlaV | |
| | GAGGTGCAGCTGGTGGAGTCTGGGGGAGGCTTGGTACAGCCTGGGGGGTCCCTGAGACTCTCCTGTGCGGCTTCTGGATATACTGTTAAGAGGCAGGCTG | |
| 1 | ---------!---------!---------!---------!---------!---------!---------!---------!---------!---------! | 100 |
| 1: | alThrTrpValArgGlnAlaProGlyLysGlyLeuGluTrpIleGlyValIleAlaSerAsnAlaAspThrTyrTyrAlaSerTrpAlaLysGlyArgPh | |
| | TCACTTGGGTCCGCCAGGCTCCAGGGAAGGGGCTGGAGTGGATCGGAGTCATTGCTAGTAATGCTGACACATACTACGCGAGCTGGGCGAAAGGCCGGTT | |
| 101 | ---------!---------!---------!---------!---------!---------!---------!---------!---------!---------! | 200 |
| 1: | eThrIleSerArgAspAsnSerLysAsnThrLeuTyrLeuGlnMetAsnSerLeuArgAlaGluAspThrAlaValTyrTyrCysAlaArgHisProGly | |
| | CACCATCTCCAGAGACAATTCCAAGAACACGCTGTATCTGCAAATGAACAGCCTGAGAGCCGAGGACACGGCCGTATATTACTGTGCCAGACATCCTGGT | |
| 201 | ---------!---------!---------!---------!---------!---------!---------!---------!---------!---------! | 300 |
| 1: | SerAlaGlyTyrGlyTyrGlnTyrTyrTyrAlaMetAspProTrpGlyGlnGlyThrLeuValThrValSerSerAlaSerThrLysGlyProSerValP | |
| | AGTGCTGGTTATGGTTATCAGTATTATTACGCCATGGACCCCTGGGGCCAAGGAACCCTGGTCACCGTCTCCTCAGCCTCCACCAAGGGCCCATCGGTCT | |
| 301 | ---------!---------!---------!---------!---------!---------!---------!---------!---------!---------! | 400 |
| 1: | heProLeuAlaProSerSerLysSerThrSerGlyGlyThrAlaAlaLeuGlyCysLeuValLysAspTyrPheProGluProValThrValSerTrpAs | |
| | TCCCCCTGGCACCCTCCTCCAAGAGCACCTCTGGGGGCACAGCGGCCCTGGGCTGCCTGGTCAAGGACTACTTCCCCGAACCGGTGACGGTGTCGTGGAA | |
| 401 | ---------!---------!---------!---------!---------!---------!---------!---------!---------!---------! | 500 |
| 1: | nSerGlyAlaLeuThrSerGlyValHisThrPheProAlaValLeuGlnSerSerGlyLeuTyrSerLeuSerSerValValThrValProSerSerSer | |
| | CTCAGGCGCCCTGACCAGCGGCGTGCACACCTTCCCGGCTGTCCTACAGTCCTCAGGACTCTACTCCCTCAGCAGCGTGGTGACCGTGCCCTCCAGCAGC | |
| 501 | ---------!---------!---------!---------!---------!---------!---------!---------!---------!---------! | 600 |
| 1: | LeuGlyThrGlnThrTyrIleCysAsnValAsnHisLysProSerAsnThrLysValAspLysLysValGluProLysSerCysAspLysThrHisThrC | |
| | TTGGGCACCCAGACCTACATCTGCAACGTGAATCACAAGCCCAGCAACACCAAGGTGGACAAGAAAGTTGAGCCCAAATCTTGTGACAAAACTCACACAT | |
| 601 | ---------!---------!---------!---------!---------!---------!---------!---------!---------!---------! | 700 |
| 1: | ysProProCysProAlaProGluLeuLeuGlyGlyProSerValPheLeuPheProProLysProLysAspThrLeuMetIleSerArgThrProGluVa | |
| | GCCCACCGTGCCCAGCACCTGAACTCCTGGGGGGACCGTCAGTCTTCCTCTTCCCCCCAAAACCCAAGGACACCCTCATGATCTCCCGGACCCCTGAGGT | |
| 801 | ---------!---------!---------!---------!---------!---------!---------!---------!---------!---------! | 800 |
| 1: | lThrCysValValValAspValSerHisGluAspProGluValLysPheAsnTrpTyrValAspGlyValGluValHisAsnAlaLysThrLysProArg | |
| | CACATGCGTGGTGGTGGACGTGAGCCACGAAGACCCTGAGGTCAAGTTCAACTGGTACGTGGACGGCGTGGAGGTGCATAATGCCAAGACAAAGCCGCGG | |
| 901 | ---------!---------!---------!---------!---------!---------!---------!---------!---------!---------! | 900 |
| 1: | GluGluGlnTyrAsnSerThrTyrArgValValSerValLeuThrValLeuHisGlnAspTrpLeuAsnGlyLysGluTyrLysCysLysValSerAsnL | |
| | GAGGAGCAGTACAACAGCACGTACCGTGTGGTCAGCGTCCTCACCGTCCTGCACCAGGACTGGCTGAATGGCAAGGAGTACAAGTGCAAGGTCTCCAACA | |
| 901 | ---------!---------!---------!---------!---------!---------!---------!---------!---------!---------! | 1000 |
| 1: | ysAlaLeuProAlaProIleGluLysThrIleSerLysAlaLysGlyGlnProArgGluProGlnValTyrThrLeuProProSerArgAspGluLeuTh | |
| | AAGCCCTCCCAGCCCCCATCGAGAAAACCATCTCCAAAGCCAAAGGGCAGCCCCGAGAACCACAGGTGTACACCCTGCCCCCATCCCGGGATGAGCTGAC | |
| 1001 | ---------!---------!---------!---------!---------!---------!---------!---------!---------!---------! | 1100 |
| 1: | rLysAsnGlnValSerLeuThrCysLeuValLysGlyPheTyrProSerAspIleAlaValGluTrpGluSerAsnGlyGlnProGluAsnAsnTyrLys | |
| | CAAGAACCAGGTCAGCCTGACCTGCCTGGTCAAAGGCTTCTATCCCAGCGACATCGCCGTGGAGTGGGAGAGCAATGGGCAGCCGGAGAACAACTACAAG | |
| 1101 | ---------!---------!---------!---------!---------!---------!---------!---------!---------!---------! | 1200 |
| 1: | ThrThrProProValLeuAspSerAspGlySerPhePheLeuTyrSerLysLeuThrValAspLysSerArgTrpGlnGlnGlyAsnValPheSerCysS | |
| | ACCACGCCTCCCGTGCTGGACTCCGACGGCTCCTTCTTCCTCTACAGCAAGCTCACCGTGGACAAGAGCAGGTGGCAGCAGGGGAACGTCTTCTCATGCT | |
| 1201 | ---------!---------!---------!---------!---------!---------!---------!---------!---------!---------! | 1300 |
| 1: | erValMetHisGluAlaLeuHisAsnHisTyrThrGlnLysSerLeuSerLeuSerProGlyLys | |
| | CCGTGATGCATGAGGCTCTGCACAACCACTACACGCAGAAGAGCCTCTCCCTGTCTCCGGGTAAA | |
| 1301 | ---------!---------!---------!---------!---------!---------!----- | 1365 |

6R6F3 SEQ ID No. 58

| | | |
|---|---|---|
| 1: | GluValGlnLeuValGluSerGlyGlyGlyLeuValGlnProGlyGlySerLeuArgLeuSerCysGluAlaSerGlyIleSerIleSerProAsnAlaV | |
| | GAGGTGCAGCTGGTGGAGTCTGGGGGAGGCTTGGTACAGCCTGGGGGGTCCCTGAGACTCTCCTGTGAGGCTTCTGGAATTAGTATTTCGCCTAATGCTG | |
| 1 | ---------!---------!---------!---------!---------!---------!---------!---------!---------!---------! | 100 |
| 1: | alThrTrpValArgGlnAlaProGlyLysGlyLeuGluTrpIleGlyValIleAlaSerAsnAlaAspThrTyrTyrAlaSerTrpAlaLysGlyArgPh | |
| | TGACTTGGGTCCGCCAGGCTCCAGGGAAGGGGCTGGAGTGGATCGGAGTCATTGCTAGTAATGCTGACACATACTACGCGAGCTGGGCGAAAGGCCGGTT | |
| 101 | ---------!---------!---------!---------!---------!---------!---------!---------!---------!---------! | 200 |
| 1: | eThrIleSerArgAspAsnSerLysAsnThrLeuTyrLeuGlnMetAsnSerLeuArgAlaGluAspThrAlaValTyrTyrCysAlaArgHisProGly | |
| | CACCATCTCCAGAGACAATTCCAAGAACACGCTGTATCTGCAAATGAACAGCCTGAGAGCCGAGGACACGGCCGTATATTACTGTGCCAGACATCCTGGT | |
| 201 | ---------!---------!---------!---------!---------!---------!---------!---------!---------!---------! | 300 |
| 1: | SerAlaGlyTyrGlyTyrGlnTyrTyrTyrAlaMetAspProTrpGlyGlnGlyThrLeuValThrValSerSerAlaSerThrLysGlyProSerValP | |
| | AGTGCTGGTTATGGTTATCAGTATTATTACGCCATGGACCCCTGGGGCCAAGGAACCCTGGTCACCGTCTCCTCAGCCTCCACCAAGGGCCCATCGGTCT | |
| 301 | ---------!---------!---------!---------!---------!---------!---------!---------!---------!---------! | 400 |
| 1: | heProLeuAlaProProSerLysSerThrSerGlyGlyThrAlaAlaLeuGlyCysLeuValLysAspTyrPheProGluProValThrValSerTrpAs | |
| | TCCCCCTGGCACCCCCCTCCAAGAGCACCTCTGGGGGCACAGCGGCCCTGGGCTGCCTGGTCAAGGACTACTTCCCCGAACCGGTGACGGTGTCGTGGAA | |
| 401 | ---------!---------!---------!---------!---------!---------!---------!---------!---------!---------! | 500 |
| 1: | nSerGlyAlaLeuThrSerGlyValHisThrPheProAlaValLeuGlnSerSerGlyLeuTyrSerLeuSerSerValValThrValProSerSerSer | |
| | CTCAGGCGCCCTGACCAGCGGCGTGCACACCTTCCCGGCTGTCCTACAGTCCTCAGGACTCTACTCCCTCAGCAGCGTGGTGACCGTGCCCTCCAGCAGC | |
| 501 | ---------!---------!---------!---------!---------!---------!---------!---------!---------!---------! | 600 |
| 1: | LeuGlyThrGlnThrTyrIleCysAsnValAsnHisLysProSerAsnThrLysValAspLysLysValGluProLysSerCysAspLysThrHisThrC | |
| | TTGGGCACCCAGACCTACATCTGCAACGTGAATCACAAGCCCAGCAACACCAAGGTGGACAAGAAAGTTGAGCCCAAATCTTGTGACAAAACTCACACAT | |
| 601 | ---------!---------!---------!---------!---------!---------!---------!---------!---------!---------! | 700 |
| 1: | ysProProCysProAlaProGluLeuLeuGlyGlyProSerValPheLeuPheProProLysProLysAspThrLeuMetIleSerArgThrProGluVa | |
| | GCCCACCGTGCCCAGCACCTGAACTCCTGGGGGGACCGTCAGTCTTCCTCTTCCCCCCAAAACCCAAGGACACCCTCATGATCTCCCGGACCCCTGAGGT | |
| 701 | ---------!---------!---------!---------!---------!---------!---------!---------!---------!---------! | 800 |
| 1: | lThrCysValValValAspValSerHisGluAspProGluValLysPheAsnTrpTyrValAspGlyValGluValHisAsnAlaLysThrLysProArg | |
| | CACATGCGTGGTGGTGGACGTGAGCCACGAAGACCCTGAGGTCAAGTTCAACTGGTACGTGGACGGCGTGGAGGTGCATAATGCCAAGACAAAGCCGCGG | |
| 801 | ---------!---------!---------!---------!---------!---------!---------!---------!---------!---------! | 900 |
| 1: | GluGluGlnTyrAsnSerThrTyrArgValValSerValLeuThrValLeuHisGlnAspTrpLeuAsnGlyLysGluTyrLysCysLysValSerAsnL | |
| | GAGGAGCAGTACAACAGCACGTACCGTGTGGTCAGCGTCCTCACCGTCCTGCACCAGGACTGGCTGAATGGCAAGGAGTACAAGTGCAAGGTCTCCAACA | |
| 901 | ---------!---------!---------!---------!---------!---------!---------!---------!---------!---------! | 1000 |
| 1: | ysAlaLeuProAlaProIleGluLysThrIleSerLysAlaLysGlyGlnProArgGluProGlnValTyrThrLeuProProSerArgAspGluLeuTh | |
| | AAGCCCTCCCAGCCCCCATCGAGAAAACCATCTCCAAAGCCAAAGGGCAGCCCCGAGAACCACAGGTGTACACCCTGCCCCCATCCCGGGATGAGCTGAC | |
| 1001 | ---------!---------!---------!---------!---------!---------!---------!---------!---------!---------! | 1100 |
| 1: | rLysAsnGlnValSerLeuThrCysLeuValLysGlyPheTyrProSerAspIleAlaValGluTrpGluSerAsnGlyGlnProGluAsnAsnTyrLys | |
| | CAAGAACCAGGTCAGCCTGACCTGCCTGGTCAAAGGCTTCTATCCCAGCGACATCGCCGTGGAGTGGGAGAGCAATGGGCAGCCGGAGAACAACTACAAG | |
| 1101 | ---------!---------!---------!---------!---------!---------!---------!---------!---------!---------! | 1200 |
| 1: | ThrThrProProValLeuAspSerAspGlySerPhePheLeuTyrSerLysLeuThrValAspLysSerArgTrpGlnGlnGlyAsnValPheSerCysS | |
| | ACCACGCCTCCCGTGCTGGACTCCGACGGCTCCTTCTTCCTCTACAGCAAGCTCACCGTGGACAAGAGCAGGTGGCAGCAGGGGAACGTCTTCTCATGCT | |
| 1201 | ---------!---------!---------!---------!---------!---------!---------!---------!---------!---------! | 1300 |
| 1: | erValMetHisGluAlaLeuHisAsnHisTyrThrGlnLysSerLeuSerLeuSerProGlyLys | |
| | CCGTGATGCATGAGGCTCTGCACAACCACTACACGCAGAAGAGCCTCTCCCTGTCTCCGGGTAAA | |
| 1301 | ---------!---------!---------!---------!---------!---------!----- | 1365 |

6R6F7 SEQ ID No. 59

| | | |
|---|---|---|
| 1: | GluValGlnLeuValGluSerGlyGlyGlyLeuValGlnProGlyGlySerLeuArgLeuSerCysGluAlaSerGlyPheThrPheProLeuGlnAlaM | |
| | GAGGTGCAGCTGGTGGAGTCTGGGGGAGGCTTGGTACAGCCTGGGGGGTCCCTGAGACTCTCCTGTGAGGCTTCTGGATTTACTTTTCCTCTTCAGGCTA | |
| 1 | ---------!---------!---------!---------!---------!---------!---------!---------!---------!---------! | 100 |
| 1: | etSerTrpValArgGlnAlaProGlyLysGlyLeuGluTrpIleGlyValIleAlaSerAsnAlaAspThrTyrTyrAlaSerTrpAlaLysGlyArgPh | |
| | TGAGTTGGGTCCGCCAGGCTCCAGGGAAGGGGCTGGAGTGGATCGGAGTCATTGCTAGTAATGCTGACACATACTACGCGAGCTGGGCGAAAGGCCGGTT | |
| 101 | ---------!---------!---------!---------!---------!---------!---------!---------!---------!---------! | 200 |
| 1: | eThrIleSerArgAspAsnSerLysAsnThrLeuTyrLeuGlnMetAsnSerLeuArgAlaGluAspThrAlaValTyrTyrCysAlaArgHisProGly | |
| | CACCATCTCCAGAGACAATTCCAAGAACACGCTGTATCTGCAAATGAACAGCCTGAGAGCCGAGGACACGGCCGTATATTACTGTGCCAGACATCCGGGT | |
| 201 | ---------!---------!---------!---------!---------!---------!---------!---------!---------!---------! | 300 |
| 1: | PheAlaGlyTyrGlyTyrGlyHisTyrTyrAlaMetAspProTrpGlyGlnGlyThrLeuValThrValSerSerAlaSerThrLysGlyProSerValP | |
| | TTTGCTGGTTATGGTTATGGGCATTATTACGCCATGGACCCCTGGGGCCAAGGAACCCTGGTCACCGTCTCCTCAGCCTCCACCAAGGGCCCATCGGTCT | |
| 301 | ---------!---------!---------!---------!---------!---------!---------!---------!---------!---------! | 400 |
| 1: | heProLeuAlaProSerSerLysSerThrSerGlyGlyThrAlaAlaLeuGlyCysLeuValLysAspTyrPheProGluProValThrValSerTrpAs | |
| | TCCCCCTGGCACCCTCCTCCAAGAGCACCTCTGGGGGCACAGCGGCCCTGGGCTGCCTGGTCAAGGACTACTTCCCCGAACCGGTGACGGTGTCGTGGAA | |
| 401 | ---------!---------!---------!---------!---------!---------!---------!---------!---------!---------! | 500 |
| 1: | nSerGlyAlaLeuThrSerGlyValHisThrPheProAlaValLeuGlnSerSerGlyLeuTyrSerLeuSerSerValValThrValProSerSerSer | |
| | CTCAGGCGCCCTGACCAGCGGCGTGCACACCTTCCCGGCTGTCCTACAGTCCTCAGGACTCTACTCCCTCAGCAGCGTGGTGACCGTGCCCTCCAGCAGC | |
| 501 | ---------!---------!---------!---------!---------!---------!---------!---------!---------!---------! | 600 |
| 1: | LeuGlyThrGlnThrTyrIleCysAsnValAsnHisLysProSerAsnThrLysValAspLysLysValGluProLysSerCysAspLysThrHisThrC | |
| | TTGGGCACCCAGACCTACATCTGCAACGTGAATCACAAGCCCAGCAACACCAAGGTGGACAAGAAAGTTGAGCCCAAATCTTGTGACAAAACTCACACAT | |
| 601 | ---------!---------!---------!---------!---------!---------!---------!---------!---------!---------! | 700 |
| 1: | ysProProCysProAlaProGluLeuLeuGlyGlyProSerValPheLeuPheProProLysProLysAspThrLeuMetIleSerArgThrProGluVa | |
| | GCCCACCGTGCCCAGCACCTGAACTCCTGGGGGGACCGTCAGTCTTCCTCTTCCCCCCAAAACCCAAGGACACCCTCATGATCTCCCGGACCCCTGAGGT | |
| 701 | ---------!---------!---------!---------!---------!---------!---------!---------!---------!---------! | 800 |
| 1: | lThrCysValValValAspValSerHisGluAspProGluValLysPheAsnTrpTyrValAspGlyValGluValHisAsnAlaLysThrLysProArg | |
| | CACATGCGTGGTGGTGGACGTGAGCCACGAAGACCCTGAGGTCAAGTTCAACTGGTACGTGGACGGCGTGGAGGTGCATAATGCCAAGACAAAGCCGCGG | |
| 801 | ---------!---------!---------!---------!---------!---------!---------!---------!---------!---------! | 900 |
| 1: | GluGluGlnTyrAsnSerThrTyrArgValValSerValLeuThrValLeuHisGlnAspTrpLeuAsnGlyLysGluTyrLysCysLysValSerAsnL | |
| | GAGGAGCAGTACAACAGCACGTACCGTGTGGTCAGCGTCCTCACCGTCCTGCACCAGGACTGGCTGAATGGCAAGGAGTACAAGTGCAAGGTCTCCAACA | |
| 901 | ---------!---------!---------!---------!---------!---------!---------!---------!---------!---------! | 1000 |
| 1: | ysAlaLeuProAlaProIleGluLysThrIleSerLysAlaLysGlyGlnProArgGluProGlnValTyrThrLeuProProSerArgAspGluLeuTh | |
| | AAGCCCTCCCAGCCCCCATCGAGAAAACCATCTCCAAAGCCAAAGGGCAGCCCCGAGAACCACAGGTGTACACCCTGCCCCCATCCCGGGATGAGCTGAC | |
| 1001 | ---------!---------!---------!---------!---------!---------!---------!---------!---------!---------! | 1100 |
| 1: | rLysAsnGlnValSerLeuThrCysLeuValLysGlyPheTyrProSerAspIleAlaValGluTrpGluSerAsnGlyGlnProGluAsnAsnTyrLys | |
| | CAAGAACCAGGTCAGCCTGACCTGCCTGGTCAAAGGCTTCTATCCCAGCGACATCGCCGTGGAGTGGGAGAGCAATGGGCAGCCGGAGAACAACTACAAG | |
| 1101 | ---------!---------!---------!---------!---------!---------!---------!---------!---------!---------! | 1200 |
| 1: | ThrThrProProValLeuAspSerAspGlySerPhePheLeuTyrSerLysLeuThrValAspLysSerArgTrpGlnGlnGlyAsnValPheSerCysS | |
| | ACCACGCCTCCCGTGCTGGACTCCGACGGCTCCTTCTTCCTCTACAGCAAGCTCACCGTGGACAAGAGCAGGTGGCAGCAGGGGAACGTCTTCTCATGCT | |
| 1201 | ---------!---------!---------!---------!---------!---------!---------!---------!---------!---------! | 1300 |
| 1: | erValMetHisGluAlaLeuHisAsnHisTyrThrGlnLysSerLeuSerLeuSerProGlyLys | |
| | CCGTGATGCATGAGGCTCTGCACAACCACTACACGCAGAAGAGCCTCTCCCTGTCTCCGGGTAAA | |
| 1301 | ---------!---------!---------!---------!---------!---------!----- | 1365 |

6R6F9 SEQ ID No. 60

| | | |
|---|---|---|
| 1: | GluValGlnLeuValGluSerGlyGlyGlyLeuValGlnProGlyGlySerLeuArgLeuSerCysGluAlaSerGlyIleAspLeuSerProGlnAlaM | |
| | GAGGTGCAGCTGGTGGAGTCTGGGGGAGGCTTGGTACAGCCTGGGGGGTCCCTGAGACTCTCCTGTGAGGCTTCTGGAATTGATCTTAGTCCTCAGGCTA | |
| 1 | ---------!---------!---------!---------!---------!---------!---------!---------!---------!---------! | 100 |
| 1: | etThrTrpValArgGlnAlaProGlyLysGlyLeuGluTrpIleGlyValIleAlaSerAsnAlaAspThrTyrTyrAlaSerTrpAlaLysGlyArgPh | |
| | TGACATGGGTCCGCCAGGCTCCAGGGAAGGGGCTGGAGTGGATCGGAGTCATTGCTAGTAATGCTGACACATACTACGCGAGCTGGGCGAAAGGCCGGTT | |
| 101 | ---------!---------!---------!---------!---------!---------!---------!---------!---------!---------! | 200 |
| 1: | eThrIleSerArgAspAsnSerLysAsnThrLeuTyrLeuGlnMetAsnSerLeuArgAlaGluAspThrAlaValTyrTyrCysAlaArgHisProGly | |
| | CACCATCTCCAGAGACAATTCCAAGAACACGCTGTATCTGCAAATGAACAGCCTGAGAGCCGAGGACACGGCCGTATATTACTGTGCCAGACATCCTGGT | |
| 201 | ---------!---------!---------!---------!---------!---------!---------!---------!---------!---------! | 300 |
| 1: | SerAlaGlyTyrGlyTyrGlnTyrTyrTyrAlaMetAspProTrpGlyGlnGlyThrLeuValThrValSerSerAlaSerThrLysGlyProSerValP | |
| | AGTGCTGGTTATGGTTATCAGTATTATTACGCCATGGACCCCTGGGGCCAAGGAACCCTGGTCACCGTCTCCTCAGCCTCCACCAAGGGCCCATCGGTCT | |
| 301 | ---------!---------!---------!---------!---------!---------!---------!---------!---------!---------! | 400 |
| 1: | heProLeuAlaProSerSerLysSerThrSerGlyGlyThrAlaAlaLeuGlyCysLeuValLysAspTyrPheProGluProValThrValSerTrpAs | |
| | TCCCCCTGGCACCCTCCTCCAAGAGCACCTCTGGGGGCACAGCGGCCCTGGGCTGCCTGGTCAAGGACTACTTCCCCGAACCGGTGACGGTGTCGTGGAA | |
| 401 | ---------!---------!---------!---------!---------!---------!---------!---------!---------!---------! | 500 |
| 1: | nSerGlyAlaLeuThrSerGlyValHisThrPheProAlaValLeuGlnSerSerGlyLeuTyrSerLeuSerSerValValThrValProSerSerSer | |
| | CTCAGGCGCCCTGACCAGCGGCGTGCACACCTTCCCGGCTGTCCTACAGTCCTCAGGACTCTACTCCCTCAGCAGCGTGGTGACCGTGCCCTCCAGCAGC | |
| 501 | ---------!---------!---------!---------!---------!---------!---------!---------!---------!---------! | 600 |
| 1: | LeuGlyThrGlnThrTyrIleCysAsnValAsnHisLysProSerAsnThrLysValAspLysLysValGluProLysSerCysAspLysThrHisThrC | |
| | TTGGGCACCCAGACCTACATCTGCAACGTGAATCACAAGCCCAGCAACACCAAGGTGGACAAGAAAGTTGAGCCCAAATCTTGTGACAAAACTCACACAT | |
| 601 | ---------!---------!---------!---------!---------!---------!---------!---------!---------!---------! | 700 |
| 1: | ysProProCysProAlaProGluLeuLeuGlyGlyProSerValPheLeuPheProProLysProLysAspThrLeuMetIleSerArgThrProGluVa | |
| | GCCCACCGTGCCCAGCACCTGAACTCCTGGGGGGACCGTCAGTCTTCCTCTTCCCCCCAAAACCCAAGGACACCCTCATGATCTCCCGGACCCCTGAGGT | |
| 701 | ---------!---------!---------!---------!---------!---------!---------!---------!---------!---------! | 800 |
| 1: | lThrCysValValValAspValSerHisGluAspProGluValLysPheAsnTrpTyrValAspGlyValGluValHisAsnAlaLysThrLysProArg | |
| | CACATGCGTGGTGGTGGACGTGAGCCACGAAGACCCTGAGGTCAAGTTCAACTGGTACGTGGACGGCGTGGAGGTGCATAATGCCAAGACAAAGCCGCGG | |
| 801 | ---------!---------!---------!---------!---------!---------!---------!---------!---------!---------! | 900 |
| 1: | GluGluGlnTyrAsnSerThrTyrArgValValSerValLeuThrValLeuHisGlnAspTrpLeuAsnGlyLysGluTyrLysCysLysValSerAsnL | |
| | GAGGAGCAGTACAACAGCACGTACCGTGTGGTCAGCGTCCTCACCGTCCTGCACCAGGACTGGCTGAATGGCAAGGAGTACAAGTGCAAGGTCTCCAACA | |
| 901 | ---------!---------!---------!---------!---------!---------!---------!---------!---------!---------! | 1000 |
| 1: | ysAlaLeuProAlaProIleGluLysThrIleSerLysAlaLysGlyGlnProArgGluProGlnValTyrThrLeuProProSerArgAspGluLeuTh | |
| | AAGCCCTCCCAGCCCCCATCGAGAAAACCATCTCCAAAGCCAAAGGGCAGCCCCGAGAACCACAGGTGTACACCCTGCCCCCATCCCGGGATGAGCTGAC | |
| 1001 | ---------!---------!---------!---------!---------!---------!---------!---------!---------!---------! | 1100 |
| 1: | rLysAsnGlnValSerLeuThrCysLeuValLysGlyPheTyrProSerAspIleAlaValGluTrpGluSerAsnGlyGlnProGluAsnAsnTyrLys | |
| | CAAGAACCAGGTCAGCCTGACCTGCCTGGTCAAAGGCTTCTATCCCAGCGACATCGCCGTGGAGTGGGAGAGCAATGGGCAGCCGGAGAACAACTACAAG | |
| 1101 | ---------!---------!---------!---------!---------!---------!---------!---------!---------!---------! | 1200 |
| 1: | ThrThrProProValLeuAspSerAspGlySerPhePheLeuTyrSerLysLeuThrValAspLysSerArgTrpGlnGlnGlyAsnValPheSerCysS | |
| | ACCACGCCTCCCGTGCTGGACTCCGACGGCTCCTTCTTCCTCTACAGCAAGCTCACCGTGGACAAGAGCAGGTGGCAGCAGGGGAACGTCTTCTCATGCT | |
| 1201 | ---------!---------!---------!---------!---------!---------!---------!---------!---------!---------! | 1300 |
| 1: | erValMetHisGluAlaLeuHisAsnHisTyrThrGlnLysSerLeuSerLeuSerProGlyLys | |
| | CCGTGATGCATGAGGCTCTGCACAACCACTACACGCAGAAGAGCCTCTCCCTGTCTCCGGGTAAA | |
| 1301 | ---------!---------!---------!---------!---------!---------!----- | 1365 |

6R6F11 SEQ ID No. 61

| | | |
|---|---|---|
| 1: | GluValGlnLeuValGluSerGlyGlyGlyLeuValGlnProGlyGlySerLeuArgLeuSerCysGluAlaSerGlyTyrThrPheAlaSerGlnAlaM | |
| | GAGGTGCAGCTGGTGGAGTCTGGGGGAGGCTTGGTACAGCCTGGGGGGTCCCTGAGACTCTCCTGTGAGGCTTCTGGATATACTTTTGCTTCGCAGGCGA | |
| 1 | ---------!---------!---------!---------!---------!---------!---------!---------!---------!---------! | 100 |
| 1: | etThrTrpValArgGlnAlaProGlyLysGlyLeuGluTrpIleGlyValIleAlaSerAsnAlaAspThrTyrTyrAlaSerTrpAlaLysGlyArgPh | |
| | TGACTTGGGTCCGCCAGGCTCCAGGGAAGGGGCTGGAGTGGATCGGAGTCATTGCTAGTAATGCTGACACATACTACGCGAGCTGGGCGAAAGGCCGGTT | |
| 101 | ---------!---------!---------!---------!---------!---------!---------!---------!---------!---------! | 200 |
| 1: | eThrIleSerArgAspAsnSerLysAsnThrLeuTyrLeuGlnMetAsnSerLeuArgAlaGluAspThrAlaValTyrTyrCysAlaArgHisProGly | |
| | CACCATCTCCAGAGACAATTCCAAGAACACGCTGTATCTGCAAATGAACAGCCTGAGAGCCGAGGACACGGCCGTATATTACTGTGCCAGACATCCGGGT | |
| 201 | ---------!---------!---------!---------!---------!---------!---------!---------!---------!---------! | 300 |
| 1: | PheAlaGlyTyrGlyTyrGlyHisTyrTyrAlaMetAspProTrpGlyGlnGlyThrLeuValThrValSerSerAlaSerThrLysGlyProSerValP | |
| | TTTGCTGGTTATGGTTATGGGCATTATTACGCCATGGACCCCTGGGGCCAAGGAACCCTGGTCACCGTCTCCTCAGCCTCCACCAAGGGCCCATCGGTCT | |
| 301 | ---------!---------!---------!---------!---------!---------!---------!---------!---------!---------! | 400 |
| 1: | heProLeuAlaProSerSerLysSerThrSerGlyGlyThrAlaAlaLeuGlyCysLeuValLysAspTyrPheProGluProValThrValSerTrpAs | |
| | TCCCCCTGGCACCCTCCTCCAAGAGCACCTCTGGGGGCACAGCGGCCCTGGGCTGCCTGGTCAAGGACTACTTCCCCGAACCGGTGACGGTGTCGTGGAA | |
| 401 | ---------!---------!---------!---------!---------!---------!---------!---------!---------!---------! | 500 |
| 1: | nSerGlyAlaLeuThrSerGlyValHisThrPheProAlaValLeuGlnSerSerGlyLeuTyrSerLeuSerSerValValThrValProSerSerSer | |
| | CTCAGGCGCCCTGACCAGCGGCGTGCACACCTTCCCGGCTGTCCTACAGTCCTCAGGACTCTACTCCCTCAGCAGCGTGGTGACCGTGCCCTCCAGCAGC | |
| 501 | ---------!---------!---------!---------!---------!---------!---------!---------!---------!---------! | 600 |
| 1: | LeuGlyThrGlnThrTyrIleCysAsnValAsnHisLysProSerAsnThrLysValAspLysLysValGluProLysSerCysAspLysThrHisThrC | |
| | TTGGGCACCCAGACCTACATCTGCAACGTGAATCACAAGCCCAGCAACACCAAGGTGGACAAGAAAGTTGAGCCCAAATCTTGTGACAAAACTCACACAT | |
| 601 | ---------!---------!---------!---------!---------!---------!---------!---------!---------!---------! | 700 |
| 1: | ysProProCysProAlaProGluLeuLeuGlyGlyProSerValPheLeuPheProProLysProLysAspThrLeuMetIleSerArgThrProGluVa | |
| | GCCCACCGTGCCCAGCACCTGAACTCCTGGGGGGACCGTCAGTCTTCCTCTTCCCCCCAAAACCCAAGGACACCCTCATGATCTCCCGGACCCCTGAGGT | |
| 701 | ---------!---------!---------!---------!---------!---------!---------!---------!---------!---------! | 800 |
| 1: | lThrCysValValValAspValSerHisGluAspProGluValLysPheAsnTrpTyrValAspGlyValGluValHisAsnAlaLysThrLysProArg | |
| | CACATGCGTGGTGGTGGACGTGAGCCACGAAGACCCTGAGGTCAAGTTCAACTGGTACGTGGACGGCGTGGAGGTGCATAATGCCAAGACAAAGCCGCGG | |
| 801 | ---------!---------!---------!---------!---------!---------!---------!---------!---------!---------! | 900 |
| 1: | GluGluGlnTyrAsnSerThrTyrArgValValSerValLeuThrValLeuHisGlnAspTrpLeuAsnGlyLysGluTyrLysCysLysValSerAsnL | |
| | GAGGAGCAGTACAACAGCACGTACCGTGTGGTCAGCGTCCTCACCGTCCTGCACCAGGACTGGCTGAATGGCAAGGAGTACAAGTGCAAGGTCTCCAACA | |
| 901 | ---------!---------!---------!---------!---------!---------!---------!---------!---------!---------! | 1000 |
| 1: | ysAlaLeuProAlaProIleGluLysThrIleSerLysAlaLysGlyGlnProArgGluProGlnValTyrThrLeuProProSerArgAspGluLeuTh | |
| | AAGCCCTCCCAGCCCCCATCGAGAAAACCATCTCCAAAGCCAAAGGGCAGCCCCGAGAACCACAGGTGTACACCCTGCCCCCATCCCGGGATGAGCTGAC | |
| 1001 | ---------!---------!---------!---------!---------!---------!---------!---------!---------!---------! | 1100 |
| 1: | rLysAsnGlnValSerLeuThrCysLeuValLysGlyPheTyrProSerAspIleAlaValGluTrpGluSerAsnGlyGlnProGluAsnAsnTyrLys | |
| | CAAGAACCAGGTCAGCCTGACCTGCCTGGTCAAAGGCTTCTATCCCAGCGACATCGCCGTGGAGTGGGAGAGCAATGGGCAGCCGGAGAACAACTACAAG | |
| 1101 | ---------!---------!---------!---------!---------!---------!---------!---------!---------!---------! | 1200 |
| 1: | ThrThrProProValLeuAspSerAspGlySerPhePheLeuTyrSerLysLeuThrValAspLysSerArgTrpGlnGlnGlyAsnValPheSerCysS | |
| | ACCACGCCTCCCGTGCTGGACTCCGACGGCTCCTTCTTCCTCTACAGCAAGCTCACCGTGGACAAGAGCAGGTGGCAGCAGGGGAACGTCTTCTCATGCT | |
| 1201 | ---------!---------!---------!---------!---------!---------!---------!---------!---------!---------! | 1300 |
| 1: | erValMetHisGluAlaLeuHisAsnHisTyrThrGlnLysSerLeuSerLeuSerProGlyLys | |
| | CCGTGATGCATGAGGCTCTGCACAACCACTACACGCAGAAGAGCCTCTCCCTGTCTCCGGGTAAA | |
| 1301 | ---------!---------!---------!---------!---------!---------!----- | 1365 |

6R6F12 SEQ ID No. 62

| | | |
|---|---|---|
| 1: | GluValGlnLeuValGluSerGlyGlyGlyLeuValGlnProGlyGlySerLeuArgLeuSerCysLysAlaSerGlyPheThrPheSerArgGlnAlaL | |
| | GAGGTGCAGCTGGTGGAGTCTGGGGGAGGCTTGGTACAGCCTGGGGGGTCCCTGAGACTCTCCTGTAAGGCTTCTGGATTTACTTTTAGTCGTCAGGCTC | |
| 1 | ---------!---------!---------!---------!---------!---------!---------!---------!---------!---------! | 100 |
| 1: | euThrTrpValArgGlnAlaProGlyLysGlyLeuGluTrpIleGlyValIleAlaSerAsnAlaAspThrTyrTyrAlaSerTrpAlaLysGlyArgPh | |
| | TCACTTGGGTCCGCCAGGCTCCAGGGAAGGGGCTGGAGTGGATCGGAGTCATTGCTAGTAATGCTGACACATACTACGCGAGCTGGGCGAAAGGCCGGTT | |
| 101 | ---------!---------!---------!---------!---------!---------!---------!---------!---------!---------! | 200 |
| 1: | eThrIleSerArgAspAsnSerLysAsnThrLeuTyrLeuGlnMetAsnSerLeuArgAlaGluAspThrAlaValTyrTyrCysAlaArgHisProGly | |
| | CACCATCTCCAGAGACAATTCCAAGAACACGCTGTATCTGCAAATGAACAGCCTGAGAGCCGAGGACACGGCCGTATATTACTGTGCCAGACATCCTGGT | |
| 201 | ---------!---------!---------!---------!---------!---------!---------!---------!---------!---------! | 300 |
| 1: | SerAlaGlyTyrGlyTyrGlnTyrTyrTyrAlaMetAspProTrpGlyGlnGlyThrLeuValThrValSerSerAlaSerThrLysGlyProSerValP | |
| | AGTGCTGGTTATGGTTATCAGTATTATTACGCCATGGACCCCTGGGGCCAAGGAACCCTGGTCACCGTCTCCTCAGCCTCCACCAAGGGCCCATCGGTCT | |
| 301 | ---------!---------!---------!---------!---------!---------!---------!---------!---------!---------! | 400 |
| 1: | heProLeuAlaProSerSerLysSerThrSerGlyGlyThrAlaAlaLeuGlyCysLeuValLysAspTyrPheProGluProValThrValSerTrpAs | |
| | TCCCCCTGGCACCCTCCTCCAAGAGCACCTCTGGGGGCACAGCGGCCCTGGGCTGCCTGGTCAAGGACTACTTCCCCGAACCGGTGACGGTGTCGTGGAA | |
| 401 | ---------!---------!---------!---------!---------!---------!---------!---------!---------!---------! | 500 |
| 1: | nSerGlyAlaLeuThrSerGlyValHisThrPheProAlaValLeuGlnSerSerGlyLeuTyrSerLeuSerSerValValThrValProSerSerSer | |
| | CTCAGGCGCCCTGACCAGCGGCGTGCACACCTTCCCGGCTGTCCTACAGTCCTCAGGACTCTACTCCCTCAGCAGCGTGGTGACCGTGCCCTCCAGCAGC | |
| 501 | ---------!---------!---------!---------!---------!---------!---------!---------!---------!---------! | 600 |
| 1: | LeuGlyThrGlnThrTyrIleCysAsnValAsnHisLysProSerAsnThrLysValAspLysLysValGluProLysSerCysAspLysThrHisThrC | |
| | TTGGGCACCCAGACCTACATCTGCAACGTGAATCACAAGCCCAGCAACACCAAGGTGGACAAGAAAGTTGAGCCCAAATCTTGTGACAAAACTCACACAT | |
| 601 | ---------!---------!---------!---------!---------!---------!---------!---------!---------!---------! | 700 |
| 1: | ysProProCysProAlaProGluLeuLeuGlyGlyProSerValPheLeuPheProProLysProLysAspThrLeuMeIleSerArgThrProGluVa | |
| | GCCCACCGTGCCCAGCACCTGAACTCCTGGGGGGACCGTCAGTCTTCCTCTTCCCCCCAAAACCCAAGGACACCCTCATGATCTCCCGGACCCCTGAGGT | |
| 701 | ---------!---------!---------!---------!---------!---------!---------!---------!---------!---------! | 800 |
| 1: | lThrCysValValValAspValSerHisGluAspProGluValLysPheAsnTrpTyrValAspGlyValGluValHisAsnAlaLysThrLysProArg | |
| | CACATGCGTGGTGGTGGACGTGAGCCACGAAGACCCTGAGGTCAAGTTCAACTGGTACGTGGACGGCGTGGAGGTGCATAATGCCAAGACAAAGCCGCGG | |
| 801 | ---------!---------!---------!---------!---------!---------!---------!---------!---------!---------! | 900 |
| 1: | GluGluGlnTyrAsnSerThrTyrArgValValSerValLeuThrValLeuHisGlnAspTrpLeuAsnGlyLysGluTyrLysCysLysValSerAsnL | |
| | GAGGAGCAGTACAACAGCACGTACCGTGTGGTCAGCGTCCTCACCGTCCTGCACCAGGACTGGCTGAATGGCAAGGAGTACAAGTGCAAGGTCTCCAACA | |
| 901 | ---------!---------!---------!---------!---------!---------!---------!---------!---------!---------! | 1000 |
| 1: | ysAlaLeuProAlaProIleGluLysThrIleSerLysAlaLysGlyGlnProArgGluProGlnValTyrThrLeuProProSerArgAspGluLeuTh | |
| | AAGCCCTCCCAGCCCCCATCGAGAAAACCATCTCCAAAGCCAAAGGGCAGCCCCGAGAACCACAGGTGTACACCCTGCCCCCATCCCGGGATGAGCTGAC | |
| 1001 | ---------!---------!---------!---------!---------!---------!---------!---------!---------!---------! | 1100 |
| 1: | rLysAsnGlnValSerLeuThrCysLeuValLysGlyPheTyrProSerAspIleAlaValGluTrpGluSerAsnGlyGlnProGluAsnAsnTyrLys | |
| | CAAGAACCAGGTCAGCCTGACCTGCCTGGTCAAAGGCTTCTATCCCAGCGACATCGCCGTGGAGTGGGAGAGCAATGGGCAGCCGGAGAACAACTACAAG | |
| 1101 | ---------!---------!---------!---------!---------!---------!---------!---------!---------!---------! | 1200 |
| 1: | ThrThrProProValLeuAspSerAspGlySerPhePheLeuTyrSerLysLeuThrValAspLysSerArgTrpGlnGlnGlyAsnValPheSerCysS | |
| | ACCACGCCTCCCGTGCTGGACTCCGACGGCTCCTTCTTCCTCTACAGCAAGCTCACCGTGGACAAGAGCAGGTGGCAGCAGGGGAACGTCTTCTCATGCT | |
| 1201 | ---------!---------!---------!---------!---------!---------!---------!---------!---------!---------! | 1300 |
| 1: | erValMetHisGluAlaLeuHisAsnHisTyrThrGlnLysSerLeuSerLeuSerProGlyLys | |
| | CCGTGATGCATGAGGCTCTGCACAACCACTACACGCAGAAGAGCCTCTCCCTGTCTCCGGGTAAA | |
| 1301 | ---------!---------!---------!---------!---------!---------!----- | 1365 |

3R6D11Q SEQ ID No. 63

| | | |
|---|---|---|
| 1: | GluValGlnLeuValGluSerGlyGlyGlyLeuValGlnLeuGlyGlySerLeuArgLeuSerCysLysAlaSerGlyTyrSerLeuAlaProGlnAlaM | |
| | GAGGTGCAGCTGGTGGAGTCTGGGGGAGGCTTGGTACAGCTTGGGGGGTCCCTGAGACTCTCCTGTAAGGCTTCTGGATATAGTCTTGCGCCGCAGGCGA | |
| 1 | ---------!---------!---------!---------!---------!---------!---------!---------!---------!---------! | 100 |
| 1: | etThrTrpValArgGlnAlaProGlyLysGlyLeuGluTrpIleGlyValIleAlaSerAsnAlaAspThrTyrTyrAlaSerTrpAlaLysGlyArgPh | |
| | TGACTTGGGTCCGCCAGGCTCCAGGGAAGGGGCTGGAGTGGATCGGAGTCATTGCTAGTAATGCTGACACATACTACGCGAGCTGGGCGAAAGGCCGGTT | |
| 101 | ---------!---------!---------!---------!---------!---------!---------!---------!---------!---------! | 200 |
| 1: | eThrIleSerArgAspAsnSerLysAsnThrLeuTyrLeuGlnMetAsnSerLeuArgAlaGluAspThrAlaValTyrTyrCysAlaArgHisProGly | |
| | CACCATCTCCAGAGACAATTCCAAGAACACGCTGTATCTGCAAATGAACAGCCTGAGAGCCGAGGACACGGCCGTATATTACTGTGCCAGACATCCGGGT | |
| 201 | ---------!---------!---------!---------!---------!---------!---------!---------!---------!---------! | 300 |
| 1: | PheAlaGlyTyrGlyTyrGlyHisTyrTyrAlaMetAspProTrpGlyGlnGlyThrLeuValThrValSerSerAlaSerThrLysGlyProSerValP | |
| | TTTGCTGGTTATGGTTATGGGCATTATTACGCCATGGACCCCTGGGGCCAAGGAACCCTGGTCACCGTCTCCTCAGCCTCCACCAAGGGCCCATCGGTCT | |
| 301 | ---------!---------!---------!---------!---------!---------!---------!---------!---------!---------! | 400 |
| 1: | heProLeuAlaProSerSerLysSerThrSerGlyGlyThrAlaAlaLeuGlyCysLeuValLysAspTyrPheProGluProValThrValSerTrpAs | |
| | TCCCCCTGGCACCCTCCTCCAAGAGCACCTCTGGGGGCACAGCGGCCCTGGGCTGCCTGGTCAAGGACTACTTCCCCGAACCGGTGACGGTGTCGTGGAA | |
| 401 | ---------!---------!---------!---------!---------!---------!---------!---------!---------!---------! | 500 |
| 1: | nSerGlyAlaLeuThrSerGlyValHisThrPheProAlaValLeuGlnSerSerGlyLeuTyrSerLeuSerSerValValThrValProSerSerSer | |
| | CTCAGGCGCCCTGACCAGCGGCGTGCACACCTTCCCGGCTGTCCTACAGTCCTCAGGACTCTACTCCCTCAGCAGCGTGGTGACCGTGCCCTCCAGCAGC | |
| 501 | ---------!---------!---------!---------!---------!---------!---------!---------!---------!---------! | 600 |
| 1: | LeuGlyThrGlnThrTyrIleCysAsnValAsnHisLysProSerAsnThrLysValAspLysLysValGluProLysSerCysAspLysThrHisThrC | |
| | TTGGGCACCCAGACCTACATCTGCAACGTGAATCACAAGCCCAGCAACACCAAGGTGGACAAGAAAGTTGAGCCCAAATCTTGTGACAAAACTCACACAT | |
| 601 | ---------!---------!---------!---------!---------!---------!---------!---------!---------!---------! | 700 |
| 1: | ysProProCysProAlaProGluLeuLeuGlyGlyProSerValPheLeuPheProProLysProLysAspThrLeuMetIleSerArgThrProGluVa | |
| | GCCCACCGTGCCCAGCACCTGAACTCCTGGGGGGACCGTCAGTCTTCCTCTTCCCCCCAAAACCCAAGGACACCCTCATGATCTCCCGGACCCCTGAGGT | |
| 701 | ---------!---------!---------!---------!---------!---------!---------!---------!---------!---------! | 800 |
| 1: | lThrCysValValValAspValSerHisGluAspProGluValLysPheAsnTrpTyrValAspGlyValGluValHisAsnAlaLysThrLysProArg | |
| | CACATGCGTGGTGGTGGACGTGAGCCACGAAGACCCTGAGGTCAAGTTCAACTGGTACGTGGACGGCGTGGAGGTGCATAATGCCAAGACAAAGCCGCGG | |
| 801 | ---------!---------!---------!---------!---------!---------!---------!---------!---------!---------! | 900 |
| 1: | GluGluGlnTyrAsnSerThrTyrArgValValSerValLeuThrValLeuHisGlnAspTrpLeuAsnGlyLysGluTyrLysCysLysValSerAsnL | |
| | GAGGAGCAGTACAACAGCACGTACCGTGTGGTCAGCGTCCTCACCGTCCTGCACCAGGACTGGCTGAATGGCAAGGAGTACAAGTGCAAGGTCTCCAACA | |
| 901 | ---------!---------!---------!---------!---------!---------!---------!---------!---------!---------! | 1000 |
| 1: | ysAlaLeuProAlaProIleGluLysThrIleSerLysAlaLysGlyGlnProArgGluProGlnValTyrThrLeuProProSerArgAspGluLeuTh | |
| | AAGCCCTCCCAGCCCCCATCGAGAAAACCATCTCCAAAGCCAAAGGGCAGCCCCGAGAACCACAGGTGTACACCCTGCCCCCATCCCGGGATGAGCTGAC | |
| 1001 | ---------!---------!---------!---------!---------!---------!---------!---------!---------!---------! | 1100 |
| 1: | rLysAsnGlnValSerLeuThrCysLeuValLysGlyPheTyrProSerAspIleAlaValGluTrpGluSerAsnGlyGlnProGluAsnAsnTyrLys | |
| | CAAGAACCAGGTCAGCCTGACCTGCCTGGTCAAAGGCTTCTATCCCAGCGACATCGCCGTGGAGTGGGAGAGCAATGGGCAGCCGGAGAACAACTACAAG | |
| 1101 | ---------!---------!---------!---------!---------!---------!---------!---------!---------!---------! | 1200 |
| 1: | ThrThrProProValLeuAspSerAspGlySerPhePheLeuTyrSerLysLeuThrValAspLysSerArgTrpGlnGlnGlyAsnValPheSerCysS | |
| | ACCACGCCTCCCGTGCTGGACTCCGACGGCTCCTTCTTCCTCTACAGCAAGCTCACCGTGGACAAGAGCAGGTGGCAGCAGGGGAACGTCTTCTCATGCT | |
| 1201 | ---------!---------!---------!---------!---------!---------!---------!---------!---------!---------! | 1300 |
| 1: | erValMetHisGluAlaLeuHisAsnHisTyrThrGlnLysSerLeuSerLeuSerProGlyLys | |
| | CCGTGATGCATGAGGCTCTGCACAACCACTACACGCAGAAGAGCCTCTCCCTGTCTCCGGGTAAA | |
| 1301 | ---------!---------!---------!---------!---------!---------!----- | 1365 |

6R6E6Q SEQ ID No. 64

| | | |
|---|---|---|
| 1: | GluValGlnLeuValGluSerGlyGlyGlyLeuValGlnProGlyGlySerLeuArgLeuSerCysLysAlaSerGlyTyrSerLeuAlaProGlnAlaM | |
| | GAGGTGCAGCTGGTGGAGTCTGGGGGAGGCTTGGTACAGCCTGGGGGGTCCCTGAGACTCTCCTGTAAGGCTTCTGGATATAGTCTTGCGCCGCAGGCGA | |
| 1 | ---------!---------!---------!---------!---------!---------!---------!---------!---------!---------! | 100 |
| 1: | etThrTrpValArgGlnAlaProGlyLysGlyLeuGluTrpIleGlyValIleAlaSerAsnAlaAspThrTyrTyrAlaSerTrpAlaLysGlyArgPh | |
| | TGACTTGGGTCCGCCAGGCTCCAGGGAAGGGGCTGGAGTGGATCGGAGTCATTGCTAGTAATGCTGACACATACTACGCGAGCTGGGCGAAAGGCCGATT | |
| 101 | ---------!---------!---------!---------!---------!---------!---------!---------!---------!---------! | 200 |
| 1: | eThrIleSerArgAspAsnSerLysAsnThrLeuTyrLeuGlnMetAsnSerLeuArgAlaGluAspThrAlaValTyrTyrCysAlaArgHisProGly | |
| | CACCATCTCCAGAGACAATTCCAAGAACACGCTGTATCTGCAAATGAACAGCCTGAGAGCCGAGGACACGGCCGTATATTACTGTGCCAGACATCCTGGT | |
| 201 | ---------!---------!---------!---------!---------!---------!---------!---------!---------!---------! | 300 |
| 1: | SerAlaGlyTyrGlyTyrGlnTyrTyrTyrAlaMetAspProTrpGlyGlnGlyThrLeuValThrValSerSerAlaSerThrLysGlyProSerValP | |
| | AGTGCTGGTTATGGTTATCAGTATTATTACGCCATGGACCCCTGGGGCCAAGGAACCCTGGTCACCGTCTCCTCAGCCTCCACCAAGGGCCCATCGGTCT | |
| 301 | ---------!---------!---------!---------!---------!---------!---------!---------!---------!---------! | 400 |
| 1: | heProLeuAlaProSerSerLysSerThrSerGlyGlyThrAlaAlaLeuGlyCysLeuValLysAspTyrPheProGluProValThrValSerTrpAs | |
| | TCCCCCTGGCACCCTCCTCCAAGAGCACCTCTGGGGGCACAGCGGCCCTGGGCTGCCTGGTCAAGGACTACTTCCCCGAACCGGTGACGGTGTCGTGGAA | |
| 401 | ---------!---------!---------!---------!---------!---------!---------!---------!---------!---------! | 500 |
| 1: | nSerGlyAlaLeuThrSerGlyValHisThrPheProAlaValLeuGlnSerSerGlyLeuTyrSerLeuSerSerValValThrValProSerSerSer | |
| | CTCAGGCGCCCTGACCAGCGGCGTGCACACCTTCCCGGCTGTCCTACAGTCCTCAGGACTCTACTCCCTCAGCAGCGTGGTGACCGTGCCCTCCAGCAGC | |
| 501 | ---------!---------!---------!---------!---------!---------!---------!---------!---------!---------! | 600 |
| 1: | LeuGlyThrGlnThrTyrIleCysAsnValAsnHisLysProSerAsnThrLysValAspLysLysValGluProLysSerCysAspLysThrHisThrC | |
| | TTGGGCACCCAGACCTACATCTGCAACGTGAATCACAAGCCCAGCAACACCAAGGTGGACAAGAAAGTTGAGCCCAAATCTTGTGACAAAACTCACACAT | |
| 601 | ---------!---------!---------!---------!---------!---------!---------!---------!---------!---------! | 700 |
| 1: | ysProProCysProAlaProGluLeuLeuGlyGlyProSerValPheLeuPheProProLysProLysAspThrLeuMetIleSerArgThrProGluVa | |
| | GCCCACCGTGCCCAGCACCTGAACTCCTGGGGGGACCGTCAGTCTTCCTCTTCCCCCCAAAACCCAAGGACACCCTCATGATCTCCCGGACCCCTGAGGT | |
| 701 | ---------!---------!---------!---------!---------!---------!---------!---------!---------!---------! | 800 |
| 1: | lThrCysValValValAspValSerHisGluAspProGluValLysPheAsnTrpTyrValAspGlyValGluValHisAsnAlaLysThrLysProArg | |
| | CACATGCGTGGTGGTGGACGTGAGCCACGAAGACCCTGAGGTCAAGTTCAACTGGTACGTGGACGGCGTGGAGGTGCATAATGCCAAGACAAAGCCGCGG | |
| 801 | ---------!---------!---------!---------!---------!---------!---------!---------!---------!---------! | 900 |
| 1: | GluGluGlnTyrAsnSerThrTyrArgValValSerValLeuThrValLeuHisGlnAspTrpLeuAsnGlyLysGluTyrLysCysLysValSerAsnL | |
| | GAGGAGCAGTACAACAGCACGTACCGTGTGGTCAGCGTCCTCACCGTCCTGCACCAGGACTGGCTGAATGGCAAGGAGTACAAGTGCAAGGTCTCCAACA | |
| 901 | ---------!---------!---------!---------!---------!---------!---------!---------!---------!---------! | 1000 |
| 1: | ysAlaLeuProAlaProIleGluLysThrIleSerLysAlaLysGlyGlnProArgGluProGlnValTyrThrLeuProProSerArgAspGluLeuTh | |
| | AAGCCCTCCCAGCCCCCATCGAGAAAACCATCTCCAAAGCCAAAGGGCAGCCCCGAGAACCACAGGTGTACACCCTGCCCCCATCCCGGGATGAGCTGAC | |
| 1001 | ---------!---------!---------!---------!---------!---------!---------!---------!---------!---------! | 1100 |
| 1: | rLysAsnGlnValSerLeuThrCysLeuValLysGlyPheTyrProSerAspIleAlaValGluTrpGluSerAsnGlyGlnProGluAsnAsnTyrLys | |
| | CAAGAACCAGGTCAGCCTGACCTGCCTGGTCAAAGGCTTCTATCCCAGCGACATCGCCGTGGAGTGGGAGAGCAATGGGCAGCCGGAGAACAACTACAAG | |
| 1101 | ---------!---------!---------!---------!---------!---------!---------!---------!---------!---------! | 1200 |
| 1: | ThrThrProProValLeuAspSerAspGlySerPhePheLeuTyrSerLysLeuThrValAspLysSerArgTrpGlnGlnGlyAsnValPheSerCysS | |
| | ACCACGCCTCCCGTGCTGGACTCCGACGGCTCCTTCTTCCTCTACAGCAAGCTCACCGTGGACAAGAGCAGGTGGCAGCAGGGGAACGTCTTCTCATGCT | |
| 1201 | ---------!---------!---------!---------!---------!---------!---------!---------!---------!---------! | 1300 |
| 1: | erValMetHisGluAlaLeuHisAsnHisTyrThrGlnLysSerLeuSerLeuSerProGlyLys | |
| | CCGTGATGCATGAGGCTCTGCACAACCACTACACGCAGAAGAGCCTCTCCCTGTCTCCGGGTAAA | |
| 1301 | ---------!---------!---------!---------!---------!---------!----- | 1365 |

6R8AE11 SEQ ID No. 65

| | | |
|---|---|---|
| 1: | GluValGlnLeuValGluSerGlyGlyGlyLeuValGlnProGlyGlySerLeuArgLeuSerCysLysAlaSerGlyPheThrPheSerArgGlnAlaL | |
| | GAGGTGCAGCTGGTGGAGTCTGGGGGAGGCTTGGTACAGCCTGGGGGGTCCCTGAGACTCTCCTGTAAGGCTTCTGGATTTACTTTTAGTCGTCAGGCTC | |
| 1 | ---------!---------!---------!---------!---------!---------!---------!---------!---------!---------! | 100 |
| 1: | euThrTrpValArgGlnAlaProGlyLysGlyLeuGluTrpIleGlyValIleAlaSerAsnAlaAspThrTyrTyrAlaSerTrpAlaLysGlyArgPh | |
| | TCACTTGGGTCCGCCAGGCTCCAGGGAAGGGGCTGGAGTGGATCGGAGTCATTGCTAGTAATGCTGACACATACTACGCGAGCTGGGCGAAAGGCCGGTT | |
| 101 | ---------!---------!---------!---------!---------!---------!---------!---------!---------!---------! | 200 |
| 1: | eThrIleSerArgAspAsnSerLysAsnThrLeuTyrLeuGlnMetAsnSerLeuArgAlaGluAspThrAlaValTyrTyrCysAlaArgHisProGly | |
| | CACCATCTCCAGAGACAATTCCAAGAACACGCTGTATCTGCAAATGAACAGCCTGAGAGCCGAGGACACGGCCGTATATTACTGTGCCAGACATCCTGGT | |
| 201 | ---------!---------!---------!---------!---------!---------!---------!---------!---------!---------! | 300 |
| 1: | SerAlaGlyTyrGlyTyrGlnTyrTyrTyrAlaMetAspProTrpGlyGlnGlyThrLeuValThrValSerSerAlaSerThrLysGlyProSerValP | |
| | AGTGCTGGTTATGGTTATCAGTATTATTACGCCATGGACCCCTGGGGCCAAGGAACCCTGGTCACCGTCTCCTCAGCCTCCACCAAGGGCCCATCGGTCT | |
| 301 | ---------!---------!---------!---------!---------!---------!---------!---------!---------!---------! | 400 |
| 1: | heProLeuAlaProSerSerLysSerThrSerGlyGlyThrAlaAlaLeuGlyCysLeuValLysAspTyrPheProGluProValThrValSerTrpAs | |
| | TCCCCCTGGCACCCTCCTCCAAGAGCACCTCTGGGGGCACAGCGGCCCTGGGCTGCCTGGTCAAGGACTACTTCCCCGAACCGGTGACGGTGTCGTGGAA | |
| 401 | ---------!---------!---------!---------!---------!---------!---------!---------!---------!---------! | 500 |
| 1: | nSerGlyAlaLeuThrSerGlyValHisThrPheProAlaValLeuGlnSerSerGlyLeuTyrSerLeuSerSerValValThrValProSerSerSer | |
| | CTCAGGCGCCCTGACCAGCGGCGTGCACACCTTCCCGGCTGTCCTACAGTCCTCAGGACTCTACTCCCTCAGCAGCGTGGTGACCGTGCCCTCCAGCAGC | |
| 501 | ---------!---------!---------!---------!---------!---------!---------!---------!---------!---------! | 600 |
| 1: | LeuGlyThrGlnThrTyrIleCysAsnValAsnHisLysProSerAsnThrLysValAspLysLysValGluProLysSerCysAspLysThrHisThrC | |
| | TTGGGCACCCAGACCTACATCTGCAACGTGAATCACAAGCCCAGCAACACCAAGGTGGACAAGAAAGTTGAGCCCAAATCTTGTGACAAAACTCACACAT | |
| 601 | ---------!---------!---------!---------!---------!---------!---------!---------!---------!---------! | 700 |
| 1: | ysProProCysProAlaProGluLeuLeuGlyGlyProSerValPheLeuPheProProLysProLysAspThrLeuMetIleSerArgThrProGluVa | |
| | GCCCACCGTGCCCAGCACCTGAACTCCTGGGGGGACCGTCAGTCTTCCTCTTCCCCCCAAAACCCAAGGACACCCTCATGATCTCCCGGACCCCTGAGGT | |
| 701 | ---------!---------!---------!---------!---------!---------!---------!---------!---------!---------! | 800 |
| 1: | LThrCysValValValAspValSerHisGluAspProGluValLysPheAsnTrpTyrValAspGlyValGluValHisAsnAlaLysThrLysProArg | |
| | CACATGCGTGGTGGTGGACGTGAGCCACGAAGACCCTGAGGTCAAGTTCAACTGGTACGTGGACGGCGTGGAGGTGCATAATGCCAAGACAAAGCCGCGG | |
| 801 | ---------!---------!---------!---------!---------!---------!---------!---------!---------!---------! | 900 |
| 1: | GluGluGlnTyrAsnSerThrTyrArgValValSerValLeuThrValLeuHisGlnAspTrpLeuAsnGlyLysGluTyrLysCysLysValSerAsnL | |
| | GAGGAGCAGTACAACAGCACGTACCGTGTGGTCAGCGTCCTCACCGTCCTGCACCAGGACTGGCTGAATGGCAAGGAGTACAAGTGCAAGGTCTCCAACA | |
| 901 | ---------!---------!---------!---------!---------!---------!---------!---------!---------!---------! | 1000 |
| 1: | ysAlaLeuProAlaProIleGluLysThrIleSerLysAlaLysGlyGlnProArgGluProGlnValTyrThrLeuProProSerArgAspGluLeuTh | |
| | AAGCCCTCCCAGCCCCCATCGAGAAAACCATCTCCAAAGCCAAAGGGCAGCCCCGAGAACCACAGGTGTACACCCTGCCCCCATCCCGGGATGAGCTGAC | |
| 1001 | ---------!---------!---------!---------!---------!---------!---------!---------!---------!---------! | 1100 |
| 1: | rLysAsnGlnValSerLeuThrCysLeuValLysGlyPheTyrProSerAspIleAlaValGluTrpGluSerAsnGlyGlnProGluAsnAsnTyrLys | |
| | CAAGAACCAGGTCAGCCTGACCTGCCTGGTCAAAGGCTTCTATCCCAGCGACATCGCCGTGGAGTGGGAGAGCAATGGGCAGCCGGAGAACAACTACAAG | |
| 1101 | ---------!---------!---------!---------!---------!---------!---------!---------!---------!---------! | 1200 |
| 1: | ThrThrProProValLeuAspSerAspGlySerPhePheLeuTyrSerLysLeuThrValAspLysSerArgTrpGlnGlnGlyAsnValPheSerCysS | |
| | ACCACGCCTCCCGTGCTGGACTCCGACGGCTCCTTCTTCCTCTACAGCAAGCTCACCGTGGACAAGAGCAGGTGGCAGCAGGGGAACGTCTTCTCATGCT | |
| 1201 | ---------!---------!---------!---------!---------!---------!---------!---------!---------!---------! | 1300 |
| 1: | erValMetHisGluAlaLeuHisAsnHisTyrThrGlnLysSerLeuSerLeuSerProGlyLys | |
| | CCGTGATGCATGAGGCTCTGCACAACCACTACACGCAGAAGAGCCTCTCCCTGTCTCCGGGTAAA | |
| 1301 | ---------!---------!---------!---------!---------!---------!----- | 1365 |

6R8AF2 SEQ ID No. 66

| | | |
|---|---|---|
| 1: | GluValGlnLeuValGluSerGlyGlyGlyLeuValGlnProGlyGlySerLeuArgLeuSerCysLysAlaSerGlyTyrSerLeuSerSerGlnAlaL | |
| | GAGGTGCAGCTGGTGGAGTCTGGGGGAGGCTTGGTACAGCCTGGGGGGTCCCTGAGACTCTCCTGTAAGGCTTCTGGATATAGTCTTAGTAGTCAGGCTC | |
| 1 | ---------!---------!---------!---------!---------!---------!---------!---------!---------!---------! | 100 |
| 1: | euThrTrpValArgGlnAlaProGlyLysGlyLeuGluTrpIleGlyValIleAlaSerAsnAlaAspThrTyrTyrAlaSerTrpAlaLysGlyArgPh | |
| | TGACTTGGGTCCGCCAGGCTCCAGGGAAGGGGCTGGAGTGGATCGGAGTCATTGCTAGTAATGCTGACACATACTACGCGAGCTGGGCGAAAGGCCGGTT | |
| 101 | ---------!---------!---------!---------!---------!---------!---------!---------!---------!---------! | 200 |
| 1: | eThrIleSerArgAspAsnSerLysAsnThrLeuTyrLeuGlnMetAsnSerLeuArgAlaGluAspThrAlaValTyrTyrCysAlaArgHisProGly | |
| | CACCATCTCCAGAGACAATTCCAAGAACACGCTGTATCTGCAAATGAACAGCCTGAGAGCCGAGGACACGGCCGTATATTACTGTGCCAGACATCCTGGT | |
| 201 | ---------!---------!---------!---------!---------!---------!---------!---------!---------!---------! | 300 |
| 1: | SerAlaGlyTyrGlyTyrGlnTyrTyrTyrAlaMetAspProTrpGlyGlnGlyThrLeuValThrValSerSerAlaSerThrLysGlyProSerValP | |
| | AGTGCTGGTTATGGTTATCAGTATTATTACGCCATGGACCCCTGGGGCCAAGGAACCCTGGTCACCGTCTCCTCAGCCTCCACCAAGGGCCCATCGGTCT | |
| 301 | ---------!---------!---------!---------!---------!---------!---------!---------!---------!---------! | 400 |
| 1: | heProLeuAlaProSerSerLysSerThrSerGlyGlyThrAlaAlaLeuGlyCysLeuValLysAspTyrPheProGluProValThrValSerTrpAs | |
| | TCCCCCTGGCACCCTCCTCCAAGAGCACCTCTGGGGGCACAGCGGCCCTGGGCTGCCTGGTCAAGGACTACTTCCCCGAACCGGTGACGGTGTCGTGGAA | |
| 401 | ---------!---------!---------!---------!---------!---------!---------!---------!---------!---------! | 500 |
| 1: | nSerGlyAlaLeuThrSerGlyValHisThrPheProAlaValLeuGlnSerSerGlyLeuTyrSerLeuSerSerValValThrValProSerSerSer | |
| | CTCAGGCGCCCTGACCAGCGGCGTGCACACCTTCCCGGCTGTCCTACAGTCCTCAGGACTCTACTCCCTCAGCAGCGTGGTGACCGTGCCCTCCAGCAGC | |
| 501 | ---------!---------!---------!---------!---------!---------!---------!---------!---------!---------! | 600 |
| 1: | LeuGlyThrGlnThrTyrIleCysAsnValAsnHisLysProSerAsnThrLysValAspLysLysValGluProLysSerCysAspLysThrHisThrC | |
| | TTGGGCACCCAGACCTACATCTGCAACGTGAATCACAAGCCCAGCAACACCAAGGTGGACAAGAAAGTTGAGCCCAAATCTTGTGACAAAACTCACACAT | |
| 601 | ---------!---------!---------!---------!---------!---------!---------!---------!---------!---------! | 700 |
| 1: | ysProProCysProAlaProGluLeuLeuGlyGlyProSerValPheLeuPheProProLysProLysAspThrLeuMetIleSerArgThrProGluVa | |
| | GCCCACCGTGCCCAGCACCTGAACTCCTGGGGGGACCGTCAGTCTTCCTCTTCCCCCCAAAACCCAAGGACACCCTCATGATCTCCCGGACCCCTGAGGT | |
| 701 | ---------!---------!---------!---------!---------!---------!---------!---------!---------!---------! | 800 |
| 1: | lThrCysValValValAspValSerHisGluAspProGluValLysPheAsnTrpTyrValAspGlyValGluValHisAsnAlaLysThrLysProArg | |
| | CACATGCGTGGTGGTGGACGTGAGCCACGAAGACCCTGAGGTCAAGTTCAACTGGTACGTGGACGGCGTGGAGGTGCATAATGCCAAGACAAAGCCGCGG | |
| 801 | ---------!---------!---------!---------!---------!---------!---------!---------!---------!---------! | 900 |
| 1: | GluGluGlnTyrAsnSerThrTyrArgValValSerValLeuThrValLeuHisGlnAspTrpLeuAsnGlyLysGluTyrLysCysLysValSerAsnL | |
| | GAGGAGCAGTACAACAGCACGTACCGTGTGGTCAGCGTCCTCACCGTCCTGCACCAGGACTGGCTGAATGGCAAGGAGTACAAGTGCAAGGTCTCCAACA | |
| 901 | ---------!---------!---------!---------!---------!---------!---------!---------!---------!---------! | 1000 |
| 1: | ysAlaLeuProAlaProIleGluLysThrIleSerLysAlaLysGlyGlnProArgGluProGlnValTyrThrLeuProProSerArgAspGluLeuTh | |
| | AAGCCCTCCCAGCCCCCATCGAGAAAACCATCTCCAAAGCCAAAGGGCAGCCCCGAGAACCACAGGTGTACACCCTGCCCCCATCCCGGGATGAGCTGAC | |
| 1001 | ---------!---------!---------!---------!---------!---------!---------!---------!---------!---------! | 1100 |
| 1: | rLysAsnGlnValSerLeuThrCysLeuValLysGlyPheTyrProSerAspIleAlaValGluTrpGluSerAsnGlyGlnProGluAsnAsnTyrLys | |
| | CAAGAACCAGGTCAGCCTGACCTGCCTGGTCAAAGGCTTCTATCCCAGCGACATCGCCGTGGAGTGGGAGAGCAATGGGCAGCCGGAGAACAACTACAAG | |
| 1101 | ---------!---------!---------!---------!---------!---------!---------!---------!---------!---------! | 1200 |
| 1: | ThrThrProProValLeuAspSerAspGlySerPhePheLeuTyrSerLysLeuThrValAspLysSerArgTrpGlnGlnGlyAsnValPheSerCysS | |
| | ACCACGCCTCCCGTGCTGGACTCCGACGGCTCCTTCTTCCTCTACAGCAAGCTCACCGTGGACAAGAGCAGGTGGCAGCAGGGGAACGTCTTCTCATGCT | |
| 1201 | ---------!---------!---------!---------!---------!---------!---------!---------!---------!---------! | 1300 |
| 1: | erValMetHisGluAlaLeuHisAsnHisTyrThrGlnLysSerLeuSerLeuSerProGlyLys | |
| | CCGTGATGCATGAGGCTCTGCACAACCACTACACGCAGAAGAGCCTCTCCCTGTCTCCGGGTAAA | |
| 1301 | ---------!---------!---------!---------!---------!---------!----- | 1365 |

6R8AE1 SEQ ID No. 67

| | | |
|---|---|---|
| 1: | GluValGlnLeuValGluSerGlyGlyGlyLeuValGlnProGlyGlySerLeuArgLeuSerCysLysAlaSerGlyTyrSerLeuAlaProGlnAlaM | |
| | GAGGTGCAGCTGGTGGAGTCTGGGGGAGGCTTGGTACAGCCTGGGGGGTCCCTGAGACTCTCCTGTAAGGCTTCTGGATATAGTCTTGCGCCGCAGGCGA | |
| 1 | ---------!---------!---------!---------!---------!---------!---------!---------!---------!---------! | 100 |
| 1: | etThrTrpValArgGlnAlaProGlyLysGlyLeuGluTrpIleGlyValIleAlaSerAsnAlaAspThrTyrTyrAlaSerTrpAlaLysGlyArgPh | |
| | TGACTTGGGTCCGCCAGGCTCCAGGGAAGGGGCTGGAGTGGATCGGAGTCATTGCTAGTAATGCTGACACATACTACGCGAGCTGGGCGAAAGGCCGGTT | |
| 101 | ---------!---------!---------!---------!---------!---------!---------!---------!---------!---------! | 200 |
| 1: | eThrIleSerArgAspAsnSerLysAsnThrLeuTyrLeuGlnMetAsnSerLeuArgAlaGluAspThrAlaValTyrTyrCysAlaArgHisProGly | |
| | CACCATCTCCAGAGACAATTCCAAGAACACGCTGTATCTGCAAATGAACAGCCTGAGAGCCGAGGACACGGCCGTATATTACTGTGCCAGACATCCTGGT | |
| 201 | ---------!---------!---------!---------!---------!---------!---------!---------!---------!---------! | 300 |
| 1: | SerAlaGlyTyrGlyTyrGlnTyrTyrTyrAlaMetAspProTrpGlyGlnGlyThrLeuValThrValSerSerAlaSerThrLysGlyProSerValP | |
| | AGTGCTGGTTATGGTTATCAGTATTATTACGCCATGGACCCCTGGGGCCAAGGAACCCTGGTCACCGTCTCCTCAGCCTCCACCAAGGGCCCATCGGTCT | |
| 301 | ---------!---------!---------!---------!---------!---------!---------!---------!---------!---------! | 400 |
| 1: | heProLeuAlaProSerSerLysSerThrSerGlyGlyThrAlaAlaLeuGlyCysLeuValLysAspTyrPheProGluProValThrValSerTrpAs | |
| | TCCCCCTGGCACCCTCCTCCAAGAGCACCTCTGGGGGCACAGCGGCCCTGGGCTGCCTGGTCAAGGACTACTTCCCCGAACCGGTGACGGTGTCGTGGAA | |
| 401 | ---------!---------!---------!---------!---------!---------!---------!---------!---------!---------! | 500 |
| 1: | nSerGlyAlaLeuThrSerGlyValHisThrPheProAlaValLeuGlnSerSerGlyLeuTyrSerLeuSerSerValValThrValProSerSerSer | |
| | CTCAGGCGCCCTGACCAGCGGCGTGCACACCTTCCCGGCTGTCCTACAGTCCTCAGGACTCTACTCCCTCAGCAGCGTGGTGACCGTGCCCTCCAGCAGC | |
| 501 | ---------!---------!---------!---------!---------!---------!---------!---------!---------!---------! | 600 |
| 1: | LeuGlyThrGlnThrTyrIleCysAsnValAsnHisLysProSerAsnThrLysValAspLysLysValGluProLysSerCysAspLysThrHisThrC | |
| | TTGGGCACCCAGACCTACATCTGCAACGTGAATCACAAGCCCAGCAACACCAAGGTGGACAAGAAAGTTGAGCCCAAATCTTGTGACAAAACTCACACAT | |
| 601 | ---------!---------!---------!---------!---------!---------!---------!---------!---------!---------! | 700 |
| 1: | ysProProCysProAlaProGluLeuLeuGlyGlyProSerValPheLeuPheProProLysProLysAspThrLeuMetIleSerArgThrProGluVa | |
| | GCCCACCGTGCCCAGCACCTGAACTCCTGGGGGGACCGTCAGTCTTCCTCTTCCCCCCAAAACCCAAGGACACCCTCATGATCTCCCGGACCCCTGAGGT | |
| 701 | ---------!---------!---------!---------!---------!---------!---------!---------!---------!---------! | 800 |
| 1: | lThrCysValValValAspValSerHisGluAspProGluValLysPheAsnTrpTyrValAspGlyValGluValHisAsnAlaLysThrLysProArg | |
| | CACATGCGTGGTGGTGGACGTGAGCCACGAAGACCCTGAGGTCAAGTTCAACTGGTACGTGGACGGCGTGGAGGTGCATAATGCCAAGACAAAGCCGCGG | |
| 801 | ---------!---------!---------!---------!---------!---------!---------!---------!---------!---------! | 900 |
| 1: | GluGluGlnTyrAsnSerThrTyrArgValValSerValLeuThrValLeuHisGlnAspTrpLeuAsnGlyLysGluTyrLysCysLysValSerAsnL | |
| | GAGGAGCAGTACAACAGCACGTACCGTGTGGTCAGCGTCCTCACCGTCCTGCACCAGGACTGGCTGAATGGCAAGGAGTACAAGTGCAAGGTCTCCAACA | |
| 901 | ---------!---------!---------!---------!---------!---------!---------!---------!---------!---------! | 1000 |
| 1: | ysAlaLeuProAlaProIleGluLysThrIleSerLysAlaLysGlyGlnProArgGluProGlnValTyrThrLeuProProSerArgAspGluLeuTh | |
| | AAGCCCTCCCAGCCCCCATCGAGAAAACCATCTCCAAAGCCAAAGGGCAGCCCCGAGAACCACAGGTGTACACCCTGCCCCCATCCCGGGATGAGCTGAC | |
| 1001 | ---------!---------!---------!---------!---------!---------!---------!---------!---------!---------! | 1100 |
| 1: | rLysAsnGlnValSerLeuThrCysLeuValLysGlyPheTyrProSerAspIleAlaValGluTrpGluSerAsnGlyGlnProGluAsnAsnTyrLys | |
| | CAAGAACCAGGTCAGCCTGACCTGCCTGGTCAAAGGCTTCTATCCCAGCGACATCGCCGTGGAGTGGGAGAGCAATGGGCAGCCGGAGAACAACTACAAG | |
| 1101 | ---------!---------!---------!---------!---------!---------!---------!---------!---------!---------! | 1200 |
| 1: | ThrThrProProValLeuAspSerAspGlySerPhePheLeuTyrSerLysLeuThrValAspLysSerArgTrpGlnGlnGlyAsnValPheSerCysS | |
| | ACCACGCCTCCCGTGCTGGACTCCGACGGCTCCTTCTTCCTCTACAGCAAGCTCACCGTGGACAAGAGCAGGTGGCAGCAGGGGAACGTCTTCTCATGCT | |
| 1201 | ---------!---------!---------!---------!---------!---------!---------!---------!---------!---------! | 1300 |
| 1: | erValMetHisGluAlaLeuHisAsnHisTyrThrGlnLysSerLeuSerLeuSerProGlyLys | |
| | CCGTGATGCATGAGGCTCTGCACAACCACTACACGCAGAAGAGCCTCTCCCTGTCTCCGGGTAAA | |
| 1301 | ---------!---------!---------!---------!---------!---------!----- | 1365 |

6R8AA2 SEQ ID No. 68

| | | |
|---|---|---|
| 1: | GluValGlnLeuValGluSerGlyGlyGlyLeuValGlnProGlyGlySerLeuArgLeuSerCysLysAlaSerGlyTyrSerLeuAsnLysGlnAlaL | |
| | GAGGTGCAGCTGGTGGAGTCTGGGGGAGGCTTGGTACAGCCTGGGGGGTCCCTGAGACTCTCCTGTAAGGCTTCTGGATATAGTCTTAATAAGCAGGCTC | |
| 1 | ---------!---------!---------!---------!---------!---------!---------!---------!---------!---------! | 100 |
| 1: | euThrTrpValArgGlnAlaProGlyLysGlyLeuGluTrpIleGlyValIleAlaSerAsnAlaAspThrTyrTyrAlaSerTrpAlaLysGlyArgPh | |
| | TCACTTGGGTCCGCCAGGCTCCAGGGAAGGGGCTGGAGTGGATCGGAGTCATTGCTAGTAATGCTGACACATACTACGCGAGCTGGGCGAAAGGCCGGTT | |
| 101 | ---------!---------!---------!---------!---------!---------!---------!---------!---------!---------! | 200 |
| 1: | eThrIleSerArgAspAsnSerLysAsnThrLeuTyrLeuGlnMetAsnSerLeuArgAlaGluAspThrAlaValTyrTyrCysAlaArgHisProGly | |
| | CACCATCTCCAGAGACAATTCCAAGAACACGCTGTATCTGCAAATGAACAGCCTGAGAGCCGAGGACACGGCCGTATATTACTGTGCCAGACATCCTGGT | |
| 201 | ---------!---------!---------!---------!---------!---------!---------!---------!---------!---------! | 300 |
| 1: | SerAlaGlyTyrGlyTyrGlnTyrTyrTyrAlaMetAspProTrpGlyGlnGlyThrLeuValThrValSerSerAlaSerThrLysGlyProSerValP | |
| | AGTGCTGGTTATGGTTATCAGTATTATTACGCCATGGACCCCTGGGGCCAAGGAACCCTGGTCACCGTCTCCTCAGCCTCCACCAAGGGCCCATCGGTCT | |
| 301 | ---------!---------!---------!---------!---------!---------!---------!---------!---------!---------! | 400 |
| 1: | heProLeuAlaProSerSerLysSerThrSerGlyAspThrAlaAlaLeuGlyCysLeuValLysAspTyrPheProGluProValThrValSerTrpAs | |
| | TCCCCCTGGCACCCTCCTCCAAGAGCACCTCTGGGGACACAGCGGCCCTGGGCTGCCTGGTCAAGGACTACTTCCCCGAACCGGTGACGGTGTCGTGGAA | |
| 401 | ---------!---------!---------!---------!---------!---------!---------!---------!---------!---------! | 500 |
| 1: | nSerGlyAlaLeuThrSerGlyValHisThrPheProAlaValLeuGlnSerSerGlyLeuTyrSerLeuSerSerValValThrValProSerSerSer | |
| | CTCAGGCGCCCTGACCAGCGGCGTGCACACCTTCCCGGCTGTCCTACAGTCCTCAGGACTCTACTCCCTCAGCAGCGTGGTGACCGTGCCCTCCAGCAGC | |
| 501 | ---------!---------!---------!---------!---------!---------!---------!---------!---------!---------! | 600 |
| 1: | LeuGlyThrGlnThrTyrIleCysAsnValAsnHisLysProSerAsnThrLysValAspLysLysValGluProLysSerCysAspLysThrHisThrC | |
| | TTGGGCACCCAGACCTACATCTGCAACGTGAATCACAAGCCCAGCAACACCAAGGTGGACAAGAAAGTTGAGCCCAAATCTTGTGACAAAACTCACACAT | |
| 601 | ---------!---------!---------!---------!---------!---------!---------!---------!---------!---------! | 700 |
| 1: | ysProProCysProAlaProGluLeuLeuGlyGlyProSerValPheLeuPheProProLysProLysAspThrLeuMetIleSerArgThrProGluVa | |
| | GCCCACCGTGCCCAGCACCTGAACTCCTGGGGGGACCGTCAGTCTTCCTCTTCCCCCCAAAACCCAAGGACACCCTCATGATCTCCCGGACCCCTGAGGT | |
| 701 | ---------!---------!---------!---------!---------!---------!---------!---------!---------!---------! | 800 |
| 1: | lThrCysValValValAspValSerHisGluAspProGluValLysPheAsnTrpTyrValAspGlyValGluValHisAsnAlaLysThrLysProArg | |
| | CACATGCGTGGTGGTGGACGTGAGCCACGAAGACCCTGAGGTCAAGTTCAACTGGTACGTGGACGGCGTGGAGGTGCATAATGCCAAGACAAAGCCGCGG | |
| 801 | ---------!---------!---------!---------!---------!---------!---------!---------!---------!---------! | 900 |
| 1: | GluGluGlnTyrAsnSerThrTyrArgValValSerValLeuThrValLeuHisGlnAspTrpLeuAsnGlyLysGluTyrLysCysLysValSerAsnL | |
| | GAGGAGCAGTACAACAGCACGTACCGTGTGGTCAGCGTCCTCACCGTCCTGCACCAGGACTGGCTGAATGGCAAGGAGTACAAGTGCAAGGTCTCCAACA | |
| 901 | ---------!---------!---------!---------!---------!---------!---------!---------!---------!---------! | 1000 |
| 1: | ysAlaLeuProAlaProIleGluLysThrIleSerLysAlaLysGlyGlnProArgGluProGlnValTyrThrLeuProProSerArgAspGluLeuTh | |
| | AAGCCCTCCCAGCCCCCATCGAGAAAACCATCTCCAAAGCCAAAGGGCAGCCCCGAGAACCACAGGTGTACACCCTGCCCCCATCCCGGGATGAGCTGAC | |
| 1001 | ---------!---------!---------!---------!---------!---------!---------!---------!---------!---------! | 1100 |
| 1: | rLysAsnGlnValSerLeuThrCysLeuValLysGlyPheTyrProSerAspIleAlaValGluTrpGluSerAsnGlyGlnProGluAsnAsnTyrLys | |
| | CAAGAACCAGGTCAGCCTGACCTGCCTGGTCAAAGGCTTCTATCCCAGCGACATCGCCGTGGAGTGGGAGAGCAATGGGCAGCCGGAGAACAACTACAAG | |
| 1101 | ---------!---------!---------!---------!---------!---------!---------!---------!---------!---------! | 1200 |
| 1: | ThrThrProProValLeuAspSerAspGlySerPhePheLeuTyrSerLysLeuThrValAspLysSerArgTrpGlnGlnGlyAsnValPheSerCysS | |
| | ACCACGCCTCCCGTGCTGGACTCCGACGGCTCCTTCTTCCTCTACAGCAAGCTCACCGTGGACAAGAGCAGGTGGCAGCAGGGGAACGTCTTCTCATGCT | |
| 1201 | ---------!---------!---------!---------!---------!---------!---------!---------!---------!---------! | 1300 |
| 1: | erValMetHisGluAlaLeuHisAsnHisTyrThrGlnLysSerLeuSerLeuSerProGlyLys | |
| | CCGTGATGCATGAGGCTCTGCACAACCACTACACGCAGAAGAGCCTCTCCCTGTCTCCGGGTAAA | |
| 1301 | ---------!---------!---------!---------!---------!---------!----- | 1365 |

6R8CG3 SEQ ID No. 69

| | | |
|---|---|---|
| 1: | GluValGlnLeuValGluSerGlyGlyGlyLeuValGlnProGlyGlySerLeuArgLeuSerCysLysAlaSerGlyIleSerLeuAlaProTyrAlaI | |
| | GAGGTGCAGCTGGTTGAGTCTGGGGGAGGCTTGGTACAGCCTGGGGGGTCCCTGAGACTCTCCTGTAAGGCTTCTGGAATTAGTCTTGCGCCGTATGCTA | |
| 1 | ---------!---------!---------!---------!---------!---------!---------!---------!---------!---------! | 100 |
| 1: | leThrTrpValArgGlnAlaProGlyLysGlyLeuGluTrpIleGlyValIleAlaSerAsnAlaAspThrTyrTyrAlaSerTrpAlaLysGlyArgPh | |
| | TCACTTGGGTCCGCCAGGCTCCAGGGAAGGGGCTGGAGTGGATCGGAGTCATTGCTAGTAATGCTGACACATACTACGCGAGCTGGGCGAAAGGCCGGTT | |
| 101 | ---------!---------!---------!---------!---------!---------!---------!---------!---------!---------! | 200 |
| 1: | eThrIleSerArgAspAsnSerLysAsnThrLeuTyrLeuGlnMetAsnSerLeuArgAlaGluAspThrAlaValTyrTyrCysAlaArgHisProGly | |
| | CACCATCTCCAGAGACAATTCCAAGAACACGCTGTATCTGCAAATGAACAGCCTGAGAGCCGAGGACACGGCCGTATATTACTGTGCCAGACATCCTGGT | |
| 201 | ---------!---------!---------!---------!---------!---------!---------!---------!---------!---------! | 300 |
| 1: | SerAlaGlyTyrGlyTyrGlnTyrTyrTyrSerMetAspProTrpGlyGlnGlyThrLeuValThrValSerSerAlaSerThrLysGlyProSerValP | |
| | AGTGCTGGTTATGGTTATCAGTATTATTATAGTATGGACCCCTGGGGCCAAGGAACCCTGGTCACCGTCTCCTCAGCCTCCACCAAGGGCCCATCGGTCT | |
| 301 | ---------!---------!---------!---------!---------!---------!---------!---------!---------!---------! | 400 |
| 1: | heProLeuAlaProSerSerLysSerThrSerGlyGlyThrAlaAlaLeuGlyCysLeuValLysAspTyrPheProGluProValThrValSerTrpAs | |
| | TCCCCCTGGCACCCTCCTCCAAGAGCACCTCTGGGGGCACAGCGGCCCTGGGCTGCCTGGTCAAGGACTACTTCCCCGAACCGGTGACGGTGTCGTGGAA | |
| 401 | ---------!---------!---------!---------!---------!---------!---------!---------!---------!---------! | 500 |
| 1: | nSerGlyAlaLeuThrSerGlyValHisThrPheProAlaValLeuGlnSerSerGlyLeuTyrSerLeuSerSerValValThrValProSerSerSer | |
| | CTCAGGCGCCCTGACCAGCGGCGTGCACACCTTCCCGGCTGTCCTACAGTCCTCAGGACTCTACTCCCTCAGCAGCGTGGTGACCGTGCCCTCCAGCAGC | |
| 501 | ---------!---------!---------!---------!---------!---------!---------!---------!---------!---------! | 600 |
| 1: | LeuGlyThrGlnThrTyrIleCysAsnValAsnHisLysProSerAsnThrLysValAspLysLysValGluProLysSerCysAspLysThrHisThrC | |
| | TTGGGCACCCAGACCTACATCTGCAACGTGAATCACAAGCCCAGCAACACCAAGGTGGACAAGAAAGTTGAGCCCAAATCTTGTGACAAAACTCACACAT | |
| 601 | ---------!---------!---------!---------!---------!---------!---------!---------!---------!---------! | 700 |
| 1: | ysProProCysProAlaProGluLeuLeuGlyGlyProSerValPheLeuPheProProLysProLysAspThrLeuMetIleSerArgThrProGluVa | |
| | GCCCACCGTGCCCAGCACCTGAACTCCTGGGGGGACCGTCAGTCTTCCTCTTCCCCCCAAAACCCAAGGACACCCTCATGATCTCCCGGACCCCTGAGGT | |
| 701 | ---------!---------!---------!---------!---------!---------!---------!---------!---------!---------! | 800 |
| 1: | lThrCysValValValAspValSerHisGluAspProGluValLysPheAsnTrpTyrValAspGlyValGluValHisAsnAlaLysThrLysProArg | |
| | CACATGCGTGGTGGTGGACGTGAGCCACGAAGACCCTGAGGTCAAGTTCAACTGGTACGTGGACGGCGTGGAGGTGCATAATGCCAAGACAAAGCCGCGG | |
| 801 | ---------!---------!---------!---------!---------!---------!---------!---------!---------!---------! | 900 |
| 1: | GluGluGlnTyrAsnSerThrTyrArgValValSerValLeuThrValLeuHisGlnAspTrpLeuAsnGlyLysGluTyrLysCysLysValSerAsnI | |
| | GAGGAGCAGTACAACAGCACGTACCGTGTGGTCAGCGTCCTCACCGTCCTGCACCAGGACTGGCTGAATGGCAAGGAGTACAAGTGCAAGGTCTCCAACA | |
| 901 | ---------!---------!---------!---------!---------!---------!---------!---------!---------!---------! | 1000 |
| 1: | ysAlaLeuProAlaProIleGluLysThrIleSerLysAlaLysGlyGlnProArgGluProGlnValTyrThrLeuProProSerArgAspGluLeuTh | |
| | AAGCCCTCCCAGCCCCCATCGAGAAAACCATCTCCAAAGCCAAAGGGCAGCCCCGAGAACCACAGGTGTACACCCTGCCCCCATCCCGGGATGAGCTGAC | |
| 1001 | ---------!---------!---------!---------!---------!---------!---------!---------!---------!---------! | 1100 |
| 1: | rLysAsnGlnValSerLeuThrCysLeuValLysGlyPheTyrProSerAspIleAlaValGluTrpGluSerAsnGlyGlnProGluAsnAsnTyrLys | |
| | CAAGAACCAGGTCAGCCTGACCTGCCTGGTCAAAGGCTTCTATCCCAGCGACATCGCCGTGGAGTGGGAGAGCAATGGGCAGCCGGAGAACAACTACAAG | |
| 1101 | ---------!---------!---------!---------!---------!---------!---------!---------!---------!---------! | 1200 |
| 1: | ThrThrProProValLeuAspSerAspGlySerPhePheLeuTyrSerLysLeuThrValAspLysSerArgTrpGlnGlnGlyAsnValPheSerCysS | |
| | ACCACGCCTCCCGTGCTGGACTCCGACGGCTCCTTCTTCCTCTACAGCAAGCTCACCGTGGACAAGAGCAGGTGGCAGCAGGGGAACGTCTTCTCATGCT | |
| 1201 | ---------!---------!---------!---------!---------!---------!---------!---------!---------!---------! | 1300 |
| 1: | erValMetHisGluAlaLeuHisAsnHisTyrThrGlnLysSerLeuSerLeuSerProGlyLys | |
| | CCGTGATGCATGAGGCTCTGCACAACCACTACACGCAGAAGAGCCTCTCCCTGTCTCCGGGTAAA | |
| 1301 | ---------!---------!---------!---------!---------!---------!----- | 1365 |

6R8CG4 SEQ ID No. 70

| | | |
|---|---|---|
| 1: | GluValGlnLeuValGluSerGlyGlyGlyLeuValGlnProGlyGlySerLeuArgLeuSerCysLysAlaSerGlyPheThrPheSerArgGlnAlaL | |
| | GAGGTGCAGCTGGTGGAGTCTGGGGGAGGCTTGGTACAGCCTGGGGGGTCCCTGAGACTCTCCTGTAAGGCTTCTGGATTTACTTTTAGTCGTCAGGCTC | |
| 1 | ---------!---------!---------!---------!---------!---------!---------!---------!---------!---------! | 100 |
| 1: | euThrTrpValArgGlnAlaProGlyLysGlyLeuGluTrpIleGlyValIleAlaSerAsnAlaAspThrTyrTyrAlaSerTrpAlaLysGlyArgPh | |
| | TCACTTGGGTCCGCCAGGCTCCAGGGAAGGGGCTGGAGTGGATCGGAGTCATTGCTAGTAATGCTGACACATACTACGCGAGCTGGGCGAAAGGCCGGTT | |
| 101 | ---------!---------!---------!---------!---------!---------!---------!---------!---------!---------! | 200 |
| 1: | eThrIleSerArgAspAsnSerLysAsnThrLeuTyrLeuGlnMetAsnSerLeuArgAlaGluAspThrAlaValTyrTyrCysAlaArgHisProGly | |
| | CACCATCTCCAGAGACAATTCCAAGAACACGCTGTATCTGCAAATGAACAGCCTGAGAGCCGAGGACACGGCCGTATATTACTGTGCCAGACATCCTGGT | |
| 201 | ---------!---------!---------!---------!---------!---------!---------!---------!---------!---------! | 300 |
| 1: | SerAlaGlyTyrGlyTyrGlnTyrTyrTyrAlaMetAspProTrpGlyGlnGlyThrLeuValThrValSerSerAlaSerThrLysGlyProSerValP | |
| | AGTGCTGGTTATGGTTATCAGTATTATTACGCCATGGACCCCTGGGGCCAAGGAACCCTGGTCACCGTCTCCTCAGCCTCCACCAAGGGCCCATCGGTCT | |
| 301 | ---------!---------!---------!---------!---------!---------!---------!---------!---------!---------! | 400 |
| 1: | heProLeuAlaProSerSerLysSerThrSerGlyGlyThrAlaAlaLeuGlyCysLeuValLysAspTyrPheProGluProValThrValSerTrpAs | |
| | TCCCCCTGGCACCCTCCTCCAAGAGCACCTCTGGGGGCACAGCGGCCCTGGGCTGCCTGGTCAAGGACTACTTCCCCGAACCGGTGACGGTGTCGTGGAA | |
| 401 | ---------!---------!---------!---------!---------!---------!---------!---------!---------!---------! | 500 |
| 1: | nSerGlyAlaLeuThrSerGlyValHisThrPheProAlaValLeuGlnSerSerGlyLeuTyrSerLeuSerSerValValThrValProSerSerSer | |
| | CTCAGGCGCCCTGACCAGCGGCGTGCACACCTTCCCGGCTGTCCTACAGTCCTCAGGACTCTACTCCCTCAGCAGCGTGGTGACCGTGCCCTCCAGCAGC | |
| 501 | ---------!---------!---------!---------!---------!---------!---------!---------!---------!---------! | 600 |
| 1: | LeuGlyThrGlnThrTyrIleCysAsnValAsnHisLysProSerAsnThrLysValAspLysLysValGluProLysSerCysAspLysThrHisThrC | |
| | TTGGGCACCCAGACCTACATCTGCAACGTGAATCACAAGCCCAGCAACACCAAGGTGGACAAGAAAGTTGAGCCCAAATCTTGTGACAAAACTCACACAT | |
| 601 | ---------!---------!---------!---------!---------!---------!---------!---------!---------!---------! | 700 |
| 1: | ysProProCysProAlaProGluLeuLeuGlyGlyProSerValPheLeuPheProProLysProLysAspThrLeuMetIleSerArgThrProGluVa | |
| | GCCCACCGTGCCCAGCACCTGAACTCCTGGGGGGACCGTCAGTCTTCCTCTTCCCCCCAAAACCCAAGGACACCCTCATGATCTCCCGGACCCCTGAGGT | |
| 701 | ---------!---------!---------!---------!---------!---------!---------!---------!---------!---------! | 800 |
| 1: | lThrCysValValValAspValSerHisGluAspProGluValLysPheAsnTrpTyrValAspGlyValGluValHisAsnAlaLysThrLysProArg | |
| | CACATGCGTGGTGGTGGACGTGAGCCACGAAGACCCTGAGGTCAAGTTCAACTGGTACGTGGACGGCGTGGAGGTGCATAATGCCAAGACAAAGCCGCGG | |
| 801 | ---------!---------!---------!---------!---------!---------!---------!---------!---------!---------! | 900 |
| 1: | GluGluGlnTyrAsnSerThrTyrArgValValSerValLeuThrValLeuHisGlnAspTrpLeuAsnGlyLysGluTyrLysCysLysValSerAsnL | |
| | GAGGAGCAGTACAACAGCACGTACCGTGTGGTCAGCGTCCTCACCGTCCTGCACCAGGACTGGCTGAATGGCAAGGAGTACAAGTGCAAGGTCTCCAACA | |
| 901 | ---------!---------!---------!---------!---------!---------!---------!---------!---------!---------! | 1000 |
| 1: | ysAlaLeuProAlaProIleGluLysThrIleSerLysAlaLysGlyGlnProArgGluProGlnValTyrThrLeuProProSerArgAspGluLeuTh | |
| | AAGCCCTCCCAGCCCCCATCGAGAAAACCATCTCCAAAGCCAAAGGGCAGCCCCGAGAACCACAGGTGTACACCCTGCCCCCATCCCGGGATGAGCTGAC | |
| 1001 | ---------!---------!---------!---------!---------!---------!---------!---------!---------!---------! | 1100 |
| 1: | rLysAsnGlnValSerLeuThrCysLeuValLysGlyPheTyrProSerAspIleAlaValGluTrpGluSerAsnGlyGlnProGluAsnAsnTyrLys | |
| | CAAGAACCAGGTCAGCCTGACCTGCCTGGTCAAAGGCTTCTATCCCAGCGACATCGCCGTGGAGTGGGAGAGCAATGGGCAGCCGGAGAACAACTACAAG | |
| 1101 | ---------!---------!---------!---------!---------!---------!---------!---------!---------!---------! | 1200 |
| 1: | ThrThrProProValLeuAspSerAspGlySerPhePheLeuTyrSerLysLeuThrValAspLysSerArgTrpGlnGlnGlyAsnValPheSerCysS | |
| | ACCACGCCTCCCGTGCTGGACTCCGACGGCTCCTTCTTCCTCTACAGCAAGCTCACCGTGGACAAGAGCAGGTGGCAGCAGGGGAACGTCTTCTCATGCT | |
| 1201 | ---------!---------!---------!---------!---------!---------!---------!---------!---------!---------! | 1300 |
| 1: | erValMetHisGluAlaLeuHisAsnHisTyrThrGlnLysSerLeuSerLeuSerProGlyLys | |
| | CCGTGATGCATGAGGCTCTGCACAACCACTACACGCAGAAGAGCCTCTCCCTGTCTCCGGGTAAA | |
| 1301 | ---------!---------!---------!---------!---------!---------!----- | 1365 |

6R8CG8 SEQ ID No. 71

| | | |
|---|---|---|
| 1: | GluValGlnLeuValGluSerGlyGlyGlyLeuValGlnLeuGlyGlySerLeuArgLeuSerCysLysAlaSerGlyTyrSerLeuAlaProGlnAlaM | |
| | GAGGTGCAGCTGGTGGAGTCTGGGGGAGGCTTGGTACAGCTTGGGGGGTCCCTGAGACTCTCCTGTAAGGCTTCTGGATATAGTCTTGCGCCGCAGGCGA | |
| 1 | ---------!---------!---------!---------!---------!---------!---------!---------!---------!---------! | 100 |
| 1: | etThrTrpValArgGlnAlaProGlyLysGlyLeuGluTrpIleGlyValIleAlaSerAsnAlaAspThrTyrTyrAlaSerTrpAlaLysGlyArgPh | |
| | TGACTTGGGTCCGCCAGGCTCCAGGGAAGGGGCTGGAGTGGATCGGAGTCATTGCTAGTAATGCTGACACATACTACGCGAGCTGGGCGAAAGGCCGATT | |
| 101 | ---------!---------!---------!---------!---------!---------!---------!---------!---------!---------! | 200 |
| 1: | eThrIleSerArgAspAsnSerLysAsnThrLeuTyrLeuGlnMetAsnSerLeuArgAlaGluAspThrAlaValTyrTyrCysAlaArgHisProGly | |
| | CACCATCTCCAGAGACAATTCCAAGAACACGCTGTATCTGCAAATGAACAGCCTGAGAGCCGAGGACACGGCCGTATATTACTGTGCCAGACATCCTGGT | |
| 201 | ---------!---------!---------!---------!---------!---------!---------!---------!---------!---------! | 300 |
| 1: | SerAlaGlyTyrGlyTyrGlnTyrTyrTyrAlaMetAspProTrpGlyGlnGlyThrLeuValThrValSerSerAlaSerThrLysGlyProSerValP | |
| | AGTGCTGGTTATGGTTATCAGTATTATTACGCCATGGACCCCTGGGGCCAAGGAACCCTGGTCACCGTCTCCTCAGCCTCCACCAAGGGCCCATCGGTCT | |
| 301 | ---------!---------!---------!---------!---------!---------!---------!---------!---------!---------! | 400 |
| 1: | heProLeuAlaProSerSerLysSerThrSerGlyGlyThrAlaAlaLeuGlyCysLeuValLysAspTyrPheProGluProValThrValSerTrpAs | |
| | TCCCCCTGGCACCCTCCTCCAAGAGCACCTCTGGGGGCACAGCGGCCCTGGGCTGCCTGGTCAAGGACTACTTCCCCGAACCGGTGACGGTGTCGTGGAA | |
| 401 | ---------!---------!---------!---------!---------!---------!---------!---------!---------!---------! | 500 |
| 1: | nSerGlyAlaLeuThrSerGlyValHisThrPheProAlaValLeuGlnSerSerGlyLeuTyrSerLeuSerSerValValThrValProSerSerSer | |
| | CTCAGGCGCCCTGACCAGCGGCGTGCACACCTTCCCGGCTGTCCTACAGTCCTCAGGACTCTACTCCCTCAGCAGCGTGGTGACCGTGCCCTCCAGCAGC | |
| 101 | ---------!---------!---------!---------!---------!---------!---------!---------!---------!---------! | 600 |
| 1: | LeuGlyThrGlnThrTyrIleCysAsnValAsnHisLysProSerAsnThrLysValAspLysLysValGluProLysSerCysAspLysThrHisThrC | |
| | TTGGGCACCCAGACCTACATCTGCAACGTGAATCACAAGCCCAGCAACACCAAGGTGGACAAGAAAGTTGAGCCCAAATCTTGTGACAAAACTCACACAT | |
| 601 | ---------!---------!---------!---------!---------!---------!---------!---------!---------!---------! | 700 |
| 1: | ysProProCysProAlaProGluLeuLeuGlyGlyProSerValPheLeuPheProProLysProLysAspThrLeuMetIleSerArgThrProGluVa | |
| | GCCCACCGTGCCCAGCACCTGAACTCCTGGGGGGACCGTCAGTCTTCCTCTTCCCCCCAAAACCCAAGGACACCCTCATGATCTCCCGGACCCCTGAGGT | |
| 701 | ---------!---------!---------!---------!---------!---------!---------!---------!---------!---------! | 800 |
| 1: | lThrCysValValValAspValSerHisGluAspProGluValLysPheAsnTrpTyrValAspGlyValGluValHisAsnAlaLysThrLysProArg | |
| | CACATGCGTGGTGGTGGACGTGAGCCACGAAGACCCTGAGGTCAAGTTCAACTGGTACGTGGACGGCGTGGAGGTGCATAATGCCAAGACAAAGCCGCGG | |
| 801 | ---------!---------!---------!---------!---------!---------!---------!---------!---------!---------! | 900 |
| 1: | GluGluGlnTyrAsnSerThrTyrArgValValSerValLeuThrValLeuHisGlnAspTrpLeuAsnGlyLysGluTyrLysCysLysValSerAsnL | |
| | GAGGAGCAGTACAACAGCACGTACCGTGTGGTCAGCGTCCTCACCGTCCTGCACCAGGACTGGCTGAATGGCAAGGAGTACAAGTGCAAGGTCTCCAACA | |
| 901 | ---------!---------!---------!---------!---------!---------!---------!---------!---------!---------! | 1000 |
| 1: | ysAlaLeuProAlaProIleGluLysThrIleSerLysAlaLysGlyGlnProArgGluProGlnValTyrThrLeuProProSerArgAspGluLeuTh | |
| | AAGCCCTCCCAGCCCCCATCGAGAAAACCATCTCCAAAGCCAAAGGGCAGCCCCGAGAACCACAGGTGTACACCCTGCCCCCATCCCGGGATGAGCTGAC | |
| 1001 | ---------!---------!---------!---------!---------!---------!---------!---------!---------!---------! | 1100 |
| 1: | rLysAsnGlnValSerLeuThrCysLeuValLysGlyPheTyrProSerAspIleAlaValGluTrpGluSerAsnGlyGlnProGluAsnAsnTyrLys | |
| | CAAGAACCAGGTCAGCCTGACCTGCCTGGTCAAAGGCTTCTATCCCAGCGACATCGCCGTGGAGTGGGAGAGCAATGGGCAGCCGGAGAACAACTACAAG | |
| 1101 | ---------!---------!---------!---------!---------!---------!---------!---------!---------!---------! | 1200 |
| 1: | ThrThrProProValLeuAspSerAspGlySerPhePheLeuTyrSerLysLeuThrValAspLysSerArgTrpGlnGlnGlyAsnValPheSerCysS | |
| | ACCACGCCTCCCGTGCTGGACTCCGACGGCTCCTTCTTCCTCTACAGCAAGCTCACCGTGGACAAGAGCAGGTGGCAGCAGGGGAACGTCTTCTCATGCT | |
| 1201 | ---------!---------!---------!---------!---------!---------!---------!---------!---------!---------! | 1300 |
| 1: | erValMetHisGluAlaLeuHisAsnHisTyrThrGlnLysSerLeuSerLeuSerProGlyLys | |
| | CCGTGATGCATGAGGCTCTGCACAACCACTACACGCAGAAGAGCCTCTCCCTGTCTCCGGGTAAA | |
| 1301 | ---------!---------!---------!---------!---------!---------!----- | 1365 |

6R8CD9 SEQ ID No. 72

| | | |
|---|---|---|
| 1: | GluValGlnLeuValGluSerGlyGlyGlyLeuValGlnProGlyGlySerLeuArgLeuSerCysLysAlaSerGlyPheThrPheSerArgGlnAlaL | |
| | GAGGTGCAGCTGGTGGAGTCTGGGGGAGGCTTGGTACAGCCTGGGGGGTCCCTGAGACTCTCCTGTAAGGCTTCTGGATTTACTTTTAGTCGTCAGGCTC | |
| 1 | ---------!---------!---------!---------!---------!---------!---------!---------!---------!---------! | 100 |
| 1: | euThrTrpValArgGlnAlaProGlyLysGlyLeuGluTrpIleGlyValIleAlaSerAsnAlaAspThrTyrTyrAlaSerTrpAlaLysGlyArgPh | |
| | TCACTTGGGTCCGCCAGGCTCCAGGGAAGGGGCTGGAGTGGATCGGAGTCATTGCTAGTAATGCTGACACATACTACGCGAGCTGGGCGAAAGGCCGGTT | |
| 101 | ---------!---------!---------!---------!---------!---------!---------!---------!---------!---------! | 200 |
| 1: | eThrIleSerArgAspAsnSerLysAsnThrLeuTyrLeuGlnMetAsnSerLeuArgAlaGluAspThrAlaValTyrTyrCysAlaArgHisProGly | |
| | CACCATCTCCAGAGACAATTCCAAGAACACGCTGTATCTGCAAATGAACAGCCTGAGAGCCGAGGACACGGCCGTATATTACTGTGCCAGACATCCTGGT | |
| 201 | ---------!---------!---------!---------!---------!---------!---------!---------!---------!---------! | 300 |
| 1: | SerAlaGlyTyrGlyTyrGlnTyrTyrTyrAlaMetAspProTrpGlyGlnGlyThrLeuValThrValSerSerAlaSerThrLysGlyProSerValP | |
| | AGTGCTGGTTATGGTTATCAGTATTATTACGCCATGGACCCCTGGGGCCAAGGAACCCTGGTCACCGTCTCCTCAGCCTCCACCAAGGGCCCATCGGTCT | |
| 301 | ---------!---------!---------!---------!---------!---------!---------!---------!---------!---------! | 400 |
| 1: | heProLeuAlaProSerSerLysSerThrSerGlyGlyThrAlaAlaLeuGlyCysLeuValLysAspTyrPheProGluProValThrValSerTrpAs | |
| | TCCCCCTGGCACCCTCCTCCAAGAGCACCTCTGGGGGCACAGCGGCCCTGGGCTGCCTGGTCAAGGACTACTTCCCCGAACCGGTGACGGTGTCGTGGAA | |
| 401 | ---------!---------!---------!---------!---------!---------!---------!---------!---------!---------! | 500 |
| 1: | nSerGlyAlaLeuThrSerGlyValHisThrPheProAlaValLeuGlnSerSerGlyLeuTyrSerLeuSerSerValValThrValProSerSerSer | |
| | CTCAGGCGCCCTGACCAGCGGCGTGCACACCTTCCCGGCTGTCCTACAGTCCTCAGGACTCTACTCCCTCAGCAGCGTGGTGACCGTGCCCTCCAGCAGC | |
| 501 | ---------!---------!---------!---------!---------!---------!---------!---------!---------!---------! | 600 |
| 1: | LeuGlyThrGlnThrTyrIleCysAsnValAsnHisLysProSerAsnThrLysValAspLysLysValGluProLysSerCysAspLysThrHisThrC | |
| | TTGGGCACCCAGACCTACATCTGCAACGTGAATCACAAGCCCAGCAACACCAAGGTGGACAAGAAAGTTGAGCCCAAATCTTGTGACAAAACTCACACAT | |
| 601 | ---------!---------!---------!---------!---------!---------!---------!---------!---------!---------! | 700 |
| 1: | ysProProCysProAlaProGluLeuLeuGlyGlyProSerValPheLeuPheProProLysProLysAspThrLeuMetIleSerArgThrProGluVa | |
| | GCCCACCGTGCCCAGCACCTGAACTCCTGGGGGGACCGTCAGTCTTCCTCTTCCCCCCAAAACCCAAGGACACCCTCATGATCTCCCGGACCCCTGAGGT | |
| 701 | ---------!---------!---------!---------!---------!---------!---------!---------!---------!---------! | 800 |
| 1: | lThrCysValValValAspValSerHisGluAspProGluValLysPheAsnTrpTyrValAspGlyValGluValHisAsnAlaLysThrLysProArg | |
| | CACATGCGTGGTGGTGGACGTGAGCCACGAAGACCCTGAGGTCAAGTTCAACTGGTACGTGGACGGCGTGGAGGTGCATAATGCCAAGACAAAGCCGCGG | |
| 801 | ---------!---------!---------!---------!---------!---------!---------!---------!---------!---------! | 900 |
| 1: | GluGluGlnTyrAsnSerThrTyrArgValValSerValLeuThrValLeuHisGlnAspTrpLeuAsnGlyLysGluTyrLysCysLysValSerAsnL | |
| | GAGGAGCAGTACAACAGCACGTACCGTGTGGTCAGCGTCCTCACCGTCCTGCACCAGGACTGGCTGAATGGCAAGGAGTACAAGTGCAAGGTCTCCAACA | |
| 901 | ---------!---------!---------!---------!---------!---------!---------!---------!---------!---------! | 1000 |
| 1: | ysAlaLeuProAlaProIleGluLysThrIleSerLysAlaLysGlyGlnProArgGluProGlnValTyrThrLeuProProSerArgAspGluLeuTh | |
| | AAGCCCTCCCAGCCCCCATCGAGAAAACCATCTCCAAAGCCAAAGGGCAGCCCCGAGAACCACAGGTGTACACCCTGCCCCCATCCCGGGATGAGCTGAC | |
| 1001 | ---------!---------!---------!---------!---------!---------!---------!---------!---------!---------! | 1100 |
| 1: | rLysAsnGlnValSerLeuThrCysLeuValLysGlyPheTyrProSerAspIleAlaValGluTrpGluSerAsnGlyGlnProGluAsnAsnTyrLys | |
| | CAAGAACCAGGTCAGCCTGACCTGCCTGGTCAAAGGCTTCTATCCCAGCGACATCGCCGTGGAGTGGGAGAGCAATGGGCAGCCGGAGAACAACTACAAG | |
| 1101 | ---------!---------!---------!---------!---------!---------!---------!---------!---------!---------! | 1200 |
| 1: | ThrThrProProValLeuAspSerAspGlySerPhePheLeuTyrSerLysLeuThrValAspLysSerArgTrpGlnGlnGlyAsnValPheSerCysS | |
| | ACCACGCCTCCCGTGCTGGACTCCGACGGCTCCTTCTTCCTCTACAGCAAGCTCACCGTGGACAAGAGCAGGTGGCAGCAGGGGAACGTCTTCTCATGCT | |
| 1201 | ---------!---------!---------!---------!---------!---------!---------!---------!---------!---------! | 1300 |
| 1: | erValMetHisGluAlaLeuHisAsnHisTyrThrGlnLysSerLeuSerLeuSerProGlyLys | |
| | CCGTGATGCATGAGGCTCTGCACAACCACTACACGCAGAAGAGCCTCTCCCTGTCTCCGGGTAAA | |
| 1301 | ---------!---------!---------!---------!---------!---------!----- | 1365 |

6R8CD10 SEQ ID No. 73

| | | |
|---|---|---|
| 1: | GluValGlnLeuValGluSerGlyGlyGlyLeuValGlnProGlyGlySerLeuArgLeuSerCysThrAlaSerGlyTyrSerIleSerHisGlnAlaV | |
| | GAGGTGCAGCTGGTGGAGTCTGGGGGAGGCTTGGTACAGCCTGGGGGGTCCCTGAGACTCTCCTGTACGGCTTCTGGATATAGTATTTCGCATCAGGCTG | |
| 1 | ---------!---------!---------!---------!---------!---------!---------!---------!---------!---------! | 100 |
| 1: | alThrTrpValArgGlnAlaProGlyLysGlyLeuGluTrpIleGlyValIleAlaSerAsnAlaAspThrTyrTyrAlaSerTrpAlaLysGlyArgPh | |
| | TGACTTGGGTCCGCCAGGCTCCAGGGAAGGGGCTGGAGTGGATCGGAGTCATTGCTAGTAATGCTGACACATACTACGCGAGCTGGGCGAAAGGCCGGTT | |
| 101 | ---------!---------!---------!---------!---------!---------!---------!---------!---------!---------! | 200 |
| 1: | eThrIleSerArgAspAsnSerLysAsnThrLeuTyrLeuGlnMetAsnSerLeuArgAlaGluAspThrAlaValTyrTyrCysAlaArgHisProGly | |
| | CACCATCTCCAGAGACAATTCCAAGAACACGCTGTATCTGCAAATGAACAGTCTGAGAGCCGAGGACACGGCCGTATATTACTGTGCCAGACATCCTGGT | |
| 201 | ---------!---------!---------!---------!---------!---------!---------!---------!---------!---------! | 300 |
| 1: | SerAlaGlyTyrGlyTyrGlnTyrTyrTyrAlaMetAspProTrpGlyGlnGlyThrLeuValThrValSerSerAlaSerThrLysGlyProSerValP | |
| | AGTGCTGGTTATGGTTATCAGTATTATTACGCCATGGACCCCTGGGGCCAAGGAACCCTGGTCACCGTCTCCTCAGCCTCCACCAAGGGCCCATCGGTCT | |
| 301 | ---------!---------!---------!---------!---------!---------!---------!---------!---------!---------! | 400 |
| 1: | heProLeuAlaProSerSerLysSerThrSerGlyGlyThrAlaAlaLeuGlyCysLeuValLysAspTyrPheProGluProValThrValSerTrpAs | |
| | TCCCCCTGGCACCCTCCTCCAAGAGCACCTCTGGGGGCACAGCGGCCCTGGGCTGCCTGGTCAAGGACTACTTCCCCGAACCGGTGACGGTGTCGTGGAA | |
| 401 | ---------!---------!---------!---------!---------!---------!---------!---------!---------!---------! | 500 |
| 1: | nSerGlyAlaLeuThrSerGlyValHisThrPheProAlaValLeuGlnSerSerGlyLeuTyrSerLeuSerSerValValThrValProSerSerSer | |
| | CTCAGGCGCCCTGACCAGCGGCGTGCACACCTTCCCGGCTGTCCTACAGTCCTCAGGACTCTACTCCCTCAGCAGCGTGGTGACCGTGCCCTCCAGCAGC | |
| 501 | ---------!---------!---------!---------!---------!---------!---------!---------!---------!---------! | 600 |
| 1: | LeuGlyThrGlnThrTyrIleCysAsnValAsnHisLysProSerAsnThrLysValAspLysLysValGluProLysSerCysAspLysThrHisThrC | |
| | TTGGGCACCCAGACCTACATCTGCAACGTGAATCACAAGCCCAGCAACACCAAGGTGGACAAGAAAGTTGAGCCCAAATCTTGTGACAAAACTCACACAT | |
| 601 | ---------!---------!---------!---------!---------!---------!---------!---------!---------!---------! | 700 |
| 1: | ysProProCysProAlaProGluLeuLeuGlyGlyProSerValPheLeuPheProProLysProLysAspThrLeuMetIleSerArgThrProGluVa | |
| | GCCCACCGTGCCCAGCACCTGAACTCCTGGGGGGACCGTCAGTCTTCCTCTTCCCCCCAAAACCCAAGGACACCCTCATGATCTCCCGGACCCCTGAGGT | |
| 701 | ---------!---------!---------!---------!---------!---------!---------!---------!---------!---------! | 800 |
| 1: | lThrCysValValValAspValSerHisGluAspProGluValLysPheAsnTrpTyrValAspGlyValGluValHisAsnAlaLysThrLysProArg | |
| | CACATGCGTGGTGGTGGACGTGAGCCACGAAGACCCTGAGGTCAAGTTCAACTGGTACGTGGACGGCGTGGAGGTGCATAATGCCAAGACAAAGCCGCGG | |
| 801 | ---------!---------!---------!---------!---------!---------!---------!---------!---------!---------! | 900 |
| 1: | GluGluGlnTyrAsnSerThrTyrArgValValSerValLeuThrValLeuHisGlnAspTrpLeuAsnGlyLysGluTyrLysCysLysValSerAsnL | |
| | GAGGAGCAGTACAACAGCACGTACCGTGTGGTCAGCGTCCTCACCGTCCTGCACCAGGACTGGCTGAATGGCAAGGAGTACAAGTGCAAGGTCTCCAACA | |
| 901 | ---------!---------!---------!---------!---------!---------!---------!---------!---------!---------! | 1000 |
| 1: | ysAlaLeuProAlaProIleGluLysThrIleSerLysAlaLysGlyGlnProArgGluProGlnValTyrThrLeuProProSerArgAspGluLeuTh | |
| | AAGCCCTCCCAGCCCCCATCGAGAAAACCATCTCCAAAGCCAAAGGGCAGCCCCGAGAACCACAGGTGTACACCCTGCCCCCATCCCGGGATGAGCTGAC | |
| 1001 | ---------!---------!---------!---------!---------!---------!---------!---------!---------!---------! | 1100 |
| 1: | rLysAsnGlnValSerLeuThrCysLeuValLysGlyPheTyrProSerAspIleAlaValGluTrpGluSerAsnGlyGlnProGluAsnAsnTyrLys | |
| | CAAGAACCAGGTCAGCCTGACCTGCCTGGTCAAAGGCTTCTATCCCAGCGACATCGCCGTGGAGTGGGAGAGCAATGGGCAGCCGGAGAACAACTACAAG | |
| 1101 | ---------!---------!---------!---------!---------!---------!---------!---------!---------!---------! | 1200 |
| 1: | ThrThrProProValLeuAspSerAspGlySerPhePheLeuTyrSerLysLeuThrValAspLysSerArgTrpGlnGlnGlyAsnValPheSerCysS | |
| | ACCACGCCTCCCGTGCTGGACTCCGACGGCTCCTTCTTCCTCTACAGCAAGCTCACCGTGGACAAGAGCAGGTGGCAGCAGGGGAACGTCTTCTCATGCT | |
| 1201 | ---------!---------!---------!---------!---------!---------!---------!---------!---------!---------! | 1300 |
| 1: | erValMetHisGluAlaLeuHisAsnHisTyrThrGlnLysSerLeuSerLeuSerProGlyLys | |
| | CCGTGATGCATGAGGCTCTGCACAACCACTACACGCAGAAGAGCCTCTCCCTGTCTCCGGGTAAA | |
| 1301 | ---------!---------!---------!---------!---------!---------!----- | 1365 |

6R8CF10 SEQ ID No. 74

| | | |
|---|---|---|
| 1: | GluValGlnLeuValGluSerGlyGlyGlyLeuValGlnProGlyGlySerLeuArgLeuSerCysLysAlaSerGlyPheThrPheSerArgGlnAlaL | |
| | GAGGTGCAGCTGGTGGAGTCTGGGGGAGGCTTGGTACAGCCTGGGGGGTCCCTGAGACTCTCCTGTAAGGCTTCTGGATTTACTTTTAGTCGTCAGGCTC | |
| 1 | ---------!---------!---------!---------!---------!---------!---------!---------!---------!---------! | 100 |
| 1: | euThrTrpValArgGlnAlaProGlyLysGlyLeuGluTrpIleGlyValIleAlaSerAsnAlaAspThrTyrTyrAlaSerTrpAlaLysGlyArgPh | |
| | TCACTTGGGTCCGCCAGGCTCCAGGGAAGGGGCTGGAGTGGATCGGAGTCATTGCTAGTAATGCTGACACATACTACGCGAGCTGGGCGAAAGGCCGGTT | |
| 101 | ---------!---------!---------!---------!---------!---------!---------!---------!---------!---------! | 200 |
| 1: | eThrIleSerArgAspAsnSerLysAsnThrLeuTyrLeuGlnMetAsnSerLeuArgAlaGluAspThrAlaValTyrTyrCysAlaArgHisProGly | |
| | CACCATCTCCAGAGACAATTCCAAGAACACGCTGTATCTGCAAATGAACAGCCTGAGAGCCGAGGACACGGCCGTATATTACTGTGCCAGACATCCTGGT | |
| 201 | ---------!---------!---------!---------!---------!---------!---------!---------!---------!---------! | 300 |
| 1: | SerAlaGlyTyrGlyTyrGlnTyrTyrTyrAlaMetAspProTrpGlyGlnGlyThrLeuValThrValSerSerAlaSerThrLysGlyProSerValP | |
| | AGTGCTGGTTATGGTTATCAGTATTATTACGCCATGGACCCCTGGGGCCAAGGAACCCTGGTCACCGTCTCCTCAGCCTCCACCAAGGGCCCATCGGTCT | |
| 301 | ---------!---------!---------!---------!---------!---------!---------!---------!---------!---------! | 400 |
| 1: | heProLeuAlaProSerSerLysSerThrSerGlyGlyThrAlaAlaLeuGlyCysLeuValLysAspTyrPheProGluProValThrValSerTrpAs | |
| | TCCCCCTGGCACCCTCCTCCAAGAGCACCTCTGGGGGCACAGCGGCCCTGGGCTGCCTGGTCAAGGACTACTTCCCCGAACCGGTGACGGTGTCGTGGAA | |
| 401 | ---------!---------!---------!---------!---------!---------!---------!---------!---------!---------! | 500 |
| 1: | nSerGlyAlaLeuThrSerGlyValHisThrPheProAlaValLeuGlnSerSerGlyLeuTyrSerLeuSerSerValValThrValProSerSerSer | |
| | CTCAGGCGCCCTGACCAGCGGCGTGCACACCTTCCCGGCTGTCCTACAGTCCTCAGGACTCTACTCCCTCAGCAGCGTGGTGACCGTGCCCTCCAGCAGC | |
| 501 | ---------!---------!---------!---------!---------!---------!---------!---------!---------!---------! | 600 |
| 1: | LeuGlyThrGlnThrTyrIleCysAsnValAsnHisLysProSerAsnThrLysValAspLysLysValGluProLysSerCysAspLysThrHisThrC | |
| | TTGGGCACCCAGACCTACATCTGCAACGTGAATCACAAGCCCAGCAACACCAAGGTGGACAAGAAAGTTGAGCCCAAATCTTGTGACAAAACTCACACAT | |
| 601 | ---------!---------!---------!---------!---------!---------!---------!---------!---------!---------! | 700 |
| 1: | ysProProCysProAlaProGluLeuLeuGlyGlyProSerValPheLeuPheProProLysProLysAspThrLeuMetIleSerArgThrProGluVa | |
| | GCCCACCGTGCCCAGCACCTGAACTCCTGGGGGGACCGTCAGTCTTCCTCTTCCCCCCAAAACCCAAGGACACCCTCATGATCTCCCGGACCCCTGAGGT | |
| 701 | ---------!---------!---------!---------!---------!---------!---------!---------!---------!---------! | 800 |
| 1: | lThrCysValValValAspValSerHisGluAspProGluValLysPheAsnTrpTyrValAspGlyValGluValHisAsnAlaLysThrLysProArg | |
| | CACATGCGTGGTGGTGGACGTGAGCCACGAAGACCCTGAGGTCAAGTTCAACTGGTACGTGGACGGCGTGGAGGTGCATAATGCCAAGACAAAGCCGCGG | |
| 801 | ---------!---------!---------!---------!---------!---------!---------!---------!---------!---------! | 900 |
| 1: | GluGluGlnTyrAsnSerThrTyrArgValValSerValLeuThrValLeuHisGlnAspTrpLeuAsnGlyLysGluTyrLysCysLysValSerAsnL | |
| | GAGGAGCAGTACAACAGCACGTACCGTGTGGTCAGCGTCCTCACCGTCCTGCACCAGGACTGGCTGAATGGCAAGGAGTACAAGTGCAAGGTCTCCAACA | |
| 901 | ---------!---------!---------!---------!---------!---------!---------!---------!---------!---------! | 1000 |
| 1: | ysAlaLeuProAlaProIleGluLysThrIleSerLysAlaLysGlyGlnProArgGluProGlnValTyrThrLeuProProSerArgAspGluLeuTh | |
| | AAGCCCTCCCAGCCCCCATCGAGAAAACCATCTCCAAAGCCAAAGGGCAGCCCCGAGAACCACAGGTGTACACCCTGCCCCCATCCCGGGATGAGCTGAC | |
| 1001 | ---------!---------!---------!---------!---------!---------!---------!---------!---------!---------! | 1100 |
| 1: | rLysAsnGlnValSerLeuThrCysLeuValLysGlyPheTyrProSerAspIleAlaValGluTrpGluSerAsnGlyGlnProGluAsnAsnTyrLys | |
| | CAAGAACCAGGTCAGCCTGACCTGCCTGGTCAAAGGCTTCTATCCCAGCGACATCGCCGTGGAGTGGGAGAGCAATGGGCAGCCGGAGAACAACTACAAG | |
| 1101 | ---------!---------!---------!---------!---------!---------!---------!---------!---------!---------! | 1200 |
| 1: | ThrThrProProValLeuAspSerAspGlySerPhePheLeuTyrSerLysLeuThrValAspLysSerArgTrpGlnGlnGlyAsnValPheSerCysS | |
| | ACCACGCCTCCCGTGCTGGACTCCGACGGCTCCTTCTTCCTCTACAGCAAGCTCACCGTGGACAAGAGCAGGTGGCAGCAGGGGAACGTCTTCTCATGCT | |
| 1201 | ---------!---------!---------!---------!---------!---------!---------!---------!---------!---------! | 1300 |
| 1: | erValMetHisGluAlaLeuHisAsnHisTyrThrGlnLysSerLeuSerLeuSerProGlyLys | |
| | CCGTGATGCATGAGGCTCTGCACAACCACTACACGCAGAAGAGCCTCTCCCTGTCTCCGGGTAAA | |
| 1301 | ---------!---------!---------!---------!---------!---------!----- | 1365 |

### CDRs of Clones Selected on BA.5 and BA.2.75.2

LCDR1, LCDR2, LCDR3, HCDR1, HCDR2, and HCDR3 of clones selected on BA.5 and BA.2.75.2 disclosed herein, i.e. SEQ ID Nos. 7-74, are shown in FIG. 3 (SEQ ID Nos. 75-278).

### Clones Selected on BQ.1.1 and XBB

### Light Chain Sequences of Clones Selected on BQ.1.1 and XBB

| 6R9A-A2 | | |
|---|---|---|
| SEQ ID No. 279 | | |
| 1: | AspIleGlnMetThrGlnSerProSerSerLeuSerAlaSerValGlyAspArgValThr | |
| | GACATCCAGATGACCCAGTCTCCATCCTCCCTGTCTGCATCTGTAGGAGACAGAGTCACC | |
| 1 | ---------!---------!---------!---------!---------!---------! | 60 |
| | CTGTAGGTCTACTGGGTCAGAGGTAGGAGGGACAGACGTAGACATCCTCTGTCTCAGTGG | |
| 1: | IleThrCysGlnAlaSerGluGluValHisTyrGlyIleAsnTrpTyrGlnGlnLysPro | |
| | ATCACTTGCCAGGCCAGTGAGGAGGTTCATTATGGGATTAATTGGTATCAGCAGAAACCA | |
| 61 | ---------!---------!---------!---------!---------!---------! | 120 |
| | TAGTGAACGGTCCGGTCACTCCTCCAAGTAATACCCTAATTAACCATAGTCGTCTTTGGT | |
| 1: | GlyLysAlaProLysLeuLeulleTyrGlyAlaAsnTyrArgValAspGlyValProSer | |
| | GGGAAAGCCCCTAAGCTCCTGATCTATGGTGCGAATTATCGTGTGGATGGGGTCCCATCA | |
| 121 | ---------!---------!---------!---------!---------!---------! | 180 |
| | CCCTTTCGGGGATTCGAGGACTAGATACCACGCTTAATAGCACACCTACCCCAGGGTAGT | |
| 1: | ArgPheSerGlySerGlySerGlyThrAspTyrThrLeuThrIleSerSerLeuGlnPro | |
| | AGGTTCAGTGGCAGTGGATCTGGGACAGATTACACTCTCACCATCAGCAGTCTGCAACCT | |
| 181 | ---------!---------!---------!---------!---------!---------! | 240 |
| | TCCAAGTCACCGTCACCTAGACCCTGTCTAATGTGAGAGTGGTAGTCGTCAGACGTTGGA | |
| 1: | GluAspPheAlaThrTyrTyrCysGlnGlnGlyTyrAsnValProThrAspPheGlyGly | |
| | GAAGATTTTGCAACTTACTACTGTCAACAAGGATATAATGTGCCTACTGATTTCGGCGGA | |
| 241 | ---------!---------!---------!---------!---------!---------! | 300 |
| | CTTCTAAAACGTTGAATGATGACAGTTGTTCCTATATTACACGGATGACTAAAGCCGCCT | |
| 1: | GlyThrLysValGlulleLysArgThrValAlaAlaProSerValPhellePheProPro | |
| | GGGACCAAGGTGGAGATCAAACGGACTGTGGCTGCACCATCTGTCTTCATCTTCCCGCCA | |
| 301 | ---------!---------!---------!---------!---------!---------! | 360 |
| | CCCTGGTTCCACCTCTAGTTTGCCTGACACCGACGTGGTAGACAGAAGTAGAAGGGCGGT | |
| 1: | SerAspGluGlnLeuLysSerGlyThrAlaSerValValCysLeuLeuAsnAsnPheTyr | |
| | TCTGATGAGCAGTTGAAATCTGGAACTGCCTCTGTTGTGTGCCTGCTGAATAACTTCTAT | |
| 361 | ---------!---------!---------!---------!---------!---------! | 420 |
| | AGACTACTCGTCAACTTTAGACCTTGACGGAGACAACACACGGACGACTTATTGAAGATA | |
| 1: | ProArgGluAlaLysValGlnTrpLysValAspAsnAlaLeuGlnSerGlyAsnSerGln | |
| | CCCAGAGAGGCCAAAGTACAGTGGAAGGTGGATAACGCCCTCCAATCGGGTAACTCCCAG | |
| 421 | ---------!---------!---------!---------!---------!---------! | 480 |
| | GGGTCTCTCCGGTTTCATGTCACCTTCCACCTATTGCGGGAGGTTAGCCCATTGAGGGTC | |
| 1: | GluSerValThrGluGlnAspSerLysAspSerThrTyrSerLeuSerSerThrLeuThr | |
| | GAGAGTGTCACAGAGCAGGACAGCAAGGACAGCACCTACAGCCTCAGCAGCACCCTGACG | |
| 481 | ---------!---------!---------!---------!---------!---------! | 540 |
| | CTCTCACAGTGTCTCGTCCTGTCGTTCCTGTCGTGGATGTCGGAGTCGTCGTGGGACTGC | |
| 1: | LeuSerLysAlaAspTyrGluLysHisLysValTyrAlaCysGluValThrHisGlnGly | |
| | CTGAGCAAAGCAGACTACGAGAAACACAAAGTCTACGCCTGCGAAGTCACCCATCAGGGC | |
| 541 | ---------!---------!---------!---------!---------!---------! | 600 |
| | GACTCGTTTCGTCTGATGCTCTTTGTGTTTCAGATGCGGACGCTTCAGTGGGTAGTCCCG | |
| 1: | LeuSerSerProValThrLysSerPheAsnArgGlyGluCys | |
| | CTGAGCTCGCCCGTCACAAAGAGCTTCAACAGGGGAGAGTGT | |
| 601 | ---------!---------!---------!---------!-- | 642 |
| | GACTCGAGCGGGCAGTGTTTCTCGAAGTTGTCCCCTCTCACA | |
| 6R9B-B11 | | |
| SEQ ID No. 280 | | |
| 1: | AspIleGlnMetThrGlnSerProSerSerLeuSerAlaSerValGlyAspArgValThr | |
| | GACATCCAGATGACCCAGTCTCCATCCTCCCTGTCTGCATCTGTAGGAGACAGAGTCACC | |
| 1 | ---------!---------!---------!---------!---------!---------! | 60 |
| | CTGTAGGTCTACTGGGTCAGAGGTAGGAGGGACAGACGTAGACATCCTCTGTCTCAGTGG | |
| 1: | IleThrCysGlnAlaSerGlnAlaIleLysTyrAlaIleAsnTrpTyrGlnGlnLysPro | |
| | ATCACTTGCCAGGCCAGTCAGGCGATTAAGTATGCGATTAATTGGTATCAGCAGAAACCA | |
| 61 | ---------!---------!---------!---------!---------!---------! | 120 |
| | TAGTGAACGGTCCGGTCAGTCCGCTAATTCATACGCTAATTAACCATAGTCGTCTTTGGT | |
| 1: | GlyLysAlaProLysLeuLeuIleTyrGlyAlaAsnTyrArgGlyProGlyValProSer | |
| | GGGAAAGCCCCTAAGCTCCTGATCTATGGTGCTAATTATCGTGGGCCGGGGGTCCCATCA | |
| 121 | ---------!---------!---------!---------!---------!---------! | 180 |
| | CCCTTTCGGGGATTCGAGGACTAGATACCACGATTAATAGCACCCGGCCCCCAGGGTAGT | |
| 1: | ArgPheSerGlySerGlySerGlyThrAspTyrThrLeuThrIleSerSerLeuGlnPro | |
| | AGGTTCAGTGGCAGTGGATCTGGGACAGATTACACTCTCACCATCAGCAGTCTGCAACCT | |
| 181 | ---------!---------!---------!---------!---------!---------! | 240 |
| | TCCAAGTCACCGTCACCTAGACCCTGTCTAATGTGAGAGTGGTAGTCGTCAGACGTTGGA | |
| 1: | GluAspPheAlaThrTyrTyrCysGlnGlnGlyTyrIleValProIleThrPheGlyGly | |
| | GAAGATTTTGCAACTTACTACTGTCAACAAGGATATATTGTGCCTATTACGTTCGGCGGA | |
| 241 | ---------!---------!---------!---------!---------!---------! | 300 |
| | CTTCTAAAACGTTGAATGATGACAGTTGTTCCTATATAACACGGATAATGCAAGCCGCCT | |
| 1: | GlyThrLysValGluIleLysArgThrValAlaAlaProSerValPheIlePheProPro | |
| | GGGACCAAGGTGGAGATCAAACGGACTGTGGCTGCACCATCTGTCTTCATCTTCCCGCCA | |
| 301 | ---------!---------!---------!---------!---------!---------! | 360 |
| | CCCTGGTTCCACCTCTAGTTTGCCTGACACCGACGTGGTAGACAGAAGTAGAAGGGCGGT | |
| 1: | SerAspGluGlnLeuLysSerGlyThrAlaSerValValCysLeuLeuAsnAsnPheTyr | |
| | TCTGATGAGCAGTTGAAATCTGGAACTGCCTCTGTTGTGTGCCTGCTGAATAACTTCTAT | |
| 361 | ---------!---------!---------!---------!---------!---------! | 420 |
| | AGACTACTCGTCAACTTTAGACCTTGACGGAGACAACACACGGACGACTTATTGAAGATA | |
| 1: | ProArgGluAlaLysValGlnTrpLysValAspAsnAlaLeuGlnSerGlyAsnSerGln | |
| | CCCAGAGAGGCCAAAGTACAGTGGAAGGTGGATAACGCCCTCCAATCGGGTAACTCCCAG | |
| 421 | ---------!---------!---------!---------!---------!---------! | 480 |
| | GGGTCTCTCCGGTTTCATGTCACCTTCCACCTATTGCGGGAGGTTAGCCCATTGAGGGTC | |
| 1: | GluSerValThrGluGlnAspSerLysAspSerThrTyrSerLeuSerSerThrLeuThr | |
| | GAGAGTGTCACAGAGCAGGACAGCAAGGACAGCACCTACAGCCTCAGCAGCACCCTGACG | |
| 481 | ---------!---------!---------!---------!---------!---------! | 540 |
| | CTCTCACAGTGTCTCGTCCTGTCGTTCCTGTCGTGGATGTCGGAGTCGTCGTGGGACTGC | |
| 1: | LeuSerLysAlaAspTyrGluLysHisLysValTyrAlaCysGluValThrHisGlnGly | |
| | CTGAGCAAAGCAGACTACGAGAAACACAAAGTCTACGCCTGCGAAGTCACCCATCAGGGC | |
| 541 | ---------!---------!---------!---------!---------!---------! | 600 |
| | GACTCGTTTCGTCTGATGCTCTTTGTGTTTCAGATGCGGACGCTTCAGTGGGTAGTCCCG | |
| 1: | LeuSerSerProValThrLysSerPheAsnArgGlyGluCys | |
| | CTGAGCTCGCCCGTCACAAAGAGCTTCAACAGGGGAGAGTGT | |
| 601 | ---------!---------!---------!---------!-- | 642 |
| | GACTCGAGCGGGCAGTGTTTCTCGAAGTTGTCCCCTCTCACA | |
| 6R9B-E9 | | |
| SEQ ID No. 281 | | |
| 1: | AspIleGlnMetThrGlnSerProSerSerLeuSerAlaSerValGlyAspArgValThr | |
| | GACATCCAGATGACCCAGTCTCCATCCTCCCTGTCTGCATCTGTAGGAGACAGAGTCACC | |
| 1 | ---------!---------!---------!---------!---------!---------! | 60 |
| | CTGTAGGTCTACTGGGTCAGAGGTAGGAGGGACAGACGTAGACATCCTCTGTCTCAGTGG | |
| 1: | IleThrCysGlnAlaSerGluAsnIleArgTyrAlaValAsnTrpTyrGlnGlnLysPro | |
| | ATCACTTGCCAGGCCAGTGAGAATATTAGGTATGCTGTTAATTGGTATCAGCAGAAACCA | |
| 61 | ---------!---------!---------!---------!---------!---------! | 120 |
| | TAGTGAACGGTCCGGTCACTCTTATAATCCATACGACAATTAACCATAGTCGTCTTTGGT | |
| 1: | GlyLysAlaProLysLeuLeuIleTyrGlyAlaAsnTyrArgValAspGlyValProSer | |
| | GGGAAAGCCCCTAAGCTCCTGATCTATGGTGCGAATTATCGTGTGGATGGGGTCCCATCA | |
| 121 | ---------!---------!---------!---------!---------!---------! | 180 |
| | CCCTTTCGGGGATTCGAGGACTAGATACCACGCTTAATAGCACACCTACCCCAGGGTAGT | |
| 1: | ArgPheSerGlySerGlySerGlyThrAspTyrThrLeuThrIleSerSerLeuGlnPro | |
| | AGGTTCAGTGGCAGTGGATCTGGGACAGATTACACTCTCACCATCAGCAGTCTGCAACCT | |
| 181 | ---------!---------!---------!---------!---------!---------! | 240 |
| | TCCAAGTCACCGTCACCTAGACCCTGTCTAATGTGAGAGTGGTAGTCGTCAGACGTTGGA | |
| 1: | GluAspPheAlaThrTyrTyrCysGlnGlnGlyTyrArgValGlnIleLeuPheGlyGly | |
| | GAAGATTTTGCAACTTACTACTGTCAACAAGGATATCGTGTTCAGATTTTGTTCGGCGGA | |
| 241 | ---------!---------!---------!---------!---------!---------! | 300 |
| | CTTCTAAAACGTTGAATGATGACAGTTGTTCCTATAGCACAAGTCTAAAACAAGCCGCCT | |
| 1: | GlyThrLysValGluIleLysArgThrValAlaAlaProSerValPheIlePheProPro | |
| | GGGACCAAGGTGGAGATCAAACGGACTGTGGCTGCACCATCTGTCTTCATCTTCCCGCCA | |
| 301 | ---------!---------!---------!---------!---------!---------! | 360 |
| | CCCTGGTTCCACCTCTAGTTTGCCTGACACCGACGTGGTAGACAGAAGTAGAAGGGCGGT | |
| 1: | SerAspGluGlnLeuLysSerGlyThrAlaSerValValCysLeuLeuAsnAsnPheTyr | |
| | TCTGACGAGCAGTTGAAATCTGGAACTGCCTCTGTTGTGTGCCTGCTGAATAACTTCTAT | |
| 361 | ---------!---------!---------!---------!---------!---------! | 420 |
| | AGACTGCTCGTCAACTTTAGACCTTGACGGAGACAACACACGGACGACTTATTGAAGATA | |
| 1: | ProArgGluAlaLysValGlnTrpLysValAspAsnAlaLeuGlnSerGlyAsnSerGln | |
| | CCCAGAGAGGCCAAAGTACAGTGGAAGGTGGATAACGCCCTCCAATCGGGTAACTCCCAG | |
| 421 | ---------!---------!---------!---------!---------!---------! | 480 |
| | GGGTCTCTCCGGTTTCATGTCACCTTCCACCTATTGCGGGAGGTTAGCCCATTGAGGGTC | |
| 1: | GluSerValThrGluGlnAspSerLysAspSerThrTyrSerLeuSerSerThrLeuThr | |
| | GAGAGTGTCACAGAGCAGGACAGCAAGGACAGCACCTACAGCCTCAGCAGCACCCTGACG | |
| 481 | ---------!---------!---------!---------!---------!---------! | 540 |
| | CTCTCACAGTGTCTCGTCCTGTCGTTCCTGTCGTGGATGTCGGAGTCGTCGTGGGACTGC | |
| 1: | LeuSerLysAlaAspTyrGluLysHisLysValTyrAlaCysGluValThrHisGlnGly | |
| | CTGAGCAAAGCAGACTACGAGAAACACAAAGTCTACGCCTGCGAAGTCACCCATCAGGGC | |
| 541 | ---------!---------!---------!---------!---------!---------! | 600 |
| | GACTCGTTTCGTCTGATGCTCTTTGTGTTTCAGATGCGGACGCTTCAGTGGGTAGTCCCG | |
| 1: | LeuSerSerProValThrLysSerPheAsnArgGlyGluCys | |
| | CTGAGCTCGCCCGTCACAAAGAGCTTCAACAGGGGAGAGTGT | |
| 601 | ---------!---------!---------!---------!-- | 642 |
| | GACTCGAGCGGGCAGTGTTTCTCGAAGTTGTCCCCTCTCACA | |
| 6R9E-A8 | | |
| SEQ ID No. 282 | | |
| 1: | AspIleGlnMetThrGlnSerProSerSerLeuSerAlaSerValGlyAspArgValThr | |
| | GACATCCAGATGACCCAGTCTCCATCCTCCCTGTCTGCATCTGTAGGAGACAGAGTCACC | |
| 1 | ---------!---------!---------!---------!---------!---------! | 60 |
| | CTGTAGGTCTACTGGGTCAGAGGTAGGAGGGACAGACGTAGACATCCTCTGTCTCAGTGG | |
| 1: | IleThrCysGlnAlaSerGluGluValHisTyrGlyIleAsnTrpTyrGlnGlnLysPro | |
| | ATCACTTGCCAGGCCAGTGAGGAGGTTCATTATGGGATTAATTGGTATCAGCAGAAACCA | |
| 61 | ---------!---------!---------!---------!---------!---------! | 120 |
| | TAGTGAACGGTCCGGTCACTCCTCCAAGTAATACCCTAATTAACCATAGTCGTCTTTGGT | |
| 1: | GlyLysAlaProLysLeuLeulleTyrGlyAlaAsnTyrArgValAspGlyValProSer | |
| | GGGAAAGCCCCTAAGCTCCTGATCTATGGTGCGAATTATCGTGTGGATGGGGTCCCATCA | |
| 121 | ---------!---------!---------!---------!---------!---------! | 180 |
| | CCCTTTCGGGGATTCGAGGACTAGATACCACGCTTAATAGCACACCTACCCCAGGGTAGT | |
| 1: | ArgPheSerGlySerGlySerGlyThrAspTyrThrLeuThrIleSerSerLeuGlnPro | |
| | AGGTTCAGTGGCAGTGGATCTGGGACAGATTACACTCTCACCATCAGCAGTCTGCAACCT | |
| 181 | ---------!---------!---------!---------!---------!---------! | 240 |
| | TCCAAGTCACCGTCACCTAGACCCTGTCTAATGTGAGAGTGGTAGTCGTCAGACGTTGGA | |
| 1: | TCCAAGTCACCGTCACCTAGACCCTGTCTAATGTGAGAGTGGTAGTCGTCAGACGTTGGA | |
| | GluAspPheAlaThrTyrTyrCysGlnGlnGlyTyrAsnValProThrAspPheGlyGly | |
| 241 | ---------!---------!---------!---------!---------!---------! | 300 |
| | CTTCTAAAACGTTGAATGATGACAGTTGTTCCTATATTACACGGATGACTAAAGCCGCCT | |
| 1: | GlyThrLysValGlulleLysArgThrValAlaAlaProSerValPhellePheProPro | |
| | GGGACCAAGGTGGAGATCAAACGGACTGTGGCTGCACCATCTGTCTTCATCTTCCCGCCA | |
| 301 | ---------!---------!---------!---------!---------!---------! | 360 |
| | CCCTGGTTCCACCTCTAGTTTGCCTGACACCGACGTGGTAGACAGAAGTAGAAGGGCGGT | |
| 1: | SerAspGluGInLeuLysSerGlyThrAlaSerValValCysLeuLeuAsnAsnPheTyr | |
| | TCTGATGAGCAGTTGAAATCTGGAACTGCCTCTGTTGTGTGCCTGCTGAATAACTTCTAT | |
| 361 | ---------!---------!---------!---------!---------!---------! | 420 |
| | AGACTACTCGTCAACTTTAGACCTTGACGGAGACAACACACGGACGACTTATTGAAGATA | |
| 1: | ProArgGluAlaLysValGlnTrpLysValAspAsnAlaLeuGlnSerGlyAsnSerGln | |
| | CCCAGAGAGGCCAAAGTACAGTGGAAGGTGGATAACGCCCTCCAATCGGGTAACTCCCAG | |
| 421 | ---------!---------!---------!---------!---------!---------! | 480 |
| | GGGTCTCTCCGGTTTCATGTCACCTTCCACCTATTGCGGGAGGTTAGCCCATTGAGGGTC | |
| 1: | GluSerValThrGluGlnAspSerLysAspSerThrTyrSerLeuSerSerThrLeuThr | |
| | GAGAGTGTCACAGAGCAGGACAGCAAGGACAGCACCTACAGCCTCAGCAGCACCCTGACG | |
| 481 | ---------!---------!---------!---------!---------!---------! | 540 |
| | CTCTCACAGTGTCTCGTCCTGTCGTTCCTGTCGTGGATGTCGGAGTCGTCGTGGGACTGC | |
| 1: | LeuSerLysAlaAspTyrGluLysHisLysValTyrAlaCysGluValThrHisGlnGly | |
| | CTGAGCAAAGCAGACTACGAGAAACACAAAGTCTACGCCTGCGAAGTCACTCATCAGGGC | |
| 541 | ---------!---------!---------!---------!---------!---------! | 600 |
| | GACTCGTTTCGTCTGATGCTCTTTGTGTTTCAGATGCGGACGCTTCAGTGAGTAGTCCCG | |
| 1: | LeuSerSerProValThrLysSerPheAsnArgGlyGluCys | |
| | CTGAGCTCGCCCGTCACAAAGAGCTTCAACAGGGGAGAGTGT | |
| 601 | ---------!---------!---------!---------!-- | 642 |
| | GACTCGAGCGGGCAGTGTTTCTCGAAGTTGTCCCCTCTCACA | |

### Heavy Chain Sequences of Clones Selected on BQ.1.1 and XBB

| 6R9A-A2 | | |
|---|---|---|
| SEQ ID No. 283 | | |
| 1: | GluValGlnLeuValGluSerGlyGlyGlyLeuValGlnProGlyGlySerLeuArgLeu | |
| | GAGGTGCAGCTGGTGGAGTCTGGGGGAGGCTTGGTACAGCCTGGGGGGTCCCTGAGACTC | |
| 1 | ---------!---------!---------!---------!---------!---------! | 60 |
| | CTCCACGTCGACCACCTCAGACCCCCTCCGAACCATGTCGGACCCCCCAGGGACTCTGAG | |
| 1: | SerCysThrAlaSerGlyPheSerPheProHisGlnAlaMetThrTrpValArgGlnAla | |
| | TCCTGTACGGCTTCTGGATTTAGTTTTCCGCATCAGGCGATGACTTGGGTCCGCCAGGCT | |
| 61 | ---------!---------!---------!---------!---------!---------! | 120 |
| | AGGACATGCCGAAGACCTAAATCAAAAGGCGTAGTCCGCTACTGAACCCAGGCGGTCCGA | |
| 1: | ProGlyLysGlyLeuGluTrpIleGlyValIleAlaSerAsnAlaAspThrTyrTyrAla | |
| | CCAGGGAAGGGGCTGGAGTGGATCGGAGTCATTGCTAGTAATGCTGACACATACTACGCG | |
| 121 | ---------!---------!---------!---------!---------!---------! | 180 |
| | GGTCCCTTCCCCGACCTCACCTAGCCTCAGTAACGATCATTACGACTGTGTATGATGCGC | |
| 1: | SerTrpAlaLysGlyArgPheThrIleSerArgAspAsnSerLysAsnThrLeuTyrLeu | |
| | AGCTGGGCGAAAGGCCGGTTCACCATCTCCAGAGACAATTCCAAGAACACGCTGTATCTG | |
| 181 | ---------!---------!---------!---------!---------!---------! | 240 |
| | TCGACCCGCTTTCCGGCCAAGTGGTAGAGGTCTCTGTTAAGGTTCTTGTGCGACATAGAC | |
| 1: | GlnMetAsnSerLeuArgAlaGluAspThrAlaValTyrTyrCysAlaArgHisProGly | |
| 241 | CAAATGAACAGCCTGAGAGCCGAGGACACGGCCGTATATTACTGTGCCAGACATCCTGGT | 300 |
| | GTTTACTTGTCGGACTCTCGGCTCCTGTGCCGGCATATAATGACACGGTCTGTAGGACCA | |
| 1: | SerAlaGlyTyrGlyTyrGlnTyrTyrTyrAlaMetAspProTrpGlyGlnGlyThrLeu | |
| | AGTGCTGGTTATGGTTATCAGTATTATTACGCCATGGACCCCTGGGGCCAAGGAACCCTG | |
| 301 | ---------!---------!---------!---------!---------!---------! | 360 |
| | TCACGACCAATACCAATAGTCATAATAATGCGGTACCTGGGGACCCCGGTTCCTTGGGAC | |
| 1: | ValThrValSerSerAlaSerThrLysGlyProSerValPheProLeuAlaProSerSer | |
| | GTCACCGTCTCCTCAGCCTCCACCAAGGGCCCATCGGTCTTCCCCCTGGCACCCTCCTCC | |
| 361 | ---------!---------!---------!---------!---------!---------! | 420 |
| | CAGTGGCAGAGGAGTCGGAGGTGGTTCCCGGGTAGCCAGAAGGGGGACCGTGGGAGGAGG | |
| 1: | LysSerThrSerGlyGlyThrAlaAlaLeuGlyCysLeuValLysAspTyrPheProGlu | |
| | AAGAGCACCTCTGGGGGCACAGCGGCCCTGGGCTGCCTGGTCAAGGACTACTTCCCCGAA | |
| 421 | ---------!---------!---------!---------!---------!---------! | 480 |
| | TTCTCGTGGAGACCCCCGTGTCGCCGGGACCCGACGGACCAGTTCCTGATGAAGGGGCTT | |
| 1: | ProValThrValSerTrpAsnSerGlyAlaLeuThrSerGlyValHisThrPheProAla | |
| | CCGGTGACGGTGTCGTGGAACTCAGGCGCCCTGACCAGCGGCGTGCACACCTTCCCGGCT | |
| 481 | ---------!---------!---------!---------!---------!---------! | 540 |
| | GGCCACTGCCACAGCACCTTGAGTCCGCGGGACTGGTCGCCGCACGTGTGGAAGGGCCGA | |
| 1: | ValLeuGlnSerSerGlyLeuTyrSerLeuSerSerValValThrValProSerSerSer | |
| | GTCCTACAGTCCTCAGGACTCTACTCCCTCAGCAGCGTGGTGACCGTGCCCTCCAGCAGC | |
| 541 | ---------!---------!---------!---------!---------!---------! | 600 |
| | CAGGATGTCAGGAGTCCTGAGATGAGGGAGTCGTCGCACCACTGGCACGGGAGGTCGTCG | |
| 1: | LeuGlyThrGlnThrTyrIleCysAsnValAsnHisLysProSerAsnThrLysValAsp | |
| | TTGGGCACCCAGACCTACATCTGCAACGTGAATCACAAGCCCAGCAACACCAAGGTGGAC | |
| 601 | ---------!---------!---------!---------!---------!---------! | 660 |
| | AACCCGTGGGTCTGGATGTAGACGTTGCACTTAGTGTTCGGGTCGTTGTGGTTCCACCTG | |
| 1: | LysLysValGluProLysSerCysAspLysThrHisThrCysProProCysProAlaPro | |
| | AAGAAAGTTGAGCCCAAATCTTGTGACAAAACTCACACATGCCCACCGTGCCCAGCACCT | |
| 661 | ---------!---------!---------!---------!---------!---------! | 720 |
| | TTCTTTCAACTCGGGTTTAGAACACTGTTTTGAGTGTGTACGGGTGGCACGGGTCGTGGA | |
| 1: | GluLeuLeuGlyGlyProSerValPheLeuPheProProLysProLysAspThrLeuMet | |
| | GAACTCCTGGGGGGACCGTCAGTCTTCCTCTTCCCCCCAAAACCCAAGGACACCCTCATG | |
| 721 | ---------!---------!---------!---------!---------!---------! | 780 |
| | CTTGAGGACCCCCCTGGCAGTCAGAAGGAGAAGGGGGGTTTTGGGTTCCTGTGGGAGTAC | |
| 1: | IleSerArgThrProGluValThrCysValValValAspValSerHisGluAspProGlu | |
| | ATCTCCCGGACCCCTGAGGTCACATGCGTGGTGGTGGACGTGAGCCACGAAGACCCTGAG | |
| 781 | ---------!---------!---------!---------!---------!---------! | 840 |
| | TAGAGGGCCTGGGGACTCCAGTGTACGCACCACCACCTGCACTCGGTGCTTCTGGGACTC | |
| 1: | ValLysPheAsnTrpTyrValAspGlyValGluValHisAsnAlaLysThrLysProArg | |
| | GTCAAGTTCAACTGGTACGTGGACGGCGTGGAGGTGCATAATGCCAAGACAAAGCCGCGG | |
| 841 | ---------!---------!---------!---------!---------!---------! | 900 |
| | CAGTTCAAGTTGACCATGCACCTGCCGCACCTCCACGTATTACGGTTCTGTTTCGGCGCC | |
| 1: | GluGluGlnTyrAsnSerThrTyrArgValValSerValLeuThrValLeuHisGlnAsp | |
| | GAGGAGCAGTACAACAGCACGTACCGTGTGGTCAGCGTCCTCACCGTCCTGCACCAGGAC | |
| 901 | ---------!---------!---------!---------!---------!---------! | 960 |
| | CTCCTCGTCATGTTGTCGTGCATGGCACACCAGTCGCAGGAGTGGCAGGACGTGGTCCTG | |
| 1: | TrpLeuAsnGlyLysGluTyrLysCysLysValSerAsnLysAlaLeuProAlaProIle | |
| | TGGCTGAATGGCAAGGAGTACAAGTGCAAGGTCTCCAACAAAGCCCTCCCAGCCCCCATC | |
| 961 | | 1020 |
| | ACCGACTTACCGTTCCTCATGTTCACGTTCCAGAGGTTGTTTCGGGAGGGTCGGGGGTAG | |
| 1: | GluLysThrIleSerLysAlaLysGlyGlnProArgGluProGlnValTyrThrLeuPro | |
| | GAGAAAACCATCTCCAAAGCCAAAGGGCAGCCCCGAGAACCACAGGTGTACACCCTGCCC | |
| 1021 | ---------!---------!---------!---------!---------!---------! | 1080 |
| | CTCTTTTGGTAGAGGTTTCGGTTTCCCGTCGGGGCTCTTGGTGTCCACATGTGGGACGGG | |
| 1: | ProSerArgAspGluLeuThrLysAsnGlnValSerLeuThrCysLeuValLysGlyPhe | |
| | CCATCCCGGGATGAGCTGACCAAGAACCAGGTCAGCCTGACCTGCCTGGTCAAAGGCTTC | |
| 1081 | ---------!---------!---------!---------!---------!---------! | 1140 |
| | GGTAGGGCCCTACTCGACTGGTTCTTGGTCCAGTCGGACTGGACGGACCAGTTTCCGAAG | |
| 1: | TyrProSerAspIleAlaValGluTrpGluSerAsnGlyGlnProGluAsnAsnTyrLys | |
| | TATCCCAGCGACATCGCCGTGGAGTGGGAGAGCAATGGGCAGCCGGAGAACAACTACAAG | |
| 1141 | ---------!---------!---------!---------!---------!---------! | 1200 |
| | ATAGGGTCGCTGTAGCGGCACCTCACCCTCTCGTTACCCGTCGGCCTCTTGTTGATGTTC | |
| 1: | ThrThrProProValLeuAspSerAspGlySerPhePheLeuTyrSerLysLeuThrVal | |
| | ACCACGCCTCCCGTGCTGGACTCCGACGGCTCCTTCTTCCTCTACAGCAAGCTCACCGTG | |
| 1201 | ---------!---------!---------!---------!---------!---------! | 1260 |
| | TGGTGCGGAGGGCACGACCTGAGGCTGCCGAGGAAGAAGGAGATGTCGTTCGAGTGGCAC | |
| 1: | AspLysSerArgTrpGlnGlnGlyAsnValPheSerCysSerValMetHisGluAlaLeu | |
| | GACAAGAGCAGGTGGCAGCAGGGGAACGTCTTCTCATGCTCCGTGATGCATGAGGCTCTG | |
| 1261 | ---------!---------!---------!---------!---------!---------! | 1320 |
| | CTGTTCTCGTCCACCGTCGTCCCCTTGCAGAAGAGTACGAGGCACTACGTACTCCGAGAC | |
| 1: | HisAsnHisTyrThrGlnLysSerLeuSerLeuSerProGlyLys | |
| | CACAACCACTACACGCAGAAGAGCCTCTCCCTGTCTCCGGGTAAA | |
| 1321 | ---------!---------!---------!---------!----- | 1365 |
| | GTGTTGGTGATGTGCGTCTTCTCGGAGAGGGACAGAGGCCCATTT | |
| 6R9B-B11 | | |
| SEQ ID No. 284 | | |
| 1: | GluValGlnLeuValGluSerGlyGlyGlyLeuValGlnProGlyGlySerLeuArgLeu | |
| | GAGGTGCAGCTGGTGGAGTCTGGGGGAGGCTTGGTACAGCCTGGGGGGTCCCTGAGACTC | |
| 1 | ---------!---------!---------!---------!---------!---------! | 60 |
| | CTCCACGTCGACCACCTCAGACCCCCTCCGAACCATGTCGGACCCCCCAGGGACTCTGAG | |
| 1: | SerCysLysAlaSerGlyPheThrPheSerArgGlnAlaLeuThrTrpValArgGlnAla | |
| | TCCTGTAAGGCTTCTGGATTTACTTTTAGTCGTCAGGCTCTCACTTGGGTCCGCCAGGCT | |
| 61 | ---------!---------!---------!---------!---------!---------! | 120 |
| | AGGACATTCCGAAGACCTAAATGAAAATCAGCAGTCCGAGAGTGAACCCAGGCGGTCCGA | |
| 1: | ProGlyLysGlyLeuGluTrpIleGlyValIleAlaSerAsnAlaAspThrTyrTyrAla | |
| | CCAGGGAAGGGGCTGGAGTGGATCGGAGTCATTGCTAGTAATGCTGACACATACTACGCG | |
| 121 | ---------!---------!---------!---------!---------!---------! | 180 |
| | GGTCCCTTCCCCGACCTCACCTAGCCTCAGTAACGATCATTACGACTGTGTATGATGCGC | |
| 1: | SerTrpAlaLysGlyArgPheThrIleSerArgAspAsnSerLysAsnThrLeuTyrLeu | |
| | AGCTGGGCGAAAGGCCGGTTCACCATCTCCAGAGACAATTCCAAGAACACGCTGTATCTG | |
| 181 | ---------!---------!---------!---------!---------!---------! | 240 |
| | TCGACCCGCTTTCCGGCCAAGTGGTAGAGGTCTCTGTTAAGGTTCTTGTGCGACATAGAC | |
| 1: | GlnMetAsnSerLeuArgAlaGluAspThrAlaValTyrTyrCysAlaArgHisProGly | |
| | CAAATGAACAGCCTGAGAGCCGAGGACACGGCCGTATATTACTGTGCCAGACATCCTGGT | |
| 241 | ---------!---------!---------!---------!---------!---------! | 300 |
| | GTTTACTTGTCGGACTCTCGGCTCCTGTGCCGGCATATAATGACACGGTCTGTAGGACCA | |
| 1: | SerAlaGlyTyrGlyTyrGlnTyrTyrTyrAlaMetAspProTrpGlyGlnGlyThrLeu | |
| | AGTGCTGGTTATGGTTATCAGTATTATTACGCCATGGACCCCTGGGGCCAAGGAACCCTG | |
| 301 | ---------!---------!---------!---------!---------!---------! | 360 |
| | TCACGACCAATACCAATAGTCATAATAATGCGGTACCTGGGGACCCCGGTTCCTTGGGAC | |
| 1: | ValThrValSerSerAlaSerThrLysGlyProSerValPheProLeuAlaProSerSer | |
| | GTCACCGTCTCCTCAGCCTCCACCAAGGGCCCATCGGTCTTCCCCCTGGCACCCTCCTCC | |
| 361 | ---------!---------!---------!---------!---------!---------! | 420 |
| | CAGTGGCAGAGGAGTCGGAGGTGGTTCCCGGGTAGCCAGAAGGGGGACCGTGGGAGGAGG | |
| 1: | LysSerThrSerGlyGlyThrAlaAlaLeuGlyCysLeuValLysAspTyrPheProGlu | |
| | AAGAGCACCTCTGGGGGCACAGCGGCCCTGGGCTGCCTGGTCAAGGACTACTTCCCCGAA | |
| 421 | ---------!---------!---------!---------!---------!---------! | 480 |
| | TTCTCGTGGAGACCCCCGTGTCGCCGGGACCCGACGGACCAGTTCCTGATGAAGGGGCTT | |
| 1: | ProValThrValSerTrpAsnSerGlyAlaLeuThrSerGlyValHisThrPheProAla | |
| | CCGGTGACGGTGTCGTGGAACTCAGGCGCCCTGACCAGCGGCGTGCACACCTTCCCGGCT | |
| 481 | ---------!---------!---------!---------!---------!---------! | 540 |
| | GGCCACTGCCACAGCACCTTGAGTCCGCGGGACTGGTCGCCGCACGTGTGGAAGGGCCGA | |
| 1: | ValLeuGlnSerSerGlyLeuTyrSerLeuSerSerValValThrValProSerSerSer | |
| | GTCCTACAGTCCTCAGGACTCTACTCCCTCAGCAGCGTGGTGACCGTGCCCTCCAGCAGC | |
| 541 | ---------!---------!---------!---------!---------!---------! | 600 |
| | CAGGATGTCAGGAGTCCTGAGATGAGGGAGTCGTCGCACCACTGGCACGGGAGGTCGTCG | |
| 1: | LeuGlyThrGlnThrTyrIleCysAsnValAsnHisLysProSerAsnThrLysValAsp | |
| | TTGGGCACCCAGACCTACATCTGCAACGTGAATCACAAGCCCAGCAACACCAAGGTGGAC | |
| 601 | ---------!---------!---------!---------!---------!---------! | 660 |
| | AACCCGTGGGTCTGGATGTAGACGTTGCACTTAGTGTTCGGGTCGTTGTGGTTCCACCTG | |
| 1: | LysLysValGluProLysSerCysAspLysThrHisThrCysProProCysProAlaPro | |
| | AAGAAAGTTGAGCCCAAATCTTGTGACAAAACTCACACATGCCCACCGTGCCCAGCACCT | |
| 661 | ---------!---------!---------!---------!---------!---------! | 720 |
| | TTCTTTCAACTCGGGTTTAGAACACTGTTTTGAGTGTGTACGGGTGGCACGGGTCGTGGA | |
| 1: | GluLeuLeuGlyGlyProSerValPheLeuPheProProLysProLysAspThrLeuMet | |
| | GAACTCCTGGGGGGACCGTCAGTCTTCCTCTTCCCCCCAAAACCCAAGGACACCCTCATG | |
| 721 | ---------!---------!---------!---------!---------!---------! | 780 |
| | CTTGAGGACCCCCCTGGCAGTCAGAAGGAGAAGGGGGGTTTTGGGTTCCTGTGGGAGTAC | |
| 1: | IleSerArgThrProGluValThrCysValValValAspValSerHisGluAspProGlu | |
| | ATCTCCCGGACCCCTGAGGTCACATGCGTGGTGGTGGACGTGAGCCACGAAGACCCTGAG | |
| 781 | ---------!---------!---------!---------!---------!---------! | 840 |
| | TAGAGGGCCTGGGGACTCCAGTGTACGCACCACCACCTGCACTCGGTGCTTCTGGGACTC | |
| 1: | ValLysPheAsnTrpTyrValAspGlyValGluValHisAsnAlaLysThrLysProArg | |
| | GTCAAGTTCAACTGGTACGTGGACGGCGTGGAGGTGCATAATGCCAAGACAAAGCCGCGG | |
| 841 | ---------!---------!---------!---------!---------!---------! | 900 |
| | CAGTTCAAGTTGACCATGCACCTGCCGCACCTCCACGTATTACGGTTCTGTTTCGGCGCC | |
| 1: | GluGluGlnTyrAsnSerThrTyrArgValValSerValLeuThrValLeuHisGlnAsp | |
| | GAGGAGCAGTACAACAGCACGTACCGTGTGGTCAGCGTCCTCACCGTCCTGCACCAGGAC | |
| 901 | ---------!---------!---------!---------!---------!---------! | 960 |
| | CTCCTCGTCATGTTGTCGTGCATGGCACACCAGTCGCAGGAGTGGCAGGACGTGGTCCTG | |
| 1: | TrpLeuAsnGlyLysGluTyrLysCysLysValSerAsnLysAlaLeuProAlaProIle | |
| | TGGCTGAATGGCAAGGAGTACAAGTGCAAGGTCTCCAACAAAGCCCTCCCAGCCCCCATC | |
| 961 | ---------!---------!---------!---------!---------!---------! | 1020 |
| | ACCGACTTACCGTTCCTCATGTTCACGTTCCAGAGGTTGTTTCGGGAGGGTCGGGGGTAG | |
| 1: | GluLysThrIleSerLysAlaLysGlyGlnProArgGluProGlnValTyrThrLeuPro | |
| | GAGAAAACCATCTCCAAAGCCAAAGGGCAGCCCCGAGAACCACAGGTGTACACCCTGCCC | |
| 1021 | ---------!---------!---------!---------!---------!---------! | 1080 |
| | CTCTTTTGGTAGAGGTTTCGGTTTCCCGTCGGGGCTCTTGGTGTCCACATGTGGGACGGG | |
| 1: | ProSerArgAspGluLeuThrLysAsnGlnValSerLeuThrCysLeuValLysGlyPhe | |
| | CCATCCCGGGATGAGCTGACCAAGAACCAGGTCAGCCTGACCTGCCTGGTCAAAGGCTTC | |
| 1081 | ---------!---------!---------!---------!---------!---------! | 1140 |
| | GGTAGGGCCCTACTCGACTGGTTCTTGGTCCAGTCGGACTGGACGGACCAGTTTCCGAAG | |
| 1: | TyrProSerAspIleAlaValGluTrpGluSerAsnGlyGlnProGluAsnAsnTyrLys | |
| | TATCCCAGCGACATCGCCGTGGAGTGGGAGAGCAATGGGCAGCCGGAGAACAACTACAAG | |
| 1141 | ---------!---------!---------!---------!---------!---------! | 1200 |
| | ATAGGGTCGCTGTAGCGGCACCTCACCCTCTCGTTACCCGTCGGCCTCTTGTTGATGTTC | |
| 1: | ThrThrProProValLeuAspSerAspGlySerPhePheLeuTyrSerLysLeuThrVal | |
| | ACCACGCCTCCCGTGCTGGACTCCGACGGCTCCTTCTTCCTCTACAGCAAGCTCACCGTG | |
| 1201 | ---------!---------!---------!---------!---------!---------! | 1260 |
| | TGGTGCGGAGGGCACGACCTGAGGCTGCCGAGGAAGAAGGAGATGTCGTTCGAGTGGCAC | |
| 1: | AspLysSerArgTrpGlnGlnGlyAsnValPheSerCysSerValMetHisGluAlaLeu | |
| | GACAAGAGCAGGTGGCAGCAGGGGAACGTCTTCTCATGCTCCGTGATGCATGAGGCTCTG | |
| 1261 | ---------!---------!---------!---------!---------!---------! | 1320 |
| | CTGTTCTCGTCCACCGTCGTCCCCTTGCAGAAGAGTACGAGGCACTACGTACTCCGAGAC | |
| 1: | HisAsnHisTyrThrGlnLysSerLeuSerLeuSerProGlyLys | |
| | CACAACCACTACACGCAGAAGAGCCTCTCCCTGTCTCCGGGTAAA | |
| 1321 | ---------!---------!---------!---------!----- | 1365 |
| | GTGTTGGTGATGTGCGTCTTCTCGGAGAGGGACAGAGGCCCATTT | |
| 6R9B-E9 | | |
| SEQ ID No. 285 | | |
| 1: | GluValGlnLeuValGluSerGlyGlyGlyLeuValGlnProGlyGlySerLeuArgLeu | |
| | GAGGTGCAGCTGGTGGAGTCTGGGGGAGGCTTGGTACAGCCTGGGGGGTCCCTGAGACTC | |
| 1 | ---------!---------!---------!---------!---------!---------! | 60 |
| | CTCCACGTCGACCACCTCAGACCCCCTCCGAACCATGTCGGACCCCCCAGGGACTCTGAG | |
| 1: | SerCysLysAlaSerGlyPheThrPheSerArgGlnAlaLeuThrTrpValArgGlnAla | |
| | TCCTGTAAGGCTTCTGGATTTACTTTTAGTCGTCAGGCTCTCACTTGGGTCCGCCAGGCT | |
| 61 | ---------!---------!---------!---------!---------!---------! | 120 |
| | AGGACATTCCGAAGACCTAAATGAAAATCAGCAGTCCGAGAGTGAACCCAGGCGGTCCGA | |
| 1: | ProGlyLysGlyLeuGluTrpIleGlyValIleAlaSerAsnAlaAspThrTyrTyrAla | |
| | CCAGGGAAGGGGCTGGAGTGGATCGGAGTCATTGCTAGTAATGCTGACACATACTACGCG | |
| 121 | ---------!---------!---------!---------!---------!---------! | 180 |
| | GGTCCCTTCCCCGACCTCACCTAGCCTCAGTAACGATCATTACGACTGTGTATGATGCGC | |
| 1: | SerTrpAlaLysGlyArgPheThrIleSerArgAspAsnSerLysAsnThrLeuTyrLeu | |
| | AGCTGGGCGAAAGGCCGGTTCACCATCTCCAGAGACAATTCCAAGAACACGCTGTATCTG | |
| 181 | ---------!---------!---------!---------!---------!---------! | 240 |
| | TCGACCCGCTTTCCGGCCAAGTGGTAGAGGTCTCTGTTAAGGTTCTTGTGCGACATAGAC | |
| 1: | GlnMetAsnSerLeuArgAlaGluAspThrAlaValTyrTyrCysAlaArgHisProGly | |
| | CAAATGAACAGCCTGAGAGCCGAGGACACGGCCGTATATTACTGTGCCAGACATCCTGGT | |
| 241 | ---------!---------!---------!---------!---------!---------! | 300 |
| | GTTTACTTGTCGGACTCTCGGCTCCTGTGCCGGCATATAATGACACGGTCTGTAGGACCA | |
| 1: | SerAlaGlyTyrGlyTyrGlnTyrTyrTyrAlaMetAspProTrpGlyGlnGlyThrLeu | |
| | AGTGCTGGTTATGGTTATCAGTATTATTACGCCATGGACCCCTGGGGCCAAGGAACCCTG | |
| 301 | ---------!---------!---------!---------!---------!---------! | 360 |
| | TCACGACCAATACCAATAGTCATAATAATGCGGTACCTGGGGACCCCGGTTCCTTGGGAC | |
| 1: | ValThrValSerSerAlaSerThrLysGlyProSerValPheProLeuAlaProSerSer | |
| | GTCACCGTCTCCTCAGCCTCCACCAAGGGCCCATCGGTCTTCCCCCTGGCACCCTCCTCC | |
| 361 | ---------!---------!---------!---------!---------!---------! | 420 |
| | CAGTGGCAGAGGAGTCGGAGGTGGTTCCCGGGTAGCCAGAAGGGGGACCGTGGGAGGAGG | |
| 1: | LysSerThrSerGlyGlyThrAlaAlaLeuGlyCysLeuValLysAspTyrPheProGlu | |
| | AAGAGCACCTCTGGGGGCACAGCGGCCCTGGGCTGCCTGGTCAAGGACTACTTCCCCGAA | |
| 421 | ---------!---------!---------!---------!---------!---------! | 480 |
| | TTCTCGTGGAGACCCCCGTGTCGCCGGGACCCGACGGACCAGTTCCTGATGAAGGGGCTT | |
| 1: | ProValThrValSerTrpAsnSerGlyAlaLeuThrSerGlyValHisThrPheProAla | |
| | CCGGTGACGGTGTCGTGGAACTCAGGCGCCCTGACCAGCGGCGTGCACACCTTCCCGGCT | |
| 481 | ---------!---------!---------!---------!---------!---------! | 540 |
| | GGCCACTGCCACAGCACCTTGAGTCCGCGGGACTGGTCGCCGCACGTGTGGAAGGGCCGA | |
| 1: | ValLeuGlnSerSerGlyLeuTyrSerLeuSerSerValValThrValProSerSerSer | |
| | GTCCTACAGTCCTCAGGACTCTACTCCCTCAGCAGCGTGGTGACCGTGCCCTCCAGCAGC | |
| 541 | ---------!---------!---------!---------!---------!---------! | 600 |
| | CAGGATGTCAGGAGTCCTGAGATGAGGGAGTCGTCGCACCACTGGCACGGGAGGTCGTCG | |
| 1: | LeuGlyThrGlnThrTyrIleCysAsnValAsnHisLysProSerAsnThrLysValAsp | |
| | TTGGGCACCCAGACCTACATCTGCAACGTGAATCACAAGCCCAGCAACACCAAGGTGGAC | |
| 601 | ---------!---------!---------!---------!---------!---------! | 660 |
| | AACCCGTGGGTCTGGATGTAGACGTTGCACTTAGTGTTCGGGTCGTTGTGGTTCCACCTG | |
| 1: | LysLysValGluProLysSerCysAspLysThrHisThrCysProProCysProAlaPro | |
| | AAGAAAGTTGAGCCCAAATCTTGTGACAAAACTCACACATGCCCACCGTGCCCAGCACCT | |
| 661 | ---------!---------!---------!---------!---------!---------! | 720 |
| | TTCTTTCAACTCGGGTTTAGAACACTGTTTTGAGTGTGTACGGGTGGCACGGGTCGTGGA | |
| 1: | GluLeuLeuGlyGlyProSerValPheLeuPheProProLysProLysAspThrLeuMet | |
| | GAACTCCTGGGGGGACCGTCAGTCTTCCTCTTCCCCCCAAAACCCAAGGACACCCTCATG | |
| 721 | ---------!---------!---------!---------!---------!---------! | 780 |
| | CTTGAGGACCCCCCTGGCAGTCAGAAGGAGAAGGGGGGTTTTGGGTTCCTGTGGGAGTAC | |
| 1: | IleSerArgThrProGluValThrCysValValValAspValSerHisGluAspProGlu | |
| | ATCTCCCGGACCCCTGAGGTCACATGCGTGGTGGTGGACGTGAGCCACGAAGACCCTGAG | |
| 781 | ---------!---------!---------!---------!---------!---------! | 840 |
| | TAGAGGGCCTGGGGACTCCAGTGTACGCACCACCACCTGCACTCGGTGCTTCTGGGACTC | |
| 1: | ValLysPheAsnTrpTyrValAspGlyValGluValHisAsnAlaLysThrLysProArg | |
| | GTCAAGTTCAACTGGTACGTGGACGGCGTGGAGGTGCATAATGCCAAGACAAAGCCGCGG | |
| 841 | ---------!---------!---------!---------!---------!---------! | 900 |
| | CAGTTCAAGTTGACCATGCACCTGCCGCACCTCCACGTATTACGGTTCTGTTTCGGCGCC | |
| 1: | GluGluGlnTyrAsnSerThrTyrArgValValSerValLeuThrValLeuHisGlnAsp | |
| | GAGGAGCAGTACAACAGCACGTACCGTGTGGTCAGCGTCCTCACCGTCCTGCACCAGGAC | |
| 901 | ---------!---------!---------!---------!---------!---------! | 960 |
| | CTCCTCGTCATGTTGTCGTGCATGGCACACCAGTCGCAGGAGTGGCAGGACGTGGTCCTG | |
| 1: | TrpLeuAsnGlyLysGluTyrLysCysLysValSerAsnLysAlaLeuProAlaProIle | |
| | TGGCTGAATGGCAAGGAGTACAAGTGCAAGGTCTCCAACAAAGCCCTCCCAGCCCCCATC | |
| 961 | ---------!---------!---------!---------!---------!---------! | 1020 |
| | ACCGACTTACCGTTCCTCATGTTCACGTTCCAGAGGTTGTTTCGGGAGGGTCGGGGGTAG | |
| 1: | GluLysThrIleSerLysAlaLysGlyGlnProArgGluProGlnValTyrThrLeuPro | |
| | GAGAAAACCATCTCCAAAGCCAAAGGGCAGCCCCGAGAACCACAGGTGTACACCCTGCCC | |
| 1021 | ---------!---------!---------!---------!---------!---------! | 1080 |
| | CTCTTTTGGTAGAGGTTTCGGTTTCCCGTCGGGGCTCTTGGTGTCCACATGTGGGACGGG | |
| 1: | ProSerArgAspGluLeuThrLysAsnGlnValSerLeuThrCysLeuValLysGlyPhe | |
| | CCATCCCGGGATGAGCTGACCAAGAACCAGGTCAGCCTGACCTGCCTGGTCAAAGGCTTC | |
| 1081 | ---------!---------!---------!---------!---------!---------! | 1140 |
| | GGTAGGGCCCTACTCGACTGGTTCTTGGTCCAGTCGGACTGGACGGACCAGTTTCCGAAG | |
| 1: | TyrProSerAspIleAlaValGluTrpGluSerAsnGlyGlnProGluAsnAsnTyrLys | |
| | TATCCCAGCGACATCGCCGTGGAGTGGGAGAGCAATGGGCAGCCGGAGAACAACTACAAG | |
| 1141 | ---------!---------!---------!---------!---------!---------! | 1200 |
| | ATAGGGTCGCTGTAGCGGCACCTCACCCTCTCGTTACCCGTCGGCCTCTTGTTGATGTTC | |
| 1: | ThrThrProProValLeuAspSerAspGlySerPhePheLeuTyrSerLysLeuThrVal | |
| | ACCACGCCTCCCGTGCTGGACTCCGACGGCTCCTTCTTCCTCTACAGCAAGCTCACCGTG | |
| 1201 | ---------!---------!---------!---------!---------!---------! | 1260 |
| | TGGTGCGGAGGGCACGACCTGAGGCTGCCGAGGAAGAAGGAGATGTCGTTCGAGTGGCAC | |
| 1: | AspLysSerArgTrpGlnGlnGlyAsnValPheSerCysSerValMetHisGluAlaLeu | |
| | GACAAGAGCAGGTGGCAGCAGGGGAACGTCTTCTCATGCTCCGTGATGCATGAGGCTCTG | |
| 1261 | ---------!---------!---------!---------!---------!---------! | 1320 |
| | CTGTTCTCGTCCACCGTCGTCCCCTTGCAGAAGAGTACGAGGCACTACGTACTCCGAGAC | |
| 1: | HisAsnHisTyrThrGlnLysSerLeuSerLeuSerProGlyLys | |
| | CACAACCACTACACGCAGAAGAGCCTCTCCCTGTCTCCGGGTAAA | |
| 1321 | ---------!---------!---------!---------!----- | 1365 |
| | 1365 | |
| | GTGTTGGTGATGTGCGTCTTCTCGGAGAGGGACAGAGGCCCATTT | |
| 6R9E-A8 | | |
| SEQ ID No. 286 | | |
| 1: | GluValGlnLeuValGluSerGlyGlyGlyLeuValGlnProGlyGlySerLeuArgLeu | |
| | GAGGTGCAGCTGGTGGAGTCTGGGGGAGGCTTGGTACAGCCTGGGGGGTCCCTGAGACTC | |
| 1 | ---------!---------!---------!---------!---------!---------! | 60 |
| | CTCCACGTCGACCACCTCAGACCCCCTCCGAACCATGTCGGACCCCCCAGGGACTCTGAG | |
| 1: | SerCysLysAlaSerGlySerThrIleSerHisGlnAlaValThrTrpValArgGlnAla | |
| | TCCTGTAAGGCTTCTGGAAGTACTATTTCTCATCAGGCTGTGACTTGGGTCCGCCAGGCT | |
| 61 | ---------!---------!---------!---------!---------!---------! | 120 |
| | AGGACATTCCGAAGACCTTCATGATAAAGAGTAGTCCGACACTGAACCCAGGCGGTCCGA | |
| 1: | ProGlyLysGlyLeuGluTrpIleGlyValIleAlaSerAsnAlaAspThrTyrTyrAla | |
| | CCAGGGAAGGGGCTGGAGTGGATCGGAGTCATTGCTAGTAATGCTGACACATACTACGCG | |
| 121 | ---------!---------!---------!---------!---------!---------! | 180 |
| | GGTCCCTTCCCCGACCTCACCTAGCCTCAGTAACGATCATTACGACTGTGTATGATGCGC | |
| 1: | SerTrpAlaLysGlyArgPheThrIleSerArgAspAsnSerLysAsnThrLeuTyrLeu | |
| | AGCTGGGCGAAAGGCCGGTTCACCATCTCCAGAGACAATTCCAAGAACACGCTGTATCTG | |
| 181 | ---------!---------!---------!---------!---------!---------! | 240 |
| | TCGACCCGCTTTCCGGCCAAGTGGTAGAGGTCTCTGTTAAGGTTCTTGTGCGACATAGAC | |
| 1: | GlnMetAsnSerLeuArgAlaGluAspThrAlaValTyrTyrCysAlaArgHisProGly | |
| | CAAATGAACAGCCTGAGAGCCGAGGACACGGCCGTATATTACTGTGCCAGACATCCTGGT | |
| 241 | ---------!---------!---------!---------!---------!---------! | 300 |
| | GTTTACTTGTCGGACTCTCGGCTCCTGTGCCGGCATATAATGACACGGTCTGTAGGACCA | |
| 1: | SerAlaGlyTyrGlyTyrGlnTyrTyrTyrAlaMetAspProTrpGlyGlnGlyThrLeu | |
| | AGTGCTGGTTATGGTTATCAGTATTATTACGCCATGGACCCCTGGGGCCAAGGAACCCTG | |
| 301 | ---------!---------!---------!---------!---------!---------! | 360 |
| | TCACGACCAATACCAATAGTCATAATAATGCGGTACCTGGGGACCCCGGTTCCTTGGGAC | |
| 1: | ValThrValSerSerAlaSerThrLysGlyProSerValPheProLeuAlaProSerSer | |
| | GTCACCGTCTCCTCAGCCTCCACCAAGGGCCCATCGGTCTTCCCCCTGGCACCCTCCTCC | |
| 361 | ---------!---------!---------!---------!---------!---------! | 420 |
| | CAGTGGCAGAGGAGTCGGAGGTGGTTCCCGGGTAGCCAGAAGGGGGACCGTGGGAGGAGG | |
| 1: | LysSerThrSerGlyGlyThrAlaAlaLeuGlyCysLeuValLysAspTyrPheProGlu | |
| | AAGAGCACCTCTGGGGGCACAGCGGCCCTGGGCTGCCTGGTCAAGGACTACTTCCCCGAA | |
| 421 | ---------!---------!---------!---------!---------!---------! | 480 |
| | TTCTCGTGGAGACCCCCGTGTCGCCGGGACCCGACGGACCAGTTCCTGATGAAGGGGCTT | |
| 1: | ProValThrValSerTrpAsnSerGlyAlaLeuThrSerGlyValHisThrPheProAla | |
| | CCGGTGACGGTGTCGTGGAACTCAGGCGCCCTGACCAGCGGCGTGCACACCTTCCCGGCT | |
| 481 | ---------!---------!---------!---------!---------!---------! | 540 |
| | GGCCACTGCCACAGCACCTTGAGTCCGCGGGACTGGTCGCCGCACGTGTGGAAGGGCCGA | |
| 1: | ValLeuGlnSerSerGlyLeuTyrSerLeuSerSerValValThrValProSerSerSer | |
| | GTCCTACAGTCCTCAGGACTCTACTCCCTCAGCAGCGTGGTGACCGTGCCCTCCAGCAGC | |
| 541 | ---------!---------!---------!---------!---------!---------! | 600 |
| | CAGGATGTCAGGAGTCCTGAGATGAGGGAGTCGTCGCACCACTGGCACGGGAGGTCGTCG | |
| 1: | LeuGlyThrGlnThrTyrIleCysAsnValAsnHisLysProSerAsnThrLysValAsp | |
| | TTGGGCACCCAGACCTACATCTGCAACGTGAATCACAAGCCCAGCAACACCAAGGTGGAC | |
| 601 | ---------!---------!---------!---------!---------!---------! | 660 |
| | AACCCGTGGGTCTGGATGTAGACGTTGCACTTAGTGTTCGGGTCGTTGTGGTTCCACCTG | |
| 1: | LysLysValGluProLysSerCysAspLysThrHisThrCysProProCysProAlaPro | |
| | AAGAAAGTTGAGCCCAAATCTTGTGACAAAACTCACACATGCCCACCGTGCCCAGCACCT | |
| 661 | ---------!---------!---------!---------!---------!---------! | 720 |
| | TTCTTTCAACTCGGGTTTAGAACACTGTTTTGAGTGTGTACGGGTGGCACGGGTCGTGGA | |
| 1: | GluLeuLeuGlyGlyProSerValPheLeuPheProProLysProLysAspThrLeuMet | |
| | GAACTCCTGGGGGGACCGTCAGTCTTCCTCTTCCCCCCAAAACCCAAGGACACCCTCATG | |
| 721 | ---------!---------!---------!---------!---------!---------! | 780 |
| | CTTGAGGACCCCCCTGGCAGTCAGAAGGAGAAGGGGGGTTTTGGGTTCCTGTGGGAGTAC | |
| 1: | IleSerArgThrProGluValThrCysValValValAspValSerHisGluAspProGlu | |
| | ATCTCCCGGACCCCTGAGGTCACATGCGTGGTGGTGGACGTGAGCCACGAAGACCCTGAG | |
| 781 | ---------!---------!---------!---------!---------!---------! | 840 |
| | TAGAGGGCCTGGGGACTCCAGTGTACGCACCACCACCTGCACTCGGTGCTTCTGGGACTC | |
| 1: | ValLysPheAsnTrpTyrValAspGlyValGluValHisAsnAlaLysThrLysProArg | |
| | GTCAAGTTCAACTGGTACGTGGACGGCGTGGAGGTGCATAATGCCAAGACAAAGCCGCGG | |
| 841 | ---------!---------!---------!---------!---------!---------! | 900 |
| | CAGTTCAAGTTGACCATGCACCTGCCGCACCTCCACGTATTACGGTTCTGTTTCGGCGCC | |
| 1: | GluGluGlnTyrAsnSerThrTyrArgValValSerValLeuThrValLeuHisGlnAsp | |
| | GAGGAGCAGTACAACAGCACGTACCGTGTGGTCAGCGTCCTCACCGTCCTGCACCAGGAC | |
| 901 | ---------!---------!---------!---------!---------!---------! | 960 |
| | CTCCTCGTCATGTTGTCGTGCATGGCACACCAGTCGCAGGAGTGGCAGGACGTGGTCCTG | |
| 1: | TrpLeuAsnGlyLysGluTyrLysCysLysValSerAsnLysAlaLeuProAlaProIle | |
| | TGGCTGAATGGCAAGGAGTACAAGTGCAAGGTCTCCAACAAAGCCCTCCCAGCCCCCATC | |
| 961 | ---------!---------!---------!---------!---------!---------! | 1020 |
| | ACCGACTTACCGTTCCTCATGTTCACGTTCCAGAGGTTGTTTCGGGAGGGTCGGGGGTAG | |
| 1: | GluLysThrIleSerLysAlaLysGlyGlnProArgGluProGlnValTyrThrLeuPro | |
| | GAGAAAACCATCTCCAAAGCCAAAGGGCAGCCCCGAGAACCACAGGTGTACACCCTGCCC | |
| 1021 | ---------!---------!---------!---------!---------!---------! | 1080 |
| | CTCTTTTGGTAGAGGTTTCGGTTTCCCGTCGGGGCTCTTGGTGTCCACATGTGGGACGGG | |
| 1: | ProSerArgAspGluLeuThrLysAsnGlnValSerLeuThrCysLeuValLysGlyPhe | |
| | CCATCCCGGGATGAGCTGACCAAGAACCAGGTCAGCCTGACCTGCCTGGTCAAAGGCTTC | |
| 1081 | ---------!---------!---------!---------!---------!---------! | 1140 |
| | GGTAGGGCCCTACTCGACTGGTTCTTGGTCCAGTCGGACTGGACGGACCAGTTTCCGAAG | |
| 1: | TyrProSerAspIleAlaValGluTrpGluSerAsnGlyGlnProGluAsnAsnTyrLys | |
| | TATCCCAGCGACATCGCCGTGGAGTGGGAGAGCAATGGGCAGCCGGAGAACAACTACAAG | |
| 1141 | ---------!---------!---------!---------!---------!---------! | 1200 |
| | ATAGGGTCGCTGTAGCGGCACCTCACCCTCTCGTTACCCGTCGGCCTCTTGTTGATGTTC | |
| 1: | ThrThrProProValLeuAspSerAspGlySerPhePheLeuTyrSerLysLeuThrVal | |
| | ACCACGCCTCCCGTGCTGGACTCCGACGGCTCCTTCTTCCTCTACAGCAAGCTCACCGTG | |
| 1201 | ---------!---------!---------!---------!---------!---------! | 1260 |
| | TGGTGCGGAGGGCACGACCTGAGGCTGCCGAGGAAGAAGGAGATGTCGTTCGAGTGGCAC | |
| 1: | AspLysSerArgTrpGlnGlnGlyAsnValPheSerCysSerValMetHisGluAlaLeu | |
| | GACAAGAGCAGGTGGCAGCAGGGGAACGTCTTCTCATGCTCCGTGATGCATGAGGCTCTG | |
| 1261 | ---------!---------!---------!---------!---------!---------! | 1320 |
| | CTGTTCTCGTCCACCGTCGTCCCCTTGCAGAAGAGTACGAGGCACTACGTACTCCGAGAC | |
| 1: | HisAsnHisTyrThrGlnLysSerLeuSerLeuSerProGlyLys | |
| | CACAACCACTACACGCAGAAGAGCCTCTCCCTGTCTCCGGGTAAA | |
| 1321 | ---------!---------!---------!---------!----- | 1365 |
| | GTGTTGGTGATGTGCGTCTTCTCGGAGAGGGACAGAGGCCCATTT | |

### CDRs of Clones Selected on BQ.1.1 and XBB

LCDR1, LCDR2, LCDR3, HCDR1, HCDR2, and HCDR3 of clones selected on BQ.1.1 and XBB disclosed herein, i.e. SEQ ID Nos. 279-286, are shown in FIG. 4 (SEQ ID Nos 287-310)

### Clone S3R4F12 Selected on BQ.1.1. XBB and XBB.1

### Light Chain Sequence of Clone S3R4F12 (SEQ ID No. 311)

| | | |
|---|---|---|
| 1: | AspIleGlnMetThrGlnSerProSerSerLeuSerAlaSerValGlyAspArgValThrIleThrCysLeuAlaSerGluGluIleHis | |
| | GACATCCAGATGACCCAGTCTCCATCCTCCCTGTCTGCATCTGTAGGAGACAGAGTCACCATCACTTGCCTGGCCAGTGAGGAGATTCAT | |
| 1 | ---------!---------!---------!---------!---------!---------!---------!---------!---------! | 60 |
| | CTGTAGGTCTACTGGGTCAGAGGTAGGAGGGACAGACGTAGACATCCTCTGTCTCAGTGGTAGTGAACGGACCGGTCACTCCTCTAAGTA | |
| 1: | TyrAlaIleAsnTrpTyrGlnGlnLysProGlyLysAlaProLysLeuLeuIleTyrGlyGlnSerHisLeuGlnAspGlyValProSer | |
| | TATGCGATTAATTGGTATCAGCAGAAACCAGGGAAAGCCCCTAAGCTCCTGATCTATGGGCAGAGTCATCTTCAGGATGGGGTCCCATCA | |
| 91 | ---------!---------!---------!---------!---------!---------!---------!---------!---------! | 180 |
| | ATACGCTAATTAACCATAGTCGTCTTTGGTCCCTTTCGGGGATTCGAGGACTAGATACCCGTCTCAGTAGAAGTCCTACCCCAGGGTAGT | |
| 1: | ArgPheSerGlySerGlySerGlyThrAspTyrThrLeuThrIleSerSerLeuGlnProGluAspPheAlaThrTyrTyrCysGlnGln | |
| | AGGTTCAGTGGCAGTGGATCTGGGACAGATTACACTCTCACCATCAGCAGTCTGCAACCTGAAGATTTTGCAACTTACTACTGTCAACAA | |
| 181 | ---------!---------!---------!---------!---------!---------!---------!---------!---------! | 270 |
| | TCCAAGTCACCGTCACCTAGACCCTGTCTAATGTGAGAGTGGTAGTCGTCAGACGTTGGACTTCTAAAACGTTGAATGATGACAGTTGTT | |
| 1: | GlyTyrValValProIleLeuPheGlyGlyGlyThrLysValGluIleLysArgThrValAlaAlaProSerValPheIlePheProPro | |
| | GGATATGTTGTGCCTATACTGTTCGGCGGAGGGACCAAGGTGGAGATCAAACGGACTGTGGCTGCACCATCTGTCTTCATCTTCCCGCCA | |
| 271 | ---------!---------!---------!---------!---------!---------!---------!---------!---------! | 360 |
| | CCTATACAACACGGATATGACAAGCCGCCTCCCTGGTTCCACCTCTAGTTTGCCTGACACCGACGTGGTAGACAGAAGTAGAAGGGCGGT | |
| 1: | SerAspGluGlnLeuLysSerGlyThrAlaSerValValCysLeuLeuAsnAsnPheTyrProArgGluAlaLysValGlnTrpLysVal | |
| | TCTGATGAGCAGTTGAAATCTGGAACTGCCTCTGTTGTGTGCCTGCTGAATAACTTCTATCCCAGAGAGGCCAAAGTACAGTGGAAGGTG | |
| 361 | ---------!---------!---------!---------!---------!---------!---------!---------!---------! | 450 |
| | AGACTACTCGTCAACTTTAGACCTTGACGGAGACAACACACGGACGACTTATTGAAGATAGGGTCTCTCCGGTTTCATGTCACCTTCCAC | |
| 1: | AspAsnAlaLeuGlnSerGlyAsnSerGlnGluSerValThrGluGlnAspSerLysAspSerThrTyrSerLeuSerSerThrLeuThr | |
| | GATAACGCCCTCCAATCGGGTAACTCCCAGGAGAGTGTCACAGAGCAGGACAGCAAGGACAGCACCTACAGCCTCAGCAGCACCCTGACG | |
| 451 | ---------!---------!---------!---------!---------!---------!---------!---------!---------! | 540 |
| | CTATTGCGGGAGGTTAGCCCATTGAGGGTCCTCTCACAGTGTCTCGTCCTGTCGTTCCTGTCGTGGATGTCGGAGTCGTCGTGGGACTGC | |
| 1: | LeuSerLysAlaAspTyrGluLysHisLysValTyrAlaCysGluValThrHisGlnGlyLeuSerSerSerValThrLysSerPheAsn | |
| | CTGAGCAAAGCAGACTACGAGAAACACAAAGTCTACGCCTGCGAAGTCACCCATCAGGGCCTGAGCTCGTCCGTCACAAAGAGCTTCAAC | |
| 541 | ---------!---------!---------!---------!---------!---------!---------!---------!---------! | 630 |
| | GACTCGTTTCGTCTGATGCTCTTTGTGTTTCAGATGCGGACGCTTCAGTGGGTAGTCCCGGACTCGAGCAGGCAGTGTTTCTCGAAGTTG | |
| 1: | ArgGlyGluCys | |
| | AGGGGAGAGTGT | |
| 631 | ---------!-- | 642 |
| | TCCCCTCTCACA | |

### Heavy Chain Sequence of Clone S3R4F12 (SEO ID No. 312)

| | | |
|---|---|---|
| 1: | GluValGlnLeuValGluSerGlyGlyGlyLeuValGlnProGlyGlySerLeuArgLeuSerCysGluAlaSerGlyTyrSerIleAla | |
| | GAGGTGCAGCTGGTGGAGTCTGGGGGAGGCTTGGTACAGCCTGGGGGGTCCCTGAGACTCTCCTGTGAGGCTTCTGGATATAGTATTGCT | |
| 1 | ---------!---------!---------!---------!---------!---------!---------!---------!---------! | 90 |
| | CTCCACGTCGACCACCTCAGACCCCCTCCGAACCATGTCGGACCCCCCAGGGACTCTGAGAGGACACTCCGAAGACCTATATCATAACGA | |
| 1: | ProGlnAlaValSerTrpValArgGlnAlaProGlyLysGlyLeuGluTrpIleGlyValIleAlaSerAsnAlaAspThrTyrTyrAla | |
| | CCGCAGGCGGTGAGTTGGGTCCGCCAGGCTCCAGGGAAGGGGCTGGAGTGGATCGGAGTCATTGCTAGTAATGCTGACACATACTACGCG | |
| 91 | ---------!---------!---------!---------!---------!---------!---------!---------!---------! | 180 |
| | GGCGTCCGCCACTCAACCCAGGCGGTCCGAGGTCCCTTCCCCGACCTCACCTAGCCTCAGTAACGATCATTACGACTGTGTATGATGCGC | |
| 1: | SerTrpAlaLysGlyArgPheThrIleSerArgAspAsnSerLysAsnThrLeuTyrLeuGlnMetAsnSerLeuArgAlaGluAspThr | |
| | AGCTGGGCGAAAGGCCGGTTCACCATCTCCAGAGACAATTCCAAGAACACGCTGTATCTGCAAATGAACAGCCTGAGAGCCGAGGACACG | |
| 181 | ---------!---------!---------!---------!---------!---------!---------!---------!---------! | 270 |
| | TCGACCCGCTTTCCGGCCAAGTGGTAGAGGTCTCTGTTAAGGTTCTTGTGCGACATAGACGTTTACTTGTCGGACTCTCGGCTCCTGTGC | |
| 1: | AlaValTyrTyrCysAlaArgHisProGlyPheAlaGlyTyrGlyTyrGlyMetTyrTyrSerMetAspProTrpGlyGlnGlyThrLeu | |
| | GCCGTATATTACTGTGCCAGACATCCGGGTTTTGCTGGTTATGGTTATGGTATGTATTATTCGATGGACCCCTGGGGCCAAGGAACCCTG | |
| 271 | ---------!---------!---------!---------!---------!---------!---------!---------!---------! | 360 |
| | CGGCATATAATGACACGGTCTGTAGGCCCAAAACGACCAATACCAATACCATACATAATAAGCTACCTGGGGACCCCGGTTCCTTGGGAC | |
| 1: | ValThrValSerSerAlaSerThrLysGlyProSerValPheProLeuAlaProSerSerLysSerThrSerGlyGlyThrAlaAlaLeu | |
| | GTCACCGTCTCCTCAGCCTCCACCAAGGGCCCATCGGTCTTCCCCCTGGCACCCTCCTCCAAGAGCACCTCTGGGGGCACAGCGGCCCTG | |
| 361 | ---------!---------!---------!---------!---------!---------!---------!---------!---------! | 450 |
| | CAGTGGCAGAGGAGTCGGAGGTGGTTCCCGGGTAGCCAGAAGGGGGACCGTGGGAGGAGGTTCTCGTGGAGACCCCCGTGTCGCCGGGAC | |
| 1: | GlyCysLeuValLysAspTyrPheProGluProValThrValSerTrpAsnSerGlyAlaLeuThrSerGlyValHisThrPheProAla | |
| | GGCTGCCTGGTCAAGGACTACTTCCCCGAACCGGTGACGGTGTCGTGGAACTCAGGCGCCCTGACCAGCGGCGTGCACACCTTCCCGGCT | |
| 451 | ---------!---------!---------!---------!---------!---------!---------!---------!---------! | 540 |
| | CCGACGGACCAGTTCCTGATGAAGGGGCTTGGCCACTGCCACAGCACCTTGAGTCCGCGGGACTGGTCGCCGCACGTGTGGAAGGGCCGA | |
| 1: | ValLeuGlnSerSerGlyLeuTyrSerLeuSerSerValValThrValProSerSerSerLeuGlyThrGlnThrTyrIleCysAsnVal | |
| | GTCCTACAGTCCTCAGGACTCTACTCCCTCAGCAGCGTGGTGACCGTGCCCTCCAGCAGCTTGGGCACCCAGACCTACATCTGCAACGTG | |
| 541 | ---------!---------!---------!---------!---------!---------!---------!---------!---------! | 630 |
| | CAGGATGTCAGGAGTCCTGAGATGAGGGAGTCGTCGCACCACTGGCACGGGAGGTCGTCGAACCCGTGGGTCTGGATGTAGACGTTGCAC | |
| 1: | AsnHisLysProSerAsnThrLysValAspLysLysValGluProLysSerCysAspLysThrHisThrCysProProCysProAlaPro | |
| | AATCACAAGCCCAGCAACACCAAGGTGGACAAGAAAGTTGAGCCCAAATCTTGTGACAAAACTCACACATGCCCACCGTGCCCAGCACCT | |
| 631 | ---------!---------!---------!---------!---------!---------!---------!---------!---------! | 720 |
| | TTAGTGTTCGGGTCGTTGTGGTTCCACCTGTTCTTTCAACTCGGGTTTAGAACACTGTTTTGAGTGTGTACGGGTGGCACGGGTCGTGGA | |
| 1: | GluLeuLeuGlyGlyProSerValPheLeuPheProProLysProLysAspThrLeuMetIleSerArgThrProGluValThrCysVal | |
| | GAACTCCTGGGGGGACCGTCAGTCTTCCTCTTCCCCCCAAAACCCAAGGACACCCTCATGATCTCCCGGACCCCTGAGGTCACATGCGTG | |
| 721 | ---------!---------!---------!---------!---------!---------!---------!---------!---------! | 810 |
| | CTTGAGGACCCCCCTGGCAGTCAGAAGGAGAAGGGGGGTTTTGGGTTCCTGTGGGAGTACTAGAGGGCCTGGGGACTCCAGTGTACGCAC | |
| 1: | ValValAspValSerHisGluAspProGluValLysPheAsnTrpTyrValAspGlyValGluValHisAsnAlaLysThrLysProArg | |
| | GTGGTGGACGTGAGCCACGAAGACCCTGAGGTCAAGTTCAACTGGTACGTGGACGGCGTGGAGGTGCATAATGCCAAGACAAAGCCGCGG | |
| 811 | ---------!---------!---------!---------!---------!---------!---------!---------!---------! | 900 |
| | CACCACCTGCACTCGGTGCTTCTGGGACTCCAGTTCAAGTTGACCATGCACCTGCCGCACCTCCACGTATTACGGTTCTGTTTCGGCGCC | |
| 1: | GluGluGlnTyrAsnSerThrTyrArgValValSerValLeuThrValLeuHisGlnAspTrpLeuAsnGlyLysGluTyrLysCysLys | |
| | GAGGAGCAGTACAACAGCACGTACCGTGTGGTCAGCGTCCTCACCGTCCTGCACCAGGACTGGCTGAATGGCAAGGAGTACAAGTGCAAG | |
| 901 | ---------!---------!---------!---------!---------!---------!---------!---------!---------! | 990 |
| | CTCCTCGTCATGTTGTCGTGCATGGCACACCAGTCGCAGGAGTGGCAGGACGTGGTCCTGACCGACTTACCGTTCCTCATGTTCACGTTC | |
| 1: | ValSerAsnLysAlaLeuProAlaProIleGluLysThrIleSerLysAlaLysGlyGlnProArgGluProGlnValTyrThrLeuPro | |
| | GTCTCCAACAAAGCCCTCCCAGCCCCCATCGAGAAAACCATCTCCAAAGCCAAAGGGCAGCCCCGAGAACCACAGGTGTACACCCTGCCC | |
| 991 | ---------!---------!---------!---------!---------!---------!---------!---------!---------! | 1080 |
| | CAGAGGTTGTTTCGGGAGGGTCGGGGGTAGCTCTTTTGGTAGAGGTTTCGGTTTCCCGTCGGGGCTCTTGGTGTCCACATGTGGGACGGG | |
| 1: | ProSerArgAspGluLeuThrLysAsnGlnValSerLeuThrCysLeuValLysGlyPheTyrProSerAspIleAlaValGluTrpGlu | |
| | CCATCCCGGGATGAGCTGACCAAGAACCAGGTCAGCCTGACCTGCCTGGTCAAAGGCTTCTATCCCAGCGACATCGCCGTGGAGTGGGAG | |
| 1081 | ---------!---------!---------!---------!---------!---------!---------!---------!---------! | 1170 |
| | GGTAGGGCCCTACTCGACTGGTTCTTGGTCCAGTCGGACTGGACGGACCAGTTTCCGAAGATAGGGTCGCTGTAGCGGCACCTCACCCTC | |
| 1: | SerAsnGlyGlnProGluAsnAsnTyrLysThrThrProProValLeuAspSerAspGlySerPhePheLeuTyrSerLysLeuThrVal | |
| | AGCAATGGGCAGCCGGAGAACAACTACAAGACCACGCCTCCCGTGCTGGACTCCGACGGCTCCTTCTTCCTCTACAGCAAGCTCACCGTG | |
| 1171 | ---------!---------!---------!---------!---------!---------!---------!---------!---------! | 1260 |
| | TCGTTACCCGTCGGCCTCTTGTTGATGTTCTGGTGCGGAGGGCACGACCTGAGGCTGCCGAGGAAGAAGGAGATGTCGTTCGAGTGGCAC | |
| 1: | AspLysSerArgTrpGlnGlnGlyAsnValPheSerCysSerValMetHisGluAlaLeuHisAsnHisTyrThrGlnLysSerLeuSer | |
| | GACAAGAGCAGGTGGCAGCAGGGGAACGTCTTCTCATGCTCCGTGATGCATGAGGCTCTGCACAACCACTACACGCAGAAGAGCCTCTCC | |
| 1261 | ---------!---------!---------!---------!---------!---------!---------!---------!---------! | 1350 |
| | CTGTTCTCGTCCACCGTCGTCCCCTTGCAGAAGAGTACGAGGCACTACGTACTCCGAGACGTGTTGGTGATGTGCGTCTTCTCGGAGAGG | |
| 1: | LeuSerProGlyLys | |
| | CTGTCTCCGGGTAAA | |
| 1351 | ---------!----- | 1365 |
| | GACAGAGGCCCATTT | |

### CDRs of Clone S3R4F12

LCDR1, LCDR2, LCDR3, HCDR1, HCDR2, and HCDR3 of clone S3R4F12 selected on BQ.1.1 and XBB disclosed herein are shown in Table 5 below (SEQ ID Nos 313-318).

TABLE 5

| | LCDR1 | LCDR2 | LCDR3 | HCDR1 | HCDR2 | HCDR3 |
|---|---|---|---|---|---|---|
| S3R4F12 | LASEEIHYAIN | YGQSHLQD | QQGYVVPIL | EASGYSIAPQAVS | IGVIASNADTYYASWAKG | ARHPGFAGYGYGMYYSMDP |
| SEQ ID No | 313 | 314 | 315 | 316 | 317 | 318 |

### CDRs of Clone Against Recent Omicron Variants

CDR sequences of clones showing neutralization of recent SARS-CoV-2 Omicron subvariants, such as HK.3 and JN.1, are listed in Table 6.

TABLE 6

| Clone ID | LCDR1 | LCDR2 | LCDR3 | HCDR1 | HCDR2 | HCDR3 |
|---|---|---|---|---|---|---|
| HxB10 | QASEEVHYGIN | YGANYRVD | QQGYNVPTD | TASGYSI SHQAVT | VIYADGASDYAS | ARHPGDSGYGYHYYYSMDP |
| | (SEQ ID 319) | (SEQ ID 320) | (SEQ ID 321) | (SEQ ID 322) | (SEQ ID 323) | (SEQ ID 324) |
| AR6-B9 | QASENIRYAVN | FGANYRWH | QQGYNVPTD | EASGSDLSHQALT | VIYADGQGDYAS | ARHPGDSGYGYHYYY SMDP |
| | (SEQ ID 325) | (SEQ ID 326) | (SEQ ID 327) | (SEQ ID 328) | (SEQ ID 329) | (SEQ ID 330) |
| BR6-C10 | QASEEVHYGIN | YGANYRVD | QQGYLVPGG | TASGYSI SHQAVT | VIYADGASDYAS | ARHPGDSGYGYHYYYSMDP |
| | (SEQ ID 331) | (SEQ ID 332) | (SEQ ID 333) | (SEQ ID 334) | (SEQ ID 335) | (SEQ ID 336) |
| CR6-C8 | QASEDIHYALN | YGMNHLMD | QQGYVVPII | AASGYTI SHQAVT | VIASNADTYYAS | ARHPGFAGYGYGMYYSMDP |
| | (SEQ ID 337) | (SEQ ID 338) | (SEQ ID 339) | (SEQ ID 340) | (SEQ ID 341) | (SEQ ID 342) |
| GR4-B12 | QASEEVHYGIN | YGANYRWH | QQGYNVPGH | TASGYSI SHQAVT | VIYSDGRSDYAS | ARHPGDSGYGYHYYYSMDP |
| | (SEQ ID 343) | (SEQ ID 344) | (SEQ ID 345) | (SEQ ID 346) | (SEQ ID 347) | (SEQ ID 348) |
| GR4-E11 | QASEEVHYGIN | YGANYRWH | QQGYNVPGH | KASGYSI SHQAVT | IIYHDGRSDYAS | ARHPGDSGYGYHYYYSMDP |
| | (SEQ ID 349) | (SEQ ID 350) | (SEQ ID 351) | (SEQ ID 352) | (SEQ ID 353) | (SEQ ID 354) |

1. An antibody or a fragment thereof that inhibits the binding of ACE2 protein with spike trimer protein of SARS-CoV-2 (Wuhan wild type) and spike trimer proteins of SARS-CoV-2 variants B.1.1.529, BA.2, BA.5, BA.4.6, BA.2.75.2, BA.2.3.20, BQ.1.1, BN.1, XBB, and XBB.1.

2. The antibody or fragment of claim 1, wherein the antibody or the fragment thereof inhibits binding of ACE2-Fc and spike trimer proteins of SARS-CoV-2 variants BQ.1.1, XBB, and XBB.1, BA.2.3.20, and combinations thereof.

3. The antibody or fragment of claim 1, wherein the antibody or the fragment thereof inhibits binding of ACE2-Fc and spike trimer proteins of SARS-CoV-2 variants BQ.1.1, XBB, and XBB.1, and combinations thereof.

4. The antibody or fragment of claim 1, wherein the antibody has an IC50 that is less than ½ of IC50 of Bebtelovimab.

5. The antibody or fragment of claim 1, wherein the antibody has an IC50 that is less than ⅓ of IC50 of Bebtelovimab.

6. The antibody or fragment of claim 1, wherein the antibody has an IC50 that is less than ⅕ of IC50 of Bebtelovimab.

7. The antibody or a fragment of claim 1, wherein the antibody or the fragment thereof binds to or interacts with a SARS-CoV-2 variant XBB at a binding or interacting site as indicated in FIG. 9A, 9B, 9C, or 9D.

8. The antibody or fragment of claim 1, wherein the antibody has an LCDR1 selected from SEQ ID NOs: 75-108, 287-290, and 313.

9. The antibody or fragment of claim 8, wherein the antibody has an LCDR2 selected from SEQ ID NOs: 109-142, 291-294, and 314.

10. The antibody or fragment of claim 9, wherein the antibody has an LCDR3 selected from SEQ ID NOs: 143-176, 295-298, and 315.

11. The antibody or fragment of claim 1, wherein the antibody has an HCDR1 selected from SEQ ID NOs: 177-210, 299-302, and 316.

12. The antibody or fragment of claim 11, wherein the antibody has an HCDR2 selected from SEQ ID NOs: 211-244, 303-306, and 317.

13. The antibody or fragment of claim 12, wherein the antibody has an HCDR3 selected from SEQ ID NOs: 245-278, 307-310, and 318.

14. The antibody or fragment of claim 1, wherein the antibody has a light chain sequence from SEQ ID NOs: 7-40, 279-282, and 311.

15. The antibody or fragment of claim 1, wherein the antibody has a heavy chain sequence from SEQ ID NOs: 41-74, 283-286, and 312.

16. The antibody or fragment of claim 1, wherein the antibody has a light chain sequence from SEQ ID NOs: 7-40, 279-282, and 311, and has a heavy chain sequence from SEQ ID NOs: 41-74, 283-286, and 312.

17. The antibody or fragment of claim 1, wherein the antibody or fragment is selected from the group consisting of 3R4-H4, 5R4-H1, 5R4-F3, 5R4-E11, 6R4-H1, 6R4-A7, 6R4-G10, 6R4-G11, 6R4-E12, 6R4-E3, 6R4-H5, 6R4-H11, 6R6-A7, 6R6-E1, 6R6-E2, 6R6-E3, 6R6-E4, 6R6-F3, 6R6-F7, 6R6-F9, 6R6-F11, 6R6-F12, 3R6-D11Q, 6R6-E6Q, 6R8A-E11, 6R8A-F2, 6R8A-E1, 6R8A-A2, 6R8C-G3, 6R8C-G4, 6R8C-G8, 6R8C-D9, 6R8C-D10, 6R8C-F10, 6R9A-A2, 6R9B-B11, 6R9B-E9, 6R9E-A8, and S3R4F12.

18. An antibody or a fragment thereof that inhibits binding of ACE2 protein with spike trimer protein of a SARS-CoV-2 variant selected from the group consisting of XBB.1.31, XBB.1.16.1, EG.5, HV.1, FL.1.5.1, HK.3, BA.2.86, BA.2.86.1, JN.1, and combinations thereof.

19. The antibody or fragment of claim 18, wherein the antibody has an LCDR1 selected from SEQ ID NOs: 319, 325, 331, 337, 343, or 349.

20. The antibody or fragment of claim 19, wherein the antibody has an LCDR2 selected from SEQ ID NOs: 320, 326, 332, 338, 344, or 350.

21-30. (canceled)
